(19) United States
(12) Patent Application Publication
(10) Pub. No.: US 2022/0340972 A1
(43) Pub. Date: Oct. 27, 2022

(54) RNA SEQUENCING TO DIAGNOSE SEPSIS AND OTHER DISEASES AND CONDITIONS

(71) Applicant: Rhode Island Hospital, Providence, RI (US)

(72) Inventors: Sean F. MONAGHAN, East Greenwich, RI (US); Gerard J. NAU, Sharon, MA (US); Alger M. FREDERICKS, East Providence, RI (US)

(21) Appl. No.: 17/723,271

(22) Filed: Apr. 18, 2022

Related U.S. Application Data
(60) Provisional application No. 63/176,531, filed on Apr. 19, 2021.
(60) Provisional application No. 63/184,583, filed on May 5, 2021.

(51) Int. Cl. C12Q 1/6883; C12Q 1/689; C12Q 1/70

(52) U.S. Cl. CPC C12Q 1/6883; C12Q 1/689; C12Q 1/701

(57) ABSTRACT

Deep RNA sequencing is a technology that provides an initial diagnostic for sepsis that can also monitor the indicia of treatment and recovery (bacterial counts reduce, physiology returns to steady-state). The invention can be used for many other hospital conditions, particularly those needing an intensive care unit stay with the attendant risk of bacterial infection, such as trauma, stroke, myocardial infarction, or major surgery.

### REFERENCE TO RELATED APPLICATIONS

This patent matter claims priority under 35 U.S.C. § 119(e), to U.S. Ser. No. 63/176,531, filed Apr. 19, 2021, and 63/184,583, filed May 5, 2021, the contents of both of which are incorporated by reference.

### STATEMENT REGARDING FEDERALLY SPONSORED RESEARCH OR DEVELOPMENT

This invention was made with government support under P20 GM103652, T32 HL134625, R35 GM142638, P20 GM121344 awarded by National Institutes of Health. The government has certain rights in the invention.

### FIELD OF THE INVENTION

This invention generally relates to chemical analysis of biological material, using nucleic acid products used in the analysis of nucleic acids, e.g., primers or probes for diseases caused by alterations of genetic material.

### BACKGROUND OF THE INVENTION

Sepsis is a life-threatening organ dysfunction due to a dysregulated host response to infection. Despite declining age-standardized incidence and mortality, sepsis remains a significant cause of health loss worldwide. Rudd et al., The Lancet, 395(10219), 200-211 (Jan. 18, 2020). Sepsis is treatable, and timely implementation of targeted interventions improves outcomes.

Sepsis is diagnosed clinically by the presence of acute infection and new organ dysfunction. Singer et al., JAMA, 315, 801-810 (February 2016). Unlike the previous concepts of septicemia or blood poisoning, the definition of sepsis extends across bacterial, fungal, viral, and parasitic pathogens. The definition focuses on the host response as the major source of morbidity and mortality. Bone et al., Chest, 101, 1644-1655 (1992). Globally, there were about 48.9 million cases of sepsis in 2017, with about 11.0 million total sepsis-related deaths worldwide, representing 19.7% (18·2-21·4). This number may be a substantial undercount. Rudd et al., The Lancet, 395(10219), 200-211 (Jan. 18, 2020). Sepsis results from an underlying infection, so sepsis is an intermediate cause of health loss. Because, according to the principles of the International Classification of Diseases (ICD), causes of death are assigned based on the underlying disorder that triggers the chain of events leading to death rather than intermediate causes, sepsis, when reported as the cause of death, are considered miscoded.

Thus, the global burden of sepsis is more significant than previously appreciated. There is substantial variation in sepsis incidence and mortality according to Healthcare Access and Quality Index (HAQ Index), Lancet, 390, 231-266 (2017)), with the highest burden in places that cannot prevent, identify, or treat sepsis. Further research is needed to understand these disparities and developing policies and practices targeting their amelioration. More robust infection-prevention measures should be assessed and implemented in areas with the highest incidence of sepsis and among populations on which sepsis has the most significant impact. The impact of sepsis is especially severe among children, so more than half of all sepsis cases worldwide in 2017 occurred among children, many neonates.

Physicians diagnose sepsis using clinical judgment under one or more clinical scores. The systemic inflammatory response syndrome (SIRS) approach assesses an inflammatory state affecting the whole body, which is the body's response to an infectious or non-infectious challenge. Jui et al. (American College of Emergency Physicians), Ch. 146: Septic Shock. in Tintinalli's Emergency Medicine: A Comprehensive Study Guide, 7th edition, (New York: McGraw-Hill, 2011). pp. 1003-14. Sepsis has both pro-inflammatory and anti-inflammatory components. The qSOFA approach simplifies the SOFA score by including only its three clinical criteria and by including any altered mentation. Singer et al., JAMA, 315, 801-810 (February 2016). qSOFA can easily and quickly be repeated serially on patients.

A culture of the bacterial infection confirms a diagnosis of sepsis. A culture diagnosis can be delayed by forty-eight hours and sometimes cannot be performed successfully. Clinical judgment sometimes misses sepsis.

Biomarkers are being developed for sepsis, but no reliable biomarkers exist. A 2013 review concluded moderate-quality evidence exists to support the use of the procalcitonin level as a method to distinguish sepsis from non-infectious causes of SIRS. Still, the level alone could not definitively make the diagnosis. Wacker et al., The Lancet Infectious Diseases. 13(5), 426-35 (May 2013). A 2012 systematic review found that soluble urokinase-type plasminogen activator receptor (SuPAR) is a nonspecific marker of inflammation and does not accurately diagnose sepsis. Backes et al. Intensive Care Medicine, 38(9): 1418-28 (September 2012).

There remains a need in the medical art for a better diagnosis of sepsis.

### SUMMARY OF THE INVENTION

The concept of diagnostics is analogous to using a fishing lure to find a single protein, gene, or RNA sequence. The invention provides an improved concept, using a fishing net to obtain all the RNA data in a sample, and use computational biology to better sort through all the data (fish) to identify patients with sepsis and the bacteria causing the immune response. The invention provides an initial diagnostic for sepsis that can also monitor the indicia of treatment and recovery (bacterial counts reduce, physiology returns to steady-state). The invention can be used for many other hospital conditions, particularly those needing an intensive care unit stay with the attendant risk of bacterial infection, such as trauma, stroke, myocardial infarction, or major surgery.

In the first embodiment, the invention provides unmapped bacterial RNA reads to identify bacteria that cause sepsis. In the second embodiment, the invention provides unmapped viral reads to identify sepsis or viral reactivation. In the third embodiment, the invention provides the use of unmapped B/T V(D)J to identify sepsis. In the fourth embodiment, the invention provides Principal Component Analysis of RNA splicing entropy to identify sepsis. In the fifth embodiment, the invention provides RNA lariats to identify sepsis. In the sixth embodiment, the invention provides a Principal Component Analysis of gene expression, alternative RNA splicing, or alternative transcription start and end to identify sepsis.

In producing the listed embodiments, one of ordinary skill in the molecular biological art uses one or more of these steps.

The first step is for one of ordinary skill in the molecular biological art to obtain RNA sequencing from a body sample. In the seventh embodiment, the body sample is a bodily fluid sample. In the eighth embodiment, the bodily fluid sample is blood. In the ninth embodiment, the target is 100,000,000 reads/sample.

The second step is for one to align the RNA sequencing data (reads) to the genome of interest. In the tenth embodiment, the reads from a human sample are aligned to a human genome. In the eleventh embodiment, the reads from a mouse sample are aligned to a mouse genome.

The third step is to select the un-mapped reads and analyze the reads using a Read Origin Protocol (ROP).

In the first embodiment, the next step is to identify bacteria present in the sample. From the ROP, one of ordinary skill in the molecular biological art identifies bacteria present in the sample. In the twelfth embodiment, one of ordinary skill in the molecular biological art or medical art uses the identified bacteria to list potential causative organisms of sepsis (product).

In the second embodiment, from the ROP, the next step is to identify the viruses present in the sample. In the thirteenth embodiment, one uses the virus identified with PCA to identify likely sepsis samples.

In the third embodiment, from the ROP, the next step is to identify the T/B cell epitopes present in the samples. In the fourteenth embodiment, one uses the T/B cell epitopes identified with PCA to identify likely sepsis samples.

Alternatively, or in combination, in the third step, one selects the mapped reads and then uses a program that enables detection and quantification of alternative RNA splicing events to identity gene expression, RNA splicing events, alternative transcription start/end, or RNA splicing entropy. In a fifteenth embodiment, the program that enables detection and quantification of alternative RNA splicing events is Whippet. In the sixteenth embodiment, one uses the gene expression changes, RNA splicing events, and alternative transcription start/end with PCA to identify likely sepsis samples. In the seventeenth embodiment, one uses the RNA splicing entropy identified with PCA to identify likely sepsis samples.

In the fifth embodiment, from the gene expression, RNA splicing events, alternative transcription start/end, or RNA splicing entropy, the next step is for one to identify RNA lariats from the mapped reads. In the eighteenth embodiment, one uses the RNA lariats with PCA to identify likely sepsis samples.

In the nineteenth embodiment, the invention provides an output product with five plots comprising bacterial RNA reads, viral reads, B/T V(D)J epitopes, RNA splicing entropy, and RNA lariat embodiments described above and a list of likely bacteria causing the infection.

RNA sequencing data be used in several ways. (1) Identification of biomarkers. Rather than need to pick a subset to test for, RNA sequencing data can identify genes with increased expression that would correlate to biomarkers of interest. (2) Identification of new biomarkers. RNA sequencing data allows for analysis of processes such as RNA splicing. The method of RNA splicing entropy can be quantified and grouped according to a Principal Component Analysis into sick or not sick. RNA lariats can also be identified in sequencing data and used as a potential biomarker. All biomarkers can be followed over time to assess for resolution of the sepsis. (3) Use of un-mapped reads in sepsis. RNA sequencing typically aligns with the genome of reference (i.e., the human genome). Reads that are not aligned to the human genome are discarded (the percentage of un-mapped reads could itself be a biomarker). These un-mapped reads could be of two major potential interests. (4) Identification of the microbe causing the infection. The unmapped reads can be referenced to the genome of disease-causing microbes (bacteria, viruses, fungi, etc.) to identify the causative organism and start treatment earlier. Serial measurements can also assess the effectiveness of treatment.

The results presented show that mice exposed to trauma separated from controls using PCA. Similarly, mice that did not survive fourteen days post exposure clustered closely together on PCA. These results show a substantial difference in global pre-mRNA processing entropy in mice exposed to trauma vs. controls, and that pre-mRNA processing entropy is useful in predicting mortality.

### BRIEF DESCRIPTION OF THE DRAWINGS

FIG. 1 is a chart showing Principal Component Analysis of samples in the blood. Three mice exposed to the trauma model were compared to three mice in the control group (total n=6). When plotting the first two principal components against each other, the exposed mice separated from the control mice. Samples clustered based on tissue type and ARDS status on the Principal Component Analysis plot, suggesting that splicing entropy can be a biomarker for ARDS status. The first two principal components plotted against each other. The percentages in parentheses represent the percent variability explained by the principal component. Circles represent control mice; squares represent mice exposed to hemorrhage followed by cecal ligation and puncture.

FIG. 2 is a chart showing a Principal Component Analysis of the survival study. Ten mice exposed to trauma were part of the survival experiment. A mortality rate of 30% was observed, which is consistent with previous studies using this model. When plotting the first two principal components against each other, the mice who did not survive closely clustered together. The first two principal components are plotted against each other. The percentages represent the percent variability explained by the principal component. The squares represent mice that died by fourteen days post CLP, circles represent mice that survived.

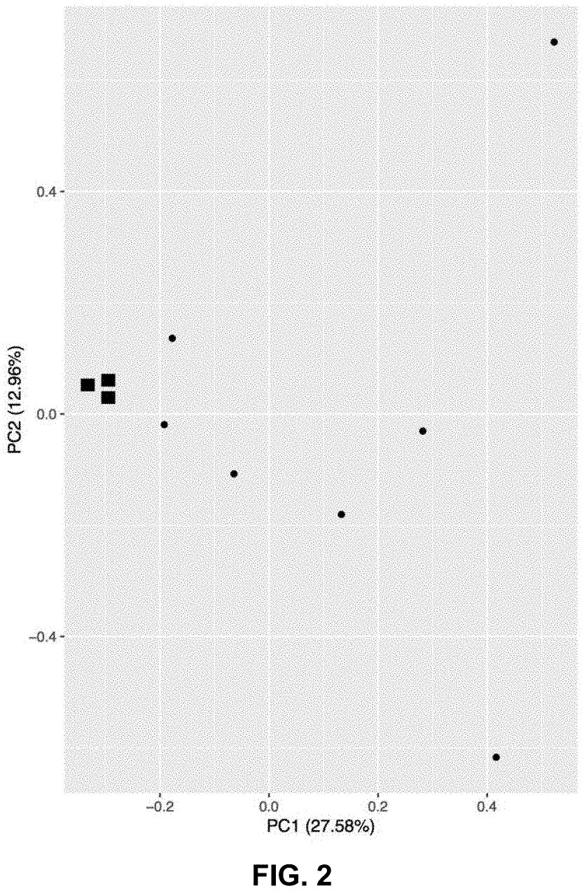

FIG. 3 confirms by real time PCR that the peak SARS-CoV-2 RNA described in this specification is higher than the rest of the N-gene in the blood of critically ill COVID-19 patients.

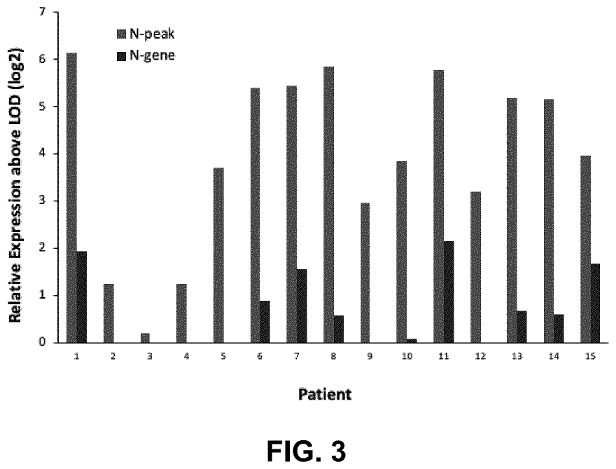

FIG. 4 is a pair of figures showing the SARS-CoV-2 reads per patient. The top panel is a bar graph showing the number of reads aligning to the SARS-CoV-2 genome from each patient. Most reads aligned to loci encoding the N protein (red bar) or the RNA dependent RNA polymerase (black bar). Bottom panel is the location where the cumulative reads from all the patients align to the SARS-CoV-2 genome. Genes encoding the RNA dependent RNA polymerase and the N protein are at positions ˜15,000 and ˜29,000, respectively.

FIG. 5 is a graph created by the principal component analysis of the >380,000 entropy values related to alternative RNA splicing and alternative transcription start/end. Patients labeled in red died from COVID-19 and surviving patients are labeled with green dots. Mortality rate above PC2=0 is 75% and below is 14% (p=0.04)

FIG. 6 (TABLE 1) is a list of the clinical and demographic data for fifteen study participants.

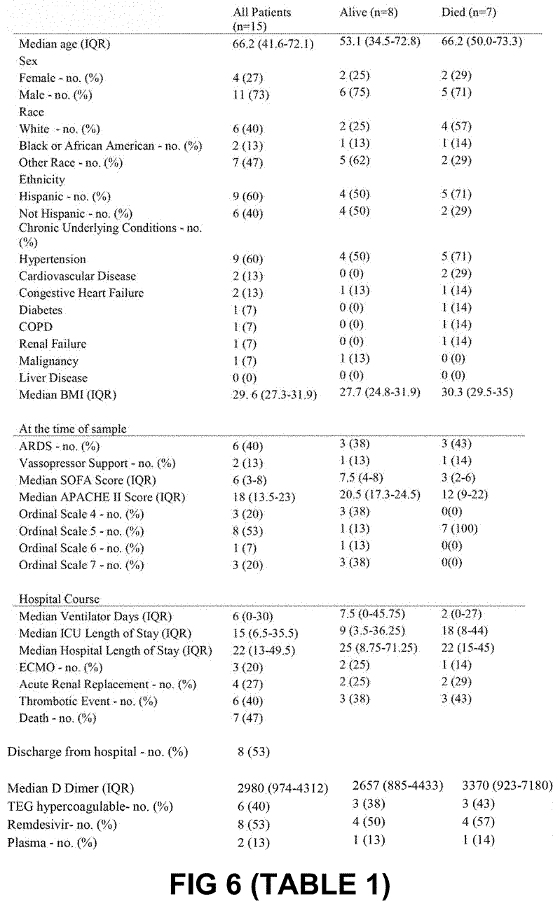

FIG. 7 (TABLE 2) is a list of the counts per patient from Kraken2 assays performed for the fifteen study participants.

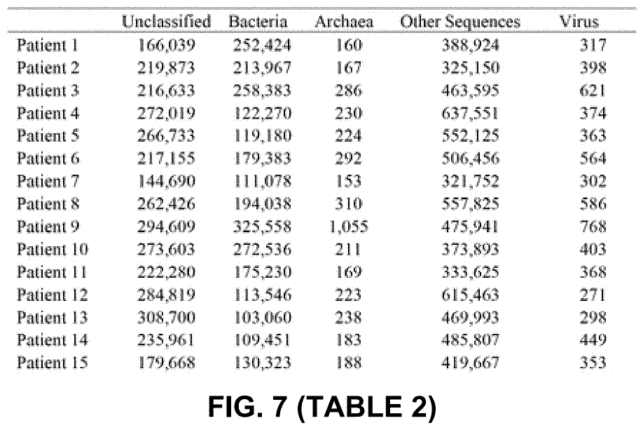

FIG. 8 (TABLE 3) is a list of the gene difference between patients that died versus patients that lived among the fifteen study participants.

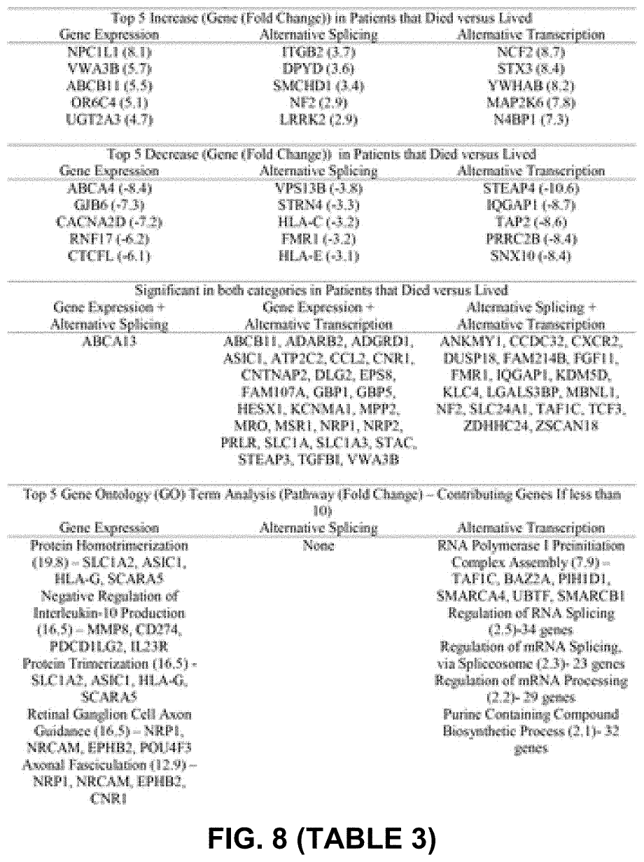

### DETAILED DESCRIPTION OF THE INVENTION

### Industrial Applicability

Despite causing death in one out of five people in the world, there is not a single standard test to diagnose sepsis. Despite declining age-standardized incidence and mortality, sepsis remains a significant cause of health loss worldwide. Rudd et al., The Lancet, 395(10219), 200-211 (Jan. 18, 2020). Sepsis patients undergo the physiology common to patients in the intensive care unit: hypotension, tachycardia, hyperthermia, and hypoxia.

Delays in treatment for sepsis impact mortality. Early identification of the differences between clinically similar patients would allow for earlier interventions (surgery, antibiotics). Using RNA sequencing technology combined with computation biology techniques to understand RNA biology the differences in these two patients could be identified. Earlier prediction of complications would also allow for triage of patients to facilities equipped to deal with them and allow for better discussions regarding expected mortality and morbidity.

It takes days to get a final diagnosis for bacterial pathogen, since culturing of the bacteria is needed. Confirming bacteremia is done microbial blood culture, but the turnaround time can lead to a delay in diagnosis. Biron et al., Biomarker Insights. 10(Suppl 4), 7-17 (Sep. 15, 2015). Procalcitonin (PCT) has been shown to correlate more closely to onset and treatment of sepsis than C-reactive protein (CRP). Vijayan et al., J. Intensive Care (Aug. 3, 2017). Much work has been done with PCT as a predictor of sepsis before symptom onset. Dolin et al., Shock, 49(4), 364-70 (April 2018). PCT has low specificity for sepsis, and is elevated in cancers, autoimmune diseases, and other physiological stressors. Bloos & Reinhart, Virulence, 5(1), 154-60 (Jan. 1, 2014).

RNA sequencing data can identify the bacteria more quickly than culture. The drop in the cost of sequencing has refocused genetic analyses from DNA to RNA sequencing. Methods to analyze this data have improved. Stark et al., Nature Reviews Genetics (2019). Compared to DNA. RNA undergoes dynamic changes by transcription and post-transcriptional processing, providing unique insight into cellular activity. RNA reflects a broader source of infectious etiologies, given that both DNA and RNA viruses have RNA genetic material, whether in the genome or by transcription of mRNA. Patients with trauma who die or have complications are expected to have different changes in expression, alternative RNA splicing, and alternative transcription start/end compared to patients who survive and do not have a complication. The differences seen in RNA biology may correlate with injury severity or predict outcomes. This invention should help direct care in trauma patients when RNA sequencing speeds increase to allow for results that are available when needed for patients in the ICU (within one hour).

RNA sequencing data related to other processes (RNA splicing entropy, gene expression, viral counts, lariat counts, etc.) provide a signature that can identify patients with sepsis. A better understanding of RNA biology in the clinical scenario of critically ill sepsis patients can have a broad impact on biomedical science. When the information in RNA sequencing data can identify patients who have not resolved the immune response to the initial sepsis, outcomes can improve.

The number of unmapped reads aligning to viral pathogenic genomes can be a biomarker of critical illness. Patients with late death should have different gene expression, alternative RNA splicing (including RNA splicing entropy), and alternative transcription start/end as compared to patients with an early death. the genes with increased alternative RNA splicing (including RNA splicing entropy), and alternative transcription start/end are expected to be different in the patients who died late compared to those who died early. These identified genes provide insight into proteins not considered in trauma patients as potential biomarkers or targets of therapeutic intervention but point to pathological mechanism not appreciated or unclear.

RNA biology before the trauma should be able to predict survivors. Mice that survive to fourteen days should have less RNA biology changes compared to mice at the early time point. This are done across three distinct background mice to account for the heterogeneity of humans and the comparability of the two most common immunological/genetic mouse model strains used. As it relates to comparing samples across mouse strains, since gene expression. RNA splicing, and alternative transcription start/end are all basic molecular functions, the results remain similar across the multiple strains.

Identification of B and T cell epitopes from the unmapped reads could be a biomarker for sepsis. Critical illness decreases the diversity of these epitopes. A resolution could signal an improvement in clinical status. Losing some epitopes could indicate immune suppression seen in critical illness.

Alternative transcription starts and end is another biological process potentially influenced by sepsis. Current technology now allows us to identify changes in transcription with RNA sequencing data. Hardwick et al., Frontiers in Genetics. 10, 709 (2019); Cass & Xiao X, Cell Systems, 9(4), 23, 393-400.e6 (October 2019). The genes that have increased difference in alternative transcription start/end could be disease treatment targets. A change to the start or end of the RNA is likely to change the ultimate endpoint of that transcript. Understanding the changes in transcription start and end would better describe the ultimate result of proteins since that were thought to be transcribed and translated could have been transcribed (with changes in the start or end) which led to nonsense mediated decay or the translation of an alternative isoform.

Genes with significant alternative splicing and high entropy in the mouse after trauma may be target for intervention. This invention can better diagnose sepsis and the microbe causing the disease. Emergency room and critical care physicians can use the invention.

### Solution: RNAs as Biomarkers of Critical Illness

While proteins have traditionally been used to reflect inflammatory load, RNAs are more specific to certain etiologies and clinical outcomes.

High through-put sequencing technologies allows for coding and non-coding RNAs (ncRNA) as markers of disease risk and progression. Next-generation sequencing (NGS) quantifies RNAs by sequencing of complementary DNA (cDNA), allowing transcriptomic analysis of mRNAs, ribosomal RNAs (rRNA), and ncRNAs. Kukurba & Montgomery, Cold Spring Harb. Protoc., 2015(11), 951-69 (Apr. 13, 2015).

Coding and non-coding RNAs have been studied as biomarkers. Less attention has been on the portion of data produced (9-20%) via RNA-sequencing that is consistently discarded when it cannot be mapped to a reference genome. Mangul et al., ROP: Dumpster diving in RNA-sequencing to find the source of 1 trillion reads across diverse adult human tissues. Genome Biol., 19 (Feb. 15, 2018).

The discovery of serum-stable circulating miRNAs allows the use of cell-free miRNAs as biomarkers of disease. Benz et al., Int. J. Mol. Sci., 17(1) (Jan. 9, 2016); Wang et al., J. Cell Physiol., 231(1), 25-30 (2016). Elevated miR-133a levels in serum correlate to poorer prognosis in ICU patients. Tacke et al., Crit. Care Med., 42(5), 1096-104 (May 2014). Groups of miRNAs delineate between different infectious etiologies, such as S. aureus and E. coli. Wu et al., PLoS One, 8(10) (2013). The lack of standardization in measuring circulating miRNA expression affects reproducibility between analyses and limited its clinical applicability. Lee et al., Mol. Diagn. Ther., 21(3), 259-68 (June 2017).

Physiologic stress induces viral reactivation by impairing the immune response and upregulating cell cycle progression pathways such as MAPK and NF-κB. Walton et al., PLoS One, 9(6), e98819 (Jun. 11, 2014); Traylen et al., Future Virol., 6(4), 451-63 (April 2011). Secretion of pro-inflammatory cytokines, such as TNF-α, functions in reactivating latent cytomegalovirus (CMV) in patients that had undergone recent stress even absent systemic inflammation. Prösch et al., Virology, 272(2), 357-65 (Jul. 5, 2000). A combination of inflammatory challenges and immune cell dysregulation has been shown to contribute to an environment that both promotes viral reactivation and maintains viremia. Walton et al., PLoS One, 9(6), e98819 (Jun. 11, 2014).

In a traumatic shock EXAMPLE, C57BL6 mice were treated by sequential hemorrhagic shock followed by cecal ligation and puncture, which induces sepsis. RNA was extracted from cellular component of lung and immune cells in blood after discarding plasma and serum. Samples were collected from both healthy and critically ill mice and sequenced via NGS at Gene Wiz in South Plainfield, N.J., USA. Reads were aligned to mm9 genome using STAR and then unmapped reads were mapped to viral genomes via ROP. Dobin et al., Bioinformatics, 29(1), 15-21 (January 2013). Mangul et al., Genome Biol., 19 (Feb. 15, 2018). Two-sample t tests were conducted to compare number of viral reads in healthy versus critically ill mouse lung and blood.

### Definitions

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skill in the art to which this invention belongs. For any apparent discrepancy between the meaning of a term in the art and a definition provided in this specification, the meaning provided in this specification shall prevail.

Acute respiratory distress syndrome (ARDS) has the medical art-defined meaning. ARDS is a type of respiratory failure characterized by rapid onset of widespread inflammation in the lungs. Symptoms include shortness of breath, rapid breathing, and bluish skin coloration. Causes may include sepsis, pancreatitis, trauma, pneumonia, and aspiration.

Alternative splicing (AS) has the molecular biological art-defined meaning. RNA splicing is a basic molecular function that occurs in all cells directly after RNA transcription, but before protein translation, in which introns are removed and exons are joined. Alternative splicing or alternative RNA splicing, or differential splicing, is a regulated process during gene expression that results in a single gene coding for multiple proteins. Exons of a gene can be included within or excluded from the final, processed messenger RNA (mRNA) produced from that gene. The proteins translated from alternatively spliced mRNAs can contain differences in their amino acid sequence and, often, in their biological functions.

Aldo/keto reductase gene has the molecular biological art-defined meaning.

Base R is an R-based computer program.

Mann Whitney U tests has the statistical art-defined meaning. The Mann-Whitney U test (also called the Mann-Whitney-Wilcoxon (MWW), Wilcoxon rank-sum test, or Wilcoxon-Mann-Whitney test) is a nonparametric test of the null hypothesis that it is equally likely that a randomly selected value from one population is less than or greater than a randomly selected value from a second population. This test Can investigate whether two independent samples were selected from populations having the same distribution.

mountainClimber is a cumulative-sum-based approach to identify alternative transcription start (ATS) and alternative polyadenylation (APA) as change points. Unlike many existing methods, mountainClimber runs on a single sample and identifies multiple ATS or APA sites anywhere in the transcript. Cass & Xiao, Cell Systems, 9(4), 23, 393-400.e6 (October 2019).

Next Generation Sequencing (NGS) has the molecular biological art-defined meaning. NGS technology is typically characterized by being highly scalable, allowing the entire genome to be sequenced at once. Usually, this is accomplished by fragmenting the genome into small pieces, randomly sampling for a fragment, and sequencing it using one of a variety of technologies.

Principal Component Analysis (PCA) has the computer-art and molecular biological art-defined meaning. Principal component analysis is a statistical procedure that uses an orthogonal transformation to convert a set of observations of possibly correlated variables (entities each of which takes on various numerical values) into a set of values of linearly uncorrelated variables called principal components.

Read origin protocol (ROP) has the computer-art meaning of is a computational protocol that aims to discover the source of all reads, including those originating from repeat sequences, recombinant B and T cell receptors, and microbial communities. The Read Origin Protocol was developed to determine what the unmapped reads represented. Mangul al., Genome Biology 19, 36 (2018). Recent development of Read Origin Protocol (ROP) has demonstrated that unmapped reads align to bacterial, viral, fungal, and B/T rearrangement genomes.

Read has the molecular biological art-defined meaning of reading sequencing results to determine nucleotide base structure.

Sepsis has the medical art-defined meaning of a life-threatening condition that arises when the body's response to infection injures its tissues and organs. Bone et al., Chest, 101, 1644-1655 (1992); Singer et al., JAMA, 315, 801-810 (February 2016).

STAR aligner is the Spliced Transcripts Alignment to a Reference (STAR), a fast RNA-seq read mapper, with support for splice-junction and fusion read detection. STAR aligns reads by finding the Maximal Mappable Prefix (MMP) hits between reads (or read pairs) and the genome, using a Suffix Array index. Different parts of a read can be mapped to different genomic positions, corresponding to splicing or RNA-fusions. The genome index includes known splice-junctions from annotated gene models, allowing for sensitive detection of spliced reads. STAR performs local alignment, automatically soft clipping ends of reads with high mismatches. Dobin et al., STAR: Ultrafast universal RNA-seq aligner. Bioinformatics, 29(1), 15-21 (January 2013).

Treatment for sepsis has the medical-art recognized meaning. Sepsis is treatable, and timely implementation of targeted interventions improves outcomes. The Mayo Clinic informs the public that several medications are used in treating sepsis and septic shock. They include antibiotics. Broad-spectrum antibiotics, which are effective against a variety of bacteria, are usually used first. After learning the results of blood tests, a doctor may switch to a different antibiotic that's targeted to fight the specific bacteria causing the infection. They include intravenous fluids and vasopressors. Other medications include low doses of corticosteroids, insulin to help maintain stable blood sugar levels, drugs that modify the immune system responses, and painkillers or sedatives.

Treatment for COVID-19 has the medical-art recognized meaning. Corticosteroids can be therapeutic. See Prescott & Rice, Corticosteroids in COVID-19 ARDS: Evidence and hope during the pandemic. JAMA, 324, 1292-1295 (2020). Other treatments are known by persons having ordinary skill in the medical art. See Waterer & Rello, Steroids and COVID-19: We need a precision approach, not one size fits all. Infectious Diseases and Therapy (2020). See also Beigel et al., Remdesivir for the treatment of Covid-19—Preliminary Report. New England Journal of Medicine (2020).

Treatment for Acute respiratory distress syndrome (ARDS) has the medical-art recognized meaning. Corticosteroids can be therapeutic. See Prescott & Rice, Corticosteroids in COVID-19 ARDS: Evidence and hope during the pandemic. JAMA, 324, 1292-1295 (2020). Other treatments are known by persons having ordinary skill in the medical art.

V(D)J recombination has the molecular biological art-defined meaning. V(D)J recombination occurs in developing lymphocytes during the early stages of T and B cell maturation, involves somatic recombination, and results in the highly diverse repertoire of antibodies/immunoglobulins and T cell receptors (TCRs) found in B cells and T cells, respectively.

Whippet (OMICS_29617) is a program that enables detection and quantification of alternative RNA splicing events of any complexity with computational requirements compatible with a laptop computer. Whippet applies the concept of lightweight algorithms to event-level splicing quantification by RNAseq. The software can facilitate the analysis of simple to complex AS events that function in normal and disease physiology. Alternative splicing events with high entropy are identified using Whippet. Sterne-Weiler et al., Molecular Cell, 72, 187-200.e186 (2018).

### Materials and Methods

Mouse strains. Mice are purchased from The Jackson Laboratory. C57BL/6J, the most popular mouse model used, exhibits a Th1/more pro-inflammatory phenotype. C57BL/6J is also the background of numerous knock out animals. BALB/cJ is also another commonly used mouse and can be the background of analyses with knockout animals but has more of a Th1/anti-inflammatory predominant repose phenotype. The CAST mouse is derived from wild mouse and genetically different from common laboratory mice. Using these three strains adjusts for the heterogeneity seen in humans.

Mouse model of sepsis: cecal ligation and puncture (CLP). A mouse model of hemorrhagic shock followed by the induction of sepsis by cecal ligation and puncture induces severe sepsis. Lomas-Neira et al., Shock, 45(2), 157-65 (2016)); Monaghan et al., Mol Med., 24(1), 32 (Jun. 18, 2018); Wu et al., PLoS One, 8(10) (2013); Monaghan et al., Annals of Surgery, 255, 158-164 (2012). Anesthetized, restrained mice in supine position catheters are inserted into both femoral arteries. Mice are bled over a 5-10-minute period to a mean blood pressure of 30 mmHg (±5 mmHg) and kept stable for 90 minutes. To achieve this level of hypotension, the mice have one mL of blood withdrawn. One mL of blood is approximately 50% of their blood volume so this correlates to class 4 hemorrhagic shock in humans. Mice are resuscitated intravenously (IV) with Ringers lactate at four times drawn blood volume. Sham hemorrhages are performed as a control in which femoral arteries ligated, but no blood is drawn to mimic the tissue destruction. The following day, sepsis is induced as a secondary challenge by cecal ligation and puncture. The timing of this secondary challenged is based on previous findings that hemorrhagic shock followed twenty-four hours by the induction of sepsis produced results in line with critical illness such as altering PaO₂ to FIO₂ ratios. The mouse model uses a double hit of hemorrhagic shock followed by cecal ligation and puncture correlates to a missed bowel injury in humans after hemorrhagic shock. This mouse model correlates with an injury severity score (ISS) of twenty-five. The dual challenge of hemorrhagic shock followed by septic shock is in line with the sepsis patients who are critically ill. Sometimes patients present with bleeding from wounds and a bowel injury missed upon initial assessment.

Sample sizes for these assays are based upon results from the inventor's previous work looking at the alternative splicing of sPD-1 and an effect size of Cohen's d=2.85 standard deviations difference between groups was calculated. With such a large effect size, power analysis poorly justifies sample size since, if the effect size is tenable, it would be exceedingly rare for assays of any sample size to fail to reach statistical significance. Small sample sizes provide poor point estimates and may be very unstable. the inventors chose a sample size of six mice per group based on feasibility and hoping to provide a reasonable point estimate for each group.

Mice of both sexes are used, because there are significant sex differences in the response to bleeding from trauma. Deitch et al., Annals of Surgery, 246(3), 447-53; discussion 53-5 (2007).

Human subjects. Patients are recruited from the Trauma Intensive Care Unit (TICU) at Rhode Island Hospital with Institutional Review Board approval and consent. The patient population at Rhode Island Hospital (a level 1 trauma center) is sufficient for this EXAMPLE. Over 3700 trauma patients were admitted to the hospital in 2018. The TICU admitted 765 patients in 2018. This would cause over 3000 patients admitted to the intensive care unit over the 4-year project. Using the advanced technology of the hospital's electronic health records (EPIC) combined with the mandated trauma registry there are streamlined efforts to recruit and retain patients. Since the mouse model correlates to an injury severity score (ISS) of twenty-five, the goal is to ensure that the average ISS for all the patients is twenty-five. Minimal risk to the patient is maintained since there is no direct benefit; the blood collected are less than 50 mL over an 8-week period and not collected more than twice a week. Blood samples from patients are taken on admission (25 mL) and during the TICU stay when a complication is developed (25 mL). This should cause the maximum for the initial 8-week period after the trauma. When the patient is recovered, at least 8 weeks after the last blood draw, a final blood draw 50 mL of are done in the outpatient setting. A power analysis was done based upon previous results from human patients. The effect size of Cohen's d=0.8 using a power of 80% and alpha of 0.05 the inventors calculated a sample size of twenty-six per group. The mortality of patients in the TICU is 5%. To enroll twenty-six patients who die after trauma, the inventors need 520 TICU patients (26/0.05=520). No enrollment is planned in the last six months to ensure adequate follow up, data collection and analysis. Fourteen % of patients in the TICU have complications after trauma. Due to the correlation to the mouse model of an ISS of twenty-five, the average ISS for the enrolled patients is targeted at twenty-five. This system recruits some patients who are not used. These patient samples are banked and not sent for RNA sequencing. After twenty-six patients who die and twenty-six patients with a complication are enrolled. The entire set of patients has an average ISS of twenty-five then recruitment conclude.

Where patients are being recruited, variables such as age, weight, and medical co-morbidities are collected and compared across groups. If these variables are different (t test or rank sum), these factors are adjusted for in the analysis by regression.

In the human studies, both sexes are recruited and analyzed in the GTEx data set. Age, weight, and other health problems are constant in the mouse assays.

Sample collection and sequencing. Mouse blood and lung samples were obtained as described. Monaghan et al., Annals of Surgery, 255, 158-164 (2012). Data for humans was obtained from GTEx by their protocols. RNA was extracted using the MasterPure Complete DNA/RNA Purification kit (epicenter, Madison Wis., USA) followed by the Globin Clear Kit (ThermoScientific, Waltham, Mass., USA). RNA was then sent to Genewiz (South Plainfield, N.J., USA) for sequencing as 1400 ng RNA in forty μL of fluid.

The GTEx Project was supported by the Common Fund of the Office of the Director of the National Institutes of Health, and by NCI, NHGRI, NHLBI, NIDA, NIMH, and NINDS and the data used for the analyses were obtained from the GTEx Portal and dbGaP accession number phs000424.v6.p1.

Cloud based computing. All computational biology work is performed on cloud-based computing by Lifespan-RI Hospital approved and supported Microsoft Azure environment. This server manages all large data sets from RNA sequencing. An intentional decision was made to use cloud-based computing for this project. Due to the depth of sequencing that is needed for RNA splicing analysis (100 million reads vs. forty million), more data is generated from both sequencing and analysis (a small study generated one terabyte of sequencing data and another terabyte from the alignment to the genome). With such a large amount of data predicted available for the EXAMPLE, the ability to expand and contract the storage space and computing power in the cloud is the ideal choice. This server stores and analyzes data from both mouse and human samples. Since RNA sequencing data is always identifiable, the data from humans are treated as though it is protected health information (PHI), even though none of the typical identifiers (such as name, date of birth, etc.) are associated with the data. The server was created in collaboration with the Information Technology department at Rhode Island Hospital to ensure data security. The cloud server is only accessible through a hospital virtual desktop and data are saved only to the Azure server or a hospital computer. Data are encrypted while stored, and when in transit to or from the hospital. Any link to typical identifiers (name, date of birth, etc.) are kept separate from the sequencing data. The cloud-based server allows for large data analysis with computing and storage needs changing on a per-use basis. The Azure server is Linux based and uses programming in R and Python. The following pipeline encompasses the typical analysis: differential expression, RNA analysis is done with Whippet. This also includes an entropy measure, and genes of interest undergo GO term analysis. Genes with alternative transcription start and end sites identified through Whippet are correlated with findings from the mountainClimber analysis.

Computational analysis and statistics. RNA sequencing data from the mouse was first checked for quality using FASTQC. RNA-sequencing data collected from the GTEx consortium, and the mouse ARDS model was analyzed with the Whippet software for differential gene processing. Alternative transcription events are those events identified by Whippet as ‘tandem transcription start site,’ ‘tandem alternative polyadenylation site,’ ‘alternative first exon,’ and ‘alternative last exon.’ Alternative RNA splicing events are those events labeled ‘core exon.’ ‘alternative acceptor splice site,’ ‘alternative donor splice site,’ and ‘retained intron.’ Alternative mRNA processing events were determined by a log 2 fold change of greater than 1.5+/−0.2. Statistical significance was calculated by the chi-square p-value of a contingency table based on 1000 simulations of the probability of each result.

Gene ontology (GO) was assessed using The Gene Ontology Resource Knowledgebase. Ashburner et al., Nature Genetics, 25, 25-29 (2000); The Gene Ontology Resource: 20 years and still GOing strong. Nucleic Acids Research, 47. D330-d338 (2019). Genes from the analyses were entered and outputs displayed. Outputs from gene ontology do not correlate with actual increase or decrease in a gene's expression but are related to expected based upon the set of genes entered.

Blood sample collection. Blood samples are collected on day 0 of ICU admission. Clinical data including COVID specific therapies was collected prospectively from the electronic medical record and participants were followed until hospital discharge or death. Ordinal scale can be collected as described by Beigel et al., New England Journal of Medicine (2020); along with sepsis and associated SOFA score, and the diagnosis of ARDS. See Singer et al., The Third International Consensus Definitions for Sepsis and Septic Shock (Sepsis-3). JAMA, 315: 801-810 (2016); Ferguson et al. The Berlin definition of ARDS. Intensive Care Medicine, 38: 1573-1582 (2012).

RNA extraction and sequencing. Whole blood can be collected in PAXgene tubes (Qiagen, Germantown, Md.) and sent to Genewiz (South Plainfield, N.J., USA) for RNA extraction, ribosomal RNA depletion and sequencing. Sequencing can be done on Illumina HiSeq machines to provide 150 base pair, paired-end reads. Libraries were prepared to have three samples per lane. Each lane provided 350 million reads ensuring each sample had >100 million reads.

Computational Biology and Statistical Analysis. All computational analysis can be done blinded to the clinical data. The data can be assessed for quality control using FastQC. Andrews. A quality control tool for high throughput sequence data. FastQC (2014). RNA sequencing data can be aligned to the human genome utilizing the STAR aligner. Dobin et al., Bioinformatics (Oxford, England), 29, 15-21 (2013). Reads that aligned to the human genome can be separated and called ‘mapped’ reads. Reads that do not align to the human genome, which are typically discarded during standard RNA sequencing analysis, were identified as ‘unmapped’ reads. The unmapped reads then align to the relevant comparator and counted per sample using Magic-BLAST. See Boratyn et al., BMC Bioinformatics, 20, 405 (2019). The unmapped reads were further analyzed with Kraken2. See Wood, Lu, & Langmead, Genome Biology, 20, 257 (2019). The analysis used the PlusPFP index to identify other bacterial, fungal, archaeal, and viral pathogens. See Kraken 2/Bracken Refseq indexes maintained by BenLangmead, which uses Kutay B. Sezginel's modified version of the minimal GitHub pages theme.

Reads that align to the human genome, the mapped reads, also can undergo analysis for gene expression, alternative RNA splicing, and alternative transcription start/end by Whippet. See Sterne-Weiler et al., Molecular Cell, 72, 187-200.e186 (2018). When comparisons are made between groups (died vs. survived) differential gene expression can be set with thresholds of both p<0.05 and +/−1.5 log 2 fold change. Alternative splicing was defined as core exon, alternative acceptor splice site, alternative donor splice site, retained intron, alternative first exon and alternative last exon. Alternative transcription start/end events can be defined as tandem transcription start site and tandem alternative polyadenylation site. Alternative RNA splicing and alternative transcription start/end events can be compared between groups. See Sterne-Weiler et al., Molecular Cell, 72, 187-200.e186 (2018). Significance was set at great than 2 log 2 fold change as described by Fredericks et al., Intensive Care Medicine (2020). Genes identified from the analysis of mapped reads can be evaluated by GO enrichment analysis (PANTHER Overrepresentation released 20200728). See Mi et al. Nature Protocols, 8, 1551-1566 (2013).

Whippet can generate an entropy value for every identified alternative splicing and transcription event of each gene. These entropy values are created with no groups used in the gene expression analysis. To visualize this data a principal component analysis (PCA) can be conducted to reduce the dimensionality of the dataset and to obtain an unsupervised overview of trends in entropy values among the samples. Raw entropy values from all samples can be concatenated into one matrix and missing values were replaced with column means. Mortality can be overlaid onto the PCA plot to assess the ability of these raw entropy values to predict this outcome in this sample set. This analysis was done in R (version 3.6.3).

Kraken 2. The following tools are compatible with both Kraken 1 and Kraken 2. Both tools assist users in analyzing and visualizing Kraken results. Bracken allows users to estimate relative abundances within a specific sample from Kraken 2 classification results. Bracken uses a Bayesian model to estimate abundance at any standard taxonomy level, including species/genus-level abundance. Pavian has also been developed as a comprehensive visualization program that can compare Kraken 2 classifications across multiple samples. KrakenTools is a suite of scripts to help analyze Kraken results. For more information, a person having ordinary skill in the biomedical art can refer to Wood, Lu, & Langmead, Improved metagenomic analysis with Kraken 2, Genome Biology (Nov. 28, 2019).

The following EXAMPLES are provided to illustrate the invention and should not be considered to limit its scope.

### Example 1

### Unmapped Bacterial Reads to Identify Bacteria Causing Sepsis

Because bacterial infections are a common cause of morbidity in trauma patients, unmapped reads that align with bacteria are useful for the diagnosis and treatment of trauma patients. Unmapped reads from RNA sequencing data provide a valuable tool for the trauma patient. The decrease in the number of bacterial reads in the blood may be due to increased immune response. Some bacteria keep constant levels between groups, which signifies a virulent pathogen.

The technique of RNA sequencing has resulted in creating massive amounts of data. The first step with public RNA sequencing data is usually to align the reads to the reference genome of interest. RNA sequences that do not align with the reference genome (10-30%) are usually discarded when they cannot be mapped.

The inventors used a mouse model of hemorrhagic shock followed by cecal ligation and puncture. The inventors isolate RNA from blood and lung samples and had the RNA sequenced using standard techniques. They compare RNA from the test mice to sham controls. They analyze the RNA data that did not map to the mouse genome. Unmapped reads aligned to common bacterial pathogens, including Acinetobacter baumannii, Escherichia coli, Klebsiella pneumoniae, Pseudomonas aeruginosa, Staphylococcus aureus, Streptococcus agalactiae, Streptococcus pneumoniae, and Streptococcus pyogenes. The inventors also identify specific genes with high read counts.

In one assay, the blood samples from the test mice exposed to trauma had fewer reads mapping to bacteria (365,974) as compared to the control mice (902,063, p=0.02). In the lung, the bacteria counts were similar. Despite an overall decrease in mapped bacterial RNA reads in the test mice, the three Streptococcus species and Staphylococcus aureus had a similar number of reads mapping between the test mice and the control mice. The most common RNA read mapped to aldo/keto reductase gene from group B strep (82793634[uid]). There was more expression of this gene in the blood of mice after trauma (15,096) compared to controls (3671, p=0.006). This difference was not seen in the lung compartment (13,691 vs. 15,996, p=0.24). In the blood of the test mice, most of the identified bacterial sequences were reduced in counts compared to the blood of the control mice (43 vs. 16).

### Example 2

### Unmapped Viral Reads to Identify Sepsis or Viral Reactivation

Unmapped data have been aligned to regions in the genomes of viruses. In critical illness, not only does the percentage of unmapped reads suggest a biomarker, but also the alignment of unmapped reads to some viral genomes. The percentage of unmapped reads in these organs during periods of critical illness can be a biomarker of severity and outcomes.

To assess the impact of critical illness on unmapped reads and their composition, the inventors expose mice (e.g., C57BL6 mice) to sequential treatment of hemorrhagic shock followed by sepsis. This treatment produces indirect acute respiratory distress syndrome (ARDS). RNA is extracted from lung and blood samples and sequenced via next-generation RNA-sequencing. Reads are aligned to the mm9 reference genome. The sources of unmapped reads were aligned by Read Origin Protocol (ROP). Changes in the viral signature of the unmapped reads are different when comparing blood to the lung.

In a second assay, the blood samples of critically ill mice averaged 31.9 million reads versus 32.1 million reads in healthy mice, and lung samples of critically ill mice averaged 33 million reads versus 33.7 million reads in healthy mice. The blood of critically ill mice had an average of 1.5 million unmapped reads (4.74%), more than the average 52,000 unmapped reads (0.16%) in the blood of healthy mice (p=0.000082). The lungs of critically ill mice had, on average, 194,331 unmapped reads (0.58%), which was more than the average 130,480 unmapped reads (0.39%) seen in the lungs of healthy mice (p=0.031665). In blood samples, unmapped reads from critically ill mice were less likely to be viral than healthy mice (average 3480 in critically ill vs. 4866 in healthy, p=0.025955). In lung samples, unmapped reads from critically ill mice were more likely to be viral than those from healthy mice (average 6959 in critically ill vs. 3877 in healthy, p=0.031959). The results were notable for higher viral loads in lungs of critically ill mice, showing that viral RNA loads can be a biomarker of critical illness.

Human correlates can translate into a clinical setting.

### Example 3

### Unmapped B/T V(D)J Use to Identify Sepsis

In immune systems, V(D)J recombination allows for a diversity of antibodies in B cells and T cell receptors in T cells. During critical illness, the variety of these recombination events reduces, but recovers. RNA sequencing better characterizes V(D)J recombination events. RNA sequencing shows more diversity in critical illness compared to what was described previously. B and T cell composition could prove to be an important marker in critical illness and predicting outcomes of sepsis.

The inventors subject mice (e.g., C57BL6 mice) to sequential of hemorrhagic shock followed by sepsis. This induces acute respiratory distress syndrome (ARDS). Lung and blood samples are collected. RNA from the samples is sequenced by next-generation sequencing. Reads from critically ill and healthy mice are aligned to GRCm38 annotation and then mapped to the V(D)J annotation by Read Origin Protocol (ROP).

In a third assay, the inventors recovered ˜thirty million reads were recovered from RNA-seq data generated from lung tissue of critically ill mice and healthy controls. Alignment with STAR aligner showed an average of 7.77% unaligned reads in the healthy control, and 8.78% unaligned reads in the samples extracted from critically ill mice. Unmapped reads then underwent a secondary alignment to assay for V(D)J recombinants. Healthy mice have an average of 629 recombinant epitopes, whereas critically ill mice had an average of only 208 recombinant epitopes. Assays were done in triplicate with littermates.

Analysis of unmapped reads shows that critical illness inhibits the generation of B cell and T cell epitopes by the immune system during critical illness. Although the percentage of unmapped reads between healthy mice and critically ill mice was not significant, the composition of B and T cell epitopes differs vastly in critically ill mice.

### Example 4

### Principal Component Analysis of RNA Splicing Entropy to Identify Sepsis

Next Generation Sequencing is useful for the diagnosis and treatment of diseases.

The effect of alternative RNA splicing before translation has not been studied much, especially in the critically ill patient. Previous work showed an association between cancer and the level of global alternative splicing entropy. Elias & Dias, Cancer Microenvironment, 1(1),131-9 (2008); Ritchie et al., PLoS Computational Biology, 4(3), e1000011 (2008). RNA splicing entropy is correlated with acute respiratory distress syndrome (ARDS) across multiple tissues. Evaluating splicing entropy can provide insights about biological processes and gene targets in the critical illness setting.

The inventors induce a mouse model of ARDS by subjecting mice to hemorrhagic shock, followed by cecal ligation and puncture. Blood and lung samples are collected from three mice undergoing ARDS and three sham controls. RNA is purified.

Next-generation RNA sequencing is performed. Alternative splicing (AS) entropy levels are determined using Whippet (v 0.11) on Julia (v 0.6.4). Principal Component Analysis (PCA) is conducted using base R (v 3.4.0). Alternative splicing events with a proportion of spliced in values between 0.05 and 0.95 are analyzed. A threshold of 1.5 is applied to determine the percentage of high entropy events. Proportions of high entropy events across tissues and experimental groups are compared using Mann Whitney U tests.

In a fourth assay, Principal Component Analysis of the blood samples was performed. Samples clustered based on tissue type and ARDS status on a Principal Component Analysis plot This result suggested that splicing entropy can serve as a biomarker for ARDS status. The inventors observed differential levels of splicing entropy across tissue types, with the most entropy in the lung.

### Example 5

### RNA Lariats to Identify Sepsis

This EXAMPLE demonstrates the collecting of RNA sequencing data from a complex tissue (blood), rather than a cell line, and uses computational biology techniques to analyze the data.

RNA splicing occurs directly after DNA transcription, but before protein translation. RNA splicing by a two-step esterification process with the formation of an intermediary lariat formed by the intron and joining of the 5′ and 3′ splice sites. Introns typically degrade rapidly.

The biology of lariats has recently been identified as important as it relates to viral biology. The DBR1 gene encodes for the only RNA debranching enzyme. Mutations of DBR1 increase susceptibility to HSV1 and increase viral brainstem infections in humans. Assessing the RNA lariat counts in the critically ill trauma patients could predict poor outcomes or prolonged immune suppression. The inventers undertook the mouse model of critical illness (CLP). Assessing for the resolution or return to a healthy level of lariat counts could be a marker to identify immune suppression or those patients at risk for a complication.

The identification of lariats from RNA sequencing data had been difficult. The William G. Fairbrother laboratory created a method to count lariats from RNA sequencing data. Taggart et al., Nature Structural & Molecular Biology, 19, 719-721 (2012).

In a fifth assay, the preliminary data suggests that in the critically ill mouse, the typical metabolism of RNA lariats is changed, resulting in an accumulation of lariats in the blood. The inventors found that the blood of mice with the critical illness have higher lariat counts compared to the control mice.

### Example 6

### Traumatic Shock

Lungs from healthy mice had an average of 3877 viral reads. Lungs from critically ill mice had on average 6956 viral reads. Blood from healthy mice had 4866 viral reads. Blood from critically ill mice had 3480 viral reads. Lungs from critically ill mice were more likely to have unmapped reads originating from viral genomes when compared to lungs from healthy mice (0.36% in critically ill, 0.21% in healthy; p-value=0.032). This could be due to critical illness leading to a compromised immune response that allows for viral reactivation and a higher viral load in lungs of critically ill mice. Traylen et al., Future Virol., 6(4), 451-63 (April 2011).

Blood of healthy mice were more likely to have unmapped reads originating from viral genomes than blood of critically ill mice (0.05% in critically ill, 0.11% in healthy; p-value=0.026). There are several explanations for why healthy mice could have increased viral loads in the blood compared to critically ill mice. Mature lymphocytes are constantly recirculating through blood and lymphatic organs. Charles et al., Immunobiol. Immune Syst. Health Dis. 5th Ed. (2001). In critical illness, the release of pro-inflammatory mediators may compound the intensity of immune surveillance, as documented in patients with systemic inflammatory response syndrome (SIRS). Duggal et al., Science Reports, 8(1), 1-11 (Jul. 5, 2018).

Change in leukocyte populations in critically ill mice may lead to a higher number of RNA-producing polymorphonucleocytes (PMN) in blood, which reduces the total viral RNA signal in critically ill mouse blood. Therefore, steps are taken to enrich for lymphocytes and monocytes to reduce RNA reads from PMNs.

This traumatic shock EXAMPLE demonstrated an association between critical illness and higher viral loads in mouse lung, lending promise to the clinical use of viral loads as a marker of critical illness.

### Example 7

### Processing RNA Sequencing Data to Aid in the Care of Sepsis Patients

More should be known about RNA biology, specifically alternative RNA splicing, in the sepsis population.

Over 90% of human genes with multiple exons require alternative splicing events to produce functional proteins. Pan et al., Nature Genetics 40, 1413-1415 (2008). RNA splicing creates a large natural source of variation of the transcribed gene to the produced protein product. RNA splicing is under exquisite control under normal conditions. Fever, hypothermia, and osmotic stress from fluid shifts can influence RNA splicing in vitro and change RNA splicing, altering protein expression. Gultyaev et al., TSitologiia i Genetika, 48, 40-44 (2014); Lemieux et al., PloS One 10, e0126654 (2015); Mahen et al., PLoS Biology 8, e1000307 (2010). Acidosis influences RNA splicing. Elias & Dias, Cancer Microenvironment, 1 131-139 (2008). Hypoxia also influences RNA splicing. Romero-Garcia et al., Experimental Lung Research 40, 12-21 (2014); Kasim et al., The Journal of Biological Chemistry, 289, 26973-26988 (2014). The effects of physiologic stress on RNA splicing should be better known. The pathological significance of changes induced RNA splicing process and proteins should be better understood.

This EXAMPLE shows the use of deep RNA sequencing data using computational biology methods (RNA splicing entropy, lariat counts, viral identification, and B and T cell epitope creation) and apply these methods to three distinct data sets: mouse of different strains undergoing sepsis, deceased sepsis patients who participated in the GTEx project, and human sepsis patients.

RNA splicing entropy after sepsis. RNA splicing is a basic molecular function in all cells. This EXAMPLE uses the global index/marker of RNA splicing called ‘RNA splicing entropy’ a calculation of the precision of RNA splicing typically occurring. The entropy and thus the disorder, is maximal when the probability of all events P (xᵢ) is equally likely and the outcome is most uncertain. This calculation is done for each type of alternative splicing event: skipped exon, retained intron, alternative donor (3′ splice site), and alternative acceptor (5′ splice site). The alternative splicing events with high entropy are identified using Whippet.

A lower percentage of RNA slicing entropy may predict increased mortality or more complications, particularly infections, in patients with sepsis. Previous work on cancer samples has shown that RNA splicing entropy is increased in the tumor compared to the healthy tissue in many cancer types. From the preliminary data in mice with and without ARDS after sepsis, RNA splicing entropy is less in the blood, 7.7% vs 10.7%, p=0.1. RNA splicing entropy was calculated for total white blood cell components of mice with critical illness caused by hemorrhage and cecal ligation and puncture and compared to controls. The RNA from blood and the lungs of mice was extracted, processed, and then subjected to deep RNA sequencing.

Obtaining this data demonstrates the ability to isolate RNA samples from the target organ tissues of interest in the mouse model system. This EXAMPLE demonstrates the ability to process the complex data using computational biology and custom scripts that result from RNA sequencing. This preliminary data suggests that the process of RNA splicing in critical illness is different compared to the controls, changes in RNA splicing entropy may be a reflection/response to or a mechanism driving pathological processes that drive mortality and morbidity in patients with sepsis. Genes with significant alternative splicing and high entropy in the mouse after sepsis may be target for intervention. These genes of interest are identified using machine-learning techniques and compared across both humans and mice.

Assessment of viral activity after sepsis. In the initial assessment of RNA sequencing data, the reads are aligned to the genome of the species the sample came from. The unmapped reads can account for up to 20% of the data and this data is typically discarded. From this Read Origin Protocol analysis of multiple data sets (including GTEx data), the inventors found their protocol accounted for 99.9% of all reads. The data typically discarded was then analyzed in a seven-step process. Two of those steps are of particular interest because of the relevance to critical care: Viral reads and B and T cell receptor rearrangement.

Identification of viruses after sepsis is a marker of immune suppression since there is data suggesting sepsis re-activates herpes infections. Cook et al., Critical Care Medicine, 31, 1923-1929 ((2003)). Much current research is focused on these mechanisms and interventions. Viral counts could correlate with immune suppression or complications. This is important because of the re-activation data. RNA sequencing data from the lungs of control mice showed fewer viral reads (3877) compared to mice after sepsis (6956, p=0.032). In the blood the opposite was true. Control had 4866 counts versus sepsis with 3480 counts (p=0.026). This difference between tissue types could be due to a multitude of reasons, such as latent infections, like CMV, in the lung. Because blood is the most accessible tissue type, the efforts for the human samples should focus on the blood.

Assessment of immune cell epitopes after sepsis. During critical illness, the immune system is activated and likely creating new receptors to respond to challenges/pathogens. These epitopes come from lymphocytes, known to be reduced in sepsis with resolution to normal levels linked to recovery. Heffernan et al., Critical Care, 16, R12 (2012). While the count of lymphocytes themselves is useful, measuring the number and diversity of the epitopes could provide further insights into immune suppression after sepsis.

In the mouse model, preliminary data shows fewer epitopes in the lung of mice after sepsis, compared to control. This demonstrates the ability to analyze data from a mouse model and characterize B and T cell epitopes via computational methods. Like lymphocytes, the production of epitopes may reduce. Recovery should correlate with a return to normal immune state.

The above-described methods to assess for immune suppression in sepsis patients by analysis of RNA sequencing data to understand RNA biology are applied to these samples.

For analysis of RNA splicing entropy, lariat counts, viral identification, and B and T cell epitope creation in the mouse model, using pilot data, using forty mice (twenty critically ill, twenty healthy controls) should have 80% power to detect a difference at a two-tailed alpha of 0.05. This method is used for each of the three mouse variants.

At the time points of twenty-four hours after cecal ligation and puncture and fourteen days after cecal ligation and puncture, mice are sacrificed, and organs procured. Organs to be collected are brain, lung, heart, kidney, liver, spleen, and blood. RNA from these samples is isolated as described below. The time point of twenty-four hours after CLP is selected as that is the time of most significant organ dysfunction. The time point of fourteen days is selected, since this is the point at which a mouse would be considered a survivor after this challenge.

RNA from blood samples in the mouse are processed using the MasterPure Complete RNA Purification (epicenter, Madison Wis., USA) kit for mice. Due to the high concentration of globin RNA in blood samples, these samples can then be further processed with the GLOBINclear Kit (epicenter, Madison Wis., USA). From blood one of skill in the molecular biological art can get 30-50 nanograms per microliter, with a total blood volume isolated from the mouse of about one mL. RNA from lung, heart, brain, kidney, liver, and spleen samples are extracted using MasterPure Complete RNA Purification kit for mice. After RNA samples are processed, the RNA was sequenced using standard techniques, for example by Deep RNA sequencing with a goal of 100,000,000 reads per sample. All samples should require at least 1400 nanograms of RNA for deep sequencing.

Human samples. Patients are recruited under Institutional Review Board approval and after consent is obtained. Blood samples are obtained from pre-existing catheters to minimize the risk. Blood samples are collected on admission and serially while the patient is in the intensive care unit. Samples are collected in PAXgene tubes and stored in an −80 C freezer until isolation of RNA for sequencing is needed. RNA sequencing is done in batches to minimize cost. For this experiment, it is expected 300 sepsis patients are recruited (average of 100 the first three years to allow analysis over the final two years of the project).

Control samples are obtained from healthy patients undergoing routine laboratory analysis at outpatient facilities. Blood from these patients is collected in PAXgene tubes and stored in an −80 C freezer until isolation of RNA for sequencing is needed. RNA sequencing is done in batches to minimize cost. Healthy controls are matched to sepsis patients based upon demographic/clinical data. Recruitment aims for 300 patients total (average 100 each year over the first three years). Sample size calculations for the recruitment of humans was done based upon initial results from the mice assays. Preliminary data from humans with sepsis shows more variation compared to the mice data. These differences from humans are accounted for by several things such as age, sex, medical co-morbidities, and variations in the timing of collection from the point of the sepsis.

RNA from blood samples from humans are processed using the MasterPure Complete RNA Purification (epicenter, Madison Wis., USA) kit for humans. Due to the high concentration of globin RNA in blood samples, these samples can then be further processed with the GLOBINclear Kit (epicenter, Madison Wis., USA). All samples require at least 1400 nanograms of RNA for deep sequencing, e.g., by Deep RNA sequencing with a goal of 100,000,000 reads per sample.

Genotype Tissue Expression (GTEx). The GTEx data has over 500 patients included with at least one sample that has undergone RNA sequencing. Extensive clinical data is available on these participants. The data can stratify the patients into early deaths (<36 hours) and late deaths (>36 hours). This classification and comparison between the groups was done as it highlights a population who could be intervened upon. The patients who die later die because of immune suppression leading to complications from sepsis. Earlier identification of immune suppression could change outcomes. The GTEx samples have been collected and undergone RNA sequencing. This sequencing data are analyzed as described above.

Innovativeness. RNA sequencing technology affords an avenue to bring precision medicine to sepsis patients. The inventors used blood samples from sepsis patients, process them and obtain RNA sequencing data of similar quality to that of cell lines or solid tissue samples. Monaghan et al., Shock, 47, 100 (2017). RNA sequencing allows for understanding not only the gene expression but also RNA biology. RNA is unstable compared to DNA. Kara & Zacharias, Biopolymers, 101, 418-427 (2014). RNA is influenced by the specific cellular environment (altered in sepsis).

Conceptual Innovation. Past work on sepsis and molecular mechanisms has been focused on gene transcription and protein expression. The process of alternative RNA splicing also can influence the expression of a protein independent of the gene expression. Chang et al., Combinatorial Chemistry & High Throughput Screening, 13, 242-252 (2010); Fredericks et al., Biomolecules, 5, 893-909 (2015).

By comparing findings in mice to humans using the publicly available RNA sequencing data from GTEx and human samples from the Intensive Care Unit, the inventors can establish the nature/type of RNA splicing common across species.

By determining the temporal relationship of changes in RNA splicing entropy, RNA lariats, viral identification, and B and T cell epitope creation with developing complications/mortality, the inventors can establish whether RNA biology can provide insight to immune suppression after sepsis.

Assessing information in the unmapped reads (viral and B/T cell epitopes) to determine clinical significance is using data that is typically discarded. This is like the use of lymphocyte counts to predict sepsis outcomes. Heffernan et al., Critical Care, 16, R12 (2012).

Technical innovation. RNA is isolated from complex tissues from both mice and humans. The isolate RNA is of high enough quality to allow for deep RNA sequencing. This analysis has only previously been done on cell line or cancer samples.

The inventors can use a series of analytical algorithms; initially, using the STAR aligner, then Whippet to assess and characterize splicing events and splicing entropy. This analysis is done across GTEx data, mice with sepsis and humans with sepsis.

The inventors can use the Read Origin Protocol as a basis. The inventors can modify as appropriate to assess viral content and B/T cell epitopes in data obtained from mouse models of sepsis, GTEx, and humans with sepsis.

The inventors can apply the scripts used previously to calculate lariat counts from RNA sequencing data. Taggart et al., Nature Structural & Molecular Biology, 19, 719-721 (2012). The RNA sequencing data is obtained from mouse models of sepsis, GTEx, and humans with sepsis.

Assaying the large amount of data that comes from RNA sequencing is commonly not successful due to several reasons. The analyses have biases for which controls are not in place, the large data should produce a statistically significant result but is it biologically and clinically significant. Using multiple biologic outputs (RNA splicing entropy, lariat counts, viral identification, and B and T cell epitope creation) across three samples (GTEx, mouse model, and humans) mitigate.

By assaying RNA splicing entropy, lariat counts, viral identification, and B and T cell epitope creation, one of ordinary skill in the molecular biological art can identify patients with this prolonged immune suppression.

Analyzing data already collected, such as using the GTEx data, and data like the unmapped reads from RNA sequencing supports creativity. This data would typically be ignored, but with the proper clinical relevance, the data can be reanalyzed and potentially find new biomarkers. The lymphocyte counts on a complete blood count with differential, a potential biomarker in the sepsis population. Heffernan et al., Critical Care, 16, R12 (2012).

Analysis of RNA sequencing data can provide one marker of the severity of the critical illness.

Evaluating RNA biology and outcomes after sepsis. Next generation RNA sequencing allows for the analysis of the RNA and assessment of not only gene expression but also other biological processes (alternative splicing, changes in transcription start and end). Correlating genomic information from high throughput sequencing technologies about a patient on arrival to the hospital with outcomes such as death and complications like infection should improve care. Since RNA is not as stable as DNA, assessing RNA are more sensitive to the physiologic stress in sepsis. The inventors can assess how the physiologic stress of sepsis influences RNA biology and alters proteins. Assaying RNA biology in critical care sepsis patients should translate to other patients with critical care after diseases.

By high throughput RNA sequencing the inventors can assay gene expression and the RNA processing events of alternative transcription start/end and alternative RNA splicing of from leukocytes in the blood. All three of these biological processes influence protein expression via generation of the RNA (gene expression), changing the beginning and end of the RNA (alternative transcription start/end), and changing the isoforms that are expressed (alternative RNA splicing). The combination of these three modalities creates a ‘transcriptomic phenotype’ and better identifies expressed proteins in the sepsis population as compared to the typical use of gene expression alone, compared to DNA, RNA is more influenced by the physiologic derangements seen in sepsis such as hypoxia and acidosis in cell culture. Elias & Dias, Cancer Microenvironment, 1(1),131-9 (2008); Kasim et al., The Journal of Biological Chemistry, 289(39), 26973-88 (2014).

In an intensive care unit, monitoring of physiology correlates to improved clinical outcome. Clinicians do not monitor how this physiology impacts RNA biology. Using high throughput sequencing, the inventors assay RNA biology in sepsis patients. The understanding of RNA biology at the time of injury should predict mortality, complications, and other outcomes in sepsis patients. Three aims are tested using a mouse model of sepsis, data from GTEx of sepsis patients, and blood from sepsis patients with correlation to outcomes.

Aim 1: Identify changes in RNA biology (gene expression, alternative transcription start/end, and alternative RNA splicing) in the blood before and after a pre-clinical mouse model of sepsis and compare to controls.

Aim 2: Using the data available from the Genotype Tissue Expression (GTEx) project correlate findings in the mouse model to these sepsis patients (81 patients).

Aim 3: Enroll critically ill sepsis patients and identify aspects of RNA biology that identify and predict outcomes (mortality, infection).

These analyses use data from high throughput sequencing and cloud computing to establish findings of RNA biology that correlate and predict outcomes in sepsis patients. This data comes from an ancestrally diverse sepsis population and can be applied to sepsis patients across the country and to multiple critically ill patient populations.

New technology has come that allows for analysis of all genes, not just those identified by the technology at the time. Tompkins, The Journal of Trauma and Acute Care Surgery. 78(4), 671-86 (2015). With RNA sequencing technology, particularly at the depth identified (80-100 million reads) needed for RNA biology assessment, the inventors can assess all genes transcribed, not just those identified as important with older technology. The analysis of all transcribed genes allows for the identification of genes that may be important for trauma, that in the past were overlooked, likely due to low transcription levels, with RNA sequencing technology the inventors can assay RNA biology (alternative transcription start/end and alternative RNA splicing), for a complete understanding of what genes are ultimately translated to functional proteins. Hardwick et al., Frontiers in Genetics, 10, 709 (2019).

Over 90% of human genes with multiple exons require alternative splicing events to produce functional proteins, creating a potentially large natural source of variation of the transcribed gene to the produced protein product. Pan et al., Nature Genetics, 40(12), 1413-5 (2008). Splicing is under exquisite control under normal conditions. Some conditions common in trauma, such as fever, hypothermia, and osmotic stress from fluid shifts can influence RNA splicing in vitro and change RNA splicing, altering protein expression. Gultyaev et al., TSitologiia i Genetika, 48(6), 40-4 (2014); Lemieux et al., PloS One, 10(5), e0126654 (2015); Mahen et al., PLoS Biology, 8(2), e1000307 (2010).

Using a mouse model of trauma caused by hemorrhage followed by cecal ligation and puncture, the inventors reported that alternative RNA splicing results in expression of varied isoforms of an immune modulating protein (programmed cell death receptor-1, PD-1). Preliminary data on RNA splicing entropy indicate that global RNA splicing is modified in the mouse model of trauma. Ritchie et al., PLoS Computational Biology, 4(3), e1000011 (2008). Increased RNA splicing entropy is also present in other pathologic conditions, such as cancers, as compared to normal tissue. Ritchie et al., PLoS Computational Biology, 4(3), e1000011 (2008). Increased entropy is characteristic of disease states and could be a marker of critical illness after sepsis.

Sepsis patients are a good population in which to assay critical illness and generalize the findings to other patients. A population of sepsis patients is an ideal group to assay genomic factors as previous research has been hindered by lack of racial and ethnic diversity. Multiple factors cause minorities to avoid healthcare. Chikani et al., Public Health Reports, 131(5), 704-10 (2016). By assaying sepsis patients, the inventors can collect data from a diverse population that is more in line with the general population and not the population that seeks healthcare. The findings are more generalizable, especially among an ancestrally diverse population.

Protocols for sepsis have improved outcomes. Rhodes et al., Intensive Care Medicine, 41(9), 1620-8 (2015). Sepsis can cause critical illness in a young population. The response to sepsis should not be influenced by co-morbidities associated with an increasingly aged population, but the inventors can collect co-morbidities to assess if there is an impact.

Genomic medicine is an ideal target for sepsis patients but is limited by sequencing technologies. Although genomic medicine is typically defined as using genomic information about an individual patient as part of their clinical care, this definition cannot be applied to sepsis patients or any critically ill patients.

Next generation RNA sequencing takes about 18 hours on an Illumina machine, but this does not include time for data analysis. Since the data are delayed until the outcome of the patient is known, data analysis can be blinded to allow for more robust conclusions, through this work, the efficiencies in computation biology can be elucidated so that when the sequencing technology speeds up, the analysis are quick enough to have a clinically relevant time frame (less than one hour) from sample acquisition to actionable result.

Thus, there is value in understanding of how stressors associated with sepsis can affect RNA biology (RNA splicing (and entropy) and alternative transcription start/end) and how changes in the RNA biology leads to altered protein product expression, contributing to potential dysfunction at a cell and tissue level.

Innovation. Past work focusing on trauma and molecular mechanisms has been focused on gene transcription and protein expression. The process of alternative RNA splicing and alternative transcription start/end both have the potential to influence the expression of a protein independent of the gene expression. Chang et al., Combinatorial Chemistry & High Throughput Screening, 13(3), 242-52 (2010); Fredericks et al., Biomolecules, 5(2), 893-909 (2015). By comparing findings in mice to humans using the publicly available RNA sequencing data from GTEx and human samples from the Trauma Intensive Care Unit the inventors can establish the nature/type of RNA biology that is common across species.

In determining the temporal relationship of changes in RNA biology with developing complications/mortality, the inventors can establish whether RNA biology can provide insight to immune suppression after sepsis.

Knowledge of RNA biology in the critically ill is useful because previous work on this process has focused largely on chronic diseases and genetic diseases.

The combination of gene expression, RNA splicing, and transcription start/end create a ‘transcriptomic phenotype’ that can be followed during the patients hospital stay.

RNA is isolated from complex tissues from both mice and humans. The isolate RNA is of high enough quality to allow for deep RNA sequencing. This analysis has only previously been done on cell line or cancer samples.

The inventors can use a series of analytical algorithms using the STAR aligner, then Whippet, to assess and characterize RNA biology. Results from Whippet are compared to mountainClimber to ensure accurate data as it pertains to alternative transcription start and end. This analysis is done across GTEx data, mice with sepsis and humans with sepsis.

Using multiple biologic outputs (alternative RNA splicing, including entropy, alternative transcription start/end) across three different samples (GTEx, mouse model, and humans in the trauma intensive care unit) should mitigate some of the potential flaws.

Preliminary data regarding trauma. In a small cohort of trauma patients from GTEx, three patients form the early death cohort (<48 hours) were compared to six patients from the late death cohort (>/=48 hours). In this comparison, 524 genes are significantly increased in the late death versus the early death. In the late death group, 2331 genes are decreased compared to the early death group. The GO terms associated with the genes that decreased expression in the late group compared to the early group are valid based upon previous research. The terms with a decrease in expected representation in the GO terms reference mitochondrial biology. This decrease in GO terms likely represents that genes are increased in expression at the early death time point. Mitochondrial molecular patterns have been a component of the early response to trauma and those genes would be increased in the early group. (37, 38) anemia occurs during trauma. In the late group, genes associated with erythrocyte development are over-represented, suggesting increase expression in the late death group compared to the early death group. These few GO terms and correlation to phenotypes of trauma, suggest use of early versus late death is a valid clinical tool. This preliminary data shows the ability to access, manage, and analyze GTEx data with clinically significant groups using modern computational biology techniques. Using GO terms allows us to prove clinical relevance. This project aims to obtain and analyze all the trauma samples from GTEx. The inventors can also use similar computational approaches with the prospectively collected data from trauma patients.

Multiple alternative RNA splicing events and alternative transcription start, and events are detected, but there are fewer that are significant. Using the same cohort as above, this preliminary date from GTEx data, alternative splicing and alternative transcription events are characterized using Whippet. Multiple events were identified to be alternative RNA splicing and alternative transcription start/end in the blood samples. When comparing the groups there were only significant differences when assessing alternative RNA splicing and not alternative transcription start and end. This data confirms that alternative RNA splicing is an active process during trauma and could predict mortality and outcomes in trauma patients, genes with changes in splicing, and potentially transcription start/end could identify useful targets. The combination of gene expression, splicing and transcription start/end could alter what proteins were thought to have increased gene expression and subsequent protein transcription have altered processing resulting in new isoforms or changes in transcription. These findings highlight the ability to access GTEx data, categorize the samples in a clinically relevant manner, and process the RNA sequencing data with advanced computational methods, such as Whippet.

RNA splicing, specifically RNA splicing entropy shows differences after trauma. From the preliminary data in mice with and without, the inventors can show that in the blood there is less RNA splicing entropy, 7.7% versus 10.7%, p=0.1. RNA splicing entropy was calculated using Whippet. The percentage of each type of splicing event with an entropy of >1.5 (Alternative Donor, Alternative Acceptor, Retained Intron, and Skipped Exon). Using the mouse model of trauma, RNA splicing entropy was calculated for total white blood cell components of mice after trauma caused by hemorrhage with cecal ligation and puncture (n=3) and compared to controls (n=3). The RNA from blood was extracted, processed, and then subjected to deep RNA sequencing. This preliminary data suggests that the process of RNA splicing in critical illness is different compared to the controls, changes in RNA splicing entropy may be a reflection/response to or a mechanism driving pathological processes that drive mortality and morbidity in patients with trauma. Obtaining this data demonstrates the ability to isolate RNA samples from the target organ tissues of interest in the mouse model system. This EXAMPLE demonstrates the ability to process the complex data using computational biology and custom scripts that result from RNA sequencing.

The trauma patients in the intensive care unit provide an ancestrally diverse population and adequate numbers to correlate mortality and other complications. The trauma intensive care unit admits over 750 patients a year with 20% of those patients coming from an ancestrally diverse background. The enrollment is in line with the general population, even though underrepresented minorities seek medical care at a reduced rate. One aspect to this invention is the correlation of the RNA sequencing data to mortality and complications.

This EXAMPLE shows the importance of not only predicting mortality, but also using RNA sequencing data to predict complications as patients with complications had a higher mortality (7.7%). Mortality could be influenced. This data shows the trauma center has the volume of patients in the intensive care unit to have an appropriately powered study.

Over four years, 520 patients can be enrolled based on sample size calculations, with fewer than the 3000 expected admissions proving feasibility.

TABLE 4

| Aim | Suggested Type of Research | Application |
|---|---|---|
| 1 | Integration of other data types, | A model organism (mouse |
| | such as environmental data, family | after trauma) will provide |
| | history, transcriptomics, | the basis for other |
| | epigenomics, functional data, or | analyses in humans after |
| | model organism data to improve | trauma. Multiple strains |
| | assessment of clinical validity or | will mimic the diverse |
| | clinical utility of genomic | human population. |
| | information. | |
| 2 | Assessment of improved | GTEx data are re-analyzed |
| | approaches for reanalyzing patient | using modern approaches and |
| | genomic data and understanding | a unique population (early |
| | its impact on clinical care. | versus late trauma deaths) |
| 3 | Evaluation of modern approaches | Trauma patients will provide |
| | to interpreting genomic data in | an ancestrally diverse |
| | ancestrally diverse populations in | population to assay this |
| | clinical settings | clinical genomic date. |

This approach uses RNA sequencing data from a mouse model of trauma, re-analysis of existing genomic data in GTEx about early versus late trauma deaths, and samples from ancestrally diverse critically ill trauma patients uniquely suited to provide clinical information applicable across many clinical scenarios, particularly critically ill patients with cancer, sepsis, stroke, or myocardial infarction. The analysis of the RNA data from next generation sequencing technology creates a ‘transcriptomic phenotype’ for each trauma patient. Understanding the RNA biology at the time of injury can predict outcomes (mortality and complications) in trauma patients. The method to test the three aims, the expected result, and the potential impact are summarized in TABLE 5.

TABLE 5

| Aim | Method | Result | Impact |
|---|---|---|---|
| 1 | Mouse model of | Changes in RNA biology | These findings provide the |
| | trauma, assessing | predict mortality after the | foundation for predicting |
| | blood before | mouse model of trauma. | mortality and complications |
| | trauma, after | The results seen at 24 | in critically ill trauma |
| | trauma, and in | hours differ from those | patients. Data seen at 24 |
| | survivors | identified at 14 days. | hours and 14 days correlate |
| | | | with patients who die early |
| | | | versus late. |
| 2 | 81 deceased | Changes in RNA biology | This are the foundation for |
| | trauma patients | are identified in early | analysis of RNA data from |
| | from GTEx, 23 | versus late trauma deaths | trauma patients during their |
| | early deaths and | and these correlate with | hospital stay. |
| | 58 late deaths | mouse data. | |
| 3 | Critically ill trauma | Changes in RNA biology | Using RNA sequencing data |
| | patients assessing | on admission predict | predict mortality and |
| | blood on | complications and | complications and enhance |
| | admission and | mortality. changes over | care of trauma patients with |
| | throughout course | the hospital course | applicability to all intensive |
| | | correlate with long-term | care unit patients. |
| | | outcomes. | |

Aim 1: Identify changes in RNA biology (gene expression, alternative transcription start/end, and alternative RNA splicing) in the blood before and after a pre-clinical mouse model of trauma and compare to controls.

Rationale: to determine if altered RNA biology in its various forms can predict outcomes, RNA sequencing data must be collected at various time points during the traumatic injury. The inventors can establish the equivalency of such a pre-clinical animal model to what is encountered clinically. The inventors previously used a mouse model of hemorrhagic shock followed my septic shock by cecal ligation and puncture (CLP). Monaghan et al., J. Transl. Med., 14(1), 312 (2016). This mouse model mimics a trauma patient with hemorrhagic shock from an extremity injury who then had a missed bowel injury resulting in severe critical illness. Using this mouse model, the inventors can obtain blood at the initial injury and assess if changes in RNA biology, to predict mortality from the severe trauma model. Using a mouse model allows for acquisition of blood samples at multiple time points (twenty-four hours after injury and in those mice that survived). The inventors can first assess if RNA biology in the blood can predict mortality, if changes in RNA biology are seen twenty-four hours after injury, and how these correlate to the RNA biology of survivors at fourteen days.

Test 1: Assess RNA sequencing data and identify genes with changes in expression, alternative RNA splicing, and alternative transcription start/end to develop the ‘transcriptomic phenotype’ from shed blood in the mouse model of trauma to predict outcomes. Mice (8-12 weeks old) undergo hemorrhagic shock followed by CLP to mimic the critical illness that a trauma would undergo after hemorrhagic shock from an extremity injury complicated by a missed small bowel injury. Mice are used from the background of C57BL/6J, BALB/cJ, and CAST to simulate the heterogeneity of humans. Each group has twenty-four (twelve sham and twelve trauma) mice for each strain based upon statistical calculations. C57BL/6J mice have a 30% survival at fourteen days. The shed blood from the hemorrhage component is collected. Although this blood is collected before the effects of hemorrhage, this time point can mimic an early time point in trauma, since the mice have undergone anesthesia and isolation/catheter insertion of the artery. RNA is isolated, sequenced and analyzed as described. The mice that survive to fourteen days can also be sacrificed and used in Test 2.

Test 2: Assess RNA sequencing data and identify genes with changes in expression, alternative RNA splicing, and alternative transcription start/end to develop the ‘transcriptomic phenotype’ from the blood of mice at twenty-four hours and fourteen days after trauma. Mice (8-12 weeks old) undergo hemorrhagic shock followed by CLP to mimic a severe trauma. Mice are used from the background of C57BL/6J, BALB/cJ, and CAST. Mice are sacrificed at twenty-four hours after CLP. Mice that survive to fourteen days are also sacrificed to assess RNA biology at that point among the survivors. Appropriate controls for each type of background mice undergo sham procedures. Based upon previous work, six mice are needed for each group. After mice are sacrificed (CO₂ overdose followed by direct cardiac puncture) at either twenty-four hours or fourteen days after CLP blood are harvested. RNA from blood samples in the mouse are processed.

Human samples. Through collaboration with the military, soldiers in combat areas could be consented to donate blood before deployment. This blood would then undergo RNA sequencing and be compared to samples collected if there was an unfortunate traumatic injury. Many previous efforts using animal models to treat diseases such as sepsis failed to translate to humans. Fink & Warren. Nature Reviews Drug Discovery, 13(10), 741-58 (2014). The inventors previously studied conditions in mice with correlation to humans. Monaghan et al., J. Transl. Med., 14(1), 312 (2016); Monaghan et al., Molecular Medicine, 24(1), 32 (2018); Monaghan et al., Journal of the American College of Surgeons, 213(3), S54-S5 (2011); Monaghan et al. Annals of Surgery 255(1), 158-84 (2012). Trauma research may have better translatable results because of the timing of the disease. In trauma, the time of the event is known. This timing correlates with the induced trauma in the mouse. In sepsis, the time point at which sepsis started in the mouse is known. In humans, the time at which sepsis starts is impossible to know, as exemplified by inability to understand when an appendix may perforate. Iacobellis et al., Seminars in Ultrasound, CT, and MR, 37(1), 31-6 (2016). This is limited because it is a controlled traumatic challenge and should produce very consistent response to trauma. In humans, no trauma is the same. The number of humans needed to detect a difference is more since the traumas are not similar. Humans have more heterogeneity adjusted for by using multiple mouse strains. The inventors can account for differences in trauma by using the Injury Severity Score. The ISS of this challenge on the mouse is twenty-five, and this is the target average ISS of patients enrolled.

Aim 2: Using the data available from the Genotype Tissue Expression (GTEx) project correlate findings in the mouse model to these trauma patients (eighty-one patients).

Rationale. Using the GTEx data, the inventors can assess RNA biology in the blood of trauma patients. The GTEx data has over 500 patients included with at least one sample that has undergone RNA sequencing. The patients in the GTEx data set have extensive clinical data available. Unfortunately, all patients in this data set are deceased. This should be considered in interpretation of the data. To adjust for the fact all patients are deceased, the inventors use the time to procurement of the RNA from the death of the patient as a variable due to adjust for RNA degradation and other metrics as suggested by the GTEx consortium. (50) Trauma patients are selected (n=81) and identified as early (<48 hours) versus late death (>/=48 hours). The inventors can compare RNA biology between trauma patients who died early versus late and compare it to findings in a mouse model of mice who died early (twenty-four hours) versus survivors (fourteen days)

Test 1: Assess RNA sequencing data and identify genes with changes in expression, alternative RNA splicing, and alternative transcription start/end to develop the ‘transcriptomic phenotype’ the blood of deceased trauma patients and compare among early and late deaths. There are 81 unique trauma patients in the data set with blood samples. These patients are aged 20-68, in line with the age of typical trauma patients. The GTEx samples have been collected and undergone RNA sequencing. RNA sequencing data are aligned to the human genome with STAR. RNA Splicing events are assessed using Whippet and characterized into one of the five alternative splicing events: skipped exon, retained intron, mutually exclusive exon, alternative 3′ splice site, and alternative 5′ splice site. Entropy calculations are completed using Whippet. Alternative transcription events from Whippet are compared to outputs from mountainClimber.

Test 2: Correlation of changes in expression, alternative RNA splicing, and alternative transcription start/end (the ‘transcriptomic phenotype’) in the blood of humans to the mouse samples. From mouse model (Aim 1) changes in expression, alternative RNA splicing, and alternative transcription are identified and these are compared to findings in the human GTEx data (Aim 2, Test 1). The mouse model data are taken from mice at twenty-four hours after CLP and at fourteen days after CLP. This data is compared to the human data of early (<48 hours) and late (>/=48 hours) death. The identical genetic background of laboratory mice (despite coming from three strains) allows for assumptions to be made about significance of changes at a higher resolution, due to the certainty of the genetic model. Simultaneously it creates uncertainty about the validity of findings, due to a lack of comparability to humans that experience conditions outside of the laboratory. Human data is plagued by an equal and opposite effect as data derived from animal models. The homogeneity of the mouse model is replaced with heterogeneity due to factors such as age, sex, co-morbidities, and differences in the trauma. By coupling the certainty provided by the homogeneity of the mouse model, and the uncertainty provided by the heterogeneity of the human model, the inventors create a powerful tool with the potential to validate results from mouse analyses in humans. Comparing events across species can identify RNA biology events and genes that are important at both the early and late time point. These findings are compared to those found in the prospective collected data from trauma patients.

Human samples. In this sample set, all the patients are dead. Since RNA is unstable compared to DNA, adjustments in the comparisons between groups during the analysis must be made for the time it took for samples to be collected and RNA isolated. The mouse work is comparing to mice that are alive but were sacrificed. The GTEx consortium, to adjust for problems associated with deceased donors, has described multiple methods. Carithers et al., Biopreservation and Biobanking, 13(5), 311-9 (2015).

Aim 3: Enroll critically ill trauma patients and identify aspects of RNA biology that identify and predict outcomes (mortality, infection).

Rationale: A current challenge with the data from the animal models is ensuring translation to humans. This aim allows for complete translation of mouse data to humans. The human population of interest are patients admitted to the Trauma Intensive Care Unit (TICU).

Test 1: Assess RNA sequencing data and identify genes with changes in expression, alternative RNA splicing, and alternative transcription start/end in the blood can be prospectively detected and use this ‘transcriptomic phenotype’ in trauma patients on arrival and be correlated to mortality. Trauma patients are recruited from the trauma intensive care unit, which has an average of over 750 patients, admitted each year (over the last three years) and an average injury severity score (ISS) of 13, but the goal is to enroll patients with an average ISS of 25 to mimic the mouse model. Blood is collected in PAXgene tubes and stored at −80 C after informed consent is obtained. Samples are collected serially while in the ICU. Blood samples from patients are taken on admission (25 mL) and during the TICU stay when a complication is developed (25 mL). This causes the maximum for the initial 8-week period after the trauma. When the patient is recovered, at least eight weeks after the last blood draw, a final blood draw 50 mL of are done, potentially in the outpatient setting. Patients who survive the trauma are compared to patients who died. Clinical information for the trauma patients is collected from the trauma registry. The trauma registry is a database required as part of verification by the American College of Surgeons to be a trauma center. The data are standardized across the entire recruitment period. RNA is isolated using the PAXgene RNA Kit. RNA was sequenced (goal 80 to 100 million reads). RNA sequencing data are aligned to the human genome using the STAR aligner. Changes in expression, alternative RNA splicing, alternative transcription start/end, and RNA splicing entropy are identified with Whippet. Alternative transcription findings are correlated with mountainClimber.

Test 2: Assess RNA sequencing data and identify genes with changes in expression, alternative RNA splicing, and alternative transcription starts and end in the blood can be prospectively detected in trauma patients on arrival and use the ‘transcriptomic phenotype’ to correlate to outcomes and complications. Patients from the trauma intensive care unit identify differences in RNA biology between the healthy controls and trauma patients predict outcomes and complications. Outcomes and complications are recorded from the medical record and are defined in the trauma registry (and decided by trained coders). The trauma registry provides some demographic data, such as injury severity score to better quantify and adjust for the severity of the trauma across patients. Outcomes to follow and use as potential for prediction include mortality, hospital length of stay, intensive care unit length of stay, ventilator free days, and discharge disposition. Complications to be recorded again are taken from the trauma registry and include items such as infections (pneumonia, surgical site infections, urinary tract infection, bacteremia, sepsis), unplanned return to the operating room, unplanned return to the intensive care unit, tracheostomy, and feeding tube placement.

Human samples: In this sample set, all the patients are critically ill. Consenting patient who are critically ill requires a proxy and this can sometimes be difficult in the unexpected nature of trauma. The inventors have past success in consenting these patients. Human heterogeneity may make finding a significant difference between two groups difficult. Drastic difference (trauma patients in the intensive care unit survive versus die and those with complications) should allow for the identification of differences in RNA biology (‘transcriptomic phenotype’). All samples for this assay come from living patients.

### Example 8

### Survival Assay

All the test mice have the traumatic injury. They are maintained for fourteen days. At fourteen days all mice are sacrificed. The survival rate at fourteen days for the double hit model is 30%. The rate goes up to 70%. Monaghan et al. Annals of Surgery 255(1), 158-64 (2012). These estimates result in an effect size of h=0.823. A sample size of twenty-four per group during analysis would exceed 80% power at a 2-tailed alpha of 0.05 by a chi-square test of independent proportions, for survival analyses the inventors use twenty-four mice per group. This are done to ensure enough power to detect if RNA splicing at the initial challenge can predict survivors. Sham mice are operated (8 from each mouse background strain) at this time to procure samples at the 14-day time point.

RNA isolation and sequencing. RNA data from GTEx is extracted and sequenced per their protocols. RNA from mouse blood samples is processed using the MasterPure Complete RNA Purification (epicenter, Madison Wis., USA) kit for mice. Due to the high concentration of globin RNA in blood samples, these samples then be further processed with the GLOBINclear Kit (epicenter, Madison Wis., USA). From blood the inventors can get approximately 30-50 nanogram per microliter, with a total blood volume isolated from the mouse of about one mL. After RNA samples are processed, they are sequenced. All samples require at least 1400 nanograms of RNA for deep sequencing. Each sample are sent out (due to advancing technologies, costs of sequencing change frequently, therefore outside facility are chosen based upon cost during sample send out) for Deep RNA sequencing with a goal of 80 million to 100 million reads per sample.

Blood from trauma patients and healthy human control samples are collected using the PAXgene tubes (PreAnalytiX, Switzerland) and isolated using the PAXgene RNA kit (PreAnalytiX, Switzerland). Since it is impossible to predict the patients who die or have a complication on admission to the ICU, banked samples are used since the cost to perform RNA sequencing on the blood of all TICU patients at Rhode Island Hospital is impossible.

Assessment of clinical information. Clinical data relevant to the patient samples are collected from the trauma registry and the electronic medical record. This allows for collection of endpoints such as mortality. ICU length of stay, hospital length of stay, ventilator days, renal failure, ARDS, pneumonia, and other infectious complications. Besides data in the chart, the inventors also perform functional assessments at follow up after discharge. These would be based upon previous work in critical illness and use the 36-item short form (SF-36). The assessment is done at the 8+ week follow up.

### Example 9

### Alternative RNA Splicing and Alternative Transcription Start/End in Acute Respiratory Distress Syndrome

The objective of this EXAMPLE is to use RNA sequencing data and analysis to identify useful gene targets in sepsis.

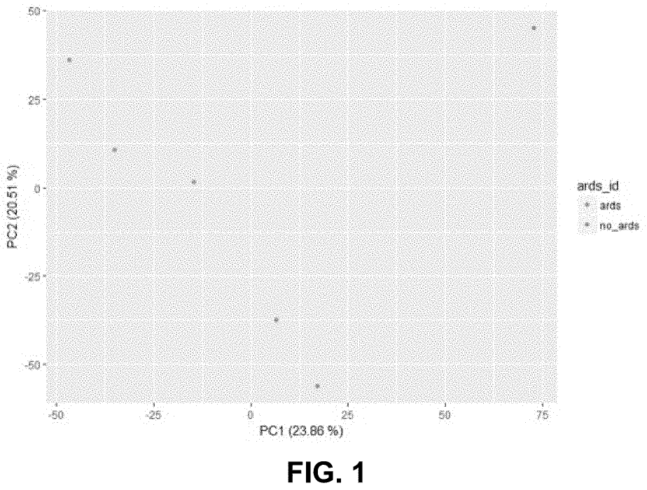

Alternatively spliced RNA arises from co/post-transcriptional events facilitated by the spliceosome, introns are removed to form the mature RNA from which protein isoforms are translated. Alternatively transcribed genes are the product of changes in promoter usage, polyadenylation signals, and RNA polymerase II interactions with DNA which can lead to changes in isoform usage like alternative splicing events. These are identified from the analysis of RNA sequencing data. Significant differentially alternatively transcribed genes and alternative spliced genes were identified and were overlapped with genes reported as ARDS related. See, Reilly et al., American Journal of Respiratory and Critical Care Medicine (2017). Of 89 reported ARDS related genes, 38 were confirmed in at least one differential category confirming that the use of humans and mice with DAD/ARDS is appropriate and robust (p=1.25e−14). Eleven previously reported genes were present in all categories. These eleven genes were evaluated for the change in alternative splicing and alternative transcription GO term enrichment analysis was performed on the eleven overlapping genes, revealing twenty significant biological processes including ontology related to aging, and response to abiotic/environmental stimuli. See FIG. 1. 1639 genes show overlap in alternative splicing and alternative transcription not previously in the literature. These genes were assessed for directionality alternative splicing and alternative transcription and GO terms. See TABLE 3 and TABLE 4.

Assaying the underlying changes in RNA processing (alternative splicing and alternative transcription start/end) not expands basic knowledge only of pathogenicity, but also provides additional targets for therapeutics. The most enriched GO term from the alternative splicing set, carboxy-terminal domain protein kinase complex (GO: 0032806) refers to phosphorylation of the CTD of RNA polymerase II, which is vital in regulating transcription and RNA processing. RNA polymerase complex binding (GO: 0000993), and transport of the SLBP Independent/Dependent mature mRNA (R-HSA-159227; R-HSA-159230) are among the most enriched. Alternative pre-mRNA splicing may have the dominate role in isoform usage in genes where expressions levels do not change, whereas alternative transcription may regulate isoform usage in genes that are more dynamically expressed during critical illness. Alternative splicing and alternative transcription may have separate roles in DAD/ARDS by regulating different genes to perform distinctive functions.

In this analysis of RNA sequencing data from deceased patients with ARDS identified by DAD and a clinically relevant mouse model of ARDS, useful genes are identified.

Overview. The inventors used RNA sequencing to identify changes in mRNA processing events (RNA splicing and transcription start/end sites) can be studied with RNA sequencing data. The inventors' strategy was to use the contrast how the processing of mRNA changes in lung and blood of patients with ARDS and compare to the lung and blood of a mouse model of ARDS.

Data. For this EXAMPLE, two main approaches were taken to obtain samples. The first was to use a validated mouse model of ARDS. Ayala et al., The American Journal of Pathology, 161, 2283-2294 (2002); Monaghan et al., Molecular Medicine (Cambridge, Mass., USA), 24, 32 (2018). All experiments were done according to guidelines from the National Institutes of Health (Bethesda, Md.). For the mouse model of ARDS. C57BL/6 male mice (The Jackson Laboratory, Bar Harbor, Me., USA) between 10 and 12 weeks of age were used. ARDS was induced in the mice by hemorrhage (non-lethal shock) followed by cecal ligation and puncture (CLP). The control group was sham hemorrhage followed by sham CLP.

The second approach was to identify patients in the GTEx Project with ARDS. All patients in the GTEx projects used in this EXAMPLE are deceased. A pathologist, blinded to the specimen ID and history, identified diffuse alveolar damage in lung samples from patients in GTEx. Most cases of clinical ARDS have diffused alveolar damage (DAD) morphologically. Zander & Farver, Pulmonary pathology e-book: A volume in foundations in diagnostic pathology series. (Elsevier Health Sciences, 2016). Classic DAD was identified based histologic features (For full description, please see supplement). Patients with evidence of diffuse alveolar damage in the lung and a corresponding blood and lung sample that had undergone RNA sequencing were placed in the ARDS group. Patients who had no evidence of diffuse alveolar damage in the pathology sample and a blood and lung sample with RNA sequencing were placed in the control group. Most cases of clinical acute lung injury (ALI) and acute respiratory distress syndrome (ARDS) have diffused alveolar damage (DAD) morphologically, which is divided into 2 phases: the acute/exudative phase and the organizing/proliferative phase. Other histologic patterns encountered in a clinical setting of ALI/ARDS include diffuse alveolar hemorrhage, acute eosinophilic pneumonia (AEP), and the acute fibrinous and organizing pneumonia (AFOP). Eight patterns of acute lung injury are evaluated in this EXAMPLE. Zander & Farver, Pulmonary pathology e-book: A volume in foundations in diagnostic pathology series. (Elsevier Health Sciences, 2016). Classic DAD was graded 1-4 based on the histologic features. Other patterns of injury were scored using a semiquantitative system for extent and histologic characteristics. For extent, grade was assigned: grade 1 (1 point): up to 10% tissue involved, grade 2 (2 points): 11-30% tissue involved, grade 3 (3 points): 31-50% tissue involved and grade 4 (4 points): >50% tissue involved. Histologic characteristics including intra-alveolar fibrin (1 point), cellular alveolar debris (I point), type II pneumocyte hyperplasia (1 point) and capillaritis/vasculitis. Total points 6 or higher were considered as DAD. Despite this complex method for categorizing diffuse alveolar damage, using this to diagnose ARDS is a major limitation. DAD could be present in other pulmonary diseases. The value RNA sequencing data from the lungs and blood of patients can provide biologic insights despite these limitations.

Results. Alternative splicing events were observed at 2-fold higher abundance as compared to alternative transcription events, yet significant alternative transcription events between groups were observed at a 6-fold higher prevalence (p=2.2e−16). Eighty-two alternative transcription events were common across all ARDS tissues (human and mouse, blood and lung, p=2.72e−16). No significant alternative splicing events were detected across all four tissues. As alternative splicing is species and tissue specific, it is unlikely to find an event that occurs in lung tissue and blood tissue in both human and mouse. GO term analysis was also performed on the significant differentially processing events.

The full list is TABLE 3 and TABLE 4 in International patent application PCT/US2021/018218, which are incorporated by reference.

### Example 10

### SARS-CoV-2 Viral Load and Nucleic Acid-Based Antiviral Therapies (RIH #332).

This invention disclosure describes a specific diagnostic. Determining which COVID-19 patients are at risk for severe disease and developing better anti-coronavirus therapies are clinical priorities. The invention leverages new information from genomic studies that demonstrate very limited, highly-specific regions of the SARS-CoV-2 genome are transcriptionally active in the bloodstream of COVID-19 patients. The invention solves the problem of measuring viral load in the blood from infected patients to assess prognosis. Viral load measurements are central to the management and prognosis of patients with HIV infection. The inventors found that using SARS-CoV-2 viral load to identify patients with more severe COVID-19, track the disease's course, and follow the response to treatment. The same sequences are used to create antiviral, interfering RNAs that block viral gene expression specific for viremia. This therapy directly blocks genes for COVID-19 pathogenesis. The invention is superior to potential competitors because the genomic data informs the design of relevant oligonucleotides that increases the assay's sensitivity.

The invention measures the amount of SARS-CoV-2 circulating in patients' blood. This amount is elevated in patients critically ill with COVID-19. The results therefore provide information on the prognosis of individual patients. Interfering RNA that specifically targets these sequences reduce these genes' expression, interrupting viral replication and the downstream consequences of COVID-19. The open reading frame encoding the N protein also includes ORF9b, which has been shown to antagonize the action of antiviral type I interferon. Blocking this region of the virus enhance the host's endogenous antiviral response.

The invention can be used by all clinicians caring for COVID-19 patients. High or increasing SARS-CoV-2 viral loads identify those at the most significant risk for severe disease. The therapy is unique compared to those approved or under clinical trial.

### Example 11

### Deep Sequencing to Identify Targets for Detecting and Treating Pathogens

This invention disclosure describes a more general approach. Diagnostic testing for specific infections depends on a substantial understanding of the underlying pathogen. Nucleic acid amplification tests (NAT) are increasingly used in clinical medicine, but developing a test is time-consuming and may miss NAT's optimal sequences. In the setting of a new pandemic like COVID-19, delays can be fatal. This invention leverages further information from deep sequencing of RNA from patients with specific infections to develop diagnostic targets and therapeutic strategies. Deep sequencing of RNA identifies the pathogen's most abundant RNA transcripts that establish a future work foundation. The approach is especially valuable for new pathogens poorly characterized because the RNA sequencing is unbiased and analyze known and unknown sequences.

The findings with SARS-CoV-2 illustrate the invention's potential. The sequencing studies demonstrate that very limited, highly specific regions of the SARS-CoV-2 genome are transcriptionally active in the bloodstream of COVID-19 patients. The invention solves the problem of measuring viral load in the blood from infected patients to assess prognosis. Viral load measurements are central to the management and prognosis of patients with HIV infection. The inventors found that using SARS-CoV-2 viral load to identify patients with more severe COVID-19, track the disease's course, and follow the response to treatment. The same sequences can be used to create antiviral, interfering RNAs that block viral gene expression specific for viremia. This therapy directly blocks genes for COVID-19 pathogenesis. The invention is superior to potential competitors that focus on DNA sequencing, which misses all possible RNA viruses like SARS-CoV-2 and influenza. Testing the most abundant RNA sequences enhance diagnostic assays' sensitivity because there can be more target sequences to measure.

The invention is a technical approach to measure pathogen RNA expression circulating in patients' blood. Computational analysis identities sequences, and these sequences can be used to develop diagnostic tests and therapeutics, such as small interfering RNAs mentioned above.

The invention can be used by academic and industry researchers who study infectious diseases pathogenesis, diagnostics, and treatment.

### Example 12

### RNA Sequencing to Assess Bacterial Gene Expression of Bacteria in the Human Host During Sepsis

Attached are the genes and counts from Acinetobacter in a patient with COVID-19. The genes with the most counts are listed. Bact Gene Exp. The Out CSEQS=Aligned out bam.

TABLE 6

| | | | | | | Out |
|---|---|---|---|---|---|---|
| Gene id | Chr | Start | End | Strand | Length | CSEQS |
| IX87_RS10825 | NZ_CP009257.1 | 2143428 | 2144498 | − | 1071 | 6072 |
| IX87_RS13765 | NZ_CP009257.1 | 2731324 | 2733000 | − | 1677 | 1621 |
| IX87_RS06655 | NZ_CP009257.1 | 1293290 | 1295281 | + | 1992 | 1486 |
| IX87_RS04495 | NZ_CP009257.1 | 875468 | 876043 | + | 576 | 1480 |
| IX87_RS01665 | NZ_CP009257.1 | 311888 | 312541 | − | 654 | 1213 |
| IX87_RS06455 | NZ_CP009257.1 | 1241219 | 1243858 | − | 2640 | 1151 |
| IX87_RS13760 | NZ_CP009257.1 | 2729698 | 2731236 | − | 1539 | 1120 |
| IX87_RS18095 | NZ_CP009257.1 | 3641468 | 3641941 | + | 474 | 1009 |
| IX87_RS14290 | NZ_CP009257.1 | 2840814 | 2842754 | − | 1941 | 1008 |
| IX87_RS09870 | NZ_CP009257.1 | 1960173 | 1963025 | − | 2853 | 995 |
| IX87_RS01755 | NZ_CP009257.1 | 329174 | 331312 | − | 2139 | 986 |
| IX87_RS09635 | NZ_CP009257.1 | 1887997 | 1889631 | − | 1635 | 879 |
| IX87_RS22220 | NZ_CP009257.1 | 1932345 | 1948691 | − | 16347 | 875 |
| IX87_RS17835 | NZ_CP009257.1 | 3585533 | 3587203 | + | 1671 | 856 |
| IX87_RS02170 | NZ_CP009257.1 | 400923 | 402596 | − | 1674 | 842 |
| IX87_RS10110 | NZ_CP009257.1 | 2012821 | 2014125 | − | 1305 | 829 |
| IX87_RS11740 | NZ_CP009257.1 | 2326862 | 2327869 | − | 1008 | 819 |
| IX87_RS09895 | NZ_CP009257.1 | 1967878 | 1969152 | + | 1275 | 806 |
| IX87_RS15045 | NZ_CP009257.1 | 3003563 | 3004957 | + | 1395 | 801 |
| IX87_RS09420 | NZ_CP009257.1 | 1842311 | 1843243 | − | 933 | 768 |
| IX87_RS06650 | NZ_CP009257.1 | 1292234 | 1293286 | + | 1053 | 764 |
| IX87_RS17750 | NZ_CP009257.1 | 3567691 | 3569478 | + | 1788 | 758 |
| IX87_RS10025 | NZ_CP009257.1 | 1992002 | 1995178 | + | 3177 | 737 |
| IX87_RS06640 | NZ_CP009257.1 | 1288710 | 1291097 | + | 2388 | 735 |
| IX87_RS04550 | NZ_CP009257.1 | 885563 | 886978 | − | 1416 | 693 |
| IX87_RS08840 | NZ_CP009257.1 | 1721583 | 1723820 | − | 2238 | 686 |
| IX87_RS20710 | NZ_CP009257.1 | 4164453 | 4166039 | + | 1587 | 683 |
| IX87_RS15035 | NZ_CP009257.1 | 3001041 | 3002585 | + | 1545 | 671 |
| IX87_RS15670 | NZ_CP009257.1 | 3126337 | 3130530 | + | 4194 | 669 |
| IX87_RS17765 | NZ_CP009257.1 | 3571341 | 3574025 | + | 2685 | 641 |
| IX87_RS15665 | NZ_CP009257.1 | 3122162 | 3126250 | + | 4089 | 621 |
| IX87_RS17795 | NZ_CP009257.1 | 3578320 | 3579918 | + | 1599 | 591 |
| IX87_RS09425 | NZ_CP009257.1 | 1843261 | 1844010 | − | 750 | 586 |
| IX87_RS09880 | NZ_CP009257.1 | 1964309 | 1966144 | − | 1836 | 574 |
| IX87_RS15625 | NZ_CP009257.1 | 3116997 | 3118187 | + | 1191 | 571 |
| IX87_RS09760 | NZ_CP009257.1 | 1921690 | 1924920 | + | 3231 | 565 |
| IX87_RS15755 | NZ_CP009257.1 | 3145473 | 3147626 | − | 2154 | 557 |
| IX87_RS09730 | NZ_CP009257.1 | 1915821 | 1917716 | − | 1896 | 553 |
| IX87_RS06375 | NZ_CP009257.1 | 1227725 | 1228243 | − | 519 | 542 |
| IX87_RS13365 | NZ_CP009257.1 | 2644473 | 2647190 | − | 2718 | 528 |
| IX87_RS07930 | NZ_CP009257.1 | 1538290 | 1539705 | − | 1416 | 517 |
| IX87_RS12725 | NZ_CP009257.1 | 2519255 | 2520769 | + | 1515 | 502 |
| IX87_RS09860 | NZ_CP009257.1 | 1957481 | 1958914 | − | 1434 | 494 |
| IX87_RS02825 | NZ_CP009257.1 | 524251 | 525132 | + | 882 | 491 |
| IX87_RS11765 | NZ_CP009257.1 | 2329533 | 2330852 | − | 1320 | 479 |
| IX87_RS08365 | NZ_CP009257.1 | 1609789 | 1610940 | + | 1152 | 470 |
| IX87_RS00340 | NZ_CP009257.1 | 44564 | 45673 | + | 1110 | 467 |
| IX87_RS13230 | NZ_CP009257.1 | 2616298 | 2617083 | + | 786 | 467 |
| IX87_RS01760 | NZ_CP009257.1 | 331491 | 331961 | − | 471 | 466 |
| IX87_RS15655 | NZ_CP009257.1 | 3120936 | 3121442 | + | 507 | 466 |
| IX87_RS09465 | NZ_CP009257.1 | 1852062 | 1853039 | + | 978 | 463 |
| IX87_RS07885 | NZ_CP009257.1 | 1533357 | 1533983 | + | 627 | 458 |
| IX87_RS00325 | NZ_CP009257.1 | 40294 | 42723 | + | 2430 | 456 |
| IX87_RS04585 | NZ_CP009257.1 | 893625 | 896204 | − | 2580 | 454 |
| IX87_RS10860 | NZ_CP009257.1 | 2150015 | 2152420 | − | 2406 | 450 |
| IX87_RS02405 | NZ_CP009257.1 | 443230 | 446997 | + | 3768 | 444 |
| IX87_RS09605 | NZ_CP009257.1 | 1880229 | 1881134 | + | 906 | 444 |
| IX87_RS17680 | NZ_CP009257.1 | 3556213 | 3559047 | − | 2835 | 434 |
| IX87_RS17800 | NZ_CP009257.1 | 3579921 | 3581417 | + | 1497 | 430 |
| IX87_RS01685 | NZ_CP009257.1 | 315690 | 316502 | + | 813 | 418 |
| IX87_RS07790 | NZ_CP009257.1 | 1508072 | 1508824 | − | 753 | 409 |
| IX87_RS06320 | NZ_CP009257.1 | 1215830 | 1217341 | + | 1512 | 405 |
| IX87_RS13990 | NZ_CP009257.1 | 2775687 | 2778371 | + | 2685 | 401 |
| IX87_RS07865 | NZ_CP009257.1 | 1527434 | 1529704 | + | 2271 | 400 |
| IX87_RS11565 | NZ_CP009257.1 | 2291470 | 2292456 | − | 987 | 399 |
| IX87_RS09855 | NZ_CP009257.1 | 1956167 | 1957333 | − | 1167 | 395 |
| IX87_RS11835 | NZ_CP009257.1 | 2335867 | 2336619 | − | 753 | 394 |
| IX87_RS16040 | NZ_CP009257.1 | 3210600 | 3212687 | + | 2088 | 394 |
| IX87_RS06465 | NZ_CP009257.1 | 1244622 | 1249127 | − | 4506 | 389 |
| IX87_RS04525 | NZ_CP009257.1 | 879769 | 880380 | − | 612 | 385 |
| IX87_RS05530 | NZ_CP009257.1 | 1072645 | 1075860 | − | 3216 | 381 |
| IX87_RS13500 | NZ_CP009257.1 | 2674288 | 2674647 | − | 360 | 381 |
| IX87_RS02305 | NZ_CP009257.1 | 425244 | 426281 | − | 1038 | 380 |
| IX87_RS18475 | NZ_CP009257.1 | 3699884 | 3702049 | + | 2166 | 380 |
| IX87_RS11755 | NZ_CP009257.1 | 2328932 | 2329288 | − | 357 | 379 |
| IX87_RS11870 | NZ_CP009257.1 | 2339714 | 2340025 | − | 312 | 376 |
| IX87_RS16280 | NZ_CP009257.1 | 3260688 | 3262844 | − | 2157 | 374 |
| IX87_RS17790 | NZ_CP009257.1 | 3576429 | 3578318 | + | 1890 | 374 |
| IX87_RS09865 | NZ_CP009257.1 | 1958977 | 1960173 | − | 1197 | 372 |
| IX87_RS11805 | NZ_CP009257.1 | 2333659 | 2334195 | − | 537 | 370 |
| IX87_RS01750 | NZ_CP009257.1 | 327889 | 329079 | − | 1191 | 365 |
| IX87_RS13190 | NZ_CP009257.1 | 2605354 | 2606253 | + | 900 | 358 |
| IX87_RS12160 | NZ_CP009257.1 | 2399736 | 2401007 | − | 1272 | 356 |
| IX87_RS16715 | NZ_CP009257.1 | 3350516 | 3352144 | + | 1629 | 352 |
| IX87_RS13300 | NZ_CP009257.1 | 2630383 | 2631150 | + | 768 | 347 |
| IX87_RS15700 | NZ_CP009257.1 | 3135012 | 3135602 | + | 591 | 347 |
| IX87_RS13360 | NZ_CP009257.1 | 2642488 | 2644470 | − | 1983 | 343 |
| IX87_RS20715 | NZ_CP009257.1 | 4166036 | 4167181 | + | 1146 | 337 |
| IX87_RS11435 | NZ_CP009257.1 | 2266603 | 2267031 | + | 429 | 335 |
| IX87_RS21505 | NZ_CP009257.1 | 4303748 | 4305118 | + | 1371 | 334 |
| IX87_RS00440 | NZ_CP009257.1 | 62067 | 63671 | − | 1605 | 330 |
| IX87_RS17320 | NZ_CP009257.1 | 3480635 | 3480973 | + | 339 | 330 |
| IX87_RS16695 | NZ_CP009257.1 | 3345765 | 3347618 | + | 1854 | 324 |
| IX87_RS06750 | NZ_CP009257.1 | 1311509 | 1313083 | + | 1575 | 320 |
| IX87_RS14750 | NZ_CP009257.1 | 2940826 | 2942109 | + | 1284 | 319 |
| IX87_RS16955 | NZ_CP009257.1 | 3402811 | 3405567 | + | 2757 | 304 |
| IX87_RS15015 | NZ_CP009257.1 | 2998731 | 2999606 | + | 876 | 300 |
| IX87_RS03345 | NZ_CP009257.1 | 627208 | 630690 | + | 3483 | 299 |
| IX87_RS16235 | NZ_CP009257.1 | 3250051 | 3253383 | − | 3333 | 295 |
| IX87_RS12010 | NZ_CP009257.1 | 2369134 | 2370084 | + | 951 | 294 |
| IX87_RS07235 | NZ_CP009257.1 | 1410286 | 1410498 | − | 213 | 289 |
| IX87_RS08390 | NZ_CP009257.1 | 1615750 | 1616997 | + | 1248 | 288 |
| IX87_RS13195 | NZ_CP009257.1 | 2606313 | 2608280 | − | 1968 | 288 |
| IX87_RS08710 | NZ_CP009257.1 | 1692357 | 1693730 | − | 1374 | 287 |
| IX87_RS15645 | NZ_CP009257.1 | 3119489 | 3119917 | + | 429 | 286 |
| IX87_RS11795 | NZ_CP009257.1 | 2332937 | 2333332 | − | 396 | 283 |
| IX87_RS17715 | NZ_CP009257.1 | 3563307 | 3563489 | − | 183 | 283 |
| IX87_RS11735 | NZ_CP009257.1 | 2326466 | 2326843 | − | 378 | 279 |
| IX87_RS17760 | NZ_CP009257.1 | 3569998 | 3571329 | + | 1332 | 279 |
| IX87_RS17145 | NZ_CP009257.1 | 3443650 | 3443844 | + | 195 | 278 |
| IX87_RS11850 | NZ_CP009257.1 | 2337251 | 2338075 | − | 825 | 275 |
| IX87_RS11685 | NZ_CP009257.1 | 2314954 | 2317008 | + | 2055 | 274 |
| IX87_RS15925 | NZ_CP009257.1 | 3177207 | 3179906 | + | 2700 | 274 |
| IX87_RS21205 | NZ_CP009257.1 | 4246489 | 4247538 | + | 1050 | 273 |
| IX87_RS12715 | NZ_CP009257.1 | 2516824 | 2517588 | + | 765 | 272 |
| IX87_RS21315 | NZ_CP009257.1 | 4268688 | 4269932 | − | 1245 | 270 |
| IX87_RS07155 | NZ_CP009257.1 | 1395265 | 1395480 | + | 216 | 266 |
| IX87_RS06275 | NZ_CP009257.1 | 1204344 | 1206950 | − | 2607 | 265 |
| IX87_RS04520 | NZ_CP009257.1 | 879360 | 879704 | + | 345 | 262 |
| IX87_RS07845 | NZ_CP009257.1 | 1521183 | 1522565 | + | 1383 | 254 |
| IX87_RS13725 | NZ_CP009257.1 | 2720466 | 2722304 | + | 1839 | 254 |
| IX87_RS11745 | NZ_CP009257.1 | 2327887 | 2328513 | − | 627 | 252 |
| IX87_RS02665 | NZ_CP009257.1 | 489716 | 490609 | − | 894 | 247 |
| IX87_RS07455 | NZ_CP009257.1 | 1456779 | 1458032 | − | 1254 | 247 |
| IX87_RS20615 | NZ_CP009257.1 | 4142534 | 4144810 | − | 2277 | 247 |
| IX87_RS11095 | NZ_CP009257.1 | 2202807 | 2204585 | + | 1779 | 246 |
| IX87_RS15370 | NZ_CP009257.1 | 3067271 | 3068719 | − | 1449 | 244 |
| IX87_RS16880 | NZ_CP009257.1 | 3387258 | 3388982 | + | 1725 | 244 |
| IX87_RS16870 | NZ_CP009257.1 | 3383613 | 3386237 | − | 2625 | 240 |
| IX87_RS09920 | NZ_CP009257.1 | 1972039 | 1973925 | + | 1887 | 239 |
| IX87_RS11250 | NZ_CP009257.1 | 2231341 | 2233242 | + | 1902 | 239 |
| IX87_RS08950 | NZ_CP009257.1 | 1748338 | 1749795 | − | 1458 | 238 |
| IX87_RS16890 | NZ_CP009257.1 | 3389506 | 3390522 | + | 1017 | 238 |
| IX87_RS08540 | NZ_CP009257.1 | 1651103 | 1653115 | − | 2013 | 237 |
| IX87_RS13720 | NZ_CP009257.1 | 2719089 | 2720453 | + | 1365 | 237 |
| IX87_RS11170 | NZ_CP009257.1 | 2216052 | 2216978 | − | 927 | 236 |
| IX87_RS16665 | NZ_CP009257.1 | 3336962 | 3338275 | + | 1314 | 236 |
| IX87_RS07785 | NZ_CP009257.1 | 1507059 | 1507934 | − | 876 | 235 |
| IX87_RS06305 | NZ_CP009257.1 | 1212164 | 1213336 | + | 1173 | 232 |
| IX87_RS09415 | NZ_CP009257.1 | 1839230 | 1841944 | − | 2715 | 232 |
| IX87_RS16660 | NZ_CP009257.1 | 3336255 | 3336860 | + | 606 | 231 |
| IX87_RS12690 | NZ_CP009257.1 | 2506859 | 2508520 | − | 1662 | 230 |
| IX87_RS01775 | NZ_CP009257.1 | 334318 | 335547 | + | 1230 | 229 |
| IX87_RS10020 | NZ_CP009257.1 | 1990739 | 1991989 | + | 1251 | 228 |
| IX87_RS09755 | NZ_CP009257.1 | 1920536 | 1921675 | + | 1140 | 227 |
| IX87_RS18140 | NZ_CP009257.1 | 3648577 | 3649527 | − | 951 | 227 |
| IX87_RS08545 | NZ_CP009257.1 | 1653366 | 1653935 | − | 570 | 225 |
| IX87_RS15750 | NZ_CP009257.1 | 3144288 | 3145460 | − | 1173 | 222 |
| IX87_RS10030 | NZ_CP009257.1 | 1995178 | 1996632 | + | 1455 | 220 |
| IX87_RS16035 | NZ_CP009257.1 | 3210073 | 3210342 | + | 270 | 218 |
| IX87_RS08585 | NZ_CP009257.1 | 1663238 | 1665856 | + | 2619 | 217 |
| IX87_RS11770 | NZ_CP009257.1 | 2330898 | 2331338 | − | 441 | 216 |
| IX87_RS11920 | NZ_CP009257.1 | 2349910 | 2352483 | + | 2574 | 216 |
| IX87_RS16835 | NZ_CP009257.1 | 3375467 | 3376198 | + | 732 | 212 |
| IX87_RS09660 | NZ_CP009257.1 | 1902424 | 1904256 | + | 1833 | 206 |
| IX87_RS10865 | NZ_CP009257.1 | 2152594 | 2153826 | − | 1233 | 206 |
| IX87_RS13315 | NZ_CP009257.1 | 2633562 | 2634899 | − | 1338 | 206 |
| IX87_RS14635 | NZ_CP009257.1 | 2913027 | 2914694 | + | 1668 | 205 |
| IX87_RS17670 | NZ_CP009257.1 | 3554601 | 3555884 | − | 1284 | 203 |
| IX87_RS11030 | NZ_CP009257.1 | 2186586 | 2187758 | + | 1173 | 202 |
| IX87_RS13385 | NZ_CP009257.1 | 2649792 | 2650967 | − | 1176 | 201 |
| IX87_RS11820 | NZ_CP009257.1 | 2334999 | 2335256 | − | 258 | 200 |
| IX87_RS11055 | NZ_CP009257.1 | 2192544 | 2194346 | + | 1803 | 199 |
| IX87_RS17690 | NZ_CP009257.1 | 3559580 | 3560296 | + | 717 | 199 |
| IX87_RS08995 | NZ_CP009257.1 | 1757654 | 1758889 | + | 1236 | 198 |
| IX87_RS12585 | NZ_CP009257.1 | 2488594 | 2488893 | + | 300 | 198 |
| IX87_RS12435 | NZ_CP009257.1 | 2453324 | 2457799 | − | 4476 | 197 |
| IX87_RS16740 | NZ_CP009257.1 | 3357561 | 3359744 | + | 2184 | 196 |
| IX87_RS09850 | NZ_CP009257.1 | 1955262 | 1956152 | − | 891 | 195 |
| IX87_RS15720 | NZ_CP009257.1 | 3139270 | 3140409 | − | 1140 | 195 |
| IX87_RS11815 | NZ_CP009257.1 | 2334538 | 2334906 | − | 369 | 194 |
| IX87_RS15030 | NZ_CP009257.1 | 3000458 | 3000994 | + | 537 | 194 |
| IX87_RS10940 | NZ_CP009257.1 | 2166865 | 2169588 | − | 2724 | 192 |
| IX87_RS13215 | NZ_CP009257.1 | 2610192 | 2611907 | − | 1716 | 192 |
| IX87_RS14160 | NZ_CP009257.1 | 2816591 | 2818351 | + | 1761 | 191 |
| IX87_RS06350 | NZ_CP009257.1 | 1224434 | 1224943 | + | 510 | 190 |
| IX87_RS11965 | NZ_CP009257.1 | 2359642 | 2360235 | − | 594 | 190 |
| IX87_RS14455 | NZ_CP009257.1 | 2873551 | 2874168 | + | 618 | 190 |
| IX87_RS15435 | NZ_CP009257.1 | 3081320 | 3082621 | − | 1302 | 190 |
| IX87_RS00480 | NZ_CP009257.1 | 70857 | 72470 | − | 1614 | 189 |
| IX87_RS04615 | NZ_CP009257.1 | 900663 | 901442 | − | 780 | 189 |
| IX87_RS06685 | NZ_CP009257.1 | 1297940 | 1298386 | + | 447 | 189 |
| IX87_RS10790 | NZ_CP009257.1 | 2135042 | 2135473 | − | 432 | 189 |
| IX87_RS12710 | NZ_CP009257.1 | 2513820 | 2516168 | − | 2349 | 188 |
| IX87_RS15040 | NZ_CP009257.1 | 3002663 | 3003532 | + | 870 | 188 |
| IX87_RS12270 | NZ_CP009257.1 | 2421741 | 2422973 | + | 1233 | 187 |
| IX87_RS02435 | NZ_CP009257.1 | 450849 | 451439 | − | 591 | 186 |
| IX87_RS12720 | NZ_CP009257.1 | 2517606 | 2519063 | + | 1458 | 186 |
| IX87_RS13755 | NZ_CP009257.1 | 2728221 | 2729633 | − | 1413 | 186 |
| IX87_RS12420 | NZ_CP009257.1 | 2450049 | 2450639 | − | 591 | 185 |
| IX87_RS09490 | NZ_CP009257.1 | 1856624 | 1859032 | − | 2409 | 184 |
| IX87_RS08860 | NZ_CP009257.1 | 1728937 | 1729419 | + | 483 | 183 |
| IX87_RS09135 | NZ_CP009257.1 | 1782073 | 1782822 | − | 750 | 183 |
| IX87_RS11655 | NZ_CP009257.1 | 2308487 | 2311087 | + | 2601 | 182 |
| IX87_RS14375 | NZ_CP009257.1 | 2856528 | 2859365 | − | 2838 | 182 |
| IX87_RS15650 | NZ_CP009257.1 | 3119921 | 3120616 | + | 696 | 182 |
| IX87_RS08735 | NZ_CP009257.1 | 1697312 | 1698382 | − | 1071 | 181 |
| IX87_RS10935 | NZ_CP009257.1 | 2166291 | 2166734 | − | 444 | 181 |
| IX87_RS12840 | NZ_CP009257.1 | 2535806 | 2536249 | + | 444 | 181 |
| IX87_RS13745 | NZ_CP009257.1 | 2726033 | 2726953 | − | 921 | 181 |
| IX87_RS17695 | NZ_CP009257.1 | 3560386 | 3561978 | + | 1593 | 181 |
| IX87_RS08730 | NZ_CP009257.1 | 1696205 | 1697299 | − | 1095 | 180 |
| IX87_RS16655 | NZ_CP009257.1 | 3334728 | 3336062 | + | 1335 | 180 |
| IX87_RS16680 | NZ_CP009257.1 | 3339859 | 3341385 | − | 1527 | 180 |
| IX87_RS10510 | NZ_CP009257.1 | 2078576 | 2080054 | + | 1479 | 179 |
| IX87_RS11680 | NZ_CP009257.1 | 2312388 | 2314820 | + | 2433 | 179 |
| IX87_RS18070 | NZ_CP009257.1 | 3638267 | 3638827 | + | 561 | 179 |
| IX87_RS11585 | NZ_CP009257.1 | 2297154 | 2298197 | + | 1044 | 178 |
| IX87_RS13895 | NZ_CP009257.1 | 2757634 | 2757996 | + | 363 | 176 |
| IX87_RS20755 | NZ_CP009257.1 | 4171530 | 4171889 | − | 360 | 175 |
| IX87_RS16760 | NZ_CP009257.1 | 3362171 | 3362602 | + | 432 | 173 |
| IX87_RS08660 | NZ_CP009257.1 | 1678795 | 1679757 | − | 963 | 172 |
| IX87_RS15920 | NZ_CP009257.1 | 3175712 | 3177196 | + | 1485 | 172 |
| IX87_RS06380 | NZ_CP009257.1 | 1228326 | 1229270 | − | 945 | 171 |
| IX87_RS10975 | NZ_CP009257.1 | 2175630 | 2176433 | − | 804 | 171 |
| IX87_RS17200 | NZ_CP009257.1 | 3453025 | 3453351 | − | 327 | 171 |
| IX87_RS18660 | NZ_CP009257.1 | 3739463 | 3741328 | + | 1866 | 171 |
| IX87_RS13850 | NZ_CP009257.1 | 2750180 | 2750782 | + | 603 | 170 |
| IX87_RS15425 | NZ_CP009257.1 | 3078971 | 3080017 | + | 1047 | 170 |
| IX87_RS11865 | NZ_CP009257.1 | 2339018 | 2339656 | − | 639 | 169 |
| IX87_RS18655 | NZ_CP009257.1 | 3739059 | 3739331 | + | 273 | 169 |
| IX87_RS03230 | NZ_CP009257.1 | 603458 | 605602 | − | 2145 | 168 |
| IX87_RS16685 | NZ_CP009257.1 | 3341612 | 3343756 | − | 2145 | 168 |
| IX87_RS17180 | NZ_CP009257.1 | 3447929 | 3450310 | + | 2382 | 168 |
| IX87_RS21240 | NZ_CP009257.1 | 4252339 | 4254843 | − | 2505 | 167 |
| IX87_RS07335 | NZ_CP009257.1 | 1429692 | 1432088 | + | 2397 | 166 |
| IX87_RS13845 | NZ_CP009257.1 | 2749271 | 2750164 | + | 894 | 166 |
| IX87_RS20560 | NZ_CP009257.1 | 4131167 | 4132804 | − | 1638 | 166 |
| IX87_RS11800 | NZ_CP009257.1 | 2333344 | 2333649 | − | 306 | 165 |
| IX87_RS18190 | NZ_CP009257.1 | 3656538 | 3657695 | − | 1158 | 165 |
| IX87_RS09250 | NZ_CP009257.1 | 1804710 | 1805273 | + | 564 | 163 |
| IX87_RS09200 | NZ_CP009257.1 | 1791383 | 1795216 | − | 3834 | 161 |
| IX87_RS14515 | NZ_CP009257.1 | 2885960 | 2886682 | − | 723 | 161 |
| IX87_RS15660 | NZ_CP009257.1 | 3121485 | 3121856 | + | 372 | 161 |
| IX87_RS17120 | NZ_CP009257.1 | 3438324 | 3440246 | + | 1923 | 161 |
| IX87_RS10970 | NZ_CP009257.1 | 2174737 | 2175633 | − | 897 | 158 |
| IX87_RS04625 | NZ_CP009257.1 | 901999 | 903318 | − | 1320 | 156 |
| IX87_RS07245 | NZ_CP009257.1 | 1411252 | 1412118 | + | 867 | 156 |
| IX87_RS14125 | NZ_CP009257.1 | 2808766 | 2811234 | − | 2469 | 156 |
| IX87_RS15020 | NZ_CP009257.1 | 2999691 | 2999936 | + | 246 | 155 |
| IX87_RS01765 | NZ_CP009257.1 | 332121 | 332495 | − | 375 | 154 |
| IX87_RS06110 | NZ_CP009257.1 | 1173040 | 1174029 | + | 990 | 154 |
| IX87_RS22850 | NZ_CP009257.1 | 2073253 | 2073426 | − | 174 | 154 |
| IX87_RS12310 | NZ_CP009257.1 | 2429566 | 2429934 | − | 369 | 154 |
| IX87_RS12535 | NZ_CP009257.1 | 2476050 | 2478605 | + | 2556 | 154 |
| IX87_RS17755 | NZ_CP009257.1 | 3569492 | 3570001 | + | 510 | 154 |
| IX87_RS05460 | NZ_CP009257.1 | 1057857 | 1059215 | + | 1359 | 152 |
| IX87_RS14520 | NZ_CP009257.1 | 2886874 | 2889060 | − | 2187 | 152 |
| IX87_RS16435 | NZ_CP009257.1 | 3292673 | 3295309 | + | 2637 | 151 |
| IX87_RS01705 | NZ_CP009257.1 | 317960 | 320992 | − | 3033 | 149 |
| IX87_RS14765 | NZ_CP009257.1 | 2943766 | 2945205 | + | 1440 | 149 |
| IX87_RS15050 | NZ_CP009257.1 | 3004975 | 3005394 | + | 420 | 149 |
| IX87_RS08830 | NZ_CP009257.1 | 1719220 | 1720476 | − | 1257 | 148 |
| IX87_RS13320 | NZ_CP009257.1 | 2635049 | 2636515 | − | 1467 | 148 |
| IX87_RS13675 | NZ_CP009257.1 | 2711283 | 2711762 | − | 480 | 148 |
| IX87_RS17125 | NZ_CP009257.1 | 3440252 | 3440803 | + | 552 | 148 |
| IX87_RS09455 | NZ_CP009257.1 | 1848695 | 1850101 | − | 1407 | 147 |
| IX87_RS03755 | NZ_CP009257.1 | 721468 | 722127 | + | 660 | 146 |
| IX87_RS15960 | NZ_CP009257.1 | 3183759 | 3186608 | − | 2850 | 146 |
| IX87_RS04610 | NZ_CP009257.1 | 899555 | 900604 | + | 1050 | 145 |
| IX87_RS16625 | NZ_CP009257.1 | 3326659 | 3327201 | − | 543 | 143 |
| IX87_RS10500 | NZ_CP009257.1 | 2077034 | 2078071 | − | 1038 | 142 |
| IX87_RS20465 | NZ_CP009257.1 | 4110862 | 4111329 | − | 468 | 141 |
| IX87_RS02175 | NZ_CP009257.1 | 402701 | 403387 | − | 687 | 140 |
| IX87_RS16640 | NZ_CP009257.1 | 3329080 | 3330777 | − | 1698 | 140 |
| IX87_RS15710 | NZ_CP009257.1 | 3136243 | 3138162 | + | 1920 | 139 |
| IX87_RS05995 | NZ_CP009257.1 | 1145226 | 1146089 | − | 864 | 138 |
| IX87_RS08615 | NZ_CP009257.1 | 1669829 | 1670977 | + | 1149 | 138 |
| IX87_RS14975 | NZ_CP009257.1 | 2991571 | 2993268 | + | 1698 | 138 |
| IX87_RS06660 | NZ_CP009257.1 | 1295286 | 1295906 | + | 621 | 137 |
| IX87_RS03320 | NZ_CP009257.1 | 620495 | 622096 | + | 1602 | 135 |
| IX87_RS15025 | NZ_CP009257.1 | 2999975 | 3000445 | + | 471 | 135 |
| IX87_RS06965 | NZ_CP009257.1 | 1353187 | 1354422 | − | 1236 | 134 |
| IX87_RS15430 | NZ_CP009257.1 | 3080125 | 3081264 | − | 1140 | 134 |
| IX87_RS16720 | NZ_CP009257.1 | 3352250 | 3353515 | − | 1266 | 134 |
| IX87_RS18795 | NZ_CP009257.1 | 3772739 | 3773848 | − | 1110 | 134 |
| IX87_RS17060 | NZ_CP009257.1 | 3426348 | 3428654 | + | 2307 | 133 |
| IX87_RS06730 | NZ_CP009257.1 | 1308625 | 1308855 | + | 231 | 132 |
| IX87_RS08705 | NZ_CP009257.1 | 1690822 | 1692354 | − | 1533 | 132 |
| IX87_RS14295 | NZ_CP009257.1 | 2842908 | 2843462 | − | 555 | 131 |
| IX87_RS16550 | NZ_CP009257.1 | 3312463 | 3312984 | − | 522 | 131 |
| IX87_RS10930 | NZ_CP009257.1 | 2164914 | 2166224 | + | 1311 | 130 |
| IX87_RS02445 | NZ_CP009257.1 | 452115 | 454103 | − | 1989 | 129 |
| IX87_RS09640 | NZ_CP009257.1 | 1889690 | 1889980 | − | 291 | 129 |
| IX87_RS12080 | NZ_CP009257.1 | 2383005 | 2385074 | + | 2070 | 129 |
| IX87_RS13095 | NZ_CP009257.1 | 2583282 | 2583908 | + | 627 | 129 |
| IX87_RS17885 | NZ_CP009257.1 | 3596806 | 3597306 | + | 501 | 129 |
| IX87_RS02300 | NZ_CP009257.1 | 423815 | 425125 | + | 1311 | 128 |
| IX87_RS06470 | NZ_CP009257.1 | 1249154 | 1251829 | − | 2676 | 127 |
| IX87_RS16115 | NZ_CP009257.1 | 3227602 | 3229314 | − | 1713 | 126 |
| IX87_RS04995 | NZ_CP009257.1 | 956206 | 957486 | + | 1281 | 125 |
| IX87_RS14760 | NZ_CP009257.1 | 2943286 | 2943603 | + | 318 | 125 |
| IX87_RS14980 | NZ_CP009257.1 | 2993314 | 2994234 | − | 921 | 124 |
| IX87_RS17630 | NZ_CP009257.1 | 3539555 | 3546541 | + | 6987 | 124 |
| IX87_RS09875 | NZ_CP009257.1 | 1963584 | 1964294 | − | 711 | 123 |
| IX87_RS10795 | NZ_CP009257.1 | 2135729 | 2137564 | − | 1836 | 123 |
| IX87_RS14655 | NZ_CP009257.1 | 2918661 | 2919875 | − | 1215 | 123 |
| IX87_RS15160 | NZ_CP009257.1 | 3026335 | 3027261 | + | 927 | 123 |
| IX87_RS15275 | NZ_CP009257.1 | 3050378 | 3050905 | + | 528 | 123 |
| IX87_RS00435 | NZ_CP009257.1 | 61525 | 62004 | + | 480 | 122 |
| IX87_RS07295 | NZ_CP009257.1 | 1419309 | 1421579 | − | 2271 | 122 |
| IX87_RS08590 | NZ_CP009257.1 | 1665915 | 1666844 | − | 930 | 122 |
| IX87_RS10455 | NZ_CP009257.1 | 2068632 | 2070062 | + | 1431 | 122 |
| IX87_RS12380 | NZ_CP009257.1 | 2441953 | 2444058 | + | 2106 | 121 |
| IX87_RS13690 | NZ_CP009257.1 | 2714044 | 2715351 | + | 1308 | 121 |
| IX87_RS20550 | NZ_CP009257.1 | 4128943 | 4130232 | − | 1290 | 121 |
| IX87_RS04605 | NZ_CP009257.1 | 898687 | 899394 | + | 708 | 120 |
| IX87_RS09590 | NZ_CP009257.1 | 1877109 | 1877492 | − | 384 | 120 |
| IX87_RS09885 | NZ_CP009257.1 | 1966157 | 1966522 | − | 366 | 120 |
| IX87_RS13390 | NZ_CP009257.1 | 2651132 | 2652394 | − | 1263 | 120 |
| IX87_RS14100 | NZ_CP009257.1 | 2802986 | 2803993 | − | 1008 | 120 |
| IX87_RS16630 | NZ_CP009257.1 | 3327243 | 3327836 | − | 594 | 119 |
| IX87_RS04640 | NZ_CP009257.1 | 906120 | 908756 | + | 2637 | 118 |
| IX87_RS06680 | NZ_CP009257.1 | 1297704 | 1297931 | + | 228 | 118 |
| IX87_RS07400 | NZ_CP009257.1 | 1444132 | 1445502 | + | 1371 | 118 |
| IX87_RS15060 | NZ_CP009257.1 | 3006201 | 3006746 | − | 546 | 118 |
| IX87_RS10515 | NZ_CP009257.1 | 2080054 | 2081523 | + | 1470 | 117 |
| IX87_RS13085 | NZ_CP009257.1 | 2580394 | 2581731 | − | 1338 | 117 |
| IX87_RS08565 | NZ_CP009257.1 | 1658954 | 1659178 | − | 225 | 116 |
| IX87_RS08885 | NZ_CP009257.1 | 1734034 | 1735878 | + | 1845 | 116 |
| IX87_RS16810 | NZ_CP009257.1 | 3372292 | 3372750 | − | 459 | 116 |
| IX87_RS04195 | NZ_CP009257.1 | 817667 | 818509 | − | 843 | 115 |
| IX87_RS11860 | NZ_CP009257.1 | 2338404 | 2339006 | − | 603 | 114 |
| IX87_RS14755 | NZ_CP009257.1 | 2942130 | 2943233 | + | 1104 | 114 |
| IX87_RS17195 | NZ_CP009257.1 | 3451435 | 3452703 | − | 1269 | 114 |
| IX87_RS18000 | NZ_CP009257.1 | 3624857 | 3625321 | + | 465 | 114 |
| IX87_RS18630 | NZ_CP009257.1 | 3734930 | 3736147 | − | 1218 | 114 |
| IX87_RS09330 | NZ_CP009257.1 | 1819197 | 1820213 | − | 1017 | 113 |
| IX87_RS15235 | NZ_CP009257.1 | 3040750 | 3042342 | + | 1593 | 113 |
| IX87_RS17080 | NZ_CP009257.1 | 3430924 | 3432357 | + | 1434 | 113 |
| IX87_RS21350 | NZ_CP009257.1 | 4274918 | 4275925 | − | 1008 | 113 |
| IX87_RS03330 | NZ_CP009257.1 | 622921 | 624912 | + | 1992 | 112 |
| IX87_RS08795 | NZ_CP009257.1 | 1710271 | 1713033 | − | 2763 | 112 |
| IX87_RS10715 | NZ_CP009257.1 | 2121848 | 2123563 | + | 1716 | 112 |
| IX87_RS11810 | NZ_CP009257.1 | 2334211 | 2334528 | − | 318 | 112 |
| IX87_RS11970 | NZ_CP009257.1 | 2360310 | 2360990 | − | 681 | 112 |
| IX87_RS16710 | NZ_CP009257.1 | 3349831 | 3350343 | + | 513 | 112 |
| IX87_RS01795 | NZ_CP009257.1 | 338085 | 339710 | + | 1626 | 111 |
| IX87_RS06755 | NZ_CP009257.1 | 1313209 | 1314495 | + | 1287 | 110 |
| IX87_RS07940 | NZ_CP009257.1 | 1541978 | 1543780 | + | 1803 | 110 |
| IX87_RS00445 | NZ_CP009257.1 | 64260 | 65249 | + | 990 | 109 |
| IX87_RS21500 | NZ_CP009257.1 | 4303312 | 4303731 | + | 420 | 109 |
| IX87_RS10720 | NZ_CP009257.1 | 2123617 | 2123958 | − | 342 | 108 |
| IX87_RS00360 | NZ_CP009257.1 | 49127 | 49789 | − | 663 | 107 |
| IX87_RS13140 | NZ_CP009257.1 | 2592881 | 2593462 | − | 582 | 107 |
| IX87_RS16705 | NZ_CP009257.1 | 3348455 | 3349813 | + | 1359 | 107 |
| IX87_RS17740 | NZ_CP009257.1 | 3566443 | 3566922 | + | 480 | 107 |
| IX87_RS18875 | NZ_CP009257.1 | 3790043 | 3790456 | − | 414 | 107 |
| IX87_RS06760 | NZ_CP009257.1 | 1314745 | 1315611 | + | 867 | 106 |
| IX87_RS08595 | NZ_CP009257.1 | 1666946 | 1667776 | − | 831 | 106 |
| IX87_RS10595 | NZ_CP009257.1 | 2096829 | 2099003 | + | 2175 | 106 |
| IX87_RS11785 | NZ_CP009257.1 | 2332025 | 2332375 | − | 351 | 106 |
| IX87_RS14270 | NZ_CP009257.1 | 2836727 | 2837935 | + | 1209 | 106 |
| IX87_RS00465 | NZ_CP009257.1 | 68138 | 69475 | + | 1338 | 105 |
| IX87_RS17150 | NZ_CP009257.1 | 3443856 | 3444215 | + | 360 | 105 |
| IX87_RS14035 | NZ_CP009257.1 | 2787999 | 2789477 | − | 1479 | 104 |
| IX87_RS15840 | NZ_CP009257.1 | 3162682 | 3163236 | − | 555 | 104 |
| IX87_RS17275 | NZ_CP009257.1 | 3470263 | 3470784 | − | 522 | 104 |
| IX87_RS18080 | NZ_CP009257.1 | 3639240 | 3640226 | + | 987 | 103 |
| IX87_RS09625 | NZ_CP009257.1 | 1886378 | 1887340 | − | 963 | 102 |
| IX87_RS10060 | NZ_CP009257.1 | 1999726 | 2002605 | + | 2880 | 102 |
| IX87_RS02755 | NZ_CP009257.1 | 505355 | 505744 | + | 390 | 101 |
| IX87_RS09155 | NZ_CP009257.1 | 1785453 | 1785869 | + | 417 | 101 |
| IX87_RS13485 | NZ_CP009257.1 | 2670961 | 2672775 | − | 1815 | 101 |
| IX87_RS04010 | NZ_CP009257.1 | 775754 | 777007 | − | 1254 | 100 |
| IX87_RS11305 | NZ_CP009257.1 | 2242018 | 2242506 | + | 489 | 100 |
| IX87_RS15260 | NZ_CP009257.1 | 3047644 | 3049323 | + | 1680 | 100 |
| IX87_RS21125 | NZ_CP009257.1 | 4231824 | 4233266 | − | 1443 | 100 |
| IX87_RS08640 | NZ_CP009257.1 | 1674171 | 1675559 | + | 1389 | 99 |
| IX87_RS09310 | NZ_CP009257.1 | 1815280 | 1816473 | − | 1194 | 99 |
| IX87_RS11750 | NZ_CP009257.1 | 2328526 | 2328912 | − | 387 | 99 |
| IX87_RS17600 | NZ_CP009257.1 | 3533883 | 3534926 | + | 1044 | 99 |
| IX87_RS02125 | NZ_CP009257.1 | 393451 | 394593 | − | 1143 | 98 |
| IX87_RS02760 | NZ_CP009257.1 | 505846 | 507063 | + | 1218 | 98 |
| IX87_RS02805 | NZ_CP009257.1 | 517579 | 518646 | + | 1068 | 98 |
| IX87_RS09735 | NZ_CP009257.1 | 1917851 | 1918501 | − | 651 | 98 |
| IX87_RS12000 | NZ_CP009257.1 | 2366196 | 2368100 | − | 1905 | 98 |
| IX87_RS09140 | NZ_CP009257.1 | 1783117 | 1783938 | + | 822 | 97 |
| IX87_RS11575 | NZ_CP009257.1 | 2293388 | 2295331 | − | 1944 | 97 |
| IX87_RS18135 | NZ_CP009257.1 | 3648181 | 3648477 | − | 297 | 96 |
| IX87_RS00670 | NZ_CP009257.1 | 116504 | 117649 | + | 1146 | 95 |
| IX87_RS11175 | NZ_CP009257.1 | 2217003 | 2219753 | − | 2751 | 95 |
| IX87_RS13400 | NZ_CP009257.1 | 2653312 | 2654238 | − | 927 | 95 |
| IX87_RS13890 | NZ_CP009257.1 | 2756711 | 2757448 | + | 738 | 95 |
| IX87_RS14675 | NZ_CP009257.1 | 2923401 | 2926007 | + | 2607 | 95 |
| IX87_RS00485 | NZ_CP009257.1 | 72501 | 73409 | − | 909 | 94 |
| IX87_RS14625 | NZ_CP009257.1 | 2910229 | 2911245 | + | 1017 | 93 |
| IX87_RS14930 | NZ_CP009257.1 | 2982645 | 2984213 | + | 1569 | 93 |
| IX87_RS17745 | NZ_CP009257.1 | 3566929 | 3567606 | + | 678 | 93 |
| IX87_RS01680 | NZ_CP009257.1 | 314924 | 315619 | + | 696 | 92 |
| IX87_RS10010 | NZ_CP009257.1 | 1987843 | 1989903 | + | 2061 | 92 |
| IX87_RS11885 | NZ_CP009257.1 | 2342407 | 2343954 | + | 1548 | 92 |
| IX87_RS12385 | NZ_CP009257.1 | 2444179 | 2444562 | + | 384 | 92 |
| IX87_RS12590 | NZ_CP009257.1 | 2489010 | 2490518 | + | 1509 | 92 |
| IX87_RS14670 | NZ_CP009257.1 | 2922244 | 2923401 | + | 1158 | 92 |
| IX87_RS14970 | NZ_CP009257.1 | 2989294 | 2991084 | − | 1791 | 92 |
| IX87_RS18090 | NZ_CP009257.1 | 3641042 | 3641278 | + | 237 | 92 |
| IX87_RS09245 | NZ_CP009257.1 | 1803402 | 1804682 | + | 1281 | 91 |
| IX87_RS13470 | NZ_CP009257.1 | 2668102 | 2668539 | − | 438 | 91 |
| IX87_RS14650 | NZ_CP009257.1 | 2916882 | 2918612 | + | 1731 | 91 |
| IX87_RS17780 | NZ_CP009257.1 | 3575606 | 3576124 | + | 519 | 90 |
| IX87_RS06095 | NZ_CP009257.1 | 1169856 | 1171424 | + | 1569 | 89 |
| IX87_RS06675 | NZ_CP009257.1 | 1297309 | 1297692 | + | 384 | 89 |
| IX87_RS09680 | NZ_CP009257.1 | 1909301 | 1909723 | + | 423 | 89 |
| IX87_RS20555 | NZ_CP009257.1 | 4130278 | 4131135 | − | 858 | 89 |
| IX87_RS00490 | NZ_CP009257.1 | 73790 | 75232 | − | 1443 | 88 |
| IX87_RS01650 | NZ_CP009257.1 | 307560 | 308978 | − | 1419 | 88 |
| IX87_RS05485 | NZ_CP009257.1 | 1061573 | 1062607 | + | 1035 | 88 |
| IX87_RS06345 | NZ_CP009257.1 | 1222538 | 1224265 | − | 1728 | 88 |
| IX87_RS08975 | NZ_CP009257.1 | 1752635 | 1754656 | − | 2022 | 88 |
| IX87_RS09075 | NZ_CP009257.1 | 1769856 | 1771247 | − | 1392 | 88 |
| IX87_RS10875 | NZ_CP009257.1 | 2155164 | 2156321 | + | 1158 | 88 |
| IX87_RS14360 | NZ_CP009257.1 | 2854844 | 2855386 | − | 543 | 88 |
| IX87_RS15480 | NZ_CP009257.1 | 3090991 | 3091527 | − | 537 | 88 |
| IX87_RS16010 | NZ_CP009257.1 | 3197970 | 3198476 | − | 507 | 88 |
| IX87_RS17595 | NZ_CP009257.1 | 3532875 | 3533861 | + | 987 | 88 |
| IX87_RS20650 | NZ_CP009257.1 | 4150285 | 4151832 | + | 1548 | 88 |
| IX87_RS11790 | NZ_CP009257.1 | 2332390 | 2332923 | − | 534 | 87 |
| IX87_RS17770 | NZ_CP009257.1 | 3574029 | 3575045 | + | 1017 | 87 |
| IX87_RS04490 | NZ_CP009257.1 | 871270 | 875127 | − | 3858 | 86 |
| IX87_RS12035 | NZ_CP009257.1 | 2374539 | 2376362 | + | 1824 | 86 |
| IX87_RS17020 | NZ_CP009257.1 | 3417125 | 3417478 | − | 354 | 86 |
| IX87_RS18560 | NZ_CP009257.1 | 3721481 | 3721747 | − | 267 | 86 |
| IX87_RS07820 | NZ_CP009257.1 | 1514528 | 1515940 | − | 1413 | 85 |
| IX87_RS08715 | NZ_CP009257.1 | 1694198 | 1694899 | + | 702 | 85 |
| IX87_RS10855 | NZ_CP009257.1 | 2148205 | 2149848 | − | 1644 | 85 |
| IX87_RS16690 | NZ_CP009257.1 | 3343800 | 3345002 | − | 1203 | 85 |
| IX87_RS09840 | NZ_CP009257.1 | 1952706 | 1953629 | − | 924 | 84 |
| IX87_RS11255 | NZ_CP009257.1 | 2233251 | 2234216 | + | 966 | 84 |
| IX87_RS16485 | NZ_CP009257.1 | 3302680 | 3302916 | − | 237 | 84 |
| IX87_RS21235 | NZ_CP009257.1 | 4251794 | 4252297 | − | 504 | 84 |
| IX87_RS05000 | NZ_CP009257.1 | 957733 | 957987 | + | 255 | 83 |
| IX87_RS09675 | NZ_CP009257.1 | 1907853 | 1909184 | + | 1332 | 83 |
| IX87_RS10520 | NZ_CP009257.1 | 2081711 | 2082271 | + | 561 | 83 |
| IX87_RS11840 | NZ_CP009257.1 | 2336621 | 2336950 | − | 330 | 83 |
| IX87_RS02810 | NZ_CP009257.1 | 518862 | 522425 | + | 3564 | 82 |
| IX87_RS11060 | NZ_CP009257.1 | 2194514 | 2196295 | + | 1782 | 82 |
| IX87_RS13750 | NZ_CP009257.1 | 2726964 | 2728169 | − | 1206 | 82 |
| IX87_RS20670 | NZ_CP009257.1 | 4155197 | 4158151 | − | 2955 | 82 |
| IX87_RS03675 | NZ_CP009257.1 | 702370 | 704472 | + | 2103 | 81 |
| IX87_RS06540 | NZ_CP009257.1 | 1266239 | 1267537 | − | 1299 | 81 |
| IX87_RS07215 | NZ_CP009257.1 | 1406727 | 1407800 | − | 1074 | 81 |
| IX87_RS09365 | NZ_CP009257.1 | 1827626 | 1828567 | + | 942 | 81 |
| IX87_RS15385 | NZ_CP009257.1 | 3071159 | 3072706 | + | 1548 | 81 |
| IX87_RS15950 | NZ_CP009257.1 | 3181583 | 3183394 | − | 1812 | 81 |
| IX87_RS17285 | NZ_CP009257.1 | 3471971 | 3472720 | − | 750 | 81 |
| IX87_RS00525 | NZ_CP009257.1 | 79790 | 80971 | + | 1182 | 80 |
| IX87_RS12315 | NZ_CP009257.1 | 2430142 | 2430882 | − | 741 | 80 |
| IX87_RS12565 | NZ_CP009257.1 | 2483874 | 2485706 | − | 1833 | 80 |
| IX87_RS17175 | NZ_CP009257.1 | 3446916 | 3447896 | + | 981 | 80 |
| IX87_RS18085 | NZ_CP009257.1 | 3640223 | 3640957 | + | 735 | 80 |
| IX87_RS22975 | NZ_CP009257.1 | 3898396 | 3898572 | + | 177 | 80 |
| IX87_RS06045 | NZ_CP009257.1 | 1155722 | 1156966 | − | 1245 | 79 |
| IX87_RS09940 | NZ_CP009257.1 | 1975763 | 1976947 | + | 1185 | 79 |
| IX87_RS09975 | NZ_CP009257.1 | 1983125 | 1983913 | − | 789 | 79 |
| IX87_RS13620 | NZ_CP009257.1 | 2699364 | 2700518 | + | 1155 | 79 |
| IX87_RS14015 | NZ_CP009257.1 | 2783359 | 2785188 | − | 1830 | 79 |
| IX87_RS17190 | NZ_CP009257.1 | 3450774 | 3451019 | − | 246 | 79 |
| IX87_RS21225 | NZ_CP009257.1 | 4250227 | 4250712 | − | 486 | 79 |
| IX87_RS01625 | NZ_CP009257.1 | 302849 | 303916 | − | 1068 | 78 |
| IX87_RS09510 | NZ_CP009257.1 | 1862092 | 1862697 | + | 606 | 78 |
| IX87_RS02660 | NZ_CP009257.1 | 488779 | 489621 | − | 843 | 77 |
| IX87_RS07825 | NZ_CP009257.1 | 1516122 | 1517150 | + | 1029 | 77 |
| IX87_RS12415 | NZ_CP009257.1 | 2448945 | 2450027 | − | 1083 | 77 |
| IX87_RS00620 | NZ_CP009257.1 | 101838 | 103385 | + | 1548 | 76 |
| IX87_RS06670 | NZ_CP009257.1 | 1296246 | 1297124 | + | 879 | 76 |
| IX87_RS09205 | NZ_CP009257.1 | 1795526 | 1796866 | + | 1341 | 76 |
| IX87_RS13105 | NZ_CP009257.1 | 2585008 | 2586456 | − | 1449 | 76 |
| IX87_RS17210 | NZ_CP009257.1 | 3455170 | 3455991 | + | 822 | 76 |
| IX87_RS20565 | NZ_CP009257.1 | 4133028 | 4133516 | + | 489 | 76 |
| IX87_RS02765 | NZ_CP009257.1 | 507091 | 509754 | + | 2664 | 75 |
| IX87_RS17040 | NZ_CP009257.1 | 3420181 | 3422988 | − | 2808 | 75 |
| IX87_RS17775 | NZ_CP009257.1 | 3575064 | 3575606 | + | 543 | 75 |
| IX87_RS21215 | NZ_CP009257.1 | 4248502 | 4249386 | + | 885 | 75 |
| IX87_RS05445 | NZ_CP009257.1 | 1054064 | 1054942 | − | 879 | 74 |
| IX87_RS05620 | NZ_CP009257.1 | 1091422 | 1091982 | + | 561 | 74 |
| IX87_RS08700 | NZ_CP009257.1 | 1689115 | 1690545 | − | 1431 | 74 |
| IX87_RS08845 | NZ_CP009257.1 | 1724221 | 1725924 | − | 1704 | 74 |
| IX87_RS21265 | NZ_CP009257.1 | 4259038 | 4259592 | − | 555 | 74 |
| IX87_RS02795 | NZ_CP009257.1 | 513691 | 515121 | + | 1431 | 73 |
| IX87_RS13405 | NZ_CP009257.1 | 2654276 | 2655724 | − | 1449 | 73 |
| IX87_RS00535 | NZ_CP009257.1 | 81977 | 82885 | + | 909 | 72 |
| IX87_RS02395 | NZ_CP009257.1 | 441109 | 442599 | + | 1491 | 72 |
| IX87_RS05555 | NZ_CP009257.1 | 1080881 | 1081588 | − | 708 | 72 |
| IX87_RS09970 | NZ_CP009257.1 | 1981735 | 1983006 | − | 1272 | 72 |
| IX87_RS10470 | NZ_CP009257.1 | 2071838 | 2073223 | + | 1386 | 72 |
| IX87_RS12820 | NZ_CP009257.1 | 2532619 | 2533308 | + | 690 | 72 |
| IX87_RS17340 | NZ_CP009257.1 | 3483415 | 3484704 | + | 1290 | 72 |
| IX87_RS07750 | NZ_CP009257.1 | 1501495 | 1502496 | + | 1002 | 71 |
| IX87_RS13310 | NZ_CP009257.1 | 2632895 | 2633560 | − | 666 | 71 |
| IX87_RS13695 | NZ_CP009257.1 | 2715590 | 2716711 | + | 1122 | 71 |
| IX87_RS08750 | NZ_CP009257.1 | 1700458 | 1703295 | − | 2838 | 70 |
| IX87_RS17465 | NZ_CP009257.1 | 3509353 | 3510573 | + | 1221 | 70 |
| IX87_RS02625 | NZ_CP009257.1 | 482924 | 483484 | + | 561 | 69 |
| IX87_RS03315 | NZ_CP009257.1 | 619298 | 620470 | + | 1173 | 69 |
| IX87_RS07830 | NZ_CP009257.1 | 1517434 | 1519899 | + | 2466 | 69 |
| IX87_RS08690 | NZ_CP009257.1 | 1686702 | 1688423 | − | 1722 | 69 |
| IX87_RS10155 | NZ_CP009257.1 | 2022241 | 2023596 | + | 1356 | 69 |
| IX87_RS10945 | NZ_CP009257.1 | 2169996 | 2170637 | + | 642 | 69 |
| IX87_RS12265 | NZ_CP009257.1 | 2419952 | 2421361 | − | 1410 | 69 |
| IX87_RS13395 | NZ_CP009257.1 | 2652455 | 2653309 | − | 855 | 69 |
| IX87_RS14525 | NZ_CP009257.1 | 2889080 | 2889508 | − | 429 | 69 |
| IX87_RS18075 | NZ_CP009257.1 | 3638902 | 3639087 | + | 186 | 69 |
| IX87_RS03740 | NZ_CP009257.1 | 719517 | 719951 | − | 435 | 68 |
| IX87_RS07755 | NZ_CP009257.1 | 1502835 | 1503404 | + | 570 | 68 |
| IX87_RS09350 | NZ_CP009257.1 | 1824482 | 1825717 | − | 1236 | 68 |
| IX87_RS11780 | NZ_CP009257.1 | 2331525 | 2332022 | − | 498 | 68 |
| IX87_RS11605 | NZ_CP009257.1 | 2300717 | 2301430 | + | 714 | 67 |
| IX87_RS14440 | NZ_CP009257.1 | 2871148 | 2871894 | − | 747 | 67 |
| IX87_RS00495 | NZ_CP009257.1 | 75422 | 76984 | − | 1563 | 66 |
| IX87_RS01770 | NZ_CP009257.1 | 332696 | 333967 | − | 1272 | 66 |
| IX87_RS02295 | NZ_CP009257.1 | 421374 | 423815 | + | 2442 | 66 |
| IX87_RS12375 | NZ_CP009257.1 | 2441466 | 2441744 | + | 279 | 66 |
| IX87_RS13225 | NZ_CP009257.1 | 2612425 | 2615922 | + | 3498 | 66 |
| IX87_RS13260 | NZ_CP009257.1 | 2621249 | 2623198 | − | 1950 | 66 |
| IX87_RS15455 | NZ_CP009257.1 | 3085152 | 3087005 | + | 1854 | 66 |
| IX87_RS15470 | NZ_CP009257.1 | 3088809 | 3090005 | + | 1197 | 66 |
| IX87_RS17230 | NZ_CP009257.1 | 3458791 | 3461562 | − | 2772 | 66 |
| IX87_RS18925 | NZ_CP009257.1 | 3796144 | 3797355 | + | 1212 | 66 |
| IX87_RS03335 | NZ_CP009257.1 | 624909 | 625814 | + | 906 | 65 |
| IX87_RS12560 | NZ_CP009257.1 | 2482371 | 2483870 | − | 1500 | 65 |
| IX87_RS17100 | NZ_CP009257.1 | 3434566 | 3435417 | + | 852 | 65 |
| IX87_RS19190 | NZ_CP009257.1 | 3849925 | 3851097 | − | 1173 | 65 |
| IX87_RS10760 | NZ_CP009257.1 | 2129379 | 2130488 | − | 1110 | 64 |
| IX87_RS12020 | NZ_CP009257.1 | 2371092 | 2373395 | − | 2304 | 64 |
| IX87_RS12395 | NZ_CP009257.1 | 2445211 | 2445675 | + | 465 | 64 |
| IX87_RS16105 | NZ_CP009257.1 | 3226225 | 3227034 | − | 810 | 64 |
| IX87_RS16285 | NZ_CP009257.1 | 3263359 | 3265653 | − | 2295 | 64 |
| IX87_RS09045 | NZ_CP009257.1 | 1764296 | 1765318 | − | 1023 | 63 |
| IX87_RS09785 | NZ_CP009257.1 | 1927221 | 1927937 | + | 717 | 63 |
| IX87_RS11560 | NZ_CP009257.1 | 2291062 | 2291307 | − | 246 | 63 |
| IX87_RS12130 | NZ_CP009257.1 | 2392685 | 2393446 | + | 762 | 63 |
| IX87_RS12215 | NZ_CP009257.1 | 2410904 | 2411479 | + | 576 | 63 |
| IX87_RS12845 | NZ_CP009257.1 | 2536305 | 2538182 | − | 1878 | 63 |
| IX87_RS14645 | NZ_CP009257.1 | 2915463 | 2916614 | + | 1152 | 63 |
| IX87_RS15530 | NZ_CP009257.1 | 3100915 | 3102348 | − | 1434 | 63 |
| IX87_RS17880 | NZ_CP009257.1 | 3594456 | 3596519 | − | 2064 | 63 |
| IX87_RS17905 | NZ_CP009257.1 | 3600653 | 3601246 | + | 594 | 63 |
| IX87_RS17990 | NZ_CP009257.1 | 3621435 | 3622472 | + | 1038 | 63 |
| IX87_RS19455 | NZ_CP009257.1 | 3905950 | 3907644 | + | 1695 | 63 |
| IX87_RS02195 | NZ_CP009257.1 | 405539 | 406252 | − | 714 | 62 |
| IX87_RS02450 | NZ_CP009257.1 | 454627 | 455793 | + | 1167 | 62 |
| IX87_RS06220 | NZ_CP009257.1 | 1193517 | 1194752 | − | 1236 | 62 |
| IX87_RS09925 | NZ_CP009257.1 | 1974023 | 1974310 | + | 288 | 62 |
| IX87_RS11180 | NZ_CP009257.1 | 2219822 | 2221090 | − | 1269 | 62 |
| IX87_RS16475 | NZ_CP009257.1 | 3300654 | 3302363 | − | 1710 | 62 |
| IX87_RS16930 | NZ_CP009257.1 | 3397274 | 3398137 | + | 864 | 62 |
| IX87_RS18865 | NZ_CP009257.1 | 3787983 | 3788573 | + | 591 | 62 |
| IX87_RS02500 | NZ_CP009257.1 | 463106 | 464107 | + | 1002 | 61 |
| IX87_RS11845 | NZ_CP009257.1 | 2336962 | 2337237 | − | 276 | 61 |
| IX87_RS13180 | NZ_CP009257.1 | 2599787 | 2602621 | + | 2835 | 61 |
| IX87_RS13490 | NZ_CP009257.1 | 2672804 | 2673571 | − | 768 | 61 |
| IX87_RS16865 | NZ_CP009257.1 | 3383076 | 3383585 | − | 510 | 61 |
| IX87_RS17950 | NZ_CP009257.1 | 3612346 | 3613755 | + | 1410 | 61 |
| IX87_RS06625 | NZ_CP009257.1 | 1283074 | 1284903 | − | 1830 | 60 |
| IX87_RS08930 | NZ_CP009257.1 | 1744251 | 1745384 | − | 1134 | 60 |
| IX87_RS10710 | NZ_CP009257.1 | 2118691 | 2121789 | + | 3099 | 60 |
| IX87_RS12075 | NZ_CP009257.1 | 2382028 | 2383005 | + | 978 | 60 |
| IX87_RS13680 | NZ_CP009257.1 | 2712016 | 2712351 | − | 336 | 60 |
| IX87_RS15640 | NZ_CP009257.1 | 3118847 | 3119380 | + | 534 | 60 |
| IX87_RS15965 | NZ_CP009257.1 | 3186923 | 3188305 | − | 1383 | 60 |
| IX87_RS16100 | NZ_CP009257.1 | 3224669 | 3226228 | − | 1560 | 60 |
| IX87_RS16390 | NZ_CP009257.1 | 3283775 | 3285061 | + | 1287 | 60 |
| IX87_RS16820 | NZ_CP009257.1 | 3373047 | 3373463 | − | 417 | 60 |
| IX87_RS03995 | NZ_CP009257.1 | 772521 | 774104 | − | 1584 | 59 |
| IX87_RS09315 | NZ_CP009257.1 | 1816609 | 1817679 | + | 1071 | 59 |
| IX87_RS16795 | NZ_CP009257.1 | 3368708 | 3369853 | + | 1146 | 59 |
| IX87_RS18880 | NZ_CP009257.1 | 3790475 | 3790744 | − | 270 | 59 |
| IX87_RS06310 | NZ_CP009257.1 | 1213531 | 1214499 | + | 969 | 58 |
| IX87_RS06665 | NZ_CP009257.1 | 1295906 | 1296235 | + | 330 | 58 |
| IX87_RS13650 | NZ_CP009257.1 | 2706154 | 2707296 | − | 1143 | 58 |
| IX87_RS14245 | NZ_CP009257.1 | 2832150 | 2833523 | − | 1374 | 58 |
| IX87_RS17330 | NZ_CP009257.1 | 3481370 | 3482626 | + | 1257 | 58 |
| IX87_RS17430 | NZ_CP009257.1 | 3503197 | 3504018 | + | 822 | 58 |
| IX87_RS20825 | NZ_CP009257.1 | 4182393 | 4185251 | + | 2859 | 58 |
| IX87_RS21285 | NZ_CP009257.1 | 4263239 | 4264732 | + | 1494 | 58 |
| IX87_RS00865 | NZ_CP009257.1 | 158091 | 159731 | + | 1641 | 57 |
| IX87_RS07300 | NZ_CP009257.1 | 1421664 | 1422773 | − | 1110 | 57 |
| IX87_RS09270 | NZ_CP009257.1 | 1807663 | 1808277 | + | 615 | 57 |
| IX87_RS10135 | NZ_CP009257.1 | 2018253 | 2019107 | + | 855 | 57 |
| IX87_RS13700 | NZ_CP009257.1 | 2716723 | 2717193 | + | 471 | 57 |
| IX87_RS15490 | NZ_CP009257.1 | 3092555 | 3093181 | − | 627 | 57 |
| IX87_RS16330 | NZ_CP009257.1 | 3271712 | 3272302 | + | 591 | 57 |
| IX87_RS20805 | NZ_CP009257.1 | 4179757 | 4180176 | + | 420 | 57 |
| IX87_RS00825 | NZ_CP009257.1 | 147950 | 148150 | + | 201 | 56 |
| IX87_RS06785 | NZ_CP009257.1 | 1319089 | 1320387 | − | 1299 | 56 |
| IX87_RS11700 | NZ_CP009257.1 | 2317849 | 2320044 | − | 2196 | 56 |
| IX87_RS11825 | NZ_CP009257.1 | 2335253 | 2335450 | − | 198 | 56 |
| IX87_RS11880 | NZ_CP009257.1 | 2341173 | 2342363 | + | 1191 | 56 |
| IX87_RS13145 | NZ_CP009257.1 | 2593514 | 2594878 | − | 1365 | 56 |
| IX87_RS15570 | NZ_CP009257.1 | 3108763 | 3109089 | + | 327 | 56 |
| IX87_RS15985 | NZ_CP009257.1 | 3192206 | 3193825 | − | 1620 | 56 |
| IX87_RS03325 | NZ_CP009257.1 | 622106 | 622909 | + | 804 | 55 |
| IX87_RS06020 | NZ_CP009257.1 | 1150241 | 1151659 | + | 1419 | 55 |
| IX87_RS06070 | NZ_CP009257.1 | 1163738 | 1165216 | − | 1479 | 55 |
| IX87_RS13965 | NZ_CP009257.1 | 2768781 | 2769893 | − | 1113 | 55 |
| IX87_RS15100 | NZ_CP009257.1 | 3014121 | 3014651 | + | 531 | 55 |
| IX87_RS15485 | NZ_CP009257.1 | 3091738 | 3092550 | − | 813 | 55 |
| IX87_RS17280 | NZ_CP009257.1 | 3470887 | 3471855 | − | 969 | 55 |
| IX87_RS18520 | NZ_CP009257.1 | 3712399 | 3714432 | + | 2034 | 55 |
| IX87_RS18625 | NZ_CP009257.1 | 3734477 | 3734863 | − | 387 | 55 |
| IX87_RS19380 | NZ_CP009257.1 | 3891187 | 3893262 | + | 2076 | 55 |
| IX87_RS00450 | NZ_CP009257.1 | 65373 | 66698 | + | 1326 | 54 |
| IX87_RS04485 | NZ_CP009257.1 | 870069 | 871175 | − | 1107 | 54 |
| IX87_RS08310 | NZ_CP009257.1 | 1594313 | 1597078 | − | 2766 | 54 |
| IX87_RS08935 | NZ_CP009257.1 | 1745400 | 1746620 | − | 1221 | 54 |
| IX87_RS09150 | NZ_CP009257.1 | 1784756 | 1785439 | + | 684 | 54 |
| IX87_RS12550 | NZ_CP009257.1 | 2479843 | 2480961 | − | 1119 | 54 |
| IX87_RS12760 | NZ_CP009257.1 | 2523736 | 2524284 | + | 549 | 54 |
| IX87_RS13410 | NZ_CP009257.1 | 2655736 | 2656833 | − | 1098 | 54 |
| IX87_RS13855 | NZ_CP009257.1 | 2750820 | 2751539 | + | 720 | 54 |
| IX87_RS17090 | NZ_CP009257.1 | 3432882 | 3433691 | + | 810 | 54 |
| IX87_RS17110 | NZ_CP009257.1 | 3435686 | 3436222 | + | 537 | 54 |
| IX87_RS17785 | NZ_CP009257.1 | 3576124 | 3576432 | + | 309 | 54 |
| IX87_RS02190 | NZ_CP009257.1 | 404418 | 405542 | − | 1125 | 53 |
| IX87_RS04000 | NZ_CP009257.1 | 774137 | 774943 | − | 807 | 53 |
| IX87_RS06090 | NZ_CP009257.1 | 1168229 | 1169620 | − | 1392 | 53 |
| IX87_RS02770 | NZ_CP009257.1 | 509780 | 510949 | + | 1170 | 52 |
| IX87_RS11455 | NZ_CP009257.1 | 2268707 | 2269363 | + | 657 | 52 |
| IX87_RS12145 | NZ_CP009257.1 | 2395858 | 2397327 | − | 1470 | 52 |
| IX87_RS14395 | NZ_CP009257.1 | 2862452 | 2863072 | − | 621 | 52 |
| IX87_RS14510 | NZ_CP009257.1 | 2885213 | 2885920 | − | 708 | 52 |
| IX87_RS16735 | NZ_CP009257.1 | 3356349 | 3357368 | − | 1020 | 52 |
| IX87_RS17235 | NZ_CP009257.1 | 3461717 | 3464524 | + | 2808 | 52 |
| IX87_RS21245 | NZ_CP009257.1 | 4254902 | 4256257 | − | 1356 | 52 |
| IX87_RS00575 | NZ_CP009257.1 | 91348 | 91986 | − | 639 | 51 |
| IX87_RS02650 | NZ_CP009257.1 | 487342 | 488085 | − | 744 | 51 |
| IX87_RS03305 | NZ_CP009257.1 | 616730 | 618424 | − | 1695 | 51 |
| IX87_RS04005 | NZ_CP009257.1 | 774955 | 775719 | − | 765 | 51 |
| IX87_RS04230 | NZ_CP009257.1 | 825773 | 826321 | − | 549 | 51 |
| IX87_RS10745 | NZ_CP009257.1 | 2126638 | 2127348 | + | 711 | 51 |
| IX87_RS12155 | NZ_CP009257.1 | 2398377 | 2399591 | − | 1215 | 51 |
| IX87_RS14640 | NZ_CP009257.1 | 2914714 | 2915466 | + | 753 | 51 |
| IX87_RS16785 | NZ_CP009257.1 | 3366689 | 3367981 | + | 1293 | 51 |
| IX87_RS21495 | NZ_CP009257.1 | 4302839 | 4303294 | + | 456 | 51 |
| IX87_RS02200 | NZ_CP009257.1 | 406303 | 406899 | − | 597 | 50 |
| IX87_RS07160 | NZ_CP009257.1 | 1395507 | 1395953 | + | 447 | 50 |
| IX87_RS07185 | NZ_CP009257.1 | 1401319 | 1402860 | − | 1542 | 50 |
| IX87_RS11960 | NZ_CP009257.1 | 2359343 | 2359630 | − | 288 | 50 |
| IX87_RS11975 | NZ_CP009257.1 | 2360990 | 2361766 | − | 777 | 50 |
| IX87_RS13370 | NZ_CP009257.1 | 2647441 | 2648133 | − | 693 | 50 |
| IX87_RS17095 | NZ_CP009257.1 | 3433695 | 3434543 | + | 849 | 50 |
| IX87_RS18115 | NZ_CP009257.1 | 3644670 | 3646451 | + | 1782 | 50 |
| IX87_RS19200 | NZ_CP009257.1 | 3852689 | 3853330 | − | 642 | 50 |
| IX87_RS07315 | NZ_CP009257.1 | 1425256 | 1426614 | − | 1359 | 49 |
| IX87_RS12165 | NZ_CP009257.1 | 2402043 | 2403476 | − | 1434 | 49 |
| IX87_RS12205 | NZ_CP009257.1 | 2408903 | 2409547 | + | 645 | 49 |
| IX87_RS14770 | NZ_CP009257.1 | 2945576 | 2947006 | + | 1431 | 49 |
| IX87_RS17910 | NZ_CP009257.1 | 3601361 | 3601930 | + | 570 | 49 |
| IX87_RS20800 | NZ_CP009257.1 | 4178264 | 4179565 | + | 1302 | 49 |
| IX87_RS01670 | NZ_CP009257.1 | 312786 | 313712 | + | 927 | 48 |
| IX87_RS02655 | NZ_CP009257.1 | 488099 | 488776 | − | 678 | 48 |
| IX87_RS07385 | NZ_CP009257.1 | 1440676 | 1441455 | + | 780 | 48 |
| IX87_RS07850 | NZ_CP009257.1 | 1522637 | 1523473 | + | 837 | 48 |
| IX87_RS08375 | NZ_CP009257.1 | 1612960 | 1613430 | − | 471 | 48 |
| IX87_RS08610 | NZ_CP009257.1 | 1669280 | 1669702 | + | 423 | 48 |
| IX87_RS14965 | NZ_CP009257.1 | 2988514 | 2989140 | − | 627 | 48 |
| IX87_RS18590 | NZ_CP009257.1 | 3725600 | 3729061 | + | 3462 | 48 |
| IX87_RS19165 | NZ_CP009257.1 | 3844727 | 3846145 | − | 1419 | 48 |
| IX87_RS07170 | NZ_CP009257.1 | 1397617 | 1398582 | − | 966 | 47 |
| IX87_RS12140 | NZ_CP009257.1 | 2394497 | 2395840 | − | 1344 | 47 |
| IX87_RS15600 | NZ_CP009257.1 | 3114826 | 3116319 | + | 1494 | 47 |
| IX87_RS16815 | NZ_CP009257.1 | 3372778 | 3373035 | − | 258 | 47 |
| IX87_RS16885 | NZ_CP009257.1 | 3388982 | 3389473 | + | 492 | 47 |
| IX87_RS17130 | NZ_CP009257.1 | 3441010 | 3441630 | + | 621 | 47 |
| IX87_RS01710 | NZ_CP009257.1 | 321262 | 322209 | + | 948 | 46 |
| IX87_RS01720 | NZ_CP009257.1 | 323079 | 323894 | + | 816 | 46 |
| IX87_RS02160 | NZ_CP009257.1 | 400074 | 400439 | − | 366 | 46 |
| IX87_RS02315 | NZ_CP009257.1 | 426770 | 427957 | − | 1188 | 46 |
| IX87_RS02970 | NZ_CP009257.1 | 555280 | 556284 | + | 1005 | 46 |
| IX87_RS10015 | NZ_CP009257.1 | 1990088 | 1990714 | + | 627 | 46 |
| IX87_RS10505 | NZ_CP009257.1 | 2078208 | 2078522 | + | 315 | 46 |
| IX87_RS11330 | NZ_CP009257.1 | 2245026 | 2245361 | + | 336 | 46 |
| IX87_RS11545 | NZ_CP009257.1 | 2288838 | 2289695 | − | 858 | 46 |
| IX87_RS12150 | NZ_CP009257.1 | 2397327 | 2398358 | − | 1032 | 46 |
| IX87_RS12695 | NZ_CP009257.1 | 2508819 | 2510096 | + | 1278 | 46 |
| IX87_RS12870 | NZ_CP009257.1 | 2540172 | 2540987 | + | 816 | 46 |
| IX87_RS16355 | NZ_CP009257.1 | 3274949 | 3275959 | + | 1011 | 46 |
| IX87_RS18600 | NZ_CP009257.1 | 3729749 | 3731218 | + | 1470 | 46 |
| IX87_RS20780 | NZ_CP009257.1 | 4175208 | 4175549 | + | 342 | 46 |
| IX87_RS09240 | NZ_CP009257.1 | 1801905 | 1803308 | + | 1404 | 45 |
| IX87_RS09265 | NZ_CP009257.1 | 1806727 | 1807503 | + | 777 | 45 |
| IX87_RS10965 | NZ_CP009257.1 | 2173454 | 2174740 | − | 1287 | 45 |
| IX87_RS11580 | NZ_CP009257.1 | 2295620 | 2297071 | + | 1452 | 45 |
| IX87_RS11620 | NZ_CP009257.1 | 2303012 | 2304259 | + | 1248 | 45 |
| IX87_RS11830 | NZ_CP009257.1 | 2335450 | 2335863 | − | 414 | 45 |
| IX87_RS15585 | NZ_CP009257.1 | 3111043 | 3113313 | + | 2271 | 45 |
| IX87_RS16135 | NZ_CP009257.1 | 3232226 | 3233242 | + | 1017 | 45 |
| IX87_RS17410 | NZ_CP009257.1 | 3499428 | 3500879 | + | 1452 | 45 |
| IX87_RS00845 | NZ_CP009257.1 | 151849 | 153471 | − | 1623 | 44 |
| IX87_RS04170 | NZ_CP009257.1 | 811981 | 814626 | + | 2646 | 44 |
| IX87_RS06645 | NZ_CP009257.1 | 1291162 | 1291917 | + | 756 | 44 |
| IX87_RS07870 | NZ_CP009257.1 | 1529796 | 1531034 | + | 1239 | 44 |
| IX87_RS08765 | NZ_CP009257.1 | 1704977 | 1706275 | − | 1299 | 44 |
| IX87_RS09690 | NZ_CP009257.1 | 1910652 | 1911500 | − | 849 | 44 |
| IX87_RS10150 | NZ_CP009257.1 | 2020674 | 2021861 | − | 1188 | 44 |
| IX87_RS12700 | NZ_CP009257.1 | 2510370 | 2511260 | + | 891 | 44 |
| IX87_RS13705 | NZ_CP009257.1 | 2717197 | 2717646 | + | 450 | 44 |
| IX87_RS14040 | NZ_CP009257.1 | 2790073 | 2790798 | + | 726 | 44 |
| IX87_RS16645 | NZ_CP009257.1 | 3331236 | 3331928 | − | 693 | 44 |
| IX87_RS17985 | NZ_CP009257.1 | 3617984 | 3621433 | + | 3450 | 44 |
| IX87_RS19460 | NZ_CP009257.1 | 3907874 | 3908983 | + | 1110 | 44 |
| IX87_RS06065 | NZ_CP009257.1 | 1161384 | 1163564 | − | 2181 | 43 |
| IX87_RS07935 | NZ_CP009257.1 | 1540440 | 1541981 | + | 1542 | 43 |
| IX87_RS09020 | NZ_CP009257.1 | 1760911 | 1761669 | − | 759 | 43 |
| IX87_RS09325 | NZ_CP009257.1 | 1818369 | 1819178 | − | 810 | 43 |
| IX87_RS12685 | NZ_CP009257.1 | 2506472 | 2506816 | + | 345 | 43 |
| IX87_RS13600 | NZ_CP009257.1 | 2695104 | 2696681 | − | 1578 | 43 |
| IX87_RS14075 | NZ_CP009257.1 | 2797731 | 2798963 | − | 1233 | 43 |
| IX87_RS15360 | NZ_CP009257.1 | 3066194 | 3066832 | − | 639 | 43 |
| IX87_RS15705 | NZ_CP009257.1 | 3135787 | 3136140 | + | 354 | 43 |
| IX87_RS18735 | NZ_CP009257.1 | 3759445 | 3761763 | + | 2319 | 43 |
| IX87_RS00545 | NZ_CP009257.1 | 83744 | 85822 | − | 2079 | 42 |
| IX87_RS00630 | NZ_CP009257.1 | 104927 | 106456 | − | 1530 | 42 |
| IX87_RS02635 | NZ_CP009257.1 | 484378 | 485127 | + | 750 | 42 |
| IX87_RS04185 | NZ_CP009257.1 | 816071 | 816742 | + | 672 | 42 |
| IX87_RS04250 | NZ_CP009257.1 | 828625 | 830046 | + | 1422 | 42 |
| IX87_RS04570 | NZ_CP009257.1 | 890142 | 892367 | + | 2226 | 42 |
| IX87_RS06155 | NZ_CP009257.1 | 1182334 | 1182777 | + | 444 | 42 |
| IX87_RS06725 | NZ_CP009257.1 | 1306512 | 1308392 | − | 1881 | 42 |
| IX87_RS09830 | NZ_CP009257.1 | 1950037 | 1951539 | − | 1503 | 42 |
| IX87_RS09835 | NZ_CP009257.1 | 1951777 | 1952676 | − | 900 | 42 |
| IX87_RS10600 | NZ_CP009257.1 | 2099104 | 2099679 | + | 576 | 42 |
| IX87_RS10830 | NZ_CP009257.1 | 2144880 | 2145395 | + | 516 | 42 |
| IX87_RS11540 | NZ_CP009257.1 | 2287963 | 2288796 | − | 834 | 42 |
| IX87_RS11890 | NZ_CP009257.1 | 2344100 | 2344864 | + | 765 | 42 |
| IX87_RS14450 | NZ_CP009257.1 | 2872658 | 2873371 | − | 714 | 42 |
| IX87_RS14950 | NZ_CP009257.1 | 2986338 | 2987285 | + | 948 | 42 |
| IX87_RS15375 | NZ_CP009257.1 | 3068863 | 3069963 | + | 1101 | 42 |
| IX87_RS16945 | NZ_CP009257.1 | 3400933 | 3401874 | + | 942 | 42 |
| IX87_RS18515 | NZ_CP009257.1 | 3710575 | 3712398 | + | 1824 | 42 |
| IX87_RS20680 | NZ_CP009257.1 | 4160723 | 4161727 | + | 1005 | 42 |
| IX87_RS21230 | NZ_CP009257.1 | 4250719 | 4251789 | − | 1071 | 42 |
| IX87_RS21345 | NZ_CP009257.1 | 4273409 | 4274803 | − | 1395 | 42 |
| IX87_RS21510 | NZ_CP009257.1 | 4305170 | 4305673 | − | 504 | 42 |
| IX87_RS00855 | NZ_CP009257.1 | 154353 | 155825 | + | 1473 | 41 |
| IX87_RS02830 | NZ_CP009257.1 | 525249 | 527255 | + | 2007 | 41 |
| IX87_RS04560 | NZ_CP009257.1 | 888440 | 889456 | − | 1017 | 41 |
| IX87_RS06690 | NZ_CP009257.1 | 1298587 | 1300032 | + | 1446 | 41 |
| IX87_RS08555 | NZ_CP009257.1 | 1655041 | 1656786 | + | 1746 | 41 |
| IX87_RS09845 | NZ_CP009257.1 | 1953837 | 1954850 | + | 1014 | 41 |
| IX87_RS10460 | NZ_CP009257.1 | 2070130 | 2070747 | − | 618 | 41 |
| IX87_RS15255 | NZ_CP009257.1 | 3045127 | 3047346 | + | 2220 | 41 |
| IX87_RS07860 | NZ_CP009257.1 | 1524349 | 1527197 | + | 2849 | 40 |
| IX87_RS07875 | NZ_CP009257.1 | 1531084 | 1532259 | − | 1176 | 40 |
| IX87_RS09890 | NZ_CP009257.1 | 1966522 | 1966923 | − | 402 | 40 |
| IX87_RS09945 | NZ_CP009257.1 | 1977213 | 1978598 | + | 1386 | 40 |
| IX87_RS12430 | NZ_CP009257.1 | 2451833 | 2453254 | − | 1422 | 40 |
| IX87_RS12640 | NZ_CP009257.1 | 2496176 | 2497132 | − | 957 | 40 |
| IX87_RS13885 | NZ_CP009257.1 | 2756381 | 2756539 | + | 159 | 40 |
| IX87_RS13920 | NZ_CP009257.1 | 2760640 | 2761668 | + | 1029 | 40 |
| IX87_RS14185 | NZ_CP009257.1 | 2822004 | 2822255 | − | 252 | 40 |
| IX87_RS14710 | NZ_CP009257.1 | 2932967 | 2933668 | − | 702 | 40 |
| IX87_RS16380 | NZ_CP009257.1 | 3280656 | 3281774 | − | 1119 | 40 |
| IX87_RS16830 | NZ_CP009257.1 | 3374745 | 3375311 | + | 567 | 40 |
| IX87_RS17815 | NZ_CP009257.1 | 3582583 | 3583470 | − | 888 | 40 |
| IX87_RS18535 | NZ_CP009257.1 | 3716546 | 3718276 | + | 1731 | 40 |
| IX87_RS21200 | NZ_CP009257.1 | 4245903 | 4246343 | + | 441 | 40 |
| IX87_RS00530 | NZ_CP009257.1 | 81071 | 81970 | + | 900 | 39 |
| IX87_RS01555 | NZ_CP009257.1 | 288317 | 289330 | + | 1014 | 39 |
| IX87_RS02790 | NZ_CP009257.1 | 513031 | 513450 | − | 420 | 39 |
| IX87_RS06410 | NZ_CP009257.1 | 1233674 | 1234378 | − | 705 | 39 |
| IX87_RS06775 | NZ_CP009257.1 | 1317562 | 1318293 | + | 732 | 39 |
| IX87_RS09230 | NZ_CP009257.1 | 1800751 | 1801449 | + | 699 | 39 |
| IX87_RS09335 | NZ_CP009257.1 | 1820350 | 1821333 | + | 984 | 39 |
| IX87_RS12675 | NZ_CP009257.1 | 2504854 | 2505252 | − | 399 | 39 |
| IX87_RS14140 | NZ_CP009257.1 | 2813630 | 2815027 | − | 1398 | 39 |
| IX87_RS15345 | NZ_CP009257.1 | 3063792 | 3065084 | + | 1293 | 39 |
| IX87_RS16230 | NZ_CP009257.1 | 3248017 | 3249858 | − | 1842 | 39 |
| IX87_RS16290 | NZ_CP009257.1 | 3265738 | 3266223 | − | 486 | 39 |
| IX87_RS16470 | NZ_CP009257.1 | 3300338 | 3300616 | + | 279 | 39 |
| IX87_RS16520 | NZ_CP009257.1 | 3307019 | 3308443 | − | 1425 | 39 |
| IX87_RS01675 | NZ_CP009257.1 | 313729 | 314775 | + | 1047 | 38 |
| IX87_RS05610 | NZ_CP009257.1 | 1089335 | 1090657 | − | 1323 | 38 |
| IX87_RS06630 | NZ_CP009257.1 | 1284903 | 1287341 | − | 2439 | 38 |
| IX87_RS07195 | NZ_CP009257.1 | 1403480 | 1404484 | − | 1005 | 38 |
| IX87_RS07285 | NZ_CP009257.1 | 1417166 | 1418626 | − | 1461 | 38 |
| IX87_RS07740 | NZ_CP009257.1 | 1499554 | 1500204 | + | 651 | 38 |
| IX87_RS09445 | NZ_CP009257.1 | 1847057 | 1848307 | − | 1251 | 38 |
| IX87_RS09965 | NZ_CP009257.1 | 1980294 | 1981667 | − | 1374 | 38 |
| IX87_RS10840 | NZ_CP009257.1 | 2146806 | 2147249 | + | 444 | 38 |
| IX87_RS11000 | NZ_CP009257.1 | 2180287 | 2181516 | − | 1230 | 38 |
| IX87_RS11400 | NZ_CP009257.1 | 2260767 | 2262101 | + | 1335 | 38 |
| IX87_RS13295 | NZ_CP009257.1; | 2628895; | 2629917; | −; − | 1095 | 38 |
| | NZ_CP009257.1 | 2629919 | 2629990 | | | |
| IX87_RS13955 | NZ_CP009257.1 | 2767310 | 2768131 | − | 822 | 38 |
| IX87_RS14265 | NZ_CP009257.1 | 2834833 | 2835954 | + | 1122 | 38 |
| IX87_RS14460 | NZ_CP009257.1 | 2874246 | 2874893 | − | 648 | 38 |
| IX87_RS16065 | NZ_CP009257.1 | 3216427 | 3217302 | − | 876 | 38 |
| IX87_RS16070 | NZ_CP009257.1 | 3217468 | 3219309 | − | 1842 | 38 |
| IX87_RS18610 | NZ_CP009257.1 | 3731624 | 3733483 | − | 1860 | 38 |
| IX87_RS21280 | NZ_CP009257.1 | 4262143 | 4263252 | + | 1110 | 38 |
| IX87_RS00560 | NZ_CP009257.1 | 87492 | 89951 | − | 2460 | 37 |
| IX87_RS04365 | NZ_CP009257.1 | 845881 | 846105 | − | 225 | 37 |
| IX87_RS08535 | NZ_CP009257.1 | 1650798 | 1651100 | − | 303 | 37 |
| IX87_RS09065 | NZ_CP009257.1 | 1767291 | 1769108 | − | 1818 | 37 |
| IX87_RS10415 | NZ_CP009257.1 | 2059846 | 2060721 | − | 876 | 37 |
| IX87_RS13545 | NZ_CP009257.1 | 2682063 | 2683946 | − | 1884 | 37 |
| IX87_RS19385 | NZ_CP009257.1 | 3893636 | 3894547 | + | 912 | 37 |
| IX87_RS02680 | NZ_CP009257.1 | 492032 | 492796 | − | 765 | 36 |
| IX87_RS02735 | NZ_CP009257.1 | 503163 | 503498 | + | 336 | 36 |
| IX87_RS03245 | NZ_CP009257.1 | 608147 | 608368 | + | 222 | 36 |
| IX87_RS04245 | NZ_CP009257.1 | 827434 | 828411 | − | 978 | 36 |
| IX87_RS07205 | NZ_CP009257.1 | 1405053 | 1406192 | − | 1140 | 36 |
| IX87_RS08900 | NZ_CP009257.1 | 1737908 | 1739044 | − | 1137 | 36 |
| IX87_RS12135 | NZ_CP009257.1 | 2393510 | 2394484 | − | 975 | 36 |
| IX87_RS12770 | NZ_CP009257.1 | 2525107 | 2525838 | + | 732 | 36 |
| IX87_RS13665 | NZ_CP009257.1 | 2709417 | 2710631 | − | 1215 | 36 |
| IX87_RS14620 | NZ_CP009257.1 | 2908566 | 2910236 | + | 1671 | 36 |
| IX87_RS15465 | NZ_CP009257.1 | 3087438 | 3088784 | + | 1347 | 36 |
| IX87_RS15635 | NZ_CP009257.1 | 3118399 | 3118839 | + | 441 | 36 |
| IX87_RS17025 | NZ_CP009257.1 | 3417608 | 3418402 | − | 795 | 36 |
| IX87_RS17470 | NZ_CP009257.1 | 3510720 | 3511784 | + | 1065 | 36 |
| IX87_RS18010 | NZ_CP009257.1 | 3626878 | 3628272 | − | 1395 | 36 |
| IX87_RS19005 | NZ_CP009257.1 | 3808773 | 3809864 | + | 1092 | 36 |
| IX87_RS20740 | NZ_CP009257.1 | 4169103 | 4169699 | − | 597 | 36 |
| IX87_RS02270 | NZ_CP009257.1 | 417143 | 418030 | − | 888 | 35 |
| IX87_RS06780 | NZ_CP009257.1 | 1318650 | 1318853 | + | 204 | 35 |
| IX87_RS11900 | NZ_CP009257.1 | 2345496 | 2346272 | + | 777 | 35 |
| IX87_RS12555 | NZ_CP009257.1 | 2480962 | 2482362 | − | 1401 | 35 |
| IX87_RS13640 | NZ_CP009257.1 | 2704006 | 2704716 | + | 711 | 35 |
| IX87_RS14150 | NZ_CP009257.1 | 2815862 | 2816254 | + | 393 | 35 |
| IX87_RS15995 | NZ_CP009257.1 | 3194518 | 3195465 | − | 948 | 35 |
| IX87_RS16365 | NZ_CP009257.1 | 3276791 | 3277762 | − | 972 | 35 |
| IX87_RS17890 | NZ_CP009257.1 | 3597452 | 3598681 | + | 1230 | 35 |
| IX87_RS18635 | NZ_CP009257.1 | 3736149 | 3736622 | − | 474 | 35 |
| IX87_RS20770 | NZ_CP009257.1 | 4173290 | 4173721 | + | 432 | 35 |
| IX87_RS20835 | NZ_CP009257.1 | 4186200 | 4187918 | + | 1719 | 35 |
| IX87_RS01535 | NZ_CP009257.1 | 282437 | 284401 | − | 1965 | 34 |
| IX87_RS02165 | NZ_CP009257.1 | 400464 | 400766 | − | 303 | 34 |
| IX87_RS02345 | NZ_CP009257.1 | 432377 | 433579 | + | 1203 | 34 |
| IX87_RS04305 | NZ_CP009257.1 | 838139 | 838954 | − | 816 | 34 |
| IX87_RS04630 | NZ_CP009257.1 | 903383 | 904549 | − | 1167 | 34 |
| IX87_RS08780 | NZ_CP009257.1 | 1707595 | 1709058 | + | 1464 | 34 |
| IX87_RS09375 | NZ_CP009257.1 | 1830264 | 1831751 | + | 1488 | 34 |
| IX87_RS10465 | NZ_CP009257.1 | 2070902 | 2071666 | + | 765 | 34 |
| IX87_RS10575 | NZ_CP009257.1 | 2092143 | 2093618 | − | 1476 | 34 |
| IX87_RS11390 | NZ_CP009257.1 | 2258128 | 2259753 | + | 1626 | 34 |
| IX87_RS11855 | NZ_CP009257.1 | 2338087 | 2338407 | − | 321 | 34 |
| IX87_RS11950 | NZ_CP009257.1 | 2357521 | 2358102 | − | 582 | 34 |
| IX87_RS11990 | NZ_CP009257.1 | 2362985 | 2364832 | − | 1848 | 34 |
| IX87_RS12180 | NZ_CP009257.1 | 2404776 | 2406053 | − | 1278 | 34 |
| IX87_RS15000 | NZ_CP009257.1 | 2996594 | 2997058 | − | 465 | 34 |
| IX87_RS15800 | NZ_CP009257.1 | 3155209 | 3155796 | + | 588 | 34 |
| IX87_RS15970 | NZ_CP009257.1 | 3188329 | 3189831 | − | 1503 | 34 |
| IX87_RS16180 | NZ_CP009257.1 | 3238496 | 3239794 | − | 1299 | 34 |
| IX87_RS16765 | NZ_CP009257.1 | 3362725 | 3363957 | + | 1233 | 34 |
| IX87_RS16825 | NZ_CP009257.1 | 3373561 | 3374619 | − | 1059 | 34 |
| IX87_RS16935 | NZ_CP009257.1 | 3398341 | 3399933 | + | 1593 | 34 |
| IX87_RS18060 | NZ_CP009257.1 | 3636801 | 3637418 | − | 618 | 34 |
| IX87_RS19195 | NZ_CP009257.1 | 3851163 | 3852572 | − | 1410 | 34 |
| IX87_RS21250 | NZ_CP009257.1 | 4256259 | 4257455 | − | 1197 | 34 |
| IX87_RS00800 | NZ_CP009257.1 | 141775 | 143769 | − | 1995 | 33 |
| IX87_RS01615 | NZ_CP009257.1 | 302073 | 302471 | + | 399 | 33 |
| IX87_RS01660 | NZ_CP009257.1 | 309655 | 311694 | − | 2040 | 33 |
| IX87_RS03860 | NZ_CP009257.1 | 746078 | 746581 | − | 504 | 33 |
| IX87_RS04235 | NZ_CP009257.1 | 826305 | 826853 | − | 549 | 33 |
| IX87_RS05395 | NZ_CP009257.1 | 1044136 | 1047255 | − | 3120 | 33 |
| IX87_RS06050 | NZ_CP009257.1 | 1157192 | 1158349 | + | 1158 | 33 |
| IX87_RS08980 | NZ_CP009257.1 | 1754836 | 1755627 | + | 792 | 33 |
| IX87_RS11635 | NZ_CP009257.1 | 2305603 | 2306244 | − | 642 | 33 |
| IX87_RS12115 | NZ_CP009257.1 | 2389861 | 2390292 | + | 432 | 33 |
| IX87_RS13415 | NZ_CP009257.1 | 2657071 | 2658015 | − | 945 | 33 |
| IX87_RS13610 | NZ_CP009257.1 | 2697449 | 2698012 | + | 564 | 33 |
| IX87_RS15845 | NZ_CP009257.1 | 3163363 | 3165030 | − | 1668 | 33 |
| IX87_RS16335 | NZ_CP009257.1 | 3272335 | 3273414 | + | 1080 | 33 |
| IX87_RS16775 | NZ_CP009257.1 | 3364764 | 3365555 | + | 792 | 33 |
| IX87_RS17205 | NZ_CP009257.1 | 3453631 | 3455151 | + | 1521 | 33 |
| IX87_RS18975 | NZ_CP009257.1 | 3803509 | 3803814 | + | 306 | 33 |
| IX87_RS18985 | NZ_CP009257.1 | 3804223 | 3805074 | + | 852 | 33 |
| IX87_RS19205 | NZ_CP009257.1 | 3853332 | 3854036 | − | 705 | 33 |
| IX87_RS20765 | NZ_CP009257.1 | 4172817 | 4173149 | + | 333 | 33 |
| IX87_RS00860 | NZ_CP009257.1 | 155921 | 157579 | + | 1659 | 32 |
| IX87_RS02420 | NZ_CP009257.1 | 449014 | 449244 | + | 231 | 32 |
| IX87_RS03175 | NZ_CP009257.1 | 591047 | 591331 | − | 285 | 32 |
| IX87_RS03990 | NZ_CP009257.1 | 770803 | 772521 | − | 1719 | 32 |
| IX87_RS04190 | NZ_CP009257.1 | 816882 | 817550 | + | 669 | 32 |
| IX87_RS04540 | NZ_CP009257.1 | 884059 | 884685 | + | 627 | 32 |
| IX87_RS06825 | NZ_CP009257.1 | 1327527 | 1329194 | − | 1668 | 32 |
| IX87_RS07350 | NZ_CP009257.1 | 1433718 | 1433888 | + | 171 | 32 |
| IX87_RS09080 | NZ_CP009257.1 | 1771463 | 1773106 | + | 1644 | 32 |
| IX87_RS09165 | NZ_CP009257.1 | 1786664 | 1787125 | + | 462 | 32 |
| IX87_RS10740 | NZ_CP009257.1 | 2125770 | 2126492 | + | 723 | 32 |
| IX87_RS10750 | NZ_CP009257.1 | 2127531 | 2128355 | + | 825 | 32 |
| IX87_RS11570 | NZ_CP009257.1 | 2292668 | 2293330 | − | 663 | 32 |
| IX87_RS11610 | NZ_CP009257.1 | 2301503 | 2302078 | + | 576 | 32 |
| IX87_RS12285 | NZ_CP009257.1 | 2424915 | 2425358 | − | 444 | 32 |
| IX87_RS13270 | NZ_CP009257.1 | 2624181 | 2625161 | + | 981 | 32 |
| IX87_RS13460 | NZ_CP009257.1 | 2666433 | 2667257 | + | 825 | 32 |
| IX87_RS13590 | NZ_CP009257.1 | 2691026 | 2694409 | − | 3384 | 32 |
| IX87_RS14380 | NZ_CP009257.1 | 2859427 | 2860428 | − | 1002 | 32 |
| IX87_RS15200 | NZ_CP009257.1 | 3034747 | 3035100 | − | 354 | 32 |
| IX87_RS15760 | NZ_CP009257.1 | 3148198 | 3149112 | + | 915 | 32 |
| IX87_RS16925 | NZ_CP009257.1 | 3396371 | 3396943 | − | 573 | 32 |
| IX87_RS17605 | NZ_CP009257.1 | 3535112 | 3535603 | + | 492 | 32 |
| IX87_RS17945 | NZ_CP009257.1 | 3611396 | 3612205 | − | 810 | 32 |
| IX87_RS18760 | NZ_CP009257.1 | 3765132 | 3767234 | + | 2103 | 32 |
| IX87_RS18960 | NZ_CP009257.1 | 3802092 | 3802358 | + | 267 | 32 |
| IX87_RS02785 | NZ_CP009257.1 | 512549 | 513034 | − | 486 | 31 |
| IX87_RS06365 | NZ_CP009257.1 | 1226044 | 1226697 | + | 654 | 31 |
| IX87_RS13685 | NZ_CP009257.1 | 2712761 | 2713984 | − | 1224 | 31 |
| IX87_RS14445 | NZ_CP009257.1 | 2871960 | 2872661 | − | 702 | 31 |
| IX87_RS15010 | NZ_CP009257.1 | 2998228 | 2998626 | + | 399 | 31 |
| IX87_RS15990 | NZ_CP009257.1 | 3193838 | 3194506 | − | 669 | 31 |
| IX87_RS17055 | NZ_CP009257.1 | 3424926 | 3426317 | + | 1392 | 31 |
| IX87_RS17220 | NZ_CP009257.1 | 3457303 | 3458139 | + | 837 | 31 |
| IX87_RS18065 | NZ_CP009257.1 | 3637508 | 3638080 | − | 573 | 31 |
| IX87_RS18935 | NZ_CP009257.1 | 3798035 | 3798364 | + | 330 | 31 |
| IX87_RS21275 | NZ_CP009257.1 | 4260458 | 4261801 | − | 1344 | 31 |
| IX87_RS05025 | NZ_CP009257.1 | 961029 | 962348 | − | 1320 | 30 |
| IX87_RS08970 | NZ_CP009257.1 | 1751954 | 1752541 | − | 588 | 30 |
| IX87_RS09260 | NZ_CP009257.1 | 1805699 | 1806670 | + | 972 | 30 |
| IX87_RS10480 | NZ_CP009257.1 | 2073566 | 2075020 | − | 1455 | 30 |
| IX87_RS12705 | NZ_CP009257.1 | 2511328 | 2513517 | − | 2190 | 30 |
| IX87_RS14240 | NZ_CP009257.1 | 2830830 | 2832080 | + | 1251 | 30 |
| IX87_RS15380 | NZ_CP009257.1 | 3069963 | 3071033 | + | 1071 | 30 |
| IX87_RS15475 | NZ_CP009257.1 | 3090056 | 3090988 | − | 933 | 30 |
| IX87_RS15590 | NZ_CP009257.1 | 3113354 | 3113983 | + | 630 | 30 |
| IX87_RS15850 | NZ_CP009257.1 | 3165183 | 3166454 | + | 1272 | 30 |
| IX87_RS16020 | NZ_CP009257.1 | 3202305 | 3205985 | + | 3681 | 30 |
| IX87_RS16445 | NZ_CP009257.1 | 3296293 | 3296865 | + | 573 | 30 |
| IX87_RS16450 | NZ_CP009257.1 | 3296982 | 3298961 | + | 1980 | 30 |
| IX87_RS16840 | NZ_CP009257.1 | 3376305 | 3377138 | + | 834 | 30 |
| IX87_RS17170 | NZ_CP009257.1 | 3445757 | 3446758 | − | 1002 | 30 |
| IX87_RS18595 | NZ_CP009257.1 | 3729262 | 3729696 | + | 435 | 30 |
| IX87_RS18620 | NZ_CP009257.1 | 3734126 | 3734446 | − | 321 | 30 |
| IX87_RS21115 | NZ_CP009257.1 | 4229107 | 4231200 | + | 2094 | 30 |
| IX87_RS03080 | NZ_CP009257.1 | 574567 | 575193 | + | 627 | 29 |
| IX87_RS04080 | NZ_CP009257.1 | 794600 | 795550 | − | 951 | 29 |
| IX87_RS04580 | NZ_CP009257.1 | 893079 | 893567 | + | 489 | 29 |
| IX87_RS06025 | NZ_CP009257.1 | 1151701 | 1152729 | − | 1029 | 29 |
| IX87_RS07135 | NZ_CP009257.1 | 1390331 | 1391839 | − | 1509 | 29 |
| IX87_RS09320 | NZ_CP009257.1 | 1817676 | 1818305 | + | 630 | 29 |
| IX87_RS09935 | NZ_CP009257.1 | 1975228 | 1975761 | + | 534 | 29 |
| IX87_RS10925 | NZ_CP009257.1 | 2164138 | 2164779 | + | 642 | 29 |
| IX87_RS12015 | NZ_CP009257.1 | 2370258 | 2371046 | + | 789 | 29 |
| IX87_RS13375 | NZ_CP009257.1 | 2648241 | 2648681 | + | 441 | 29 |
| IX87_RS14135 | NZ_CP009257.1 | 2812384 | 2813133 | − | 750 | 29 |
| IX87_RS20635 | NZ_CP009257.1 | 4147315 | 4148835 | + | 1521 | 29 |
| IX87_RS00375 | NZ_CP009257.1 | 51997 | 54015 | + | 2019 | 28 |
| IX87_RS02330 | NZ_CP009257.1 | 429103 | 430266 | + | 1164 | 28 |
| IX87_RS02720 | NZ_CP009257.1 | 500533 | 501624 | + | 1092 | 28 |
| IX87_RS05335 | NZ_CP009257.1 | 1029021 | 1030466 | + | 1446 | 28 |
| IX87_RS06300 | NZ_CP009257.1 | 1210221 | 1211933 | − | 1713 | 28 |
| IX87_RS06695 | NZ_CP009257.1 | 1300065 | 1301135 | + | 1071 | 28 |
| IX87_RS07140 | NZ_CP009257.1 | 1392018 | 1392899 | + | 882 | 28 |
| IX87_RS07210 | NZ_CP009257.1 | 1406215 | 1406670 | − | 456 | 28 |
| IX87_RS07230 | NZ_CP009257.1 | 1409031 | 1410200 | − | 1170 | 28 |
| IX87_RS09980 | NZ_CP009257.1 | 1984041 | 1984688 | − | 648 | 28 |
| IX87_RS10705 | NZ_CP009257.1 | 2117557 | 2118675 | + | 1119 | 28 |
| IX87_RS10960 | NZ_CP009257.1 | 2172393 | 2173463 | − | 1071 | 28 |
| IX87_RS11040 | NZ_CP009257.1 | 2189378 | 2190064 | − | 687 | 28 |
| IX87_RS11045 | NZ_CP009257.1 | 2190084 | 2191742 | − | 1659 | 28 |
| IX87_RS11555 | NZ_CP009257.1 | 2290486 | 2291013 | + | 528 | 28 |
| IX87_RS11980 | NZ_CP009257.1 | 2361763 | 2362581 | − | 819 | 28 |
| IX87_RS12005 | NZ_CP009257.1 | 2368404 | 2369006 | + | 603 | 28 |
| IX87_RS12320 | NZ_CP009257.1 | 2430928 | 2431476 | − | 549 | 28 |
| IX87_RS12610 | NZ_CP009257.1 | 2492421 | 2493305 | + | 885 | 28 |
| IX87_RS13380 | NZ_CP009257.1 | 2648779 | 2649681 | − | 903 | 28 |
| IX87_RS13495 | NZ_CP009257.1 | 2673584 | 2674129 | − | 546 | 28 |
| IX87_RS14130 | NZ_CP009257.1 | 2811287 | 2812369 | − | 1083 | 28 |
| IX87_RS15105 | NZ_CP009257.1 | 3014688 | 3015116 | + | 429 | 28 |
| IX87_RS15195 | NZ_CP009257.1 | 3034100 | 3034600 | + | 501 | 28 |
| IX87_RS15440 | NZ_CP009257.1 | 3082866 | 3083681 | − | 816 | 28 |
| IX87_RS16045 | NZ_CP009257.1 | 3212772 | 3213983 | + | 1212 | 28 |
| IX87_RS16160 | NZ_CP009257.1 | 3236100 | 3236510 | + | 411 | 28 |
| IX87_RS16410 | NZ_CP009257.1 | 3288317 | 3290227 | − | 1911 | 28 |
| IX87_RS16650 | NZ_CP009257.1 | 3332025 | 3334313 | − | 2289 | 28 |
| IX87_RS17050 | NZ_CP009257.1 | 3424098 | 3424916 | + | 819 | 28 |
| IX87_RS18105 | NZ_CP009257.1 | 3642853 | 3643527 | + | 675 | 28 |
| IX87_RS20645 | NZ_CP009257.1 | 4149512 | 4150183 | − | 672 | 28 |
| IX87_RS00365 | NZ_CP009257.1 | 49867 | 51117 | − | 1251 | 27 |
| IX87_RS01630 | NZ_CP009257.1 | 304069 | 304524 | − | 456 | 27 |
| IX87_RS03765 | NZ_CP009257.1 | 722980 | 724137 | − | 1158 | 27 |
| IX87_RS03815 | NZ_CP009257.1 | 732520 | 735201 | − | 2682 | 27 |
| IX87_RS05490 | NZ_CP009257.1 | 1062592 | 1063254 | + | 663 | 27 |
| IX87_RS05615 | NZ_CP009257.1 | 1090725 | 1091357 | − | 633 | 27 |
| IX87_RS06385 | NZ_CP009257.1 | 1229277 | 1231226 | − | 1950 | 27 |
| IX87_RS06560 | NZ_CP009257.1 | 1271031 | 1272167 | − | 1137 | 27 |
| IX87_RS08805 | NZ_CP009257.1 | 1714120 | 1715514 | − | 1395 | 27 |
| IX87_RS11395 | NZ_CP009257.1 | 2259754 | 2260563 | − | 810 | 27 |
| IX87_RS12495 | NZ_CP009257.1 | 2468319 | 2468978 | − | 660 | 27 |
| IX87_RS13465 | NZ_CP009257.1 | 2667713 | 2668063 | + | 351 | 27 |
| IX87_RS13605 | NZ_CP009257.1 | 2696850 | 2697389 | + | 540 | 27 |
| IX87_RS15540 | NZ_CP009257.1 | 3103545 | 3104279 | + | 735 | 27 |
| IX87_RS16400 | NZ_CP009257.1 | 3286484 | 3287419 | − | 936 | 27 |
| IX87_RS16530 | NZ_CP009257.1 | 3309296 | 3310186 | + | 891 | 27 |
| IX87_RS16565 | NZ_CP009257.1 | 3314709 | 3315551 | − | 843 | 27 |
| IX87_RS18945 | NZ_CP009257.1 | 3799276 | 3800400 | + | 1125 | 27 |
| IX87_RS20795 | NZ_CP009257.1 | 4177117 | 4177911 | − | 795 | 27 |
| IX87_RS01645 | NZ_CP009257.1 | 306637 | 307545 | − | 909 | 26 |
| IX87_RS02255 | NZ_CP009257.1 | 414290 | 416017 | − | 1728 | 26 |
| IX87_RS02800 | NZ_CP009257.1 | 515587 | 516894 | + | 1308 | 26 |
| IX87_RS04225 | NZ_CP009257.1 | 825027 | 825773 | − | 747 | 26 |
| IX87_RS05300 | NZ_CP009257.1 | 1021581 | 1022816 | − | 1236 | 26 |
| IX87_RS05540 | NZ_CP009257.1 | 1077605 | 1079248 | + | 1644 | 26 |
| IX87_RS07020 | NZ_CP009257.1 | 1363028 | 1363261 | + | 234 | 26 |
| IX87_RS07225 | NZ_CP009257.1 | 1408613 | 1408993 | + | 381 | 26 |
| IX87_RS07260 | NZ_CP009257.1 | 1412820 | 1413605 | + | 786 | 26 |
| IX87_RS08325 | NZ_CP009257.1 | 1601239 | 1602045 | + | 807 | 26 |
| IX87_RS08850 | NZ_CP009257.1 | 1726097 | 1727416 | + | 1320 | 26 |
| IX87_RS09745 | NZ_CP009257.1 | 1919039 | 1919575 | + | 537 | 26 |
| IX87_RS09985 | NZ_CP009257.1 | 1984977 | 1985234 | − | 258 | 26 |
| IX87_RS12245 | NZ_CP009257.1 | 2417012 | 2417380 | − | 369 | 26 |
| IX87_RS12365 | NZ_CP009257.1 | 2439691 | 2440641 | − | 951 | 26 |
| IX87_RS13950 | NZ_CP009257.1 | 2766605 | 2767258 | − | 654 | 26 |
| IX87_RS14610 | NZ_CP009257.1 | 2906314 | 2907189 | + | 876 | 26 |
| IX87_RS14630 | NZ_CP009257.1 | 2911289 | 2912659 | − | 1371 | 26 |
| IX87_RS14925 | NZ_CP009257.1 | 2981040 | 2982257 | − | 1218 | 26 |
| IX87_RS15890 | NZ_CP009257.1 | 3173363 | 3174166 | + | 804 | 26 |
| IX87_RS16055 | NZ_CP009257.1 | 3214571 | 3215527 | − | 957 | 26 |
| IX87_RS16195 | NZ_CP009257.1 | 3242618 | 3243913 | − | 1296 | 26 |
| IX87_RS16850 | NZ_CP009257.1 | 3378625 | 3380619 | − | 1995 | 26 |
| IX87_RS17240 | NZ_CP009257.1 | 3464514 | 3465431 | − | 918 | 26 |
| IX87_RS18210 | NZ_CP009257.1 | 3660381 | 3660854 | − | 474 | 26 |
| IX87_RS18685 | NZ_CP009257.1 | 3746588 | 3747961 | − | 1374 | 26 |
| IX87_RS18940 | NZ_CP009257.1 | 3798374 | 3798970 | + | 597 | 26 |
| IX87_RS20760 | NZ_CP009257.1 | 4172105 | 4172338 | + | 234 | 26 |
| IX87_RS21220 | NZ_CP009257.1 | 4249442 | 4250230 | − | 789 | 26 |
| IX87_RS21355 | NZ_CP009257.1 | 4275991 | 4276359 | + | 369 | 26 |
| IX87_RS01800 | NZ_CP009257.1 | 339703 | 340731 | + | 1029 | 25 |
| IX87_RS02275 | NZ_CP009257.1 | 418027 | 418809 | − | 783 | 25 |
| IX87_RS02555 | NZ_CP009257.1 | 473120 | 474037 | + | 918 | 25 |
| IX87_RS05550 | NZ_CP009257.1 | 1080014 | 1080877 | − | 864 | 25 |
| IX87_RS08630 | NZ_CP009257.1 | 1672235 | 1673368 | + | 1134 | 25 |
| IX87_RS08880 | NZ_CP009257.1 | 1732783 | 1734033 | + | 1251 | 25 |
| IX87_RS09215 | NZ_CP009257.1 | 1797566 | 1798570 | + | 1005 | 25 |
| IX87_RS09345 | NZ_CP009257.1 | 1823803 | 1824489 | − | 687 | 25 |
| IX87_RS09655 | NZ_CP009257.1 | 1901094 | 1902032 | − | 939 | 25 |
| IX87_RS10005 | NZ_CP009257.1 | 1986851 | 1987828 | + | 978 | 25 |
| IX87_RS11370 | NZ_CP009257.1 | 2254737 | 2255240 | + | 504 | 25 |
| IX87_RS12500 | NZ_CP009257.1 | 2468994 | 2470076 | − | 1083 | 25 |
| IX87_RS13615 | NZ_CP009257.1 | 2698026 | 2699336 | + | 1311 | 25 |
| IX87_RS13975 | NZ_CP009257.1 | 2770515 | 2773640 | − | 3126 | 25 |
| IX87_RS16005 | NZ_CP009257.1 | 3196529 | 3197947 | + | 1419 | 25 |
| IX87_RS16275 | NZ_CP009257.1 | 3259569 | 3260621 | + | 1053 | 25 |
| IX87_RS16635 | NZ_CP009257.1 | 3327836 | 3328996 | − | 1161 | 25 |
| IX87_RS18120 | NZ_CP009257.1 | 3646451 | 3647011 | + | 561 | 25 |
| IX87_RS18900 | NZ_CP009257.1 | 3792449 | 3792649 | + | 201 | 25 |
| IX87_RS20660 | NZ_CP009257.1 | 4152646 | 4153983 | + | 1338 | 25 |
| IX87_RS02320 | NZ_CP009257.1 | 428096 | 428479 | + | 384 | 24 |
| IX87_RS03665 | NZ_CP009257.1 | 700843 | 701157 | − | 315 | 24 |
| IX87_RS04315 | NZ_CP009257.1 | 839662 | 839877 | − | 216 | 24 |
| IX87_RS04590 | NZ_CP009257.1 | 896505 | 897311 | + | 807 | 24 |
| IX87_RS06080 | NZ_CP009257.1 | 1165968 | 1167227 | − | 1260 | 24 |
| IX87_RS06770 | NZ_CP009257.1 | 1316709 | 1317404 | + | 696 | 24 |
| IX87_RS07165 | NZ_CP009257.1 | 1396045 | 1397472 | − | 1428 | 24 |
| IX87_RS07310 | NZ_CP009257.1 | 1423947 | 1425092 | − | 1146 | 24 |
| IX87_RS07795 | NZ_CP009257.1 | 1509034 | 1509861 | + | 828 | 24 |
| IX87_RS08835 | NZ_CP009257.1 | 1720677 | 1721465 | + | 789 | 24 |
| IX87_RS09370 | NZ_CP009257.1 | 1828805 | 1829884 | − | 1080 | 24 |
| IX87_RS09595 | NZ_CP009257.1 | 1877699 | 1878604 | − | 906 | 24 |
| IX87_RS09775 | NZ_CP009257.1 | 1926318 | 1926755 | + | 438 | 24 |
| IX87_RS09790 | NZ_CP009257.1 | 1927934 | 1929658 | − | 1725 | 24 |
| IX87_RS10050 | NZ_CP009257.1 | 1998020 | 1998826 | − | 807 | 24 |
| IX87_RS11080 | NZ_CP009257.1 | 2198122 | 2199585 | − | 1464 | 24 |
| IX87_RS11425 | NZ_CP009257.1 | 2264611 | 2265423 | − | 813 | 24 |
| IX87_RS11915 | NZ_CP009257.1 | 2348762 | 2349634 | − | 873 | 24 |
| IX87_RS12965 | NZ_CP009257.1 | 2556772 | 2557653 | − | 882 | 24 |
| IX87_RS13120 | NZ_CP009257.1 | 2589460 | 2590902 | − | 1443 | 24 |
| IX87_RS13815 | NZ_CP009257.1 | 2742868 | 2743647 | + | 780 | 24 |
| IX87_RS14025 | NZ_CP009257.1 | 2786496 | 2787800 | + | 1305 | 24 |
| IX87_RS14915 | NZ_CP009257.1 | 2979708 | 2980349 | − | 642 | 24 |
| IX87_RS15390 | NZ_CP009257.1 | 3072722 | 3073906 | + | 1185 | 24 |
| IX87_RS15975 | NZ_CP009257.1 | 3189841 | 3190614 | − | 774 | 24 |
| IX87_RS16015 | NZ_CP009257.1 | 3198648 | 3202283 | + | 3636 | 24 |
| IX87_RS17315 | NZ_CP009257.1 | 3478991 | 3480466 | + | 1476 | 24 |
| IX87_RS17335 | NZ_CP009257.1 | 3482626 | 3483309 | + | 684 | 24 |
| IX87_RS17425 | NZ_CP009257.1 | 3502154 | 3502840 | + | 687 | 24 |
| IX87_RS17805 | NZ_CP009257.1 | 3581518 | 3582153 | + | 636 | 24 |
| IX87_RS17855 | NZ_CP009257.1 | 3589778 | 3590923 | − | 1146 | 24 |
| IX87_RS17930 | NZ_CP009257.1 | 3606721 | 3609171 | + | 2451 | 24 |
| IX87_RS18165 | NZ_CP009257.1 | 3650001 | 3650834 | − | 834 | 24 |
| IX87_RS18185 | NZ_CP009257.1 | 3654514 | 3656379 | − | 1866 | 24 |
| IX87_RS18510 | NZ_CP009257.1 | 3708769 | 3710559 | + | 1791 | 24 |
| IX87_RS18980 | NZ_CP009257.1 | 3803807 | 3804193 | + | 387 | 24 |
| IX87_RS19000 | NZ_CP009257.1 | 3807756 | 3808760 | + | 1005 | 24 |
| IX87_RS20605 | NZ_CP009257.1 | 4140685 | 4141740 | − | 1056 | 24 |
| IX87_RS20665 | NZ_CP009257.1 | 4154025 | 4155176 | − | 1152 | 24 |
| IX87_RS03670 | NZ_CP009257.1 | 701166 | 702104 | − | 939 | 23 |
| IX87_RS05310 | NZ_CP009257.1 | 1023693 | 1025123 | + | 1431 | 23 |
| IX87_RS05545 | NZ_CP009257.1 | 1079248 | 1079991 | + | 744 | 23 |
| IX87_RS05625 | NZ_CP009257.1 | 1091979 | 1092263 | + | 285 | 23 |
| IX87_RS07220 | NZ_CP009257.1 | 1407834 | 1408409 | − | 576 | 23 |
| IX87_RS08625 | NZ_CP009257.1 | 1671742 | 1672230 | + | 489 | 23 |
| IX87_RS08855 | NZ_CP009257.1 | 1727515 | 1728801 | + | 1287 | 23 |
| IX87_RS09060 | NZ_CP009257.1 | 1766393 | 1767220 | − | 828 | 23 |
| IX87_RS09910 | NZ_CP009257.1 | 1970874 | 1971398 | − | 525 | 23 |
| IX87_RS09930 | NZ_CP009257.1 | 1974494 | 1975147 | + | 654 | 23 |
| IX87_RS11430 | NZ_CP009257.1 | 2265447 | 2266430 | − | 984 | 23 |
| IX87_RS13110 | NZ_CP009257.1 | 2586453 | 2587745 | − | 1293 | 23 |
| IX87_RS13265 | NZ_CP009257.1 | 2623493 | 2624068 | + | 576 | 23 |
| IX87_RS14370 | NZ_CP009257.1 | 2856005 | 2856535 | − | 531 | 23 |
| IX87_RS15495 | NZ_CP009257.1 | 3093257 | 3095134 | − | 1878 | 23 |
| IX87_RS16755 | NZ_CP009257.1 | 3361857 | 3362057 | + | 201 | 23 |
| IX87_RS16780 | NZ_CP009257.1 | 3365577 | 3366692 | + | 1116 | 23 |
| IX87_RS18525 | NZ_CP009257.1 | 3714445 | 3715383 | + | 939 | 23 |
| IX87_RS18550 | NZ_CP009257.1 | 3719775 | 3720632 | + | 858 | 23 |
| IX87_RS20745 | NZ_CP009257.1 | 4169889 | 4170995 | − | 1107 | 23 |
| IX87_RS20810 | NZ_CP009257.1 | 4180436 | 4180681 | + | 246 | 23 |
| IX87_RS00625 | NZ_CP009257.1 | 103429 | 104763 | − | 1335 | 22 |
| IX87_RS02130 | NZ_CP009257.1 | 394677 | 395654 | − | 978 | 22 |
| IX87_RS03500 | NZ_CP009257.1 | 667769 | 668491 | − | 723 | 22 |
| IX87_RS03760 | NZ_CP009257.1 | 722445 | 722900 | + | 456 | 22 |
| IX87_RS03865 | NZ_CP009257.1 | 746631 | 748112 | − | 1482 | 22 |
| IX87_RS04180 | NZ_CP009257.1 | 815567 | 815890 | + | 324 | 22 |
| IX87_RS04260 | NZ_CP009257.1 | 830546 | 831175 | − | 630 | 22 |
| IX87_RS04555 | NZ_CP009257.1 | 887192 | 888436 | − | 1245 | 22 |
| IX87_RS05605 | NZ_CP009257.1 | 1088095 | 1089303 | − | 1209 | 22 |
| IX87_RS06000 | NZ_CP009257.1 | 1146413 | 1146985 | + | 573 | 22 |
| IX87_RS06745 | NZ_CP009257.1 | 1311150 | 1311419 | + | 270 | 22 |
| IX87_RS06805 | NZ_CP009257.1 | 1323261 | 1324628 | − | 1368 | 22 |
| IX87_RS09130 | NZ_CP009257.1 | 1781059 | 1781982 | + | 924 | 22 |
| IX87_RS09145 | NZ_CP009257.1 | 1784017 | 1784727 | + | 711 | 22 |
| IX87_RS09650 | NZ_CP009257.1 | 1900355 | 1901113 | − | 759 | 22 |
| IX87_RS10590 | NZ_CP009257.1 | 2095487 | 2096701 | − | 1215 | 22 |
| IX87_RS11245 | NZ_CP009257.1 | 2230960 | 2231289 | + | 330 | 22 |
| IX87_RS11290 | NZ_CP009257.1 | 2239186 | 2239665 | + | 480 | 22 |
| IX87_RS12400 | NZ_CP009257.1 | 2445716 | 2447344 | − | 1629 | 22 |
| IX87_RS13645 | NZ_CP009257.1 | 2704726 | 2706084 | + | 1359 | 22 |
| IX87_RS13660 | NZ_CP009257.1 | 2708418 | 2709266 | − | 849 | 22 |
| IX87_RS13870 | NZ_CP009257.1 | 2753075 | 2754340 | − | 1266 | 22 |
| IX87_RS14115 | NZ_CP009257.1 | 2807693 | 2808250 | + | 558 | 22 |
| IX87_RS14325 | NZ_CP009257.1 | 2848112 | 2848597 | + | 486 | 22 |
| IX87_RS14365 | NZ_CP009257.1 | 2855521 | 2856003 | − | 483 | 22 |
| IX87_RS15330 | NZ_CP009257.1 | 3060680 | 3062077 | + | 1398 | 22 |
| IX87_RS15555 | NZ_CP009257.1 | 3106078 | 3106917 | + | 840 | 22 |
| IX87_RS15955 | NZ_CP009257.1 | 3183394 | 3183762 | − | 369 | 22 |
| IX87_RS16430 | NZ_CP009257.1 | 3292188 | 3292637 | + | 450 | 22 |
| IX87_RS16540 | NZ_CP009257.1 | 3311378 | 3311998 | + | 621 | 22 |
| IX87_RS18175 | NZ_CP009257.1 | 3651436 | 3653073 | − | 1638 | 22 |
| IX87_RS18640 | NZ_CP009257.1 | 3736733 | 3737380 | − | 648 | 22 |
| IX87_RS20290 | NZ_CP009257.1 | 4076022 | 4076738 | + | 717 | 22 |
| IX87_RS20630 | NZ_CP009257.1 | 4146077 | 4147132 | − | 1056 | 22 |
| IX87_RS20790 | NZ_CP009257.1 | 4176163 | 4177065 | + | 903 | 22 |
| IX87_RS00840 | NZ_CP009257.1 | 149665 | 151731 | − | 2067 | 21 |
| IX87_RS04175 | NZ_CP009257.1 | 814691 | 815221 | + | 531 | 21 |
| IX87_RS06620 | NZ_CP009257.1 | 1281827 | 1283074 | − | 1248 | 21 |
| IX87_RS06815 | NZ_CP009257.1 | 1325905 | 1326633 | − | 729 | 21 |
| IX87_RS09275 | NZ_CP009257.1 | 1808267 | 1808587 | + | 321 | 21 |
| IX87_RS10495 | NZ_CP009257.1 | 2076160 | 2077017 | − | 858 | 21 |
| IX87_RS10555 | NZ_CP009257.1 | 2086872 | 2087870 | − | 999 | 21 |
| IX87_RS10775 | NZ_CP009257.1 | 2133402 | 2134226 | + | 825 | 21 |
| IX87_RS11240 | NZ_CP009257.1 | 2229728 | 2230861 | + | 1134 | 21 |
| IX87_RS11945 | NZ_CP009257.1 | 2357105 | 2357506 | − | 402 | 21 |
| IX87_RS12210 | NZ_CP009257.1 | 2409670 | 2410788 | + | 1119 | 21 |
| IX87_RS13860 | NZ_CP009257.1 | 2751732 | 2751935 | + | 204 | 21 |
| IX87_RS15935 | NZ_CP009257.1 | 3180364 | 3180768 | − | 405 | 21 |
| IX87_RS16670 | NZ_CP009257.1 | 3338423 | 3339139 | + | 717 | 21 |
| IX87_RS16700 | NZ_CP009257.1 | 3347639 | 3348259 | + | 621 | 21 |
| IX87_RS16790 | NZ_CP009257.1 | 3368010 | 3368708 | + | 699 | 21 |
| IX87_RS00850 | NZ_CP009257.1 | 153725 | 154360 | + | 636 | 20 |
| IX87_RS02465 | NZ_CP009257.1 | 456673 | 457233 | − | 561 | 20 |
| IX87_RS03235 | NZ_CP009257.1 | 605657 | 607066 | − | 1410 | 20 |
| IX87_RS03680 | NZ_CP009257.1 | 704516 | 705895 | − | 1380 | 20 |
| IX87_RS04075 | NZ_CP009257.1 | 793538 | 793996 | − | 459 | 20 |
| IX87_RS05010 | NZ_CP009257.1 | 958689 | 959078 | − | 390 | 20 |
| IX87_RS06040 | NZ_CP009257.1 | 1154918 | 1155421 | − | 504 | 20 |
| IX87_RS06715 | NZ_CP009257.1 | 1303340 | 1304890 | − | 1551 | 20 |
| IX87_RS07240 | NZ_CP009257.1 | 1410873 | 1411118 | + | 246 | 20 |
| IX87_RS07340 | NZ_CP009257.1 | 1432109 | 1432660 | + | 552 | 20 |
| IX87_RS09305 | NZ_CP009257.1 | 1814561 | 1815259 | − | 699 | 20 |
| IX87_RS09615 | NZ_CP009257.1 | 1881832 | 1884927 | − | 3096 | 20 |
| IX87_RS09740 | NZ_CP009257.1 | 1918701 | 1919024 | + | 324 | 20 |
| IX87_RS09765 | NZ_CP009257.1 | 1925008 | 1925484 | + | 477 | 20 |
| IX87_RS10125 | NZ_CP009257.1 | 2016851 | 2017657 | + | 807 | 20 |
| IX87_RS10435 | NZ_CP009257.1 | 2064195 | 2064995 | + | 801 | 20 |
| IX87_RS10580 | NZ_CP009257.1 | 2094041 | 2094244 | − | 204 | 20 |
| IX87_RS10665 | NZ_CP009257.1 | 2106785 | 2107921 | + | 1137 | 20 |
| IX87_RS10810 | NZ_CP009257.1 | 2140199 | 2141659 | + | 1461 | 20 |
| IX87_RS10835 | NZ_CP009257.1 | 2145504 | 2146679 | + | 1176 | 20 |
| IX87_RS10880 | NZ_CP009257.1 | 2156493 | 2157698 | + | 1206 | 20 |
| IX87_RS11155 | NZ_CP009257.1 | 2213291 | 2214046 | − | 756 | 20 |
| IX87_RS11340 | NZ_CP009257.1 | 2247266 | 2248639 | + | 1374 | 20 |
| IX87_RS11905 | NZ_CP009257.1 | 2346396 | 2347346 | + | 951 | 20 |
| IX87_RS12370 | NZ_CP009257.1 | 2440764 | 2441393 | + | 630 | 20 |
| IX87_RS12605 | NZ_CP009257.1 | 2490884 | 2492302 | + | 1419 | 20 |
| IX87_RS12805 | NZ_CP009257.1 | 2529934 | 2530692 | + | 759 | 20 |
| IX87_RS13150 | NZ_CP009257.1 | 2595025 | 2596107 | − | 1083 | 20 |
| IX87_RS14080 | NZ_CP009257.1 | 2799025 | 2800152 | + | 1128 | 20 |
| IX87_RS14320 | NZ_CP009257.1 | 2846826 | 2847896 | + | 1071 | 20 |
| IX87_RS14780 | NZ_CP009257.1 | 2948111 | 2949628 | + | 1518 | 20 |
| IX87_RS15190 | NZ_CP009257.1 | 3031408 | 3033786 | + | 2379 | 20 |
| IX87_RS15725 | NZ_CP009257.1 | 3140768 | 3141472 | − | 705 | 20 |
| IX87_RS16240 | NZ_CP009257.1 | 3254106 | 3255038 | + | 933 | 20 |
| IX87_RS16855 | NZ_CP009257.1 | 3380622 | 3381959 | − | 1338 | 20 |
| IX87_RS18170 | NZ_CP009257.1 | 3650853 | 3651431 | − | 579 | 20 |
| IX87_RS18180 | NZ_CP009257.1 | 3653234 | 3654517 | + | 1284 | 20 |
| IX87_RS18650 | NZ_CP009257.1 | 3738539 | 3738889 | + | 351 | 20 |
| IX87_RS21120 | NZ_CP009257.1 | 4231326 | 4231769 | + | 444 | 20 |
| IX87_RS01655 | NZ_CP009257.1 | 309178 | 309630 | − | 453 | 19 |
| IX87_RS01690 | NZ_CP009257.1 | 316505 | 316777 | + | 273 | 19 |
| IX87_RS02325 | NZ_CP009257.1 | 428479 | 429072 | + | 594 | 19 |
| IX87_RS02440 | NZ_CP009257.1 | 451654 | 452061 | + | 408 | 19 |
| IX87_RS03300 | NZ_CP009257.1 | 616001 | 616672 | − | 672 | 19 |
| IX87_RS03310 | NZ_CP009257.1 | 618536 | 619129 | − | 594 | 19 |
| IX87_RS06255 | NZ_CP009257.1 | 1201373 | 1201930 | − | 558 | 19 |
| IX87_RS06290 | NZ_CP009257.1 | 1208869 | 1209696 | + | 828 | 19 |
| IX87_RS06565 | NZ_CP009257.1 | 1272186 | 1272956 | − | 771 | 19 |
| IX87_RS07855 | NZ_CP009257.1 | 1523492 | 1524214 | + | 723 | 19 |
| IX87_RS08575 | NZ_CP009257.1 | 1660477 | 1660878 | − | 402 | 19 |
| IX87_RS08665 | NZ_CP009257.1 | 1679905 | 1680762 | − | 858 | 19 |
| IX87_RS08775 | NZ_CP009257.1 | 1707046 | 1707429 | − | 384 | 19 |
| IX87_RS08925 | NZ_CP009257.1 | 1743568 | 1744224 | + | 657 | 19 |
| IX87_RS09385 | NZ_CP009257.1 | 1832949 | 1834019 | + | 1071 | 19 |
| IX87_RS09450 | NZ_CP009257.1 | 1848330 | 1848554 | − | 225 | 19 |
| IX87_RS11375 | NZ_CP009257.1 | 2255369 | 2256469 | + | 1101 | 19 |
| IX87_RS12185 | NZ_CP009257.1 | 2406181 | 2406975 | + | 795 | 19 |
| IX87_RS12745 | NZ_CP009257.1 | 2521886 | 2522269 | − | 384 | 19 |
| IX87_RS12750 | NZ_CP009257.1 | 2522372 | 2522965 | + | 594 | 19 |
| IX87_RS13210 | NZ_CP009257.1 | 2609332 | 2610012 | − | 681 | 19 |
| IX87_RS13555 | NZ_CP009257.1 | 2684754 | 2686097 | + | 1344 | 19 |
| IX87_RS13625 | NZ_CP009257.1 | 2700607 | 2701992 | + | 1386 | 19 |
| IX87_RS15110 | NZ_CP009257.1 | 3015189 | 3017303 | + | 2115 | 19 |
| IX87_RS16260 | NZ_CP009257.1 | 3257191 | 3257868 | − | 678 | 19 |
| IX87_RS16465 | NZ_CP009257.1 | 3299692 | 3300225 | + | 534 | 19 |
| IX87_RS16535 | NZ_CP009257.1 | 3310617 | 3311333 | + | 717 | 19 |
| IX87_RS17995 | NZ_CP009257.1 | 3622567 | 3624603 | + | 2037 | 19 |
| IX87_RS18195 | NZ_CP009257.1 | 3657767 | 3658819 | + | 1053 | 19 |
| IX87_RS18580 | NZ_CP009257.1 | 3724097 | 3725029 | + | 933 | 19 |
| IX87_RS20655 | NZ_CP009257.1 | 4151977 | 4152627 | + | 651 | 19 |
| IX87_RS21260 | NZ_CP009257.1 | 4258280 | 4259032 | − | 753 | 19 |
| IX87_RS00345 | NZ_CP009257.1 | 45729 | 47132 | − | 1404 | 18 |
| IX87_RS00580 | NZ_CP009257.1 | 92247 | 93272 | + | 1026 | 18 |
| IX87_RS00830 | NZ_CP009257.1 | 148262 | 148777 | − | 516 | 18 |
| IX87_RS02355 | NZ_CP009257.1 | 434269 | 434805 | + | 537 | 18 |
| IX87_RS03625 | NZ_CP009257.1 | 692808 | 694097 | − | 1290 | 18 |
| IX87_RS05535 | NZ_CP009257.1 | 1076093 | 1077472 | + | 1380 | 18 |
| IX87_RS06085 | NZ_CP009257.1 | 1167300 | 1168154 | − | 855 | 18 |
| IX87_RS06225 | NZ_CP009257.1 | 1195205 | 1197475 | + | 2271 | 18 |
| IX87_RS06400 | NZ_CP009257.1 | 1232832 | 1233140 | + | 309 | 18 |
| IX87_RS06475 | NZ_CP009257.1 | 1252040 | 1253362 | + | 1323 | 18 |
| IX87_RS06545 | NZ_CP009257.1 | 1267596 | 1268372 | − | 777 | 18 |
| IX87_RS06880 | NZ_CP009257.1 | 1339625 | 1341112 | + | 1488 | 18 |
| IX87_RS07330 | NZ_CP009257.1 | 1428598 | 1429506 | − | 909 | 18 |
| IX87_RS08305 | NZ_CP009257.1 | 1593642 | 1594217 | + | 576 | 18 |
| IX87_RS08635 | NZ_CP009257.1 | 1673373 | 1674116 | + | 744 | 18 |
| IX87_RS08910 | NZ_CP009257.1 | 1740194 | 1741240 | + | 1047 | 18 |
| IX87_RS10800 | NZ_CP009257.1 | 2137743 | 2139101 | + | 1359 | 18 |
| IX87_RS10805 | NZ_CP009257.1 | 2139164 | 2140177 | + | 1014 | 18 |
| IX87_RS11300 | NZ_CP009257.1 | 2241612 | 2241926 | − | 315 | 18 |
| IX87_RS11440 | NZ_CP009257.1 | 2267044 | 2267430 | + | 387 | 18 |
| IX87_RS11730 | NZ_CP009257.1 | 2324794 | 2326284 | − | 1491 | 18 |
| IX87_RS11910 | NZ_CP009257.1 | 2347389 | 2348654 | − | 1266 | 18 |
| IX87_RS12045 | NZ_CP009257.1 | 2377336 | 2377590 | + | 255 | 18 |
| IX87_RS12280 | NZ_CP009257.1 | 2424047 | 2424907 | + | 861 | 18 |
| IX87_RS13710 | NZ_CP009257.1 | 2717662 | 2718579 | + | 918 | 18 |
| IX87_RS13995 | NZ_CP009257.1 | 2778672 | 2779967 | + | 1296 | 18 |
| IX87_RS14425 | NZ_CP009257.1 | 2868500 | 2869855 | − | 1356 | 18 |
| IX87_RS14995 | NZ_CP009257.1 | 2995833 | 2996621 | − | 789 | 18 |
| IX87_RS15055 | NZ_CP009257.1 | 3005456 | 3006055 | − | 600 | 18 |
| IX87_RS15420 | NZ_CP009257.1 | 3078123 | 3078758 | − | 636 | 18 |
| IX87_RS15545 | NZ_CP009257.1 | 3104368 | 3105297 | + | 930 | 18 |
| IX87_RS15740 | NZ_CP009257.1 | 3142867 | 3143514 | + | 648 | 18 |
| IX87_RS16170 | NZ_CP009257.1 | 3237555 | 3237887 | − | 333 | 18 |
| IX87_RS16405 | NZ_CP009257.1 | 3287539 | 3288171 | − | 633 | 18 |
| IX87_RS16975 | NZ_CP009257.1 | 3408238 | 3408747 | − | 510 | 18 |
| IX87_RS17255 | NZ_CP009257.1 | 3467904 | 3468335 | − | 432 | 18 |
| IX87_RS17510 | NZ_CP009257.1 | 3516339 | 3517559 | + | 1221 | 18 |
| IX87_RS18690 | NZ_CP009257.1 | 3748198 | 3748476 | − | 279 | 18 |
| IX87_RS21270 | NZ_CP009257.1 | 4259678 | 4260406 | − | 729 | 18 |
| IX87_RS00685 | NZ_CP009257.1 | 119799 | 121121 | − | 1323 | 17 |
| IX87_RS00795 | NZ_CP009257.1 | 140502 | 141761 | − | 1260 | 17 |
| IX87_RS01695 | NZ_CP009257.1 | 316861 | 317148 | + | 288 | 17 |
| IX87_RS01860 | NZ_CP009257.1 | 351716 | 352207 | − | 492 | 17 |
| IX87_RS03585 | NZ_CP009257.1 | 684169 | 685185 | − | 1017 | 17 |
| IX87_RS04155 | NZ_CP009257.1 | 808403 | 809752 | − | 1350 | 17 |
| IX87_RS06735 | NZ_CP009257.1 | 1309012 | 1309917 | + | 906 | 17 |
| IX87_RS07320 | NZ_CP009257.1 | 1427114 | 1428127 | + | 1014 | 17 |
| IX87_RS07735 | NZ_CP009257.1 | 1498718 | 1499530 | + | 813 | 17 |
| IX87_RS08695 | NZ_CP009257.1 | 1688558 | 1689043 | + | 486 | 17 |
| IX87_RS08870 | NZ_CP009257.1 | 1731160 | 1731900 | + | 741 | 17 |
| IX87_RS10145 | NZ_CP009257.1 | 2019603 | 2020514 | − | 912 | 17 |
| IX87_RS10850 | NZ_CP009257.1 | 2147727 | 2148212 | − | 486 | 17 |
| IX87_RS11320 | NZ_CP009257.1 | 2243500 | 2243937 | − | 438 | 17 |
| IX87_RS11380 | NZ_CP009257.1 | 2256484 | 2256963 | + | 480 | 17 |
| IX87_RS11600 | NZ_CP009257.1 | 2299888 | 2300559 | + | 672 | 17 |
| IX87_RS11935 | NZ_CP009257.1 | 2354429 | 2356474 | + | 2046 | 17 |
| IX87_RS11955 | NZ_CP009257.1 | 2358260 | 2359273 | + | 1014 | 17 |
| IX87_RS12220 | NZ_CP009257.1 | 2411674 | 2413824 | + | 2151 | 17 |
| IX87_RS12680 | NZ_CP009257.1 | 2505401 | 2506093 | + | 693 | 17 |
| IX87_RS13220 | NZ_CP009257.1 | 2611909 | 2612241 | − | 333 | 17 |
| IX87_RS13475 | NZ_CP009257.1 | 2668689 | 2669600 | + | 912 | 17 |
| IX87_RS13840 | NZ_CP009257.1 | 2748297 | 2748941 | − | 645 | 17 |
| IX87_RS14120 | NZ_CP009257.1 | 2808336 | 2808728 | − | 393 | 17 |
| IX87_RS23185 | NZ_CP009257.1 | 2813130 | 2813532 | − | 403 | 17 |
| IX87_RS15365 | NZ_CP009257.1 | 3066871 | 3067278 | − | 408 | 17 |
| IX87_RS15560 | NZ_CP009257.1 | 3107008 | 3108120 | + | 1113 | 17 |
| IX87_RS16125 | NZ_CP009257.1 | 3230161 | 3231027 | + | 867 | 17 |
| IX87_RS16395 | NZ_CP009257.1 | 3285118 | 3286218 | − | 1101 | 17 |
| IX87_RS17245 | NZ_CP009257.1 | 3465441 | 3466262 | − | 822 | 17 |
| IX87_RS17415 | NZ_CP009257.1 | 3500848 | 3501696 | + | 849 | 17 |
| IX87_RS18055 | NZ_CP009257.1 | 3635781 | 3636548 | − | 768 | 17 |
| IX87_RS20600 | NZ_CP009257.1 | 4139721 | 4140638 | + | 918 | 17 |
| IX87_RS20725 | NZ_CP009257.1 | 4167297 | 4167596 | + | 300 | 17 |
| IX87_RS01560 | NZ_CP009257.1 | 289356 | 290471 | + | 1116 | 16 |
| IX87_RS02150 | NZ_CP009257.1 | 398301 | 399212 | + | 912 | 16 |
| IX87_RS02335 | NZ_CP009257.1 | 430604 | 431170 | + | 567 | 16 |
| IX87_RS02390 | NZ_CP009257.1 | 439995 | 440702 | + | 708 | 16 |
| IX87_RS02470 | NZ_CP009257.1 | 457632 | 458456 | + | 825 | 16 |
| IX87_RS02560 | NZ_CP009257.1 | 474150 | 474614 | + | 465 | 16 |
| IX87_RS03835 | NZ_CP009257.1 | 737468 | 741292 | − | 3825 | 16 |
| IX87_RS03955 | NZ_CP009257.1 | 765035 | 765466 | + | 432 | 16 |
| IX87_RS04340 | NZ_CP009257.1 | 842645 | 843316 | + | 672 | 16 |
| IX87_RS05020 | NZ_CP009257.1 | 959919 | 960917 | − | 999 | 16 |
| IX87_RS06740 | NZ_CP009257.1 | 1309931 | 1310773 | + | 843 | 16 |
| IX87_RS07030 | NZ_CP009257.1 | 1364909 | 1366477 | − | 1569 | 16 |
| IX87_RS07745 | NZ_CP009257.1 | 1500333 | 1501475 | + | 1143 | 16 |
| IX87_RS08320 | NZ_CP009257.1 | 1600180 | 1601229 | + | 1050 | 16 |
| IX87_RS08550 | NZ_CP009257.1 | 1654009 | 1655025 | + | 1017 | 16 |
| IX87_RS08820 | NZ_CP009257.1 | 1718217 | 1718627 | + | 411 | 16 |
| IX87_RS08865 | NZ_CP009257.1 | 1729523 | 1730965 | + | 1443 | 16 |
| IX87_RS08875 | NZ_CP009257.1 | 1731900 | 1732796 | + | 897 | 16 |
| IX87_RS08985 | NZ_CP009257.1 | 1755797 | 1757233 | + | 1437 | 16 |
| IX87_RS09180 | NZ_CP009257.1 | 1788588 | 1789387 | + | 800 | 16 |
| IX87_RS09440 | NZ_CP009257.1 | 1846206 | 1847051 | − | 846 | 16 |
| IX87_RS09750 | NZ_CP009257.1 | 1919635 | 1920222 | − | 588 | 16 |
| IX87_RS09995 | NZ_CP009257.1 | 1985661 | 1985954 | + | 294 | 16 |
| IX87_RS10430 | NZ_CP009257.1 | 2063021 | 2063839 | − | 819 | 16 |
| IX87_RS10605 | NZ_CP009257.1 | 2099837 | 2102383 | + | 2547 | 16 |
| IX87_RS11005 | NZ_CP009257.1 | 2181500 | 2182159 | − | 660 | 16 |
| IX87_RS11035 | NZ_CP009257.1 | 2187872 | 2189314 | + | 1443 | 16 |
| IX87_RS11145 | NZ_CP009257.1 | 2211485 | 2212519 | − | 1035 | 16 |
| IX87_RS11225 | NZ_CP009257.1 | 2226677 | 2227714 | + | 1038 | 16 |
| IX87_RS11590 | NZ_CP009257.1 | 2298207 | 2298683 | + | 477 | 16 |
| IX87_RS11710 | NZ_CP009257.1 | 2320876 | 2321961 | − | 1086 | 16 |
| IX87_RS11775 | NZ_CP009257.1 | 2331342 | 2331518 | − | 177 | 16 |
| IX87_RS23140 | NZ_CP009257.1 | 2362708 | 2362878 | − | 171 | 16 |
| IX87_RS12340 | NZ_CP009257.1 | 2432811 | 2436569 | − | 3759 | 16 |
| IX87_RS13100 | NZ_CP009257.1 | 2583974 | 2584903 | + | 930 | 16 |
| IX87_RS13875 | NZ_CP009257.1 | 2754446 | 2755567 | − | 1122 | 16 |
| IX87_RS14210 | NZ_CP009257.1 | 2824359 | 2825684 | − | 1326 | 16 |
| IX87_RS14480 | NZ_CP009257.1 | 2878199 | 2878915 | + | 717 | 16 |
| IX87_RS14795 | NZ_CP009257.1 | 2952273 | 2953400 | + | 1128 | 16 |
| IX87_RS14890 | NZ_CP009257.1 | 2976866 | 2977174 | + | 309 | 16 |
| IX87_RS14900 | NZ_CP009257.1 | 2977655 | 2978497 | + | 843 | 16 |
| IX87_RS15320 | NZ_CP009257.1 | 3059777 | 3060004 | − | 228 | 16 |
| IX87_RS15515 | NZ_CP009257.1 | 3098158 | 3099489 | + | 1332 | 16 |
| IX87_RS15695 | NZ_CP009257.1 | 3133259 | 3134683 | + | 1425 | 16 |
| IX87_RS15980 | NZ_CP009257.1 | 3190768 | 3192081 | − | 1314 | 16 |
| IX87_RS16075 | NZ_CP009257.1 | 3219386 | 3221614 | − | 2229 | 16 |
| IX87_RS16420 | NZ_CP009257.1 | 3290553 | 3291563 | − | 1011 | 16 |
| IX87_RS16555 | NZ_CP009257.1 | 3313085 | 3314140 | − | 1056 | 16 |
| IX87_RS16580 | NZ_CP009257.1 | 3316480 | 3317298 | − | 819 | 16 |
| IX87_RS16725 | NZ_CP009257.1 | 3353833 | 3355128 | + | 1296 | 16 |
| IX87_RS16800 | NZ_CP009257.1 | 3370041 | 3371450 | + | 1410 | 16 |
| IX87_RS16895 | NZ_CP009257.1 | 3390853 | 3392907 | + | 2055 | 16 |
| IX87_RS17045 | NZ_CP009257.1 | 3423152 | 3424075 | + | 924 | 16 |
| IX87_RS17225 | NZ_CP009257.1 | 3458161 | 3458730 | + | 570 | 16 |
| IX87_RS17420 | NZ_CP009257.1 | 3501697 | 3501969 | − | 273 | 16 |
| IX87_RS17860 | NZ_CP009257.1 | 3590964 | 3592607 | − | 1644 | 16 |
| IX87_RS17925 | NZ_CP009257.1 | 3606002 | 3606727 | + | 726 | 16 |
| IX87_RS18545 | NZ_CP009257.1 | 3719097 | 3719465 | − | 369 | 16 |
| IX87_RS18855 | NZ_CP009257.1 | 3786513 | 3787406 | − | 894 | 16 |
| IX87_RS19390 | NZ_CP009257.1 | 3894556 | 3895314 | + | 759 | 16 |
| IX87_RS20590 | NZ_CP009257.1 | 4136998 | 4139022 | − | 2025 | 16 |
| IX87_RS20750 | NZ_CP009257.1 | 4171103 | 4171429 | − | 327 | 16 |
| IX87_RS21255 | NZ_CP009257.1 | 4257456 | 4258280 | − | 825 | 16 |
| IX87_RS21290 | NZ_CP009257.1 | 4265008 | 4265661 | + | 654 | 16 |
| IX87_RS02285 | NZ_CP009257.1 | 419551 | 420240 | − | 690 | 15 |
| IX87_RS02415 | NZ_CP009257.1 | 447680 | 448765 | + | 1086 | 15 |
| IX87_RS06390 | NZ_CP009257.1 | 1231219 | 1231692 | − | 474 | 15 |
| IX87_RS06580 | NZ_CP009257.1 | 1274979 | 1276067 | − | 1089 | 15 |
| IX87_RS07290 | NZ_CP009257.1 | 1418798 | 1419181 | − | 384 | 15 |
| IX87_RS09990 | NZ_CP009257.1 | 1985253 | 1985564 | − | 312 | 15 |
| IX87_RS11295 | NZ_CP009257.1 | 2239689 | 2241560 | + | 1872 | 15 |
| IX87_RS11410 | NZ_CP009257.1 | 2262526 | 2263200 | + | 675 | 15 |
| IX87_RS13280 | NZ_CP009257.1 | 2625845 | 2627137 | − | 1293 | 15 |
| IX87_RS13715 | NZ_CP009257.1 | 2718557 | 2719072 | + | 516 | 15 |
| IX87_RS14010 | NZ_CP009257.1 | 2781756 | 2783045 | − | 1290 | 15 |
| IX87_RS14905 | NZ_CP009257.1 | 2978508 | 2978894 | + | 387 | 15 |
| IX87_RS15215 | NZ_CP009257.1 | 3037314 | 3038705 | − | 1392 | 15 |
| IX87_RS15595 | NZ_CP009257.1 | 3114049 | 3114717 | + | 669 | 15 |
| IX87_RS16385 | NZ_CP009257.1 | 3281964 | 3283085 | + | 1122 | 15 |
| IX87_RS16770 | NZ_CP009257.1 | 3364006 | 3364773 | + | 768 | 15 |
| IX87_RS18530 | NZ_CP009257.1 | 3715391 | 3716533 | + | 1143 | 15 |
| IX87_RS18825 | NZ_CP009257.1 | 3779494 | 3780669 | + | 1176 | 15 |
| IX87_RS20620 | NZ_CP009257.1 | 4144820 | 4145233 | − | 414 | 15 |
| IX87_RS20830 | NZ_CP009257.1 | 4185266 | 4186213 | + | 948 | 15 |
| IX87_RS00385 | NZ_CP009257.1 | 54608 | 55177 | − | 570 | 14 |
| IX87_RS00835 | NZ_CP009257.1 | 149039 | 149653 | − | 615 | 14 |
| IX87_RS02250 | NZ_CP009257.1 | 413277 | 414287 | − | 1011 | 14 |
| IX87_RS02370 | NZ_CP009257.1 | 436066 | 437913 | + | 1848 | 14 |
| IX87_RS02425 | NZ_CP009257.1 | 449280 | 450026 | − | 747 | 14 |
| IX87_RS02960 | NZ_CP009257.1 | 552370 | 553188 | − | 819 | 14 |
| IX87_RS03725 | NZ_CP009257.1 | 715654 | 716337 | − | 684 | 14 |
| IX87_RS04135 | NZ_CP009257.1 | 804011 | 804448 | + | 438 | 14 |
| IX87_RS04240 | NZ_CP009257.1 | 826891 | 827430 | − | 540 | 14 |
| IX87_RS04600 | NZ_CP009257.1 | 897825 | 898247 | − | 423 | 14 |
| IX87_RS05345 | NZ_CP009257.1 | 1032352 | 1032711 | + | 360 | 14 |
| IX87_RS05525 | NZ_CP009257.1 | 1070794 | 1072506 | − | 1713 | 14 |
| IX87_RS06315 | NZ_CP009257.1 | 1214496 | 1215686 | − | 1191 | 14 |
| IX87_RS06480 | NZ_CP009257.1 | 1253366 | 1254001 | + | 636 | 14 |
| IX87_RS06635 | NZ_CP009257.1 | 1287727 | 1288563 | − | 837 | 14 |
| IX87_RS06820 | NZ_CP009257.1 | 1326827 | 1327537 | − | 711 | 14 |
| IX87_RS07305 | NZ_CP009257.1 | 1422959 | 1423903 | + | 945 | 14 |
| IX87_RS08330 | NZ_CP009257.1 | 1602055 | 1602750 | + | 696 | 14 |
| IX87_RS08350 | NZ_CP009257.1 | 1607890 | 1608438 | + | 549 | 14 |
| IX87_RS08745 | NZ_CP009257.1 | 1699385 | 1700401 | + | 1017 | 14 |
| IX87_RS08945 | NZ_CP009257.1 | 1747454 | 1748188 | − | 735 | 14 |
| IX87_RS08990 | NZ_CP009257.1 | 1757267 | 1757446 | − | 180 | 14 |
| IX87_RS09290 | NZ_CP009257.1 | 1811508 | 1812737 | − | 1230 | 14 |
| IX87_RS09585 | NZ_CP009257.1 | 1875905 | 1877017 | + | 1113 | 14 |
| IX87_RS09600 | NZ_CP009257.1 | 1879195 | 1879971 | + | 777 | 14 |
| IX87_RS10055 | NZ_CP009257.1 | 1998852 | 1999529 | − | 678 | 14 |
| IX87_RS10420 | NZ_CP009257.1 | 2060924 | 2061922 | − | 999 | 14 |
| IX87_RS10885 | NZ_CP009257.1 | 2157719 | 2159854 | + | 2136 | 14 |
| IX87_RS11085 | NZ_CP009257.1 | 2199661 | 2201736 | − | 2076 | 14 |
| IX87_RS11270 | NZ_CP009257.1 | 2236415 | 2237326 | + | 912 | 14 |
| IX87_RS11280 | NZ_CP009257.1 | 2238230 | 2238844 | + | 615 | 14 |
| IX87_RS11895 | NZ_CP009257.1 | 2344950 | 2345387 | + | 438 | 14 |
| IX87_RS11930 | NZ_CP009257.1 | 2353593 | 2354408 | − | 816 | 14 |
| IX87_RS12630 | NZ_CP009257.1 | 2494742 | 2495545 | − | 804 | 14 |
| IX87_RS12835 | NZ_CP009257.1 | 2534471 | 2535574 | − | 1104 | 14 |
| IX87_RS13550 | NZ_CP009257.1 | 2683961 | 2684542 | − | 582 | 14 |
| IX87_RS13630 | NZ_CP009257.1 | 2702049 | 2702882 | − | 834 | 14 |
| IX87_RS14090 | NZ_CP009257.1 | 2801180 | 2801515 | − | 336 | 14 |
| IX87_RS14505 | NZ_CP009257.1 | 2883617 | 2885167 | + | 1551 | 14 |
| IX87_RS14730 | NZ_CP009257.1 | 2936463 | 2936843 | + | 381 | 14 |
| IX87_RS14910 | NZ_CP009257.1 | 2979110 | 2979700 | + | 591 | 14 |
| IX87_RS15080 | NZ_CP009257.1 | 3009761 | 3010681 | + | 921 | 14 |
| IX87_RS15500 | NZ_CP009257.1 | 3095393 | 3096454 | − | 1062 | 14 |
| IX87_RS15575 | NZ_CP009257.1 | 3109422 | 3110165 | + | 744 | 14 |
| IX87_RS15690 | NZ_CP009257.1 | 3132276 | 3133154 | + | 879 | 14 |
| IX87_RS15895 | NZ_CP009257.1 | 3174179 | 3174508 | + | 330 | 14 |
| IX87_RS16085 | NZ_CP009257.1 | 3222992 | 3223573 | + | 582 | 14 |
| IX87_RS17105 | NZ_CP009257.1 | 3435372 | 3435683 | + | 312 | 14 |
| IX87_RS17250 | NZ_CP009257.1 | 3466359 | 3467849 | + | 1491 | 14 |
| IX87_RS17665 | NZ_CP009257.1 | 3550841 | 3553765 | − | 2925 | 14 |
| IX87_RS17820 | NZ_CP009257.1 | 3583557 | 3584318 | + | 762 | 14 |
| IX87_RS17955 | NZ_CP009257.1 | 3613807 | 3614541 | − | 735 | 14 |
| IX87_RS18045 | NZ_CP009257.1 | 3634223 | 3635146 | − | 924 | 14 |
| IX87_RS18830 | NZ_CP009257.1 | 3780666 | 3781313 | − | 648 | 14 |
| IX87_RS18995 | NZ_CP009257.1 | 3806314 | 3807519 | + | 1206 | 14 |
| IX87_RS19015 | NZ_CP009257.1 | 3811511 | 3812263 | + | 753 | 14 |
| IX87_RS19045 | NZ_CP009257.1 | 3818492 | 3819895 | + | 1404 | 14 |
| IX87_RS19185 | NZ_CP009257.1 | 3848969 | 3849865 | + | 897 | 14 |
| IX87_RS19220 | NZ_CP009257.1 | 3855898 | 3856725 | + | 828 | 14 |
| IX87_RS19250 | NZ_CP009257.1 | 3860208 | 3861551 | + | 1344 | 14 |
| IX87_RS19425 | NZ_CP009257.1 | 3899825 | 3901384 | − | 1560 | 14 |
| IX87_RS19445 | NZ_CP009257.1 | 3903984 | 3904349 | + | 366 | 14 |
| IX87_RS19840 | NZ_CP009257.1 | 3987786 | 3988604 | + | 819 | 14 |
| IX87_RS21295 | NZ_CP009257.1 | 4265774 | 4266694 | + | 921 | 14 |
| IX87_RS21410 | NZ_CP009257.1 | 4283395 | 4284435 | − | 1041 | 14 |
| IX87_RS00370 | NZ_CP009257.1 | 51242 | 51760 | − | 519 | 13 |
| IX87_RS00585 | NZ_CP009257.1 | 93299 | 94306 | + | 1008 | 13 |
| IX87_RS02225 | NZ_CP009257.1 | 409724 | 410332 | − | 609 | 13 |
| IX87_RS02290 | NZ_CP009257.1 | 420240 | 421253 | − | 1014 | 13 |
| IX87_RS03295 | NZ_CP009257.1 | 614474 | 615757 | − | 1284 | 13 |
| IX87_RS05315 | NZ_CP009257.1 | 1025152 | 1025922 | + | 771 | 13 |
| IX87_RS05505 | NZ_CP009257.1 | 1065705 | 1066949 | − | 1245 | 13 |
| IX87_RS06355 | NZ_CP009257.1 | 1224959 | 1225678 | + | 720 | 13 |
| IX87_RS06460 | NZ_CP009257.1 | 1244117 | 1244479 | − | 363 | 13 |
| IX87_RS08920 | NZ_CP009257.1 | 1742438 | 1743601 | + | 1164 | 13 |
| IX87_RS09380 | NZ_CP009257.1 | 1831764 | 1832885 | + | 1122 | 13 |
| IX87_RS09685 | NZ_CP009257.1 | 1909743 | 1910411 | − | 669 | 13 |
| IX87_RS11150 | NZ_CP009257.1 | 2212527 | 2213294 | − | 768 | 13 |
| IX87_RS11415 | NZ_CP009257.1 | 2263285 | 2263728 | + | 444 | 13 |
| IX87_RS11690 | NZ_CP009257.1 | 2317049 | 2317258 | + | 210 | 13 |
| IX87_RS11925 | NZ_CP009257.1 | 2352527 | 2353513 | − | 987 | 13 |
| IX87_RS12410 | NZ_CP009257.1 | 2448391 | 2448951 | − | 561 | 13 |
| IX87_RS13285 | NZ_CP009257.1 | 2627306 | 2628364 | − | 1059 | 13 |
| IX87_RS13970 | NZ_CP009257.1 | 2769998 | 2770375 | − | 378 | 13 |
| IX87_RS14145 | NZ_CP009257.1 | 2815698 | 2815832 | + | 135 | 13 |
| IX87_RS14960 | NZ_CP009257.1 | 2987802 | 2988470 | + | 669 | 13 |
| IX87_RS15350 | NZ_CP009257.1 | 3065127 | 3065786 | + | 660 | 13 |
| IX87_RS15525 | NZ_CP009257.1 | 3100636 | 3100905 | − | 270 | 13 |
| IX87_RS15915 | NZ_CP009257.1 | 3175150 | 3175674 | + | 525 | 13 |
| IX87_RS16320 | NZ_CP009257.1 | 3269500 | 3270933 | + | 1434 | 13 |
| IX87_RS16525 | NZ_CP009257.1 | 3308556 | 3309248 | + | 693 | 13 |
| IX87_RS16860 | NZ_CP009257.1 | 3382067 | 3383056 | − | 990 | 13 |
| IX87_RS17010 | NZ_CP009257.1 | 3414546 | 3416000 | + | 1455 | 13 |
| IX87_RS17085 | NZ_CP009257.1 | 3432354 | 3432836 | + | 483 | 13 |
| IX87_RS00335 | NZ_CP009257.1 | 43617 | 44393 | + | 777 | 12 |
| IX87_RS00475 | NZ_CP009257.1 | 70257 | 70805 | + | 549 | 12 |
| IX87_RS00550 | NZ_CP009257.1 | 85927 | 86604 | − | 678 | 12 |
| IX87_RS00595 | NZ_CP009257.1 | 96763 | 97401 | − | 639 | 12 |
| IX87_RS00770 | NZ_CP009257.1 | 137106 | 137918 | + | 813 | 12 |
| IX87_RS00785 | NZ_CP009257.1 | 139109 | 139897 | + | 789 | 12 |
| IX87_RS01620 | NZ_CP009257.1 | 302526 | 302849 | − | 324 | 12 |
| IX87_RS01635 | NZ_CP009257.1 | 304777 | 305625 | − | 849 | 12 |
| IX87_RS01855 | NZ_CP009257.1 | 351451 | 351714 | − | 264 | 12 |
| IX87_RS02375 | NZ_CP009257.1 | 437913 | 438353 | + | 441 | 12 |
| IX87_RS02780 | NZ_CP009257.1 | 511704 | 512549 | − | 846 | 12 |
| IX87_RS02890 | NZ_CP009257.1 | 537206 | 538096 | + | 891 | 12 |
| IX87_RS02965 | NZ_CP009257.1 | 553842 | 555287 | + | 1446 | 12 |
| IX87_RS03085 | NZ_CP009257.1 | 575227 | 576087 | + | 861 | 12 |
| IX87_RS03155 | NZ_CP009257.1 | 587823 | 588683 | − | 861 | 12 |
| IX87_RS03350 | NZ_CP009257.1 | 630752 | 631555 | − | 804 | 12 |
| IX87_RS03580 | NZ_CP009257.1 | 683587 | 684156 | − | 570 | 12 |
| IX87_RS03620 | NZ_CP009257.1 | 691789 | 692754 | − | 966 | 12 |
| IX87_RS03645 | NZ_CP009257.1 | 697691 | 698464 | − | 774 | 12 |
| IX87_RS05280 | NZ_CP009257.1 | 1017741 | 1019147 | − | 1407 | 12 |
| IX87_RS05305 | NZ_CP009257.1 | 1022952 | 1023374 | + | 423 | 12 |
| IX87_RS05320 | NZ_CP009257.1 | 1025982 | 1026701 | + | 720 | 12 |
| IX87_RS05455 | NZ_CP009257.1 | 1055561 | 1057591 | − | 2031 | 12 |
| IX87_RS05515 | NZ_CP009257.1 | 1068702 | 1069712 | − | 1011 | 12 |
| IX87_RS06125 | NZ_CP009257.1 | 1175449 | 1176621 | + | 1173 | 12 |
| IX87_RS06550 | NZ_CP009257.1 | 1268542 | 1269120 | + | 579 | 12 |
| IX87_RS06765 | NZ_CP009257.1 | 1315687 | 1316712 | + | 1026 | 12 |
| IX87_RS07880 | NZ_CP009257.1 | 1532453 | 1533211 | + | 759 | 12 |
| IX87_RS08560 | NZ_CP009257.1 | 1656783 | 1658891 | + | 2109 | 12 |
| IX87_RS08570 | NZ_CP009257.1 | 1659372 | 1660343 | − | 972 | 12 |
| IX87_RS08620 | NZ_CP009257.1 | 1671077 | 1671622 | + | 546 | 12 |
| IX87_RS08685 | NZ_CP009257.1 | 1685182 | 1686561 | − | 1380 | 12 |
| IX87_RS09090 | NZ_CP009257.1 | 1773716 | 1774315 | − | 600 | 12 |
| IX87_RS09095 | NZ_CP009257.1 | 1774325 | 1775395 | − | 1071 | 12 |
| IX87_RS09175 | NZ_CP009257.1 | 1787888 | 1788478 | − | 591 | 12 |
| IX87_RS09195 | NZ_CP009257.1 | 1790095 | 1791261 | − | 1167 | 12 |
| IX87_RS10130 | NZ_CP009257.1 | 2017763 | 2018224 | + | 462 | 12 |
| IX87_RS10525 | NZ_CP009257.1 | 2082349 | 2082804 | − | 456 | 12 |
| IX87_RS10560 | NZ_CP009257.1 | 2088139 | 2089332 | − | 1194 | 12 |
| IX87_RS10645 | NZ_CP009257.1 | 2104382 | 2104885 | − | 504 | 12 |
| IX87_RS11275 | NZ_CP009257.1 | 2237334 | 2238185 | − | 852 | 12 |
| IX87_RS11500 | NZ_CP009257.1 | 2279953 | 2280516 | − | 564 | 12 |
| IX87_RS11640 | NZ_CP009257.1 | 2306284 | 2306781 | − | 498 | 12 |
| IX87_RS11995 | NZ_CP009257.1 | 2365243 | 2366073 | − | 831 | 12 |
| IX87_RS12110 | NZ_CP009257.1 | 2389347 | 2389766 | + | 420 | 12 |
| IX87_RS12540 | NZ_CP009257.1 | 2478623 | 2479447 | − | 825 | 12 |
| IX87_RS12635 | NZ_CP009257.1 | 2495819 | 2496157 | + | 339 | 12 |
| IX87_RS12755 | NZ_CP009257.1 | 2522965 | 2523582 | + | 618 | 12 |
| IX87_RS13655 | NZ_CP009257.1 | 2707570 | 2708421 | − | 852 | 12 |
| IX87_RS13735 | NZ_CP009257.1 | 2723214 | 2723852 | − | 639 | 12 |
| IX87_RS13780 | NZ_CP009257.1 | 2734929 | 2736041 | + | 1113 | 12 |
| IX87_RS14020 | NZ_CP009257.1 | 2785537 | 2786499 | + | 963 | 12 |
| IX87_RS14465 | NZ_CP009257.1 | 2875030 | 2875668 | − | 639 | 12 |
| IX87_RS14665 | NZ_CP009257.1 | 2921094 | 2921978 | + | 885 | 12 |
| IX87_RS14895 | NZ_CP009257.1 | 2977186 | 2977578 | + | 393 | 12 |
| IX87_RS14955 | NZ_CP009257.1 | 2987282 | 2987689 | + | 408 | 12 |
| IX87_RS15280 | NZ_CP009257.1 | 3051012 | 3052328 | − | 1317 | 12 |
| IX87_RS15835 | NZ_CP009257.1 | 3162242 | 3162682 | − | 441 | 12 |
| IX87_RS15855 | NZ_CP009257.1 | 3166531 | 3166857 | + | 327 | 12 |
| IX87_RS15885 | NZ_CP009257.1 | 3171369 | 3173081 | − | 1713 | 12 |
| IX87_RS15940 | NZ_CP009257.1 | 3180781 | 3181056 | − | 276 | 12 |
| IX87_RS16845 | NZ_CP009257.1 | 3377208 | 3378602 | − | 1395 | 12 |
| IX87_RS17030 | NZ_CP009257.1 | 3418419 | 3419285 | − | 867 | 12 |
| IX87_RS17735 | NZ_CP009257.1 | 3565317 | 3565943 | − | 627 | 12 |
| IX87_RS17960 | NZ_CP009257.1 | 3614557 | 3615309 | − | 753 | 12 |
| IX87_RS18050 | NZ_CP009257.1 | 3635188 | 3635784 | − | 597 | 12 |
| IX87_RS18585 | NZ_CP009257.1 | 3725109 | 3725465 | + | 357 | 12 |
| IX87_RS18605 | NZ_CP009257.1 | 3731267 | 3731605 | − | 339 | 12 |
| IX87_RS18665 | NZ_CP009257.1 | 3741368 | 3742333 | − | 966 | 12 |
| IX87_RS18755 | NZ_CP009257.1 | 3764136 | 3764756 | + | 621 | 12 |
| IX87_RS19030 | NZ_CP009257.1 | 3814958 | 3815920 | + | 963 | 12 |
| IX87_RS19110 | NZ_CP009257.1 | 3831820 | 3833169 | − | 1350 | 12 |
| IX87_RS19180 | NZ_CP009257.1 | 3847835 | 3848731 | + | 897 | 12 |
| IX87_RS20580 | NZ_CP009257.1 | 4135765 | 4136481 | − | 717 | 12 |
| IX87_RS20625 | NZ_CP009257.1 | 4145243 | 4146001 | − | 759 | 12 |
| IX87_RS20775 | NZ_CP009257.1 | 4173772 | 4174980 | − | 1209 | 12 |
| IX87_RS21140 | NZ_CP009257.1 | 4235999 | 4237177 | − | 1179 | 12 |
| IX87_RS21180 | NZ_CP009257.1 | 4243959 | 4244252 | − | 294 | 12 |
| IX87_RS00895 | NZ_CP009257.1 | 163669 | 165546 | − | 1878 | 11 |
| IX87_RS01610 | NZ_CP009257.1 | 301524 | 301961 | − | 438 | 11 |
| IX87_RS02565 | NZ_CP009257.1 | 474867 | 476282 | + | 1416 | 11 |
| IX87_RS03280 | NZ_CP009257.1 | 611881 | 612171 | + | 291 | 11 |
| IX87_RS03635 | NZ_CP009257.1 | 695359 | 696897 | − | 1539 | 11 |
| IX87_RS04500 | NZ_CP009257.1 | 876236 | 876451 | + | 216 | 11 |
| IX87_RS06285 | NZ_CP009257.1 | 1207744 | 1208835 | + | 1092 | 11 |
| IX87_RS09070 | NZ_CP009257.1 | 1769283 | 1769735 | − | 453 | 11 |
| IX87_RS09390 | NZ_CP009257.1 | 1834016 | 1835107 | + | 1092 | 11 |
| IX87_RS09665 | NZ_CP009257.1 | 1904298 | 1905815 | − | 1518 | 11 |
| IX87_RS10690 | NZ_CP009257.1 | 2115807 | 2116169 | − | 363 | 11 |
| IX87_RS10895 | NZ_CP009257.1 | 2160289 | 2161149 | + | 861 | 11 |
| IX87_RS11495 | NZ_CP009257.1 | 2278653 | 2279816 | − | 1164 | 11 |
| IX87_RS11695 | NZ_CP009257.1 | 2317270 | 2317701 | + | 432 | 11 |
| IX87_RS11940 | NZ_CP009257.1 | 2356467 | 2357102 | + | 636 | 11 |
| IX87_RS12325 | NZ_CP009257.1 | 2431496 | 2431747 | − | 252 | 11 |
| IX87_RS12915 | NZ_CP009257.1 | 2550523 | 2550933 | − | 411 | 11 |
| IX87_RS13305 | NZ_CP009257.1 | 2631194 | 2632894 | − | 1701 | 11 |
| IX87_RS13455 | NZ_CP009257.1 | 2665744 | 2666394 | − | 651 | 11 |
| IX87_RS13945 | NZ_CP009257.1 | 2765375 | 2766565 | + | 1191 | 11 |
| IX87_RS14990 | NZ_CP009257.1 | 2995020 | 2995829 | − | 810 | 11 |
| IX87_RS15210 | NZ_CP009257.1 | 3036478 | 3037236 | − | 759 | 11 |
| IX87_RS15415 | NZ_CP009257.1 | 3076544 | 3078067 | − | 1524 | 11 |
| IX87_RS16985 | NZ_CP009257.1 | 3410236 | 3411420 | − | 1185 | 11 |
| IX87_RS17270 | NZ_CP009257.1 | 3469412 | 3470236 | − | 825 | 11 |
| IX87_RS17390 | NZ_CP009257.1 | 3496513 | 3497139 | + | 627 | 11 |
| IX87_RS18100 | NZ_CP009257.1 | 3641988 | 3642755 | − | 768 | 11 |
| IX87_RS18130 | NZ_CP009257.1 | 3647579 | 3648160 | − | 582 | 11 |
| IX87_RS18615 | NZ_CP009257.1 | 3733522 | 3734040 | − | 519 | 11 |
| IX87_RS18775 | NZ_CP009257.1 | 3768473 | 3769528 | + | 1056 | 11 |
| IX87_RS19255 | NZ_CP009257.1 | 3861585 | 3862370 | + | 786 | 11 |
| IX87_RS20575 | NZ_CP009257.1 | 4134651 | 4135745 | − | 1095 | 11 |
| IX87_RS21380 | NZ_CP009257.1 | 4278378 | 4279781 | + | 1404 | 11 |
| IX87_RS00395 | NZ_CP009257.1 | 55826 | 56491 | − | 666 | 10 |
| IX87_RS00820 | NZ_CP009257.1 | 146453 | 147451 | − | 999 | 10 |
| IX87_RS01570 | NZ_CP009257.1 | 291456 | 291908 | − | 453 | 10 |
| IX87_RS02185 | NZ_CP009257.1 | 403908 | 404411 | − | 504 | 10 |
| IX87_RS02240 | NZ_CP009257.1 | 411514 | 412494 | − | 981 | 10 |
| IX87_RS02385 | NZ_CP009257.1 | 439526 | 439900 | + | 375 | 10 |
| IX87_RS02430 | NZ_CP009257.1 | 450213 | 450803 | + | 591 | 10 |
| IX87_RS02610 | NZ_CP009257.1 | 480900 | 481763 | − | 864 | 10 |
| IX87_RS02815 | NZ_CP009257.1 | 522464 | 523279 | + | 816 | 10 |
| IX87_RS02920 | NZ_CP009257.1 | 543306 | 544101 | − | 796 | 10 |
| IX87_RS03135 | NZ_CP009257.1 | 582536 | 584497 | + | 1962 | 10 |
| IX87_RS03640 | NZ_CP009257.1 | 696900 | 697691 | − | 792 | 10 |
| IX87_RS03650 | NZ_CP009257.1 | 698473 | 699534 | − | 1062 | 10 |
| IX87_RS03805 | NZ_CP009257.1 | 730021 | 731385 | − | 1365 | 10 |
| IX87_RS04065 | NZ_CP009257.1 | 791722 | 792969 | + | 1248 | 10 |
| IX87_RS04300 | NZ_CP009257.1 | 836916 | 837374 | − | 459 | 10 |
| IX87_RS05220 | NZ_CP009257.1 | 1006056 | 1006769 | + | 714 | 10 |
| IX87_RS05325 | NZ_CP009257.1 | 1026767 | 1027474 | + | 708 | 10 |
| IX87_RS05580 | NZ_CP009257.1 | 1084582 | 1085184 | + | 603 | 10 |
| IX87_RS06705 | NZ_CP009257.1 | 1301939 | 1302718 | − | 780 | 10 |
| IX87_RS06830 | NZ_CP009257.1 | 1329202 | 1330008 | − | 807 | 10 |
| IX87_RS07275 | NZ_CP009257.1 | 1415264 | 1415917 | + | 654 | 10 |
| IX87_RS08645 | NZ_CP009257.1 | 1675642 | 1676283 | + | 642 | 10 |
| IX87_RS08740 | NZ_CP009257.1 | 1698523 | 1699218 | + | 696 | 10 |
| IX87_RS09810 | NZ_CP009257.1 | 1930889 | 1932196 | − | 1308 | 10 |
| IX87_RS09950 | NZ_CP009257.1 | 1978637 | 1979197 | − | 561 | 10 |
| IX87_RS10675 | NZ_CP009257.1 | 2108418 | 2112938 | − | 4521 | 10 |
| IX87_RS10920 | NZ_CP009257.1 | 2163505 | 2164011 | − | 507 | 10 |
| IX87_RS11075 | NZ_CP009257.1 | 2197481 | 2197927 | + | 447 | 10 |
| IX87_RS11260 | NZ_CP009257.1 | 2234280 | 2235533 | − | 1254 | 10 |
| IX87_RS11420 | NZ_CP009257.1 | 2263771 | 2264610 | − | 840 | 10 |
| IX87_RS11450 | NZ_CP009257.1 | 2268261 | 2268689 | + | 429 | 10 |
| IX87_RS11625 | NZ_CP009257.1 | 2304280 | 2305194 | − | 915 | 10 |
| IX87_RS12060 | NZ_CP009257.1 | 2379693 | 2380955 | − | 1263 | 10 |
| IX87_RS12240 | NZ_CP009257.1 | 2416652 | 2416966 | + | 315 | 10 |
| IX87_RS12575 | NZ_CP009257.1 | 2486050 | 2486973 | − | 924 | 10 |
| IX87_RS13075 | NZ_CP009257.1 | 2579131 | 2579514 | − | 384 | 10 |
| IX87_RS13080 | NZ_CP009257.1 | 2579562 | 2580389 | − | 828 | 10 |
| IX87_RS13155 | NZ_CP009257.1 | 2596370 | 2597044 | + | 675 | 10 |
| IX87_RS13505 | NZ_CP009257.1 | 2674837 | 2675856 | + | 1020 | 10 |
| IX87_RS13730 | NZ_CP009257.1 | 2722390 | 2723190 | − | 801 | 10 |
| IX87_RS13805 | NZ_CP009257.1 | 2741370 | 2742002 | + | 633 | 10 |
| IX87_RS13810 | NZ_CP009257.1 | 2742020 | 2742568 | + | 549 | 10 |
| IX87_RS14155 | NZ_CP009257.1 | 2816265 | 2816585 | + | 321 | 10 |
| IX87_RS14220 | NZ_CP009257.1 | 2827087 | 2828247 | − | 1161 | 10 |
| IX87_RS14315 | NZ_CP009257.1 | 2846289 | 2846615 | − | 327 | 10 |
| IX87_RS14785 | NZ_CP009257.1 | 2949640 | 2950530 | + | 891 | 10 |
| IX87_RS15085 | NZ_CP009257.1 | 3011073 | 3011642 | − | 570 | 10 |
| IX87_RS15445 | NZ_CP009257.1 | 3083828 | 3084748 | − | 921 | 10 |
| IX87_RS15790 | NZ_CP009257.1 | 3152376 | 3154175 | + | 1800 | 10 |
| IX87_RS15815 | NZ_CP009257.1 | 3157576 | 3158349 | − | 774 | 10 |
| IX87_RS15945 | NZ_CP009257.1 | 3181053 | 3181580 | − | 528 | 10 |
| IX87_RS16060 | NZ_CP009257.1 | 3215678 | 3216289 | − | 612 | 10 |
| IX87_RS16255 | NZ_CP009257.1 | 3256528 | 3257133 | + | 606 | 10 |
| IX87_RS16325 | NZ_CP009257.1 | 3270952 | 3271599 | + | 648 | 10 |
| IX87_RS16545 | NZ_CP009257.1 | 3311998 | 3312402 | + | 405 | 10 |
| IX87_RS17345 | NZ_CP009257.1 | 3484774 | 3485859 | + | 1086 | 10 |
| IX87_RS17435 | NZ_CP009257.1 | 3504119 | 3504514 | + | 396 | 10 |
| IX87_RS17615 | NZ_CP009257.1 | 3537246 | 3538541 | − | 1296 | 10 |
| IX87_RS18030 | NZ_CP009257.1 | 3631610 | 3632767 | + | 1158 | 10 |
| IX87_RS18125 | NZ_CP009257.1 | 3647067 | 3647447 | − | 381 | 10 |
| IX87_RS18200 | NZ_CP009257.1 | 3658830 | 3659567 | + | 738 | 10 |
| IX87_RS18555 | NZ_CP009257.1 | 3720690 | 3721424 | + | 735 | 10 |
| IX87_RS18745 | NZ_CP009257.1 | 3762142 | 3763665 | + | 1524 | 10 |
| IX87_RS18990 | NZ_CP009257.1 | 3805090 | 3806277 | + | 1188 | 10 |
| IX87_RS19210 | NZ_CP009257.1 | 3854282 | 3855163 | − | 882 | 10 |
| IX87_RS19960 | NZ_CP009257.1 | 4010432 | 4011925 | − | 1494 | 10 |
| IX87_RS20430 | NZ_CP009257.1 | 4104684 | 4105805 | − | 1122 | 10 |
| IX87_RS20475 | NZ_CP009257.1 | 4114472 | 4115791 | − | 1320 | 10 |
| IX87_RS20540 | NZ_CP009257.1 | 4127164 | 4127514 | − | 351 | 10 |
| IX87_RS20640 | NZ_CP009257.1 | 4148832 | 4149512 | − | 681 | 10 |
| IX87_RS21335 | NZ_CP009257.1 | 4272336 | 4272797 | + | 462 | 10 |
| IX87_RS21340 | NZ_CP009257.1 | 4272979 | 4273275 | + | 297 | 10 |
| IX87_RS00500 | NZ_CP009257.1 | 77208 | 77696 | + | 489 | 9 |
| IX87_RS00890 | NZ_CP009257.1 | 162280 | 163614 | + | 1335 | 9 |
| IX87_RS01590 | NZ_CP009257.1 | 294654 | 298250 | + | 3597 | 9 |
| IX87_RS02265 | NZ_CP009257.1 | 416458 | 417093 | − | 636 | 9 |
| IX87_RS03590 | NZ_CP009257.1 | 685352 | 685945 | − | 594 | 9 |
| IX87_RS03660 | NZ_CP009257.1 | 700075 | 700830 | − | 756 | 9 |
| IX87_RS05005 | NZ_CP009257.1 | 958057 | 958623 | − | 567 | 9 |
| IX87_RS05100 | NZ_CP009257.1 | 978078 | 979748 | + | 1671 | 9 |
| IX87_RS05500 | NZ_CP009257.1 | 1063532 | 1065646 | + | 2115 | 9 |
| IX87_RS06510 | NZ_CP009257.1 | 1258118 | 1260772 | − | 2655 | 9 |
| IX87_RS07035 | NZ_CP009257.1 | 1366588 | 1367970 | − | 1383 | 9 |
| IX87_RS07835 | NZ_CP009257.1 | 1520003 | 1520386 | + | 384 | 9 |
| IX87_RS08410 | NZ_CP009257.1 | 1619982 | 1621577 | − | 1596 | 9 |
| IX87_RS08800 | NZ_CP009257.1 | 1713156 | 1713971 | − | 816 | 9 |
| IX87_RS09210 | NZ_CP009257.1 | 1796958 | 1797557 | + | 600 | 9 |
| IX87_RS09770 | NZ_CP009257.1 | 1925563 | 1926195 | + | 633 | 9 |
| IX87_RS10040 | NZ_CP009257.1 | 1997100 | 1997534 | + | 435 | 9 |
| IX87_RS10140 | NZ_CP009257.1 | 2019175 | 2019543 | − | 369 | 9 |
| IX87_RS11020 | NZ_CP009257.1 | 2185105 | 2185818 | + | 714 | 9 |
| IX87_RS11535 | NZ_CP009257.1 | 2286877 | 2287944 | − | 1068 | 9 |
| IX87_RS12195 | NZ_CP009257.1 | 2407574 | 2408137 | − | 564 | 9 |
| IX87_RS12875 | NZ_CP009257.1 | 2541074 | 2541376 | + | 303 | 9 |
| IX87_RS13170 | NZ_CP009257.1 | 2597907 | 2598884 | − | 978 | 9 |
| IX87_RS13520 | NZ_CP009257.1 | 2676804 | 2678171 | + | 1368 | 9 |
| IX87_RS15205 | NZ_CP009257.1 | 3035421 | 3036476 | − | 1056 | 9 |
| IX87_RS15550 | NZ_CP009257.1 | 3105302 | 3106069 | + | 768 | 9 |
| IX87_RS16940 | NZ_CP009257.1 | 3400003 | 3400845 | + | 843 | 9 |
| IX87_RS17455 | NZ_CP009257.1 | 3508405 | 3508725 | + | 321 | 9 |
| IX87_RS17810 | NZ_CP009257.1 | 3582270 | 3582563 | + | 294 | 9 |
| IX87_RS17865 | NZ_CP009257.1 | 3592604 | 3593173 | − | 570 | 9 |
| IX87_RS18025 | NZ_CP009257.1 | 3630328 | 3631608 | + | 1281 | 9 |
| IX87_RS18495 | NZ_CP009257.1 | 3703539 | 3704420 | − | 882 | 9 |
| IX87_RS18570 | NZ_CP009257.1 | 3722638 | 3723144 | + | 507 | 9 |
| IX87_RS19845 | NZ_CP009257.1 | 3988611 | 3989285 | + | 675 | 9 |
| IX87_RS21135 | NZ_CP009257.1 | 4234497 | 4235951 | + | 1455 | 9 |
| IX87_RS00290 | NZ_CP009257.1 | 29786 | 30103 | − | 318 | 8 |
| IX87_RS00390 | NZ_CP009257.1 | 55187 | 55801 | − | 615 | 8 |
| IX87_RS00570 | NZ_CP009257.1 | 90692 | 91315 | + | 624 | 8 |
| IX87_RS00810 | NZ_CP009257.1 | 144434 | 144964 | − | 531 | 8 |
| IX87_RS01575 | NZ_CP009257.1 | 292288 | 292665 | + | 378 | 8 |
| IX87_RS01745 | NZ_CP009257.1 | 326804 | 327685 | − | 882 | 8 |
| IX87_RS01780 | NZ_CP009257.1 | 335566 | 336018 | + | 453 | 8 |
| IX87_RS01810 | NZ_CP009257.1 | 341009 | 341923 | − | 915 | 8 |
| IX87_RS01820 | NZ_CP009257.1 | 343529 | 344644 | − | 1116 | 8 |
| IX87_RS02205 | NZ_CP009257.1 | 407130 | 408089 | − | 960 | 8 |
| IX87_RS02280 | NZ_CP009257.1 | 418831 | 419463 | − | 633 | 8 |
| IX87_RS02495 | NZ_CP009257.1 | 461960 | 463018 | + | 1059 | 8 |
| IX87_RS02835 | NZ_CP009257.1 | 527309 | 528895 | − | 1587 | 8 |
| IX87_RS02910 | NZ_CP009257.1 | 541550 | 542431 | − | 882 | 8 |
| IX87_RS02925 | NZ_CP009257.1 | 544111 | 545151 | − | 1041 | 8 |
| IX87_RS02980 | NZ_CP009257.1 | 556532 | 557950 | + | 1419 | 8 |
| IX87_RS03340 | NZ_CP009257.1 | 626062 | 627003 | − | 942 | 8 |
| IX87_RS03850 | NZ_CP009257.1 | 743694 | 745505 | − | 1812 | 8 |
| IX87_RS04265 | NZ_CP009257.1 | 831560 | 832153 | − | 594 | 8 |
| IX87_RS05055 | NZ_CP009257.1 | 967998 | 968621 | − | 624 | 8 |
| IX87_RS05150 | NZ_CP009257.1 | 990657 | 991115 | − | 459 | 8 |
| IX87_RS06075 | NZ_CP009257.1 | 1165342 | 1165920 | + | 579 | 8 |
| IX87_RS06370 | NZ_CP009257.1 | 1226736 | 1227593 | − | 858 | 8 |
| IX87_RS06555 | NZ_CP009257.1 | 1269167 | 1270810 | − | 1644 | 8 |
| IX87_RS06700 | NZ_CP009257.1 | 1301188 | 1301898 | − | 711 | 8 |
| IX87_RS06710 | NZ_CP009257.1 | 1302721 | 1303317 | − | 597 | 8 |
| IX87_RS23040 | NZ_CP009257.1 | 1305554 | 1306123 | + | 570 | 8 |
| IX87_RS07810 | NZ_CP009257.1 | 1513071 | 1514285 | + | 1215 | 8 |
| IX87_RS08440 | NZ_CP009257.1 | 1627450 | 1630317 | + | 2868 | 8 |
| IX87_RS08650 | NZ_CP009257.1 | 1676333 | 1678519 | − | 2187 | 8 |
| IX87_RS08670 | NZ_CP009257.1 | 1680821 | 1682191 | − | 1371 | 8 |
| IX87_RS08790 | NZ_CP009257.1 | 1709830 | 1710213 | + | 384 | 8 |
| IX87_RS08890 | NZ_CP009257.1 | 1735856 | 1736761 | + | 906 | 8 |
| IX87_RS08940 | NZ_CP009257.1 | 1746777 | 1747421 | − | 645 | 8 |
| IX87_RS09105 | NZ_CP009257.1 | 1776473 | 1777480 | + | 1008 | 8 |
| IX87_RS09280 | NZ_CP009257.1 | 1808577 | 1808966 | + | 390 | 8 |
| IX87_RS09360 | NZ_CP009257.1 | 1826884 | 1827369 | − | 486 | 8 |
| IX87_RS09905 | NZ_CP009257.1 | 1969819 | 1970751 | + | 933 | 8 |
| IX87_RS10035 | NZ_CP009257.1 | 1996742 | 1997092 | + | 351 | 8 |
| IX87_RS10120 | NZ_CP009257.1 | 2016046 | 2016672 | + | 627 | 8 |
| IX87_RS10425 | NZ_CP009257.1 | 2061969 | 2062862 | − | 894 | 8 |
| IX87_RS10450 | NZ_CP009257.1 | 2067335 | 2068033 | + | 699 | 8 |
| IX87_RS10530 | NZ_CP009257.1 | 2082951 | 2084018 | − | 1068 | 8 |
| IX87_RS10540 | NZ_CP009257.1 | 2084936 | 2085991 | − | 1056 | 8 |
| IX87_RS10585 | NZ_CP009257.1 | 2094755 | 2095459 | + | 705 | 8 |
| IX87_RS10680 | NZ_CP009257.1 | 2113085 | 2115163 | − | 2079 | 8 |
| IX87_RS10765 | NZ_CP009257.1 | 2130498 | 2132585 | − | 2088 | 8 |
| IX87_RS10770 | NZ_CP009257.1 | 2132760 | 2133326 | − | 567 | 8 |
| IX87_RS11015 | NZ_CP009257.1 | 2184454 | 2185032 | + | 579 | 8 |
| IX87_RS11230 | NZ_CP009257.1 | 2227868 | 2228896 | + | 1029 | 8 |
| IX87_RS11315 | NZ_CP009257.1 | 2243076 | 2243498 | − | 423 | 8 |
| IX87_RS11445 | NZ_CP009257.1 | 2267601 | 2268254 | + | 654 | 8 |
| IX87_RS11485 | NZ_CP009257.1 | 2276030 | 2277835 | − | 1806 | 8 |
| IX87_RS11505 | NZ_CP009257.1 | 2280536 | 2280886 | − | 351 | 8 |
| IX87_RS11720 | NZ_CP009257.1 | 2323127 | 2323372 | − | 246 | 8 |
| IX87_RS13115 | NZ_CP009257.1 | 2587857 | 2589401 | + | 1545 | 8 |
| IX87_RS13235 | NZ_CP009257.1 | 2617126 | 2617776 | − | 651 | 8 |
| IX87_RS13595 | NZ_CP009257.1 | 2694574 | 2695089 | − | 516 | 8 |
| IX87_RS13795 | NZ_CP009257.1 | 2738329 | 2739717 | − | 1389 | 8 |
| IX87_RS13820 | NZ_CP009257.1 | 2743713 | 2744768 | − | 1056 | 8 |
| IX87_RS14085 | NZ_CP009257.1 | 2800280 | 2801125 | + | 846 | 8 |
| IX87_RS14110 | NZ_CP009257.1 | 2805510 | 2807441 | + | 1932 | 8 |
| IX87_RS14170 | NZ_CP009257.1 | 2819844 | 2820107 | − | 264 | 8 |
| IX87_RS14200 | NZ_CP009257.1 | 2823617 | 2824144 | − | 528 | 8 |
| IX87_RS14435 | NZ_CP009257.1 | 2870520 | 2870876 | − | 357 | 8 |
| IX87_RS14495 | NZ_CP009257.1 | 2881958 | 2882803 | − | 846 | 8 |
| IX87_RS15250 | NZ_CP009257.1 | 3043654 | 3044973 | + | 1320 | 8 |
| IX87_RS15325 | NZ_CP009257.1 | 3060284 | 3060622 | + | 339 | 8 |
| IX87_RS15340 | NZ_CP009257.1 | 3062710 | 3063795 | + | 1086 | 8 |
| IX87_RS15860 | NZ_CP009257.1 | 3166959 | 3167708 | + | 750 | 8 |
| IX87_RS15870 | NZ_CP009257.1 | 3168655 | 3169251 | − | 597 | 8 |
| IX87_RS16080 | NZ_CP009257.1 | 3221763 | 3222968 | + | 1206 | 8 |
| IX87_RS16295 | NZ_CP009257.1 | 3266406 | 3267056 | + | 651 | 8 |
| IX87_RS16310 | NZ_CP009257.1 | 3268045 | 3268905 | − | 861 | 8 |
| IX87_RS16360 | NZ_CP009257.1 | 3275979 | 3276776 | − | 798 | 8 |
| IX87_RS16575 | NZ_CP009257.1 | 3316047 | 3316406 | − | 360 | 8 |
| IX87_RS16620 | NZ_CP009257.1 | 3325867 | 3326490 | − | 624 | 8 |
| IX87_RS16675 | NZ_CP009257.1 | 3339164 | 3339718 | − | 555 | 8 |
| IX87_RS16950 | NZ_CP009257.1 | 3401874 | 3402686 | + | 813 | 8 |
| IX87_RS16980 | NZ_CP009257.1 | 3409083 | 3410087 | − | 1005 | 8 |
| IX87_RS17000 | NZ_CP009257.1 | 3413080 | 3414261 | + | 1182 | 8 |
| IX87_RS17075 | NZ_CP009257.1 | 3430359 | 3430769 | + | 411 | 8 |
| IX87_RS17135 | NZ_CP009257.1 | 3441683 | 3442111 | + | 429 | 8 |
| IX87_RS17185 | NZ_CP009257.1 | 3450307 | 3450603 | + | 297 | 8 |
| IX87_RS17450 | NZ_CP009257.1 | 3507464 | 3508255 | + | 792 | 8 |
| IX87_RS17495 | NZ_CP009257.1 | 3512483 | 3513478 | − | 996 | 8 |
| IX87_RS17550 | NZ_CP009257.1 | 3526663 | 3527592 | − | 930 | 8 |
| IX87_RS17825 | NZ_CP009257.1 | 3584366 | 3584785 | − | 420 | 8 |
| IX87_RS17830 | NZ_CP009257.1 | 3585078 | 3585305 | + | 228 | 8 |
| IX87_RS17965 | NZ_CP009257.1 | 3615340 | 3616143 | + | 804 | 8 |
| IX87_RS18565 | NZ_CP009257.1 | 3721960 | 3722625 | + | 666 | 8 |
| IX87_RS18670 | NZ_CP009257.1 | 3742505 | 3743728 | + | 1224 | 8 |
| IX87_RS18845 | NZ_CP009257.1 | 3784215 | 3785339 | + | 1125 | 8 |
| IX87_RS19010 | NZ_CP009257.1 | 3809961 | 3811190 | − | 1230 | 8 |
| IX87_RS19025 | NZ_CP009257.1 | 3813607 | 3814593 | − | 987 | 8 |
| IX87_RS19040 | NZ_CP009257.1 | 3816955 | 3818481 | + | 1527 | 8 |
| IX87_RS19055 | NZ_CP009257.1 | 3820737 | 3821795 | + | 1059 | 8 |
| IX87_RS19105 | NZ_CP009257.1 | 3831128 | 3831736 | + | 609 | 8 |
| IX87_RS19155 | NZ_CP009257.1 | 3840662 | 3841948 | − | 1287 | 8 |
| IX87_RS19795 | NZ_CP009257.1 | 3979005 | 3979748 | + | 744 | 8 |
| IX87_RS20315 | NZ_CP009257.1 | 4080278 | 4081198 | + | 921 | 8 |
| IX87_RS20675 | NZ_CP009257.1 | 4158388 | 4160484 | − | 2097 | 8 |
| IX87_RS20705 | NZ_CP009257.1 | 4164259 | 4164453 | + | 195 | 8 |
| IX87_RS21170 | NZ_CP009257.1 | 4242876 | 4243325 | + | 450 | 8 |
| IX87_RS21310 | NZ_CP009257.1 | 4268015 | 4268641 | + | 627 | 8 |
| IX87_RS21370 | NZ_CP009257.1 | 4277076 | 4277387 | + | 312 | 8 |
| IX87_RS21385 | NZ_CP009257.1 | 4279778 | 4280260 | − | 483 | 8 |
| IX87_RS21390 | NZ_CP009257.1 | 4280469 | 4281704 | − | 1236 | 8 |
| IX87_RS00350 | NZ_CP009257.1 | 47404 | 48195 | + | 792 | 7 |
| IX87_RS00470 | NZ_CP009257.1 | 69523 | 70035 | − | 513 | 7 |
| IX87_RS01545 | NZ_CP009257.1 | 286256 | 287407 | − | 1152 | 7 |
| IX87_RS01595 | NZ_CP009257.1 | 298452 | 299144 | − | 693 | 7 |
| IX87_RS02180 | NZ_CP009257.1 | 403395 | 403838 | − | 444 | 7 |
| IX87_RS02410 | NZ_CP009257.1 | 447103 | 447576 | + | 474 | 7 |
| IX87_RS02540 | NZ_CP009257.1 | 470347 | 471366 | + | 1020 | 7 |
| IX87_RS02705 | NZ_CP009257.1 | 497884 | 498717 | + | 834 | 7 |
| IX87_RS02820 | NZ_CP009257.1 | 523272 | 524087 | + | 816 | 7 |
| IX87_RS04505 | NZ_CP009257.1 | 876516 | 877103 | − | 588 | 7 |
| IX87_RS06030 | NZ_CP009257.1 | 1152729 | 1153859 | − | 1131 | 7 |
| IX87_RS06415 | NZ_CP009257.1 | 1234479 | 1234697 | − | 219 | 7 |
| IX87_RS07270 | NZ_CP009257.1 | 1414385 | 1415158 | − | 774 | 7 |
| IX87_RS08675 | NZ_CP009257.1 | 1682215 | 1683594 | − | 1380 | 7 |
| IX87_RS09160 | NZ_CP009257.1 | 1785960 | 1786646 | + | 687 | 7 |
| IX87_RS09460 | NZ_CP009257.1 | 1850591 | 1851979 | + | 1389 | 7 |
| IX87_RS10950 | NZ_CP009257.1 | 2171022 | 2171798 | + | 777 | 7 |
| IX87_RS10980 | NZ_CP009257.1 | 2176678 | 2177727 | − | 1050 | 7 |
| IX87_RS11160 | NZ_CP009257.1 | 2214043 | 2215071 | − | 1029 | 7 |
| IX87_RS11475 | NZ_CP009257.1 | 2273474 | 2275087 | − | 1614 | 7 |
| IX87_RS12190 | NZ_CP009257.1 | 2406991 | 2407524 | + | 534 | 7 |
| IX87_RS13480 | NZ_CP009257.1 | 2669887 | 2670969 | + | 1083 | 7 |
| IX87_RS14165 | NZ_CP009257.1 | 2818442 | 2819797 | + | 1356 | 7 |
| IX87_RS14230 | NZ_CP009257.1 | 2828823 | 2829779 | − | 957 | 7 |
| IX87_RS14725 | NZ_CP009257.1 | 2936060 | 2936434 | + | 375 | 7 |
| IX87_RS14945 | NZ_CP009257.1 | 2985876 | 2986181 | + | 306 | 7 |
| IX87_RS14985 | NZ_CP009257.1 | 2994392 | 2995018 | + | 627 | 7 |
| IX87_RS15005 | NZ_CP009257.1 | 2997255 | 2998058 | + | 804 | 7 |
| IX87_RS15270 | NZ_CP009257.1 | 3049840 | 3050250 | + | 411 | 7 |
| IX87_RS15580 | NZ_CP009257.1 | 3110165 | 3111001 | + | 837 | 7 |
| IX87_RS15685 | NZ_CP009257.1 | 3131661 | 3132158 | − | 498 | 7 |
| IX87_RS16000 | NZ_CP009257.1 | 3195614 | 3196321 | − | 708 | 7 |
| IX87_RS16120 | NZ_CP009257.1 | 3229501 | 3230112 | + | 612 | 7 |
| IX87_RS17035 | NZ_CP009257.1 | 3419296 | 3420084 | − | 789 | 7 |
| IX87_RS18895 | NZ_CP009257.1 | 3791894 | 3792319 | + | 426 | 7 |
| IX87_RS21305 | NZ_CP009257.1 | 4267411 | 4267893 | − | 483 | 7 |
| IX87_RS00295 | NZ_CP009257.1 | 30397 | 30738 | − | 342 | 6 |
| IX87_RS00300 | NZ_CP009257.1 | 30998 | 31564 | + | 567 | 6 |
| IX87_RS00330 | NZ_CP009257.1 | 43025 | 43504 | + | 480 | 6 |
| IX87_RS00405 | NZ_CP009257.1 | 57030 | 58730 | − | 1701 | 6 |
| IX87_RS00635 | NZ_CP009257.1 | 107022 | 110708 | + | 3687 | 6 |
| IX87_RS01715 | NZ_CP009257.1 | 322329 | 323060 | + | 732 | 6 |
| IX87_RS02155 | NZ_CP009257.1 | 399209 | 399907 | − | 699 | 6 |
| IX87_RS02230 | NZ_CP009257.1 | 410370 | 411143 | − | 774 | 6 |
| IX87_RS02380 | NZ_CP009257.1 | 438462 | 439487 | + | 1026 | 6 |
| IX87_RS02475 | NZ_CP009257.1 | 458526 | 459071 | − | 546 | 6 |
| IX87_RS02490 | NZ_CP009257.1 | 460441 | 461901 | − | 1461 | 6 |
| IX87_RS02640 | NZ_CP009257.1 | 485159 | 485629 | − | 471 | 6 |
| IX87_RS02730 | NZ_CP009257.1 | 502283 | 502990 | + | 708 | 6 |
| IX87_RS02880 | NZ_CP009257.1 | 535481 | 536167 | − | 687 | 6 |
| IX87_RS02940 | NZ_CP009257.1 | 546489 | 547664 | + | 1176 | 6 |
| IX87_RS03040 | NZ_CP009257.1 | 567533 | 568207 | + | 675 | 6 |
| IX87_RS03200 | NZ_CP009257.1 | 598376 | 598987 | + | 612 | 6 |
| IX87_RS03225 | NZ_CP009257.1 | 602525 | 603403 | + | 879 | 6 |
| IX87_RS03655 | NZ_CP009257.1 | 699558 | 700058 | − | 501 | 6 |
| IX87_RS03700 | NZ_CP009257.1 | 711163 | 711915 | − | 753 | 6 |
| IX87_RS03745 | NZ_CP009257.1 | 720113 | 720565 | − | 453 | 6 |
| IX87_RS03875 | NZ_CP009257.1 | 748625 | 749317 | − | 693 | 6 |
| IX87_RS03985 | NZ_CP009257.1 | 769280 | 770500 | − | 1221 | 6 |
| IX87_RS21960 | NZ_CP009257.1 | 794248 | 794442 | − | 195 | 6 |
| IX87_RS04200 | NZ_CP009257.1 | 818665 | 819276 | − | 612 | 6 |
| IX87_RS04290 | NZ_CP009257.1 | 835076 | 836182 | − | 1107 | 6 |
| IX87_RS04380 | NZ_CP009257.1 | 847999 | 848355 | + | 357 | 6 |
| IX87_RS04515 | NZ_CP009257.1 | 877600 | 879186 | + | 1587 | 6 |
| IX87_RS04635 | NZ_CP009257.1 | 904825 | 905850 | − | 1026 | 6 |
| IX87_RS05030 | NZ_CP009257.1 | 962671 | 962868 | − | 198 | 6 |
| IX87_RS05035 | NZ_CP009257.1 | 963267 | 964817 | − | 1551 | 6 |
| IX87_RS05165 | NZ_CP009257.1 | 993543 | 994994 | + | 1452 | 6 |
| IX87_RS05170 | NZ_CP009257.1 | 995076 | 996962 | + | 1887 | 6 |
| IX87_RS05195 | NZ_CP009257.1 | 1000565 | 1001569 | + | 1005 | 6 |
| IX87_RS05510 | NZ_CP009257.1 | 1067135 | 1068724 | − | 1590 | 6 |
| IX87_RS05565 | NZ_CP009257.1 | 1082264 | 1083100 | + | 837 | 6 |
| IX87_RS06215 | NZ_CP009257.1 | 1192967 | 1193218 | − | 252 | 6 |
| IX87_RS06280 | NZ_CP009257.1 | 1207269 | 1207526 | + | 258 | 6 |
| IX87_RS06295 | NZ_CP009257.1 | 1209818 | 1210183 | − | 366 | 6 |
| IX87_RS06340 | NZ_CP009257.1 | 1221390 | 1222112 | + | 723 | 6 |
| IX87_RS06420 | NZ_CP009257.1 | 1234951 | 1235403 | + | 453 | 6 |
| IX87_RS06485 | NZ_CP009257.1 | 1254048 | 1254596 | − | 549 | 6 |
| IX87_RS06575 | NZ_CP009257.1 | 1274158 | 1274979 | − | 822 | 6 |
| IX87_RS06795 | NZ_CP009257.1 | 1321605 | 1322723 | + | 1119 | 6 |
| IX87_RS06810 | NZ_CP009257.1 | 1324979 | 1325890 | + | 912 | 6 |
| IX87_RS07015 | NZ_CP009257.1 | 1361755 | 1362729 | − | 975 | 6 |
| IX87_RS07250 | NZ_CP009257.1 | 1412230 | 1412595 | + | 366 | 6 |
| IX87_RS07255 | NZ_CP009257.1 | 1412609 | 1412806 | + | 198 | 6 |
| IX87_RS07435 | NZ_CP009257.1 | 1449643 | 1450673 | − | 1031 | 6 |
| IX87_RS07460 | NZ_CP009257.1 | 1458278 | 1459507 | − | 1230 | 6 |
| IX87_RS07920 | NZ_CP009257.1 | 1536780 | 1537658 | − | 879 | 6 |
| IX87_RS08340 | NZ_CP009257.1 | 1603751 | 1606126 | − | 2376 | 6 |
| IX87_RS08345 | NZ_CP009257.1 | 1606128 | 1607627 | − | 1500 | 6 |
| IX87_RS08385 | NZ_CP009257.1 | 1614377 | 1615450 | − | 1074 | 6 |
| IX87_RS08480 | NZ_CP009257.1 | 1640472 | 1642319 | − | 1848 | 6 |
| IX87_RS23050 | NZ_CP009257.1 | 1646337 | 1646513 | − | 177 | 6 |
| IX87_RS08600 | NZ_CP009257.1 | 1667891 | 1668658 | − | 768 | 6 |
| IX87_RS08825 | NZ_CP009257.1 | 1718628 | 1719119 | + | 492 | 6 |
| IX87_RS09000 | NZ_CP009257.1 | 1758942 | 1759352 | − | 411 | 6 |
| IX87_RS09025 | NZ_CP009257.1 | 1761964 | 1762608 | − | 645 | 6 |
| IX87_RS09030 | NZ_CP009257.1 | 1762615 | 1763340 | − | 726 | 6 |
| IX87_RS09050 | NZ_CP009257.1 | 1765330 | 1766022 | − | 693 | 6 |
| IX87_RS09235 | NZ_CP009257.1 | 1801449 | 1801901 | + | 453 | 6 |
| IX87_RS09285 | NZ_CP009257.1 | 1809057 | 1811492 | − | 2436 | 6 |
| IX87_RS09340 | NZ_CP009257.1 | 1821286 | 1823718 | − | 2433 | 6 |
| IX87_RS09355 | NZ_CP009257.1 | 1825965 | 1826873 | − | 909 | 6 |
| IX87_RS23120 | NZ_CP009257.1 | 1828601 | 1828747 | + | 147 | 6 |
| IX87_RS09725 | NZ_CP009257.1 | 1914838 | 1915680 | − | 843 | 6 |
| IX87_RS09795 | NZ_CP009257.1 | 1929655 | 1930005 | − | 351 | 6 |
| IX87_RS09915 | NZ_CP009257.1 | 1971474 | 1971680 | − | 207 | 6 |
| IX87_RS10115 | NZ_CP009257.1 | 2014671 | 2015891 | − | 1221 | 6 |
| IX87_RS10570 | NZ_CP009257.1 | 2091003 | 2091938 | − | 936 | 6 |
| IX87_RS10655 | NZ_CP009257.1 | 2105509 | 2106417 | + | 909 | 6 |
| IX87_RS10735 | NZ_CP009257.1 | 2125104 | 2125553 | − | 450 | 6 |
| IX87_RS10890 | NZ_CP009257.1 | 2160019 | 2160273 | + | 255 | 6 |
| IX87_RS10985 | NZ_CP009257.1 | 2177848 | 2178723 | + | 876 | 6 |
| IX87_RS10995 | NZ_CP009257.1 | 2179765 | 2180277 | − | 513 | 6 |
| IX87_RS11025 | NZ_CP009257.1 | 2185847 | 2186377 | − | 531 | 6 |
| IX87_RS11065 | NZ_CP009257.1 | 2196414 | 2196896 | + | 483 | 6 |
| IX87_RS11235 | NZ_CP009257.1 | 2228983 | 2229552 | + | 570 | 6 |
| IX87_RS11550 | NZ_CP009257.1 | 2289813 | 2290418 | + | 606 | 6 |
| IX87_RS11705 | NZ_CP009257.1 | 2320232 | 2320798 | − | 567 | 6 |
| IX87_RS12290 | NZ_CP009257.1 | 2425568 | 2426344 | − | 777 | 6 |
| IX87_RS12505 | NZ_CP009257.1 | 2470105 | 2470674 | − | 570 | 6 |
| IX87_RS12580 | NZ_CP009257.1 | 2487196 | 2488161 | − | 966 | 6 |
| IX87_RS12825 | NZ_CP009257.1 | 2533487 | 2533729 | − | 243 | 6 |
| IX87_RS13000 | NZ_CP009257.1 | 2562690 | 2563433 | − | 744 | 6 |
| IX87_RS13015 | NZ_CP009257.1 | 2565605 | 2566852 | + | 1248 | 6 |
| IX87_RS13090 | NZ_CP009257.1 | 2582132 | 2583004 | − | 873 | 6 |
| IX87_RS13255 | NZ_CP009257.1 | 2620660 | 2621202 | + | 543 | 6 |
| IX87_RS13345 | NZ_CP009257.1 | 2639019 | 2641184 | + | 2166 | 6 |
| IX87_RS13635 | NZ_CP009257.1 | 2703079 | 2703750 | + | 672 | 6 |
| IX87_RS13740 | NZ_CP009257.1 | 2723952 | 2725778 | − | 1827 | 6 |
| IX87_RS13900 | NZ_CP009257.1 | 2758137 | 2759171 | + | 1035 | 6 |
| IX87_RS13980 | NZ_CP009257.1 | 2773643 | 2774743 | − | 1101 | 6 |
| IX87_RS13985 | NZ_CP009257.1 | 2774893 | 2775516 | + | 624 | 6 |
| IX87_RS14275 | NZ_CP009257.1 | 2837985 | 2838371 | − | 387 | 6 |
| IX87_RS14305 | NZ_CP009257.1 | 2844627 | 2845493 | + | 867 | 6 |
| IX87_RS14310 | NZ_CP009257.1 | 2845885 | 2846115 | + | 231 | 6 |
| IX87_RS14775 | NZ_CP009257.1 | 2947064 | 2947945 | − | 882 | 6 |
| IX87_RS14805 | NZ_CP009257.1 | 2954208 | 2955233 | + | 1026 | 6 |
| IX87_RS14860 | NZ_CP009257.1 | 2968746 | 2972396 | + | 3651 | 6 |
| IX87_RS15075 | NZ_CP009257.1 | 3009005 | 3009655 | + | 651 | 6 |
| IX87_RS15095 | NZ_CP009257.1 | 3012844 | 3013998 | − | 1155 | 6 |
| IX87_RS15155 | NZ_CP009257.1 | 3025514 | 3026056 | + | 543 | 6 |
| IX87_RS15165 | NZ_CP009257.1 | 3027318 | 3027926 | − | 609 | 6 |
| IX87_RS15335 | NZ_CP009257.1 | 3062236 | 3062694 | + | 459 | 6 |
| IX87_RS15795 | NZ_CP009257.1 | 3154296 | 3155090 | + | 795 | 6 |
| IX87_RS15810 | NZ_CP009257.1 | 3156770 | 3157381 | + | 612 | 6 |
| IX87_RS16140 | NZ_CP009257.1 | 3233266 | 3233910 | + | 645 | 6 |
| IX87_RS16145 | NZ_CP009257.1 | 3233907 | 3234101 | − | 195 | 6 |
| IX87_RS16370 | NZ_CP009257.1 | 3277821 | 3279074 | − | 1254 | 6 |
| IX87_RS16375 | NZ_CP009257.1 | 3279143 | 3280507 | − | 1365 | 6 |
| IX87_RS16440 | NZ_CP009257.1 | 3295397 | 3296155 | + | 759 | 6 |
| IX87_RS16595 | NZ_CP009257.1 | 3320762 | 3322942 | − | 2181 | 6 |
| IX87_RS16745 | NZ_CP009257.1 | 3359883 | 3361181 | + | 1299 | 6 |
| IX87_RS16970 | NZ_CP009257.1 | 3407337 | 3408230 | + | 894 | 6 |
| IX87_RS17140 | NZ_CP009257.1 | 3442151 | 3443401 | − | 1251 | 6 |
| IX87_RS17350 | NZ_CP009257.1 | 3485863 | 3487218 | − | 1356 | 6 |
| IX87_RS17395 | NZ_CP009257.1 | 3497162 | 3497689 | + | 528 | 6 |
| IX87_RS18005 | NZ_CP009257.1 | 3625617 | 3626810 | + | 1194 | 6 |
| IX87_RS18675 | NZ_CP009257.1 | 3743848 | 3745053 | − | 1206 | 6 |
| IX87_RS18710 | NZ_CP009257.1 | 3752760 | 3754199 | + | 1440 | 6 |
| IX87_RS18720 | NZ_CP009257.1 | 3756062 | 3757972 | + | 1911 | 6 |
| IX87_RS18840 | NZ_CP009257.1 | 3782447 | 3784204 | + | 1758 | 6 |
| IX87_RS18870 | NZ_CP009257.1 | 3788682 | 3789788 | − | 1107 | 6 |
| IX87_RS18920 | NZ_CP009257.1 | 3794949 | 3796034 | − | 1086 | 6 |
| IX87_RS18930 | NZ_CP009257.1 | 3797533 | 3797769 | + | 237 | 6 |
| IX87_RS19065 | NZ_CP009257.1 | 3822824 | 3824095 | − | 1272 | 6 |
| IX87_RS19095 | NZ_CP009257.1 | 3829786 | 3830640 | + | 855 | 6 |
| IX87_RS19120 | NZ_CP009257.1 | 3834183 | 3835916 | + | 1734 | 6 |
| IX87_RS19175 | NZ_CP009257.1 | 3846836 | 3847057 | + | 222 | 6 |
| IX87_RS23065 | NZ_CP009257.1 | 3855311 | 3855556 | − | 246 | 6 |
| IX87_RS19420 | NZ_CP009257.1 | 3899229 | 3899816 | + | 588 | 6 |
| IX87_RS19490 | NZ_CP009257.1 | 3913584 | 3913958 | − | 375 | 6 |
| IX87_RS19530 | NZ_CP009257.1 | 3920822 | 3921196 | − | 375 | 6 |
| IX87_RS19800 | NZ_CP009257.1 | 3979780 | 3980853 | + | 1074 | 6 |
| IX87_RS20470 | NZ_CP009257.1 | 4111960 | 4114389 | − | 2430 | 6 |
| IX87_RS20595 | NZ_CP009257.1 | 4139158 | 4139718 | + | 561 | 6 |
| IX87_RS21110 | NZ_CP009257.1 | 4228285 | 4228500 | + | 216 | 6 |
| IX87_RS21210 | NZ_CP009257.1 | 4247592 | 4248074 | + | 483 | 6 |
| IX87_RS21415 | NZ_CP009257.1 | 4284663 | 4286936 | − | 2274 | 6 |
| IX87_RS21425 | NZ_CP009257.1 | 4287713 | 4290493 | − | 2781 | 6 |
| IX87_RS21435 | NZ_CP009257.1 | 4292147 | 4294681 | − | 2535 | 6 |
| IX87_RS01790 | NZ_CP009257.1 | 337140 | 337700 | + | 561 | 5 |
| IX87_RS01830 | NZ_CP009257.1 | 345673 | 347325 | − | 1653 | 5 |
| IX87_RS02350 | NZ_CP009257.1 | 433585 | 434265 | + | 681 | 5 |
| IX87_RS03250 | NZ_CP009257.1 | 608474 | 608791 | + | 318 | 5 |
| IX87_RS03630 | NZ_CP009257.1 | 694138 | 695346 | − | 1209 | 5 |
| IX87_RS03825 | NZ_CP009257.1 | 735701 | 736468 | − | 768 | 5 |
| IX87_RS03830 | NZ_CP009257.1 | 736471 | 737430 | − | 960 | 5 |
| IX87_RS04620 | NZ_CP009257.1 | 901559 | 901876 | − | 318 | 5 |
| IX87_RS05210 | NZ_CP009257.1 | 1003285 | 1004196 | + | 912 | 5 |
| IX87_RS06055 | NZ_CP009257.1 | 1158349 | 1158783 | + | 435 | 5 |
| IX87_RS06405 | NZ_CP009257.1 | 1233238 | 1233681 | + | 444 | 5 |
| IX87_RS06985 | NZ_CP009257.1 | 1358149 | 1358475 | + | 327 | 5 |
| IX87_RS07725 | NZ_CP009257.1 | 1497016 | 1497951 | + | 936 | 5 |
| IX87_RS08475 | NZ_CP009257.1 | 1638371 | 1640401 | + | 2031 | 5 |
| IX87_RS08785 | NZ_CP009257.1 | 1709094 | 1709726 | − | 633 | 5 |
| IX87_RS09220 | NZ_CP009257.1 | 1798775 | 1800109 | + | 1335 | 5 |
| IX87_RS09505 | NZ_CP009257.1 | 1861272 | 1862054 | + | 783 | 5 |
| IX87_RS09580 | NZ_CP009257.1 | 1875038 | 1875532 | − | 495 | 5 |
| IX87_RS10095 | NZ_CP009257.1 | 2008941 | 2010275 | − | 1335 | 5 |
| IX87_RS10490 | NZ_CP009257.1 | 2075682 | 2076173 | − | 492 | 5 |
| IX87_RS10565 | NZ_CP009257.1 | 2089570 | 2090805 | − | 1236 | 5 |
| IX87_RS10955 | NZ_CP009257.1 | 2171849 | 2172373 | + | 525 | 5 |
| IX87_RS11140 | NZ_CP009257.1 | 2210598 | 2211488 | + | 891 | 5 |
| IX87-RS11165 | NZ_CP009257.1 | 2215094 | 2215984 | − | 891 | 5 |
| IX87_RS12275 | NZ_CP009257.1 | 2423072 | 2423956 | + | 885 | 5 |
| IX87_RS12470 | NZ_CP009257.1 | 2463128 | 2463988 | − | 861 | 5 |
| IX87_RS12985 | NZ_CP009257.1 | 2560212 | 2561114 | + | 903 | 5 |
| IX87_RS13175 | NZ_CP009257.1 | 2598888 | 2599490 | − | 603 | 5 |
| IX87_RS13290 | NZ_CP009257.1 | 2628393 | 2628695 | − | 303 | 5 |
| IX87_RS14475 | NZ_CP009257.1 | 2876898 | 2878040 | + | 1143 | 5 |
| IX87_RS15315 | NZ_CP009257.1 | 3058224 | 3059711 | − | 1488 | 5 |
| IX87_RS15355 | NZ_CP009257.1 | 3065845 | 3066063 | − | 219 | 5 |
| IX87_RS16050 | NZ_CP009257.1 | 3214230 | 3214559 | − | 330 | 5 |
| IX87_RS16200 | NZ_CP009257.1 | 3243998 | 3244414 | − | 417 | 5 |
| IX87_RS17520 | NZ_CP009257.1 | 3519159 | 3520388 | + | 1230 | 5 |
| IX87_RS17720 | NZ_CP009257.1 | 3563636 | 3564466 | − | 831 | 5 |
| IX87_RS22945 | NZ_CP009257.1 | 3587332 | 3587502 | + | 171 | 5 |
| IX87_RS18020 | NZ_CP009257.1 | 3629503 | 3630237 | + | 735 | 5 |
| IX87_RS18575 | NZ_CP009257.1 | 3723169 | 3723951 | + | 783 | 5 |
| IX87_RS19130 | NZ_CP009257.1 | 3836202 | 3837632 | + | 1431 | 5 |
| IX87_RS19170 | NZ_CP009257.1 | 3846559 | 3846744 | + | 186 | 5 |
| IX87_RS19395 | NZ_CP009257.1 | 3895311 | 3895595 | + | 285 | 5 |
| IX87_RS19405 | NZ_CP009257.1 | 3896763 | 3897815 | + | 1053 | 5 |
| IX87_RS19810 | NZ_CP009257.1 | 3982048 | 3983076 | − | 1029 | 5 |
| IX87_RS19955 | NZ_CP009257.1 | 4009604 | 4010371 | − | 768 | 5 |
| IX87_RS20040 | NZ_CP009257.1 | 4027179 | 4028966 | + | 1788 | 5 |
| IX87_RS20490 | NZ_CP009257.1 | 4118218 | 4118847 | − | 630 | 5 |
| IX87_RS20495 | NZ_CP009257.1 | 4118862 | 4119587 | − | 726 | 5 |
| IX87_RS20530 | NZ_CP009257.1 | 4125461 | 4126132 | − | 672 | 5 |
| IX87_RS20535 | NZ_CP009257.1 | 4126482 | 4127198 | − | 717 | 5 |
| IX87_RS21320 | NZ_CP009257.1 | 4270344 | 4270835 | − | 492 | 5 |
| IX87_RS21365 | NZ_CP009257.1 | 4276772 | 4277056 | + | 285 | 5 |
| IX87_RS00430 | NZ_CP009257.1 | 61013 | 61321 | + | 309 | 4 |
| IX87_RS00605 | NZ_CP009257.1 | 98347 | 100023 | + | 1677 | 4 |
| IX87_RS22750 | NZ_CP009257.1 | 101137 | 101301 | + | 165 | 4 |
| IX87_RS00695 | NZ_CP009257.1 | 122017 | 123315 | + | 1299 | 4 |
| IX87_RS00725 | NZ_CP009257.1 | 129505 | 130329 | − | 825 | 4 |
| IX87_RS00790 | NZ_CP009257.1 | 140000 | 140404 | − | 405 | 4 |
| IX87_RS00885 | NZ_CP009257.1 | 161396 | 162274 | + | 879 | 4 |
| IX87_RS01565 | NZ_CP009257.1 | 290463 | 291368 | − | 906 | 4 |
| IX87_RS01585 | NZ_CP009257.1 | 293376 | 294644 | + | 1269 | 4 |
| IX87_RS01600 | NZ_CP009257.1 | 299241 | 300308 | + | 1068 | 4 |
| IX87_RS01605 | NZ_CP009257.1 | 300336 | 301454 | + | 1119 | 4 |
| IX87_RS01700 | NZ_CP009257.1 | 317191 | 317811 | − | 621 | 4 |
| IX87_RS01725 | NZ_CP009257.1 | 323939 | 324394 | − | 456 | 4 |
| IX87_RS01815 | NZ_CP009257.1 | 342013 | 343464 | − | 1452 | 4 |
| IX87_RS01840 | NZ_CP009257.1 | 348562 | 349542 | + | 981 | 4 |
| IX87_RS02120 | NZ_CP009257.1 | 392315 | 393274 | − | 960 | 4 |
| IX87_RS02135 | NZ_CP009257.1 | 395818 | 396792 | + | 975 | 4 |
| IX87_RS02245 | NZ_CP009257.1 | 412506 | 413267 | − | 762 | 4 |
| IX87_RS23030 | NZ_CP009257.1 | 455917 | 456057 | − | 141 | 4 |
| IX87_RS02875 | NZ_CP009257.1 | 533496 | 535370 | − | 1875 | 4 |
| IX87_RS02895 | NZ_CP009257.1 | 538135 | 538884 | − | 750 | 4 |
| IX87_RS03050 | NZ_CP009257.1 | 569412 | 570005 | − | 594 | 4 |
| IX87_RS03090 | NZ_CP009257.1 | 576125 | 577045 | − | 921 | 4 |
| IX87_RS03150 | NZ_CP009257.1 | 586874 | 587740 | − | 867 | 4 |
| IX87_RS03205 | NZ_CP009257.1 | 599059 | 599670 | − | 612 | 4 |
| IX87_RS03210 | NZ_CP009257.1 | 599953 | 600189 | + | 237 | 4 |
| IX87_RS03285 | NZ_CP009257.1 | 612173 | 613078 | − | 906 | 4 |
| IX87_RS03690 | NZ_CP009257.1 | 708069 | 709649 | + | 1581 | 4 |
| IX87_RS03695 | NZ_CP009257.1 | 709808 | 711097 | + | 1290 | 4 |
| IX87_RS03735 | NZ_CP009257.1 | 717720 | 718655 | − | 936 | 4 |
| IX87_RS03800 | NZ_CP009257.1 | 729197 | 730003 | − | 807 | 4 |
| IX87_RS03810 | NZ_CP009257.1 | 731402 | 732496 | − | 1095 | 4 |
| IX87_RS04140 | NZ_CP009257.1 | 804543 | 805847 | − | 1305 | 4 |
| IX87_RS04150 | NZ_CP009257.1 | 807340 | 808272 | − | 933 | 4 |
| IX87_RS04165 | NZ_CP009257.1 | 811239 | 811706 | + | 468 | 4 |
| IX87_RS04355 | NZ_CP009257.1 | 844207 | 845469 | + | 1263 | 4 |
| IX87_RS04395 | NZ_CP009257.1 | 849860 | 852331 | − | 2472 | 4 |
| IX87_RS04400 | NZ_CP009257.1 | 852498 | 852698 | + | 201 | 4 |
| IX87_RS04470 | NZ_CP009257.1 | 865642 | 866835 | + | 1194 | 4 |
| IX87_RS04595 | NZ_CP009257.1 | 897308 | 897733 | − | 426 | 4 |
| IX87_RS05015 | NZ_CP009257.1 | 959318 | 959875 | + | 558 | 4 |
| IX87_RS05065 | NZ_CP009257.1 | 970168 | 971001 | + | 834 | 4 |
| IX87_RS05085 | NZ_CP009257.1 | 974697 | 976184 | + | 1488 | 4 |
| IX87_RS05275 | NZ_CP009257.1 | 1016197 | 1017657 | + | 1461 | 4 |
| IX87_RS05295 | NZ_CP009257.1 | 1020490 | 1021584 | − | 1095 | 4 |
| IX87_RS05340 | NZ_CP009257.1 | 1030612 | 1031970 | + | 1359 | 4 |
| IX87_RS05585 | NZ_CP009257.1 | 1085242 | 1085883 | − | 642 | 4 |
| IX87_RS05600 | NZ_CP009257.1 | 1086866 | 1088098 | − | 1233 | 4 |
| IX87_RS06015 | NZ_CP009257.1 | 1148484 | 1149821 | + | 1338 | 4 |
| IX87_RS06060 | NZ_CP009257.1 | 1158832 | 1161060 | − | 2229 | 4 |
| IX87_RS06105 | NZ_CP009257.1 | 1172016 | 1172909 | + | 894 | 4 |
| IX87_RS06130 | NZ_CP009257.1 | 1176695 | 1177414 | − | 720 | 4 |
| IX87_RS06200 | NZ_CP009257.1 | 1191055 | 1191438 | − | 384 | 4 |
| IX87_RS06250 | NZ_CP009257.1 | 1200847 | 1201344 | + | 498 | 4 |
| IX87_RS06270 | NZ_CP009257.1 | 1203470 | 1204156 | − | 687 | 4 |
| IX87_RS06520 | NZ_CP009257.1 | 1261430 | 1263469 | − | 2040 | 4 |
| IX87_RS06570 | NZ_CP009257.1 | 1273122 | 1274150 | + | 1029 | 4 |
| IX87_RS06590 | NZ_CP009257.1 | 1277217 | 1278119 | + | 903 | 4 |
| IX87_RS06605 | NZ_CP009257.1 | 1279612 | 1280616 | + | 1005 | 4 |
| IX87_RS06610 | NZ_CP009257.1 | 1280763 | 1281326 | + | 564 | 4 |
| IX87_RS06860 | NZ_CP009257.1 | 1335895 | 1336797 | − | 903 | 4 |
| IX87_RS06955 | NZ_CP009257.1 | 1351437 | 1352135 | − | 699 | 4 |
| IX87_RS06970 | NZ_CP009257.1 | 1354783 | 1355931 | + | 1149 | 4 |
| IX87_RS06975 | NZ_CP009257.1 | 1356089 | 1356925 | + | 837 | 4 |
| IX87_RS07010 | NZ_CP009257.1 | 1360973 | 1361767 | − | 795 | 4 |
| IX87_RS07130 | NZ_CP009257.1 | 1389541 | 1390344 | − | 804 | 4 |
| IX87_RS07200 | NZ_CP009257.1 | 1404574 | 1405053 | − | 480 | 4 |
| IX87_RS07265 | NZ_CP009257.1 | 1413742 | 1414365 | − | 624 | 4 |
| IX87_RS07345 | NZ_CP009257.1 | 1432682 | 1433581 | − | 900 | 4 |
| IX87_RS07355 | NZ_CP009257.1 | 1433954 | 1435312 | − | 1359 | 4 |
| IX87_RS07360 | NZ_CP009257.1 | 1435309 | 1435971 | − | 663 | 4 |
| IX87_RS07365 | NZ_CP009257.1 | 1436079 | 1436438 | − | 360 | 4 |
| IX87_RS07370 | NZ_CP009257.1 | 1436520 | 1438277 | − | 1758 | 4 |
| IX87_RS07375 | NZ_CP009257.1 | 1438565 | 1439833 | + | 1269 | 4 |
| IX87_RS07380 | NZ_CP009257.1 | 1439843 | 1440571 | − | 729 | 4 |
| IX87_RS07390 | NZ_CP009257.1 | 1441510 | 1442379 | − | 870 | 4 |
| IX87_RS07430 | NZ_CP009257.1 | 1448683 | 1449468 | − | 786 | 4 |
| IX87_RS07730 | NZ_CP009257.1 | 1497948 | 1498721 | + | 774 | 4 |
| IX87_RS07925 | NZ_CP009257.1 | 1537655 | 1538164 | − | 510 | 4 |
| IX87_RS08455 | NZ_CP009257.1 | 1634109 | 1635077 | − | 969 | 4 |
| IX87_RS08500 | NZ_CP009257.1 | 1645659 | 1646345 | + | 687 | 4 |
| IX87_RS08515 | NZ_CP009257.1 | 1648046 | 1649026 | − | 981 | 4 |
| IX87_RS08755 | NZ_CP009257.1 | 1703440 | 1704078 | − | 639 | 4 |
| IX87_RS08770 | NZ_CP009257.1 | 1706318 | 1706929 | − | 612 | 4 |
| IX87_RS08810 | NZ_CP009257.1 | 1715712 | 1716593 | − | 882 | 4 |
| IX87_RS08915 | NZ_CP009257.1 | 1741284 | 1742309 | − | 1026 | 4 |
| IX87_RS09005 | NZ_CP009257.1 | 1759538 | 1759975 | + | 438 | 4 |
| IX87_RS09035 | NZ_CP009257.1 | 1763354 | 1764067 | − | 714 | 4 |
| IX87_RS09100 | NZ_CP009257.1 | 1775397 | 1776209 | − | 813 | 4 |
| IX87_RS09110 | NZ_CP009257.1 | 1777511 | 1778119 | + | 609 | 4 |
| IX87_RS09430 | NZ_CP009257.1 | 1844447 | 1845334 | − | 888 | 4 |
| IX87_RS09485 | NZ_CP009257.1 | 1855753 | 1856544 | − | 792 | 4 |
| IX87_RS09495 | NZ_CP009257.1 | 1859427 | 1860593 | + | 1167 | 4 |
| IX87_RS09565 | NZ_CP009257.1 | 1869643 | 1873641 | − | 3999 | 4 |
| IX87_RS09670 | NZ_CP009257.1 | 1905970 | 1907478 | − | 1509 | 4 |
| IX87_RS10045 | NZ_CP009257.1 | 1997531 | 1997965 | − | 435 | 4 |
| IX87_RS22855 | NZ_CP009257.1 | 2093718 | 2093858 | − | 141 | 4 |
| IX87_RS10650 | NZ_CP009257.1 | 2105036 | 2105404 | − | 369 | 4 |
| IX87_RS10725 | NZ_CP009257.1 | 2124170 | 2124514 | − | 345 | 4 |
| IX87_RS10780 | NZ_CP009257.1 | 2134246 | 2134536 | + | 291 | 4 |
| IX87_RS11325 | NZ_CP009257.1 | 2243938 | 2244834 | − | 897 | 4 |
| IX87_RS11385 | NZ_CP009257.1 | 2257011 | 2258021 | − | 1011 | 4 |
| IX87_RS11480 | NZ_CP009257.1 | 2275098 | 2276024 | − | 927 | 4 |
| IX87_RS11615 | NZ_CP009257.1 | 2302131 | 2302754 | − | 624 | 4 |
| IX87_RS11660 | NZ_CP009257.1 | 2311147 | 2311740 | − | 594 | 4 |
| IX87_RS12440 | NZ_CP009257.1 | 2458260 | 2458472 | − | 213 | 4 |
| IX87_RS12510 | NZ_CP009257.1 | 2470686 | 2472851 | − | 2166 | 4 |
| IX87_RS12515 | NZ_CP009257.1 | 2472914 | 2473441 | − | 528 | 4 |
| IX87_RS12520 | NZ_CP009257.1 | 2473452 | 2474192 | − | 741 | 4 |
| IX87_RS12525 | NZ_CP009257.1 | 2474189 | 2474830 | − | 642 | 4 |
| IX87_RS12730 | NZ_CP009257.1 | 2520900 | 2521331 | + | 432 | 4 |
| IX87_RS12795 | NZ_CP009257.1 | 2528229 | 2528858 | − | 630 | 4 |
| IX87_RS12800 | NZ_CP009257.1 | 2528871 | 2529821 | − | 951 | 4 |
| IX87_RS13185 | NZ_CP009257.1 | 2602655 | 2604361 | − | 1707 | 4 |
| IX87_RS13330 | NZ_CP009257.1 | 2637108 | 2637905 | − | 798 | 4 |
| IX87_RS13350 | NZ_CP009257.1 | 2641243 | 2641818 | − | 576 | 4 |
| IX87_RS13525 | NZ_CP009257.1 | 2678294 | 2679019 | + | 726 | 4 |
| IX87_RS13770 | NZ_CP009257.1 | 2733252 | 2733791 | − | 540 | 4 |
| IX87_RS13775 | NZ_CP009257.1 | 2733824 | 2734576 | − | 753 | 4 |
| IX87_RS13785 | NZ_CP009257.1 | 2736075 | 2737340 | − | 1266 | 4 |
| IX87_RS13925 | NZ_CP009257.1 | 2761671 | 2763095 | − | 1425 | 4 |
| IX87_RS14045 | NZ_CP009257.1 | 2790856 | 2791433 | − | 578 | 4 |
| IX87_RS14180 | NZ_CP009257.1 | 2821253 | 2821951 | + | 699 | 4 |
| IX87_RS14225 | NZ_CP009257.1 | 2828269 | 2828634 | − | 366 | 4 |
| IX87_RS14250 | NZ_CP009257.1 | 2833765 | 2834127 | + | 363 | 4 |
| IX87_RS14260 | NZ_CP009257.1 | 2834311 | 2834823 | + | 513 | 4 |
| IX87_RS14485 | NZ_CP009257.1 | 2879204 | 2881372 | + | 2169 | 4 |
| IX87_RS14735 | NZ_CP009257.1 | 2936888 | 2938852 | − | 1965 | 4 |
| IX87_RS14855 | NZ_CP009257.1 | 2964762 | 2968721 | + | 3960 | 4 |
| IX87_RS14940 | NZ_CP009257.1 | 2985288 | 2985815 | + | 528 | 4 |
| IX87_RS15065 | NZ_CP009257.1 | 3006868 | 3007632 | − | 765 | 4 |
| IX87_RS15300 | NZ_CP009257.1 | 3054994 | 3055872 | − | 879 | 4 |
| IX87_RS15510 | NZ_CP009257.1 | 3097157 | 3098062 | + | 906 | 4 |
| IX87_RS15730 | NZ_CP009257.1 | 3141650 | 3142408 | + | 759 | 4 |
| IX87_RS15865 | NZ_CP009257.1 | 3167744 | 3168658 | + | 915 | 4 |
| IX87_RS16215 | NZ_CP009257.1 | 3246651 | 3247547 | + | 897 | 4 |
| IX87_RS16250 | NZ_CP009257.1 | 3255783 | 3256478 | + | 696 | 4 |
| IX87_RS16480 | NZ_CP009257.1 | 3302512 | 3302667 | − | 156 | 4 |
| IX87_RS16585 | NZ_CP009257.1 | 3317401 | 3318171 | + | 771 | 4 |
| IX87_RS16730 | NZ_CP009257.1 | 3355181 | 3356023 | − | 843 | 4 |
| IX87_RS16750 | NZ_CP009257.1 | 3361206 | 3361757 | + | 552 | 4 |
| IX87_RS16875 | NZ_CP009257.1 | 3386424 | 3386753 | − | 330 | 4 |
| IX87_RS17005 | NZ_CP009257.1 | 3414219 | 3414533 | + | 315 | 4 |
| IX87_RS17065 | NZ_CP009257.1 | 3428699 | 3429472 | − | 774 | 4 |
| IX87_RS17070 | NZ_CP009257.1 | 3429584 | 3430390 | + | 807 | 4 |
| IX87_RS17115 | NZ_CP009257.1 | 3436284 | 3437912 | − | 1629 | 4 |
| IX87_RS17215 | NZ_CP009257.1 | 3455928 | 3457331 | + | 1404 | 4 |
| IX87_RS17325 | NZ_CP009257.1 | 3481112 | 3481363 | + | 252 | 4 |
| IX87_RS17355 | NZ_CP009257.1 | 3487576 | 3488418 | + | 843 | 4 |
| IX87_RS17360 | NZ_CP009257.1 | 3488719 | 3489552 | + | 834 | 4 |
| IX87_RS17385 | NZ_CP009257.1 | 3495850 | 3496398 | − | 549 | 4 |
| IX87_RS17440 | NZ_CP009257.1 | 3504555 | 3506489 | + | 1935 | 4 |
| IX87_RS17870 | NZ_CP009257.1 | 3593352 | 3593975 | + | 624 | 4 |
| IX87_RS17920 | NZ_CP009257.1 | 3603944 | 3605986 | + | 2043 | 4 |
| IX87_RS18035 | NZ_CP009257.1 | 3632764 | 3633513 | + | 750 | 4 |
| IX87_RS18110 | NZ_CP009257.1 | 3643593 | 3644252 | − | 660 | 4 |
| IX87_RS18485 | NZ_CP009257.1 | 3702668 | 3703081 | − | 414 | 4 |
| IX87_RS18540 | NZ_CP009257.1 | 3718309 | 3719043 | + | 735 | 4 |
| IX87_RS18700 | NZ_CP009257.1 | 3749529 | 3751313 | + | 1785 | 4 |
| IX87_RS18705 | NZ_CP009257.1 | 3751306 | 3752739 | + | 1434 | 4 |
| IX87_RS18725 | NZ_CP009257.1 | 3757985 | 3758755 | + | 771 | 4 |
| IX87_RS18835 | NZ_CP009257.1 | 3781432 | 3782454 | + | 1023 | 4 |
| IX87_RS18910 | NZ_CP009257.1 | 3793553 | 3793927 | + | 375 | 4 |
| IX87_RS18965 | NZ_CP009257.1 | 3802489 | 3802830 | + | 342 | 4 |
| IX87_RS19020 | NZ_CP009257.1 | 3812310 | 3813449 | − | 1140 | 4 |
| IX87_RS19035 | NZ_CP009257.1 | 3815934 | 3816953 | + | 1020 | 4 |
| IX87_RS19160 | NZ_CP009257.1 | 3842467 | 3844671 | + | 2205 | 4 |
| IX87_RS19350 | NZ_CP009257.1 | 3880532 | 3882313 | − | 1782 | 4 |
| IX87_RS22560 | NZ_CP009257.1 | 3893486 | 3893560 | + | 75 | 4 |
| IX87_RS19400 | NZ_CP009257.1 | 3895592 | 3896746 | + | 1155 | 4 |
| IX87_RS19450 | NZ_CP009257.1 | 3904427 | 3905590 | − | 1164 | 4 |
| IX87_RS19785 | NZ_CP009257.1 | 3974562 | 3977672 | − | 3111 | 4 |
| IX87_RS19910 | NZ_CP009257.1 | 3998320 | 3999291 | − | 972 | 4 |
| IX87_RS20010 | NZ_CP009257.1 | 4020405 | 4021289 | + | 885 | 4 |
| IX87_RS20295 | NZ_CP009257.1 | 4076806 | 4077567 | − | 762 | 4 |
| IX87_RS20300 | NZ_CP009257.1 | 4077682 | 4078596 | − | 915 | 4 |
| IX87_RS20450 | NZ_CP009257.1 | 4109024 | 4109890 | − | 867 | 4 |
| IX87_RS20480 | NZ_CP009257.1 | 4115851 | 4117302 | − | 1452 | 4 |
| IX87_RS21300 | NZ_CP009257.1 | 4266890 | 4267414 | + | 525 | 4 |
| IX87_RS21395 | NZ_CP009257.1 | 4281704 | 4282627 | − | 924 | 4 |
| IX87_RS21400 | NZ_CP009257.1 | 4282651 | 4283136 | − | 486 | 4 |
| IX87_RS22740 | NZ_CP009257.1 | 73402 | 73533 | − | 132 | 3 |
| IX87_RS00520 | NZ_CP009257.1 | 79533 | 79697 | + | 165 | 3 |
| IX87_RS00590 | NZ_CP009257.1 | 94329 | 96593 | − | 2265 | 3 |
| IX87_RS00690 | NZ_CP009257.1 | 121300 | 121782 | − | 483 | 3 |
| IX87_RS01785 | NZ_CP009257.1 | 336189 | 337049 | + | 861 | 3 |
| IX87_RS01825 | NZ_CP009257.1 | 344687 | 345661 | − | 975 | 3 |
| IX87_RS02260 | NZ_CP009257.1 | 416014 | 416442 | − | 429 | 3 |
| IX87_RS02600 | NZ_CP009257.1 | 479905 | 480468 | − | 564 | 3 |
| IX87_RS02955 | NZ_CP009257.1 | 550956 | 552251 | − | 1296 | 3 |
| IX87_RS04545 | NZ_CP009257.1 | 884696 | 885475 | − | 780 | 3 |
| IX87_RS04575 | NZ_CP009257.1 | 892394 | 892810 | − | 417 | 3 |
| IX87_RS06035 | NZ_CP009257.1 | 1153864 | 1154898 | − | 1035 | 3 |
| IX87_RS06115 | NZ_CP009257.1 | 1174102 | 1174752 | + | 651 | 3 |
| IX87_RS06160 | NZ_CP009257.1 | 1183037 | 1184623 | + | 1587 | 3 |
| IX87_RS06165 | NZ_CP009257.1 | 1184697 | 1184921 | − | 225 | 3 |
| IX87_RS22815 | NZ_CP009257.1 | 1186438 | 1186569 | − | 132 | 3 |
| IX87_RS06335 | NZ_CP009257.1 | 1220410 | 1221225 | + | 816 | 3 |
| IX87_RS06615 | NZ_CP009257.1 | 1281366 | 1281830 | − | 465 | 3 |
| IX87_RS06800 | NZ_CP009257.1 | 1322781 | 1323233 | − | 453 | 3 |
| IX87_RS22130 | NZ_CP009257.1 | 1339112 | 1339234 | + | 123 | 3 |
| IX87_RS07180 | NZ_CP009257.1 | 1399351 | 1401240 | − | 1890 | 3 |
| IX87_RS07325 | NZ_CP009257.1 | 1428329 | 1428601 | − | 273 | 3 |
| IX87_RS07465 | NZ_CP009257.1 | 1459757 | 1460443 | + | 687 | 3 |
| IX87_RS08485 | NZ_CP009257.1 | 1642714 | 1643574 | + | 861 | 3 |
| IX87_RS09085 | NZ_CP009257.1 | 1773161 | 1773577 | − | 417 | 3 |
| IX87_RS09550 | NZ_CP009257.1 | 1868079 | 1868498 | − | 420 | 3 |
| IX87_RS10100 | NZ_CP009257.1 | 2010301 | 2010975 | − | 675 | 3 |
| IX87_RS10535 | NZ_CP009257.1 | 2084143 | 2084892 | + | 750 | 3 |
| IX87_RS10685 | NZ_CP009257.1 | 2115210 | 2115746 | − | 537 | 3 |
| IX87_RS10695 | NZ_CP009257.1 | 2116193 | 2116576 | − | 384 | 3 |
| IX87_RS10730 | NZ_CP009257.1 | 2124606 | 2124962 | − | 357 | 3 |
| IX87_RS23055 | NZ_CP009257.1 | 2143160 | 2143303 | + | 144 | 3 |
| IX87_RS11310 | NZ_CP009257.1 | 2242542 | 2243036 | + | 495 | 3 |
| IX87_RS11470 | NZ_CP009257.1 | 2272274 | 2273431 | − | 1158 | 3 |
| IX87_RS11490 | NZ_CP009257.1 | 2278022 | 2278621 | + | 600 | 3 |
| IX87_RS11760 | NZ_CP009257.1 | 2329396 | 2329512 | − | 117 | 3 |
| IX87_RS12200 | NZ_CP009257.1 | 2408277 | 2408744 | − | 468 | 3 |
| IX87_RS12300 | NZ_CP009257.1 | 2427423 | 2428457 | + | 1035 | 3 |
| IX87_RS12765 | NZ_CP009257.1 | 2524371 | 2525012 | + | 642 | 3 |
| IX87_RS14715 | NZ_CP009257.1 | 2934471 | 2935172 | + | 702 | 3 |
| IX87_RS14935 | NZ_CP009257.1 | 2984279 | 2985067 | + | 789 | 3 |
| IX87_RS15785 | NZ_CP009257.1 | 3151712 | 3152242 | + | 531 | 3 |
| IX87_RS15875 | NZ_CP009257.1 | 3169253 | 3170113 | − | 861 | 3 |
| IX87_RS16415 | NZ_CP009257.1 | 3290260 | 3290505 | + | 246 | 3 |
| IX87_RS16570 | NZ_CP009257.1 | 3315674 | 3316036 | − | 363 | 3 |
| IX87_RS17265 | NZ_CP009257.1 | 3469020 | 3469397 | − | 378 | 3 |
| IX87_RS22940 | NZ_CP009257.1 | 3492449 | 3493825 | + | 1377 | 3 |
| IX87_RS17400 | NZ_CP009257.1 | 3497822 | 3498487 | + | 666 | 3 |
| IX87_RS18715 | NZ_CP009257.1 | 3754211 | 3756052 | + | 1842 | 3 |
| IX87_RS20275 | NZ_CP009257.1 | 4071722 | 4072720 | − | 999 | 3 |
| IX87_RS20685 | NZ_CP009257.1 | 4161744 | 4162052 | + | 309 | 3 |
| IX87_RS21175 | NZ_CP009257.1 | 4243371 | 4243907 | + | 537 | 3 |
| IX87_RS21555 | NZ_CP009257.1 | 4311526 | 4312824 | + | 1299 | 3 |
| IX87_RS00305 | NZ_CP009257.1 | 31675 | 32454 | − | 780 | 2 |
| IX87_RS00310 | NZ_CP009257.1 | 32512 | 38628 | − | 6117 | 2 |
| IX87_RS00380 | NZ_CP009257.1 | 54015 | 54566 | + | 552 | 2 |
| IX87_RS00415 | NZ_CP009257.1 | 59191 | 59511 | − | 321 | 2 |
| IX87_RS00555 | NZ_CP009257.1 | 86681 | 87490 | + | 810 | 2 |
| IX87_RS00565 | NZ_CP009257.1 | 89955 | 90647 | − | 693 | 2 |
| IX87_RS00600 | NZ_CP009257.1 | 97559 | 98176 | + | 618 | 2 |
| IX87_RS00610 | NZ_CP009257.1 | 100128 | 101027 | + | 900 | 2 |
| IX87_RS00650 | NZ_CP009257.1 | 112668 | 113996 | − | 1329 | 2 |
| IX87_RS00675 | NZ_CP009257.1 | 117717 | 118919 | − | 1203 | 2 |
| IX87_RS00735 | NZ_CP009257.1 | 130911 | 132134 | − | 1224 | 2 |
| IX87_RS00805 | NZ_CP009257.1 | 144006 | 144347 | − | 342 | 2 |
| IX87_RS00900 | NZ_CP009257.1 | 165706 | 166413 | − | 708 | 2 |
| IX87_RS00995 | NZ_CP009257.1 | 189648 | 189932 | + | 285 | 2 |
| IX87_RS01540 | NZ_CP009257.1 | 284726 | 286249 | − | 1524 | 2 |
| IX87_RS21840 | NZ_CP009257.1 | 324502 | 324717 | − | 216 | 2 |
| IX87_RS01740 | NZ_CP009257.1 | 325895 | 326566 | − | 672 | 2 |
| IX87_RS02145 | NZ_CP009257.1 | 397655 | 398251 | + | 597 | 2 |
| IX87_RS02235 | NZ_CP009257.1 | 411161 | 411502 | − | 342 | 2 |
| IX87_RS02340 | NZ_CP009257.1 | 431645 | 431953 | + | 309 | 2 |
| IX87_RS02460 | NZ_CP009257.1 | 456424 | 456609 | + | 186 | 2 |
| IX87_RS02480 | NZ_CP009257.1 | 459068 | 459661 | − | 594 | 2 |
| IX87_RS02515 | NZ_CP009257.1 | 465225 | 465761 | + | 537 | 2 |
| IX87_RS02535 | NZ_CP009257.1 | 467813 | 470350 | + | 2538 | 2 |
| IX87_RS02545 | NZ_CP009257.1 | 471943 | 472122 | + | 180 | 2 |
| IX87_RS02550 | NZ_CP009257.1 | 472162 | 473013 | − | 852 | 2 |
| IX87_RS02575 | NZ_CP009257.1 | 476867 | 477766 | + | 900 | 2 |
| IX87_RS02630 | NZ_CP009257.1 | 483670 | 484215 | + | 546 | 2 |
| IX87_RS02700 | NZ_CP009257.1 | 497040 | 497870 | + | 831 | 2 |
| IX87_RS02725 | NZ_CP009257.1 | 501669 | 502190 | − | 522 | 2 |
| IX87_RS02850 | NZ_CP009257.1 | 530235 | 531137 | + | 903 | 2 |
| IX87_RS02915 | NZ_CP009257.1 | 542440 | 543309 | − | 870 | 2 |
| IX87_RS02985 | NZ_CP009257.1 | 558048 | 558965 | + | 918 | 2 |
| IX87_RS03030 | NZ_CP009257.1 | 564641 | 566320 | − | 1680 | 2 |
| IX87_RS03075 | NZ_CP009257.1 | 573542 | 574447 | + | 906 | 2 |
| IX87_RS03180 | NZ_CP009257.1 | 591475 | 592434 | − | 960 | 2 |
| IX87_RS03190 | NZ_CP009257.1 | 596061 | 597707 | + | 1647 | 2 |
| IX87_RS03195 | NZ_CP009257.1 | 597764 | 598219 | − | 456 | 2 |
| IX87_RS03215 | NZ_CP009257.1 | 600250 | 601485 | − | 1236 | 2 |
| IX87_RS03255 | NZ_CP009257.1 | 608876 | 609097 | − | 222 | 2 |
| IX87_RS03260 | NZ_CP009257.1 | 609185 | 609466 | − | 282 | 2 |
| IX87_RS03495 | NZ_CP009257.1 | 666641 | 667678 | − | 1038 | 2 |
| IX87_RS03510 | NZ_CP009257.1 | 670023 | 670838 | + | 816 | 2 |
| IX87_RS03615 | NZ_CP009257.1 | 691197 | 691802 | − | 606 | 2 |
| IX87_RS03770 | NZ_CP009257.1 | 724271 | 725449 | − | 1179 | 2 |
| IX87_RS03780 | NZ_CP009257.1 | 725947 | 726594 | − | 648 | 2 |
| IX87_RS03785 | NZ_CP009257.1 | 726767 | 727618 | + | 852 | 2 |
| IX87_RS03790 | NZ_CP009257.1 | 727621 | 728574 | − | 954 | 2 |
| IX87_RS03795 | NZ_CP009257.1 | 728582 | 729184 | − | 603 | 2 |
| IX87_RS03880 | NZ_CP009257.1 | 750406 | 750807 | + | 402 | 2 |
| IX87_RS03930 | NZ_CP009257.1 | 757712 | 758248 | + | 537 | 2 |
| IX87_RS03935 | NZ_CP009257.1 | 758288 | 761893 | − | 3606 | 2 |
| IX87_RS04015 | NZ_CP009257.1 | 777353 | 778264 | − | 912 | 2 |
| IX87_RS04085 | NZ_CP009257.1 | 795801 | 796499 | + | 699 | 2 |
| IX87_RS04145 | NZ_CP009257.1 | 806112 | 806753 | − | 642 | 2 |
| IX87_RS04160 | NZ_CP009257.1 | 810259 | 811119 | − | 861 | 2 |
| IX87_RS04210 | NZ_CP009257.1 | 819961 | 821151 | − | 1191 | 2 |
| IX87_RS04215 | NZ_CP009257.1 | 821148 | 823289 | − | 2142 | 2 |
| IX87_RS04325 | NZ_CP009257.1 | 841072 | 841617 | − | 546 | 2 |
| IX87_RS04330 | NZ_CP009257.1 | 841655 | 842044 | − | 390 | 2 |
| IX87_RS04385 | NZ_CP009257.1 | 848948 | 849355 | − | 408 | 2 |
| IX87_RS04390 | NZ_CP009257.1 | 849383 | 849784 | − | 402 | 2 |
| IX87_RS04445 | NZ_CP009257.1 | 860567 | 861526 | − | 960 | 2 |
| IX87_RS04475 | NZ_CP009257.1 | 867303 | 868661 | + | 1359 | 2 |
| IX87_RS04510 | NZ_CP009257.1 | 877140 | 877358 | − | 219 | 2 |
| IX87_RS04655 | NZ_CP009257.1 | 911427 | 912071 | + | 645 | 2 |
| IX87_RS04660 | NZ_CP009257.1 | 912186 | 912680 | + | 495 | 2 |
| IX87_RS04820 | NZ_CP009257.1 | 939842 | 940483 | − | 642 | 2 |
| IX87_RS05060 | NZ_CP009257.1 | 969146 | 970105 | + | 960 | 2 |
| IX87_RS05070 | NZ_CP009257.1 | 971025 | 972548 | + | 1524 | 2 |
| IX87_RS05080 | NZ_CP009257.1 | 973547 | 974560 | − | 1014 | 2 |
| IX87_RS05160 | NZ_CP009257.1 | 992666 | 993505 | + | 840 | 2 |
| IX87_RS05225 | NZ_CP009257.1 | 1006834 | 1007589 | − | 756 | 2 |
| IX87_RS05235 | NZ_CP009257.1 | 1008410 | 1009018 | − | 609 | 2 |
| IX87_RS05245 | NZ_CP009257.1 | 1010733 | 1012280 | + | 1548 | 2 |
| IX87_RS05330 | NZ_CP009257.1 | 1027670 | 1029001 | + | 1332 | 2 |
| IX87_RS05590 | NZ_CP009257.1 | 1085949 | 1086350 | + | 402 | 2 |
| IX87_RS05595 | NZ_CP009257.1 | 1086405 | 1086656 | − | 252 | 2 |
| IX87_RS05985 | NZ_CP009257.1 | 1143190 | 1143459 | + | 270 | 2 |
| IX87_RS05990 | NZ_CP009257.1 | 1143465 | 1144727 | − | 1263 | 2 |
| IX87_RS06190 | NZ_CP009257.1 | 1188167 | 1190323 | − | 2157 | 2 |
| IX87_RS06210 | NZ_CP009257.1 | 1192083 | 1192553 | − | 471 | 2 |
| IX87_RS06260 | NZ_CP009257.1 | 1202008 | 1202691 | − | 684 | 2 |
| IX87_RS06325 | NZ_CP009257.1 | 1217563 | 1218999 | + | 1437 | 2 |
| IX87_RS06395 | NZ_CP009257.1 | 1231842 | 1232720 | + | 879 | 2 |
| IX87_RS06435 | NZ_CP009257.1 | 1237552 | 1237953 | − | 402 | 2 |
| IX87_RS06440 | NZ_CP009257.1 | 1238209 | 1239408 | + | 1200 | 2 |
| IX87_RS06445 | NZ_CP009257.1 | 1239434 | 1240417 | − | 984 | 2 |
| IX87_RS06450 | NZ_CP009257.1 | 1240556 | 1240990 | − | 435 | 2 |
| IX87_RS06535 | NZ_CP009257.1 | 1265843 | 1266223 | − | 381 | 2 |
| IX87_RS06600 | NZ_CP009257.1 | 1278974 | 1279477 | + | 504 | 2 |
| IX87_RS06790 | NZ_CP009257.1 | 1320816 | 1321382 | − | 567 | 2 |
| IX87_RS06895 | NZ_CP009257.1 | 1342847 | 1345345 | − | 2499 | 2 |
| IX87_RS06915 | NZ_CP009257.1 | 1347316 | 1347852 | − | 537 | 2 |
| IX87_RS06960 | NZ_CP009257.1 | 1352232 | 1353134 | + | 903 | 2 |
| IX87_RS06995 | NZ_CP009257.1 | 1359106 | 1359468 | − | 363 | 2 |
| IX87_RS07005 | NZ_CP009257.1 | 1360399 | 1360998 | − | 600 | 2 |
| IX87_RS07025 | NZ_CP009257.1 | 1363409 | 1364806 | − | 1398 | 2 |
| IX87_RS07040 | NZ_CP009257.1 | 1368068 | 1368985 | + | 918 | 2 |
| IX87_RS07145 | NZ_CP009257.1 | 1393003 | 1394106 | + | 1104 | 2 |
| IX87_RS07150 | NZ_CP009257.1 | 1394109 | 1395119 | − | 1011 | 2 |
| IX87_RS07445 | NZ_CP009257.1 | 1452098 | 1455277 | + | 3180 | 2 |
| IX87_RS07450 | NZ_CP009257.1 | 1455290 | 1456738 | + | 1449 | 2 |
| IX87_RS22825 | NZ_CP009257.1 | 1458093 | 1458248 | + | 156 | 2 |
| IX87_RS07760 | NZ_CP009257.1 | 1503961 | 1504413 | + | 453 | 2 |
| IX87_RS07775 | NZ_CP009257.1 | 1505584 | 1506408 | − | 825 | 2 |
| IX87_RS07780 | NZ_CP009257.1 | 1506507 | 1506992 | − | 486 | 2 |
| IX87_RS07905 | NZ_CP009257.1 | 1534603 | 1535046 | + | 444 | 2 |
| IX87_RS07985 | NZ_CP009257.1 | 1551863 | 1556662 | − | 4800 | 2 |
| IX87_RS08020 | NZ_CP009257.1 | 1560231 | 1560555 | − | 325 | 2 |
| IX87_RS08380 | NZ_CP009257.1 | 1613576 | 1614370 | − | 795 | 2 |
| IX87_RS08395 | NZ_CP009257.1 | 1617116 | 1618285 | − | 1170 | 2 |
| IX87_RS08430 | NZ_CP009257.1 | 1624698 | 1625567 | − | 870 | 2 |
| IX87_RS08525 | NZ_CP009257.1 | 1649867 | 1650292 | − | 426 | 2 |
| IX87_RS08815 | NZ_CP009257.1 | 1716708 | 1717868 | + | 1161 | 2 |
| IX87_RS08905 | NZ_CP009257.1 | 1739381 | 1740082 | + | 702 | 2 |
| IX87_RS08955 | NZ_CP009257.1 | 1750012 | 1750959 | − | 948 | 2 |
| IX87_RS09010 | NZ_CP009257.1 | 1759990 | 1760469 | − | 480 | 2 |
| IX87_RS09055 | NZ_CP009257.1 | 1765994 | 1766368 | − | 375 | 2 |
| IX87_RS09410 | NZ_CP009257.1 | 1837533 | 1839179 | + | 1647 | 2 |
| IX87_RS09435 | NZ_CP009257.1 | 1845497 | 1846120 | + | 624 | 2 |
| IX87_RS09470 | NZ_CP009257.1 | 1853098 | 1854072 | − | 975 | 2 |
| IX87_RS09475 | NZ_CP009257.1 | 1854069 | 1855136 | − | 1068 | 2 |
| IX87_RS09480 | NZ_CP009257.1 | 1855241 | 1855633 | − | 393 | 2 |
| IX87_RS09500 | NZ_CP009257.1 | 1860582 | 1861148 | − | 567 | 2 |
| IX87_RS09610 | NZ_CP009257.1 | 1881184 | 1881759 | − | 576 | 2 |
| IX87_RS09620 | NZ_CP009257.1 | 1885191 | 1886333 | + | 1143 | 2 |
| IX87_RS09630 | NZ_CP009257.1 | 1887447 | 1887821 | + | 375 | 2 |
| IX87_RS09780 | NZ_CP009257.1 | 1926810 | 1927139 | − | 330 | 2 |
| IX87_RS09955 | NZ_CP009257.1 | 1979314 | 1979694 | + | 381 | 2 |
| IX87_RS10075 | NZ_CP009257.1 | 2004693 | 2005598 | − | 906 | 2 |
| IX87_RS10105 | NZ_CP009257.1 | 2010990 | 2012639 | − | 1650 | 2 |
| IX87_RS10485 | NZ_CP009257.1 | 2075134 | 2075694 | − | 561 | 2 |
| IX87_RS10660 | NZ_CP009257.1 | 2106516 | 2106659 | + | 144 | 2 |
| IX87_RS10700 | NZ_CP009257.1 | 2116898 | 2117497 | + | 600 | 2 |
| IX87_RS10755 | NZ_CP009257.1 | 2128390 | 2129313 | − | 924 | 2 |
| IX87_RS10815 | NZ_CP009257.1 | 2141936 | 2143141 | + | 1206 | 2 |
| IX87_RS10870 | NZ_CP009257.1 | 2153982 | 2155100 | − | 1119 | 2 |
| IX87_RS10905 | NZ_CP009257.1 | 2161611 | 2162327 | − | 717 | 2 |
| IX87_RS10990 | NZ_CP009257.1 | 2178732 | 2179727 | − | 996 | 2 |
| IX87_RS11070 | NZ_CP009257.1 | 2196933 | 2197316 | − | 384 | 2 |
| IX87_RS11265 | NZ_CP009257.1 | 2235634 | 2236353 | + | 720 | 2 |
| IX87_RS11285 | NZ_CP009257.1 | 2238879 | 2239181 | + | 303 | 2 |
| IX87_RS11405 | NZ_CP009257.1 | 2262144 | 2262515 | + | 372 | 2 |
| IX87_RS11460 | NZ_CP009257.1 | 2269468 | 2271408 | − | 1941 | 2 |
| IX87_RS11465 | NZ_CP009257.1 | 2271427 | 2272263 | − | 837 | 2 |
| IX87_RS11725 | NZ_CP009257.1 | 2323572 | 2324744 | + | 1173 | 2 |
| IX87_RS12040 | NZ_CP009257.1 | 2376434 | 2377081 | − | 648 | 2 |
| IX87_RS12050 | NZ_CP009257.1 | 2377660 | 2378760 | − | 1101 | 2 |
| IX87_RS12055 | NZ_CP009257.1 | 2378893 | 2379690 | − | 798 | 2 |
| IX87_RS12120 | NZ_CP009257.1 | 2390392 | 2391747 | − | 1356 | 2 |
| IX87_RS12125 | NZ_CP009257.1 | 2391783 | 2392091 | − | 309 | 2 |
| IX87_RS12230 | NZ_CP009257.1 | 2415461 | 2415826 | − | 366 | 2 |
| IX87_RS12235 | NZ_CP009257.1 | 2416051 | 2416638 | + | 588 | 2 |
| IX87_RS12295 | NZ_CP009257.1 | 2426364 | 2427269 | − | 906 | 2 |
| IX87_RS12355 | NZ_CP009257.1 | 2438494 | 2439054 | − | 561 | 2 |
| IX87_RS12425 | NZ_CP009257.1 | 2450771 | 2451658 | − | 888 | 2 |
| IX87_RS12530 | NZ_CP009257.1 | 2474830 | 2475852 | − | 1023 | 2 |
| IX87_RS12570 | NZ_CP009257.1 | 2485718 | 2486050 | − | 333 | 2 |
| IX87_RS12660 | NZ_CP009257.1 | 2503088 | 2503414 | − | 327 | 2 |
| IX87_RS12810 | NZ_CP009257.1 | 2530734 | 2531372 | − | 639 | 2 |
| IX87_RS12815 | NZ_CP009257.1 | 2531482 | 2532486 | + | 1005 | 2 |
| IX87_RS12960 | NZ_CP009257.1 | 2556489 | 2556707 | + | 219 | 2 |
| IX87_RS12990 | NZ_CP009257.1 | 2561226 | 2562227 | + | 1002 | 2 |
| IX87_RS13010 | NZ_CP009257.1 | 2564546 | 2565466 | − | 921 | 2 |
| IX87_RS13130 | NZ_CP009257.1 | 2591825 | 2592520 | + | 696 | 2 |
| IX87_RS13165 | NZ_CP009257.1 | 2597488 | 2597841 | − | 354 | 2 |
| IX87_RS13250 | NZ_CP009257.1 | 2620141 | 2620554 | − | 414 | 2 |
| IX87_RS13275 | NZ_CP009257.1 | 2625141 | 2625848 | − | 708 | 2 |
| IX87_RS13355 | NZ_CP009257.1 | 2642067' | 2642339 | − | 273 | 2 |
| IX87_RS13420 | NZ_CP009257.1 | 2658176 | 2658388 | + | 213 | 2 |
| IX87_RS13530 | NZ_CP009257.1 | 2679072 | 2680229 | − | 1158 | 2 |
| IX87_RS13535 | NZ_CP009257.1 | 2680429 | 2681439 | + | 1011 | 2 |
| IX87_RS13560 | NZ_CP009257.1 | 2686163 | 2686519 | − | 357 | 2 |
| IX87_RS13570 | NZ_CP009257.1 | 2686998 | 2687317 | + | 320 | 2 |
| IX87_RS13670 | NZ_CP009257.1 | 2710800 | 2711243 | + | 444 | 2 |
| IX87_RS13880 | NZ_CP009257.1 | 2755728 | 2756186 | − | 459 | 2 |
| IX87_RS13905 | NZ_CP009257.1 | 2759219 | 2759755 | + | 537 | 2 |
| IX87_RS13910 | NZ_CP009257.1 | 2759765 | 2760010 | + | 246 | 2 |
| IX87_RS13940 | NZ_CP009257.1 | 2764567 | 2765370 | + | 804 | 2 |
| IX87_RS14105 | NZ_CP009257.1 | 2804249 | 2805253 | − | 1005 | 2 |
| IX87_RS14280 | NZ_CP009257.1 | 2838473 | 2839378 | − | 906 | 2 |
| IX87_RS14300 | NZ_CP009257.1 | 2843591 | 2844519 | − | 929 | 2 |
| IX87_RS14355 | NZ_CP009257.1 | 2854472 | 2854798 | + | 327 | 2 |
| IX87_RS14390 | NZ_CP009257.1 | 2861851 | 2862417 | + | 567 | 2 |
| IX87_RS14415 | NZ_CP009257.1 | 2866143 | 2867126 | − | 984 | 2 |
| IX87_RS14430 | NZ_CP009257.1 | 2869976 | 2870296 | − | 321 | 2 |
| IX87_RS14685 | NZ_CP009257.1 | 2927022 | 2927597 | + | 576 | 2 |
| IX87_RS14705 | NZ_CP009257.1 | 2931641 | 2932870 | + | 1230 | 2 |
| IX87_RS14720 | NZ_CP009257.1 | 2935284 | 2935949 | + | 666 | 2 |
| IX87_RS14740 | NZ_CP009257.1 | 2939054 | 2940199 | + | 1146 | 2 |
| IX87_RS14820 | NZ_CP009257.1 | 2957391 | 2957807 | − | 417 | 2 |
| IX87_RS14865 | NZ_CP009257.1 | 2972393 | 2973667 | + | 1275 | 2 |
| IX87_RS14875 | NZ_CP009257.1 | 2975648 | 2976340 | + | 693 | 2 |
| IX87_RS14920 | NZ_CP009257.1 | 2980543 | 2980980 | + | 438 | 2 |
| IX87_RS15070 | NZ_CP009257.1 | 3007654 | 3008868 | − | 1215 | 2 |
| IX87_RS15120 | NZ_CP009257.1 | 3017633 | 3018847 | − | 1215 | 2 |
| IX87_RS15175 | NZ_CP009257.1 | 3028815 | 3029300 | − | 486 | 2 |
| IX87_RS15180 | NZ_CP009257.1 | 3029382 | 3029786 | + | 405 | 2 |
| IX87_RS15230 | NZ_CP009257.1 | 3039800 | 3040405 | − | 606 | 2 |
| IX87_RS15245 | NZ_CP009257.1 | 3042927 | 3043571 | + | 645 | 2 |
| IX87_RS15295 | NZ_CP009257.1 | 3054124 | 3054951 | + | 828 | 2 |
| IX87_RS15305 | NZ_CP009257.1 | 3055888 | 3056664 | − | 777 | 2 |
| IX87_RS15535 | NZ_CP009257.1 | 3102339 | 3103478 | + | 1140 | 2 |
| IX87_RS15805 | NZ_CP009257.1 | 3155919 | 3156773 | + | 855 | 2 |
| IX87_RS15930 | NZ_CP009257.1 | 3179906 | 3180307 | + | 402 | 2 |
| IX87_RS16025 | NZ_CP009257.1 | 3206094 | 3207845 | + | 1752 | 2 |
| IX87_RS16030 | NZ_CP009257.1 | 3208562 | 3209860 | − | 1299 | 2 |
| IX87_RS16110 | NZ_CP009257.1 | 3227249 | 3227506 | + | 258 | 2 |
| IX87_RS16205 | NZ_CP009257.1 | 3244577 | 3245731 | + | 1155 | 2 |
| IX87_RS16210 | NZ_CP009257.1 | 3245783 | 3246538 | − | 756 | 2 |
| IX87_RS16315 | NZ_CP009257.1 | 3269023 | 3269271 | − | 249 | 2 |
| IX87_RS16560 | NZ_CP009257.1 | 3314155 | 3314664 | − | 510 | 2 |
| IX87_RS16615 | NZ_CP009257.1 | 3324800 | 3325675 | + | 876 | 2 |
| IX87_RS17015 | NZ_CP009257.1 | 3416053 | 3416874 | − | 822 | 2 |
| IX87_RS17260 | NZ_CP009257.1 | 3468422 | 3468859 | − | 438 | 2 |
| IX87_RS17445 | NZ_CP009257.1 | 3506503 | 3507231 | + | 729 | 2 |
| IX87_RS17460 | NZ_CP009257.1 | 3508737 | 3509216 | + | 480 | 2 |
| IX87_RS17500 | NZ_CP009257.1 | 3513881 | 3515056 | + | 1176 | 2 |
| IX87_RS17535 | NZ_CP009257.1 | 3523126 | 3524055 | − | 930 | 2 |
| IX87_RS17545 | NZ_CP009257.1 | 3525074 | 3526384 | − | 1311 | 2 |
| IX87_RS17675 | NZ_CP009257.1 | 3556022 | 3556156 | − | 135 | 2 |
| IX87_RS17685 | NZ_CP009257.1 | 3559377 | 3559583 | + | 207 | 2 |
| IX87_RS17845 | NZ_CP009257.1 | 3587515 | 3588714 | − | 1200 | 2 |
| IX87_RS17940 | NZ_CP009257.1 | 3610367' | 3611290 | − | 924 | 2 |
| IX87_RS18015 | NZ_CP009257.1 | 3628296 | 3629147 | − | 852 | 2 |
| IX87_RS18205 | NZ_CP009257.1 | 3659788 | 3660339 | + | 552 | 2 |
| IX87_RS18490 | NZ_CP009257.1 | 3703101 | 3703523 | − | 423 | 2 |
| IX87_RS18695 | NZ_CP009257.1 | 3748691 | 3749155 | + | 465 | 2 |
| IX87_RS18765 | NZ_CP009257.1 | 3767339 | 3767965 | + | 627 | 2 |
| IX87_RS18770 | NZ_CP009257.1 | 3767953 | 3768471 | + | 519 | 2 |
| IX87_RS18790 | NZ_CP009257.1 | 3771920 | 3772663 | + | 744 | 2 |
| IX87_RS18800 | NZ_CP009257.1 | 3773945 | 3774832 | − | 888 | 2 |
| IX87_RS18815 | NZ_CP009257.1 | 3776813 | 3777040 | − | 228 | 2 |
| IX87_RS18860 | NZ_CP009257.1 | 3787422 | 3787883 | + | 462 | 2 |
| IX87_RS19060 | NZ_CP009257.1 | 3821843 | 3822754 | − | 912 | 2 |
| IX87_RS19075 | NZ_CP009257.1 | 3824992 | 3826647 | + | 1656 | 2 |
| IX87_RS19140 | NZ_CP009257.1 | 3838523 | 3839362 | + | 840 | 2 |
| IX87_RS19260 | NZ_CP009257.1 | 3862694 | 3863680 | + | 987 | 2 |
| IX87_RS19295 | NZ_CP009257.1 | 3870370 | 3871428 | − | 1059 | 2 |
| IX87_RS19305 | NZ_CP009257.1 | 3872918 | 3873409 | − | 492 | 2 |
| IX87_RS19315 | NZ_CP009257.1 | 3875050 | 3875400 | + | 351 | 2 |
| IX87_RS19330 | NZ_CP009257.1 | 3877071 | 3877691 | + | 621 | 2 |
| IX87_RS19415 | NZ_CP009257.1 | 3898826 | 3899062 | + | 237 | 2 |
| IX87_RS19430 | NZ_CP009257.1 | 3901817 | 3902284 | + | 468 | 2 |
| IX87_RS19900 | NZ_CP009257.1 | 3997189 | 3997629 | + | 441 | 2 |
| IX87_RS19920 | NZ_CP009257.1 | 4000571 | 4001809 | − | 1239 | 2 |
| IX87_RS19950 | NZ_CP009257.1 | 4008136 | 4009488 | − | 1353 | 2 |
| IX87_RS19965 | NZ_CP009257.1 | 4011937 | 4012821 | − | 885 | 2 |
| IX87_RS19985 | NZ_CP009257.1 | 4015182 | 4015946 | − | 765 | 2 |
| IX87_RS20000 | NZ_CP009257.1 | 4018405 | 4019457 | − | 1053 | 2 |
| IX87_RS20335 | NZ_CP009257.1 | 4084321 | 4085100 | + | 780 | 2 |
| IX87_RS20370 | NZ_CP009257.1 | 4093669 | 4094406 | − | 738 | 2 |
| IX87_RS20380 | NZ_CP009257.1 | 4094913 | 4096190 | − | 1278 | 2 |
| IX87_RS20485 | NZ_CP009257.1 | 4117322 | 4118149 | − | 828 | 2 |
| IX87_RS20570 | NZ_CP009257.1 | 4133800 | 4134597 | + | 798 | 2 |
| IX87_RS20690 | NZ_CP009257.1 | 4162078 | 4162686 | + | 609 | 2 |
| IX87_RS20735 | NZ_CP009257.1 | 4168206 | 4168877 | − | 672 | 2 |
| IX87_RS20785 | NZ_CP009257.1 | 4175596 | 4175919 | − | 324 | 2 |
| IX87_RS20815 | NZ_CP009257.1 | 4180715 | 4181170 | − | 456 | 2 |
| IX87_RS21130 | NZ_CP009257.1 | 4233621 | 4234250 | + | 630 | 2 |
| IX87_RS21155 | NZ_CP009257.1 | 4239278 | 4240024 | − | 747 | 2 |
| IX87_RS21360 | NZ_CP009257.1 | 4276404 | 4276754 | + | 351 | 2 |
| IX87_RS21375 | NZ_CP009257.1 | 4277392 | 4278243 | − | 852 | 2 |
| IX87_RS21420 | NZ_CP009257.1 | 4287087 | 4287716 | − | 630 | 2 |
| IX87_RS21485 | NZ_CP009257.1 | 4299933 | 4300943 | + | 1011 | 2 |
| IX87_RS21515 | NZ_CP009257.1 | 4305821 | 4306984 | − | 1164 | 2 |
| IX87_RS00070 | NZ_CP009257.1 | 6580 | 7008 | − | 429 | 1 |
| IX87_RS00950 | NZ_CP009257.1 | 176584 | 177474 | − | 891 | 1 |
| IX87_RS01580 | NZ_CP009257.1 | 292739 | 293251 | − | 513 | 1 |
| IX87_RS01890 | NZ_CP009257.1 | 355224 | 361472 | − | 6249 | 1 |
| IX87_RS02215 | NZ_CP009257.1 | 408790 | 409170 | − | 381 | 1 |
| IX87_RS02220 | NZ_CP009257.1 | 409160 | 409714 | − | 555 | 1 |
| IX87_RS02530 | NZ_CP009257.1 | 467021 | 467761 | + | 741 | 1 |
| IX87_RS02570 | NZ_CP009257.1 | 476340 | 476777 | − | 438 | 1 |
| IX87_RS02870 | NZ_CP009257.1 | 533277 | 533438 | − | 162 | 1 |
| IX87_RS02900 | NZ_CP009257.1 | 539282 | 540751 | + | 1470 | 1 |
| IX87_RS22760 | NZ_CP009257.1 | 556299 | 556433 | + | 135 | 1 |
| IX87_RS03750 | NZ_CP009257.1 | 720754 | 721041 | − | 288 | 1 |
| IX87_RS03885 | NZ_CP009257.1 | 750903 | 751847 | + | 945 | 1 |
| IX87_RS22795 | NZ_CP009257.1 | 756062 | 756187 | − | 126 | 1 |
| IX87_RS04345 | NZ_CP009257.1 | 843468 | 843701 | − | 234 | 1 |
| IX87_RS04715 | NZ_CP009257.1 | 925346 | 925750 | − | 405 | 1 |
| IX87_RS04720 | NZ_CP009257.1 | 925843 | 926025 | − | 183 | 1 |
| IX87_RS05140 | NZ_CP009257.1 | 988872 | 989816 | + | 945 | 1 |
| IX87_RS05145 | NZ_CP009257.1 | 989858 | 990550 | − | 693 | 1 |
| IX87_RS22030 | NZ_CP009257.1 | 1012406 | 1012627 | + | 222 | 1 |
| IX87_RS05825 | NZ_CP009257.1 | 1123748 | 1124176 | − | 429 | 1 |
| IX87_RS06265 | NZ_CP009257.1 | 1202701 | 1203462 | − | 762 | 1 |
| IX87_RS06360 | NZ_CP009257.1 | 1225686 | 1225904 | − | 219 | 1 |
| IX87_RS06990 | NZ_CP009257.1 | 1358472 | 1358942 | − | 471 | 1 |
| IX87_RS08025 | NZ_CP009257.1 | 1560840 | 1561244 | − | 405 | 1 |
| IX87_RS08030 | NZ_CP009257.1 | 1561337 | 1561519 | − | 183 | 1 |
| IX87_RS08895 | NZ_CP009257.1 | 1736769 | 1737905 | − | 1137 | 1 |
| IX87_RS09125 | NZ_CP009257.1 | 1779926 | 1780987 | + | 1062 | 1 |
| IX87_RS09225 | NZ_CP009257.1 | 1800320 | 1800727 | + | 408 | 1 |
| IX87_RS09960 | NZ_CP009257.1 | 1979744 | 1980217 | − | 474 | 1 |
| IX87_RS10785 | NZ_CP009257.1 | 2134547 | 2134900 | − | 354 | 1 |
| IX87_RS11050 | NZ_CP009257.1 | 2191874 | 2192254 | + | 381 | 1 |
| IX87_RS11715 | NZ_CP009257.1 | 2322731 | 2323120 | − | 390 | 1 |
| IX87_RS11875 | NZ_CP009257.1 | 2340277 | 2341005 | − | 729 | 1 |
| IX87_RS12305 | NZ_CP009257.1 | 2428572 | 2429513 | + | 942 | 1 |
| IX87_RS12545 | NZ_CP009257.1 | 2479450 | 2479782 | − | 333 | 1 |
| IX87_RS12645 | NZ_CP009257.1 | 2497265 | 2500423 | − | 3159 | 1 |
| IX87_RS12785 | NZ_CP009257.1 | 2527485 | 2527796 | − | 312 | 1 |
| IX87_RS12790 | NZ_CP009257.1 | 2527803 | 2528192 | − | 390 | 1 |
| IX87_RS13510 | NZ_CP009257.1 | 2675956 | 2676459 | + | 504 | 1 |
| IX87_RS13540 | NZ_CP009257.1 | 2681415 | 2681939 | + | 525 | 1 |
| IX87_RS14000 | NZ_CP009257.1 | 2780047 | 2780937 | + | 891 | 1 |
| IX87_RS14540 | NZ_CP009257.1 | 2892269 | 2893309 | + | 1041 | 1 |
| IX87_RS15090 | NZ_CP009257.1 | 3011693 | 3012823 | − | 1131 | 1 |
| IX87_RS15395 | NZ_CP009257.1 | 3073910 | 3074341 | − | 432 | 1 |
| IX87_RS15450 | NZ_CP009257.1 | 3084892 | 3085155 | + | 264 | 1 |
| IX87_RS16090 | NZ_CP009257.1 | 3223840 | 3224184 | + | 345 | 1 |
| IX87_RS16095 | NZ_CP009257.1 | 3224329 | 3224667 | + | 339 | 1 |
| IX87_RS16165 | NZ_CP009257.1 | 3236746 | 3237126 | + | 381 | 1 |
| IX87_RS16245 | NZ_CP009257.1 | 3255035 | 3255709 | + | 675 | 1 |
| IX87_RS16340 | NZ_CP009257.1 | 3273468 | 3273875 | − | 408 | 1 |
| IX87_RS16345 | NZ_CP009257.1 | 3274044 | 3274412 | + | 369 | 1 |
| IX87_RS16350 | NZ_CP009257.1 | 3274464 | 3274685 | − | 222 | 1 |
| IX87_RS17405 | NZ_CP009257.1 | 3498499 | 3499290 | + | 792 | 1 |
| IX87_RS18780 | NZ_CP009257.1 | 3769521 | 3770120 | + | 600 | 1 |
| IX87_RS19485 | NZ_CP009257.1 | 3913207 | 3913581 | − | 375 | 1 |
| IX87_RS19525 | NZ_CP009257.1 | 3920445 | 3920819 | − | 375 | 1 |
| IX87_RS19705 | NZ_CP009257.1 | 3956110 | 3956868 | − | 759 | 1 |
| IX87_RS20330 | NZ_CP009257.1 | 4083096 | 4084310 | + | 1215 | 1 |
| IX87_RS20520 | NZ_CP009257.1 | 4123565 | 4124776 | − | 1212 | 1 |
| IX87_RS20585 | NZ_CP009257.1 | 4136520 | 4136927 | − | 408 | 1 |
| IX87_RS22670 | NZ_CP009257.1 | 4226744 | 4226836 | + | 93 | 1 |
| IX87_RS21185 | NZ_CP009257.1 | 4244367 | 4244603 | − | 237 | 1 |
| IX87_RS21615 | NZ_CP009257.1 | 4323255 | 4327616 | − | 4362 | 1 |

### Example 13

RNA Sequencing to Identify Bacterial Resistance in Humans with Sepsis

Attached are the bacterial resistance genes identified in one patient with COVID-19. Resistance Gene.

| | ARO | | | | |
|---|---|---|---|---|---|
| | Ontology | | # of | | Antibiotic |
| | ID | Mutation | Reads | Primary Species | Resistance |
| Nucleotide | 3002618 | aadA21 | 2 | n/a | n/a |
| Protein | 3001028 | TEM-162 | 2 | n/a | n/a |
| Homolog | 3002884 | iri | 2 | n/a | n/a |
| | 3001037 | TEM-171 | 2 | n/a | n/a |
| | 3000976 | TEM-113 | 2 | n/a | n/a |
| | 3000167 | tet(C) | 2 | n/a | n/a |
| | 3000833 | evgS | 2 | n/a | n/a |
| | 3003548 | mdtN | 2 | n/a | n/a |
| | 3001023 | TEM-157 | 2 | n/a | n/a |
| | 3002621 | aadA24 | 2 | n/a | n/a |
| | 3002620 | aadA23 | 4 | n/a | n/a |
| | 3000979 | TEM-116 | 4 | n/a | n/a |
| | 3002655 | APH(4)-Ia | 4 | n/a | n/a |
| | 3002641 | APH(3′)-Ia | 4 | n/a | n/a |
| | 3002601 | aadA | 4 | n/a | n/a |
| | 3002619 | aadA22 | 4 | n/a | n/a |
| | 3004089 | AND(3″)-IIa | 4 | n/a | n/a |
| | 3001044 | TEM-181 | 4 | n/a | n/a |
| | 3005036 | BLMT | 4 | n/a | n/a |
| | 3000805 | OprN | 6 | n/a | n/a |
| | 3002644 | APH(3′)-IIa | 8 | n/a | n/a |
| Nucleotide | 3004114 | porin | 2 | n/a | n/a |
| Protein | | OmpC | | | |
| Knockout | | | | | |
| Nucleotide | n/a | n/a | n/a | n/a | n/a |
| Protein | | | | | |
| Over- | | | | | |
| expression | | | | | |
| Nucleotide | 3003817 | DNA gyrase | 2 | Acinetobacter | Fluoroquinolones |
| Protein | 3003974 | DNA gyrase | 2 | Cutibacterium | Fluoroquinolones |
| Variant | | | | | |
| Nucleotide | 3005083 | 23s rRNA | 2 | Thermus | Pleuromutilins |
| rRNA | | | | Thermophilus | |
| | 3004170 | 23s rRNA | 4 | Streptococcus | Macrolides |
| | 3003372 | 16S rRNA | 4 | Escherichia | Spectinomycin |
| | | (rrsH) | | | |
| | 3003512 | 16S rRNA | 6 | Salmonella | Spectinomycin |
| | | (rrsD) | | | |
| | 3004836 | 23S rRNA | 6 | Neisseria | Azithromycin |
| | 3004181 | 23S rRNA | 8 | Streptococcus | Macrolides, |
| | | | | | streptogramins |
| | 3003493 | 16S rRNA | 10 | Pasteurella | Spectinomycin |
| | 3004058 | 23S rRNA | 14 | Staphylococcus | Linezolid |
| | 3004131 | 23S rRNA | 16 | Escherichia | Macrolides |
| | 3004149 | 23S rRNA | 16 | Escherichia | Clindamycin |
| | 3004160 | 23S rRNA | 16 | Escherichia | Clarithromycin |
| | 3004173 | 23S rRNA | 16 | Escherichia | Oxazolidinone |
| | 3004150 | 23s rRNA | 16 | Escherichia | Chloramphenicol |
| | 3003495 | 16S rRNA | 24 | Neisseria | Spectinomycin |
| | 3003499 | 16S rRNA | 24 | Cutibacterium | Tetracyclines |
| | 3004138 | 23S rRNA | 48 | Moraxella | Macrolides |
| | 3004161 | 23S rRNA | 58 | Propionibacteria | Macrolides |
| | 3003480 | 16S rRNA | 98 | Mycobacterium | Streptomycin |
| | 3004853 | 16S rRNA | 98 | Mycobacterium | Capreomycin |
| | 3003436 | 16S rRNA | 98 | Mycobacterium | Kanamycin |
| | 3003437 | 16S rRNA | 98 | Mycobacterium | Viomycin |
| | 3003481 | 16S rRNA | 98 | Mycobacterium | Amikacin |
| | 3004171 | 23S rRNA | 284 | Streptomyces | Macrolides |
| | 3003540 | 16S rRNA | 332 | Mycolicibacterium | Hygromycin B |
| | | (rrsB) | | | |
| | 3003497 | 16S rRNA | 436 | Neisseria | Spectinomycin |
| | 3003514 | 16S rRNA | 450 | Mycobacteroides | Amikacin |
| | 3003515 | 16S rRNA | 450 | Mycobacteroides | Kanamycin A |
| | 3003516 | 16S rRNA | 450 | Mycobacteroides | Tobramycin |
| | 3003517 | 16S rRNA | 450 | Mycobacteroides | Gentamicin C |
| | 3003518 | 16S rRNA | 450 | Mycobacteroides | Neomycin |
| | 3003541 | 16S rRNA | 452 | Mycolicibacterium | Streptomycin |
| | | (rrsB) | | | |
| | 3003542 | 16S rRNA | 452 | Mycolicibacterium | Kanamycin A |
| | | (rrsB) | | | |
| | 3003545 | 16S rRNA | 452 | Mycolicibacterium | Neomycin |
| | | (rrsB) | | | |
| | 3003547 | 16S rRNA | 452 | Mycolicibacterium | Viomycin |
| | | (rrsB) | | | |
| | 3003539 | 16S rRNA | 466 | Mycolicibacterium | Hygromycin B |
| | | (rrsA) | | | |
| | 3003543 | 16S rRNA | 466 | Mycolicibacterium | Kanamycin A |
| | | (rrsA) | | | |
| | 3003544 | 16S rRNA | 466 | Mycolicibacterium | Neomycin |
| | | (rrsA) | | | |
| | 3003546 | 16S rRNA | 466 | Mycolicibacterium | Viomycin |
| | | (rrsA) | | | |
| | 3004164 | 23S rRNA | 580 | Mycobacterium | Clarithromycin |
| | | | | avium | |
| | 3004166 | 23S rRNA | 608 | Mycobacterium | Clarithromycin |
| | | | | intracellulare | |
| | 3004167 | 23S rRNA | 608 | Mycobacterium | Azithromycin |
| | | | | intracellulare | |
| | 3003236 | 16S rRNA | 770 | Mycobacteroides | Kanamycin |
| | 3003237 | 16S rRNA | 770 | Mycobacteroides | Tobramycin |
| | 3003238 | 16S rRNA | 770 | Mycobacteroides | Neomycin |
| | 3003239 | 16S rRNA | 770 | Mycobacteroides | Amikacin |
| | 3003240 | 16S rRNA | 770 | Mycobacteroides | Gentamicin |
| | 3004937 | 23S rRNA | 838 | Mycobacterium | Capreomycin |
| | 3004168 | 23S rRNA | 960 | Mycobacterium | Clarithromycin |
| | 3004165 | 23S rRNA | 1278 | Mycobacteroides | Clarithromycin |
| | | | | chelonae | |
| | 3004169 | 23S rRNA | 1410 | Mycolicibacterium | Clarithromycin |
| | 3004163 | 23S rRNA | 1724 | Mycobacteroides | Clarithromycin |
| | | | | abscessus | |

### Example 14

Deep RNA Sequencing of Intensive Care Unit Patients with COVID-19

Purpose: COVID-19 has impacted millions of patients across the world. Molecular testing occurring now identifies the presence of the virus at the sampling site: nasopharynx, nares, or oral cavity. RNA sequencing has the potential to establish both the presence of the virus and define the host's response in COVID-19.

Methods: Single center, prospective study of patients with COVID-19 admitted to the intensive care unit where deep RNA sequencing (>100 million reads) of peripheral blood with computational biology analysis was done. All patients had positive SARS-CoV-2 PCR. Clinical data was prospectively collected.

Results: The inventors enrolled fifteen patients at a single hospital. Patients were critically ill with a mortality of 47% and 67% were on a ventilator. All the patients had the SARS-CoV-2 RNA identified in the blood in addition to RNA from other viruses, bacteria, and archaea. The expression of many immune modulating genes, including PD-L1 and PD-L2, were significantly different in patients who died from COVID-19. Some proteins were influenced by alternative transcription and splicing events, as seen in HLA-C, HLA-E. NRP1 and NRP2. Entropy calculated from alternative RNA splicing and transcription start/end predicted mortality in these patients.

Conclusions: Current upper respiratory tract testing for COVID-19 only determines if the virus is present. Deep RNA sequencing with appropriate computational biology may provide important prognostic information and point to therapeutic foci to be precisely targeted in future studies.

Introduction: Severe acute respiratory syndrome coronavirus 2 (SARS-CoV-2) causing coronavirus disease 2019 (COVID-19) has led to millions of cases worldwide. Dong, Du, & Gardner, The Lancet Infectious Diseases (2020). Current testing is by polymerase chain reaction to detect viral RNA in the nares. Sethuraman, Jeremiah, & Ryo, JAMA (2020). This provides no insight into the host response. Patients with COVID-19 that require intensive care unit (ICU) care are sick and difficult to manage, thus, there is a need for other diagnostic tests during the hospital stay to assist the clinicians.

Deep RNA sequencing refers to a process of sequencing where (at least) 100 million reads of sequence are generated per sample. Deep sequencing allows for the study of low abundance RNA and biologic processes beyond gene expression. Typically. RNA sequencing data is aligned to the genome of interest, such as aligning to human genes when the sample comes from a human. Reads that do not align to the genome of interest are usually discarded. When the RNA sequencing is performed with this large number of reads, it could be used to identify the presence of specific pathogens in the blood by aligning the reads that would have been discarded to other genomes of interest. In COVID-19, sequencing reads of SARS-CoV-2 may provide insight into the biology of the virus during active illness. In addition, secondary infections could be identified, potentially allowing for better, pathogen-directed antibiotic treatment.

The host response to the virus is responsible for some of the morbidity and mortality observed. Bouadma et al., Journal of Clinical Immunology, 1-11 (2020). Acute respiratory distress syndrome (ARDS) is the most common complication encountered with COVID-19. Bouadma et al., Journal of Clinical Immunology, 1-11 (2020). The laboratory has shown that there are significant changes in alternative RNA splicing and transcription start and end in ARDS as assessed by deep RNA sequencing. Fredericks et al., Intensive Care Medicine (2020). These changes are thought to be due to the physiology of ARDS, e.g., hypoxia and acidosis, which are known to influence splicing. Whether this occurs in patients infected by COVID-19 is not known.

While RNA sequencing can be used to measure immune modulating gene expression, an alternative approach is the evaluation of global entropy, or disorder in the processing of RNA. Sterne-Weiler et al., Molecular Cell 72, 187-200.e186 (2018). The inventors found that this entropy metric combined with Principal Component Analysis (PCA) can be leveraged to distinguish COVID-19 patients that develop life-threatening illness from those likely to recover.

Here the inventors examine deep RNA sequencing data from patients in the ICU with COVID-19 to characterize both pathogens and host responses. The inventors evaluate the sequences for the presence of the SARS-CoV-2 virus and other potential infectious agents. The host response to COVID-19 is also characterized. The long-term goal is to combine these measurements to better assist clinical decision-making.

Study design, Population and Setting: The study enrolled ICU participants at a single tertiary care hospital evidence of SARS-CoV-2 infection based on positive PCR from the nasopharynx documented during admission. All participants, or their appropriate surrogate, provided informed consent as approved by the Institutional Review Board (Approval #: 411616). Blood samples were collected on day 0 of ICU admission. Clinical data including COVID specific therapies was collected prospectively from the electronic medical record and participants were followed until hospital discharge or death. Ordinal scale was collected as previously described by Beigel et al., New England Journal of Medicine (2020). See also sepsis and associated SOFA score. Singer et al., The third international consensus definitions for sepsis and septic shock (sepsis-3). JAMA, 315, 801-810 (2016). Also, with the diagnosis of ARDS. Ferguson et al., The Berlin definition of ARDS: An expanded rationale, justification, and supplementary material. Intensive Care Medicine, 38, 1573-1582 (2012).

RNA extraction and sequencing. Whole blood was collected in PAXgene tubes (Qiagen, Germantown, Md., USA) and sent to Genewiz (South Plainfield, N.J., USA) for RNA extraction, ribosomal RNA depletion and sequencing. Sequencing was done on Illumina HiSeq machines to provide 150 base pair, paired-end reads. Libraries were prepared to have three samples per lane. Each lane provided 350 million reads ensuring each sample had >100 million reads. Raw data was returned on password protected external hard drives to ensure the security of the genomic data.

Computational Bioloqy and Statistical Analysis. All computational analysis was done blinded to the clinical data. The data was assessed for quality control using FastQC. See Andrews, A quality control tool for high throughput sequence data. FastQC. In: Editor\hat{ }Book A quality control tool for high throughput sequence data. FastQC (2014). RNA sequencing data was aligned to the human genome utilizing the STAR aligner. Dobin et al., Bioinformatics (Oxford, England) 29: 15-21 (2013). Reads that aligned to the human genome were separated and are now referred to as ‘mapped’ reads. Reads that did not align to the human genome, which are typically discarded during standard RNA sequencing analysis, were identified as ‘unmapped’ reads. The unmapped reads then aligned to the SARS-CoV-2 genome (NC_045512) and counted per sample using Magic-BLAST. Boratyn et al., BMC Bioinformatics, 20, 405 (2019). A coverage map of the SARS-CoV-2 genome was generated using al the subjects to identify the gene expression patterns of the virus in critically ill COVID-19 patients. The unmapped reads were further analyzed with Kraken2 using the PlusPFP index to identify other bacterial, fungal, archaeal, and viral pathogens. See Wood, Lu, & Langmead, Genome Biology, 20, 257 (2019).

Reads that aligned to the human genome, the mapped reads, also underwent analysis for gene expression, alternative RNA splicing, and alternative transcription start/end via Whippet. Sterne-Weiler et al., Molecular Cell 72, 187-200.e186 (2018). When comparisons were made between groups (died vs. survived) differential gene expression was set with thresholds of both p<0.05 and +/−1.5 log₂ fold change. Alternative splicing was defined as core exon, alternative acceptor splice site, alternative donor splice site, retained intron, alternative first exon and alternative last exon. Alternative transcription start/end events were defined as tandem transcription start site and tandem alternative polyadenylation site. Alternative RNA splicing and alternative transcription start/end events were also compared between groups. Sterne-Weiler et al., Molecular Cell 72, 187-200.e186 (2018). Significance was set at great than 2 log 2 fold change as previously described by Fredericks et al., Intensive Care Medicine (2020). Genes identified from the analysis of mapped reads were then evaluated by GO enrichment analysis (PANTHER Overrepresentation released 20200728). See Mi, Muruganujan, Casagrande, & Thomas. Nature Protocols. 8, 1551-1566 (2013).

Whippet was also used to generate an entropy value for every identified alternative splicing and transcription event of each gene. These entropy values are created without the need for groups used in the gene expression analysis. To visualize this data a principal component analysis (PCA) was conducted to reduce the dimensionality of the dataset and to obtain an unsupervised overview of trends in entropy values among the samples. Raw entropy values from all samples were concatenated into one matrix and missing values were replaced with column means. Mortality was then overlaid onto the PCA plot to assess the ability of these raw entropy values to predict this outcome in this sample set. This analysis was done in R (version 3.6.3).

Study Population. Participant Characteristics, and RNA sequencing: Fifteen participants were enrolled and had blood samples drawn on the first day of their ICU stay. Clinical and demographic data is reported in TABLE 1. Most participants were male (73%). There was a diverse distribution in terms of race (60% not white) and ethnicity (60% Hispanic). The most common co-morbidity was hypertension, and the median BMI was almost 30. Forty percent of participants had ARDS at the time the samples was drawn, and the patients were distributed across the top of the ordinal scale with a score of 5 as the most common in 53% of the patients. Most participants required a ventilator (67%) and 20% progressed to extracorporeal membrane oxygenation (ECMO); 27% required renal replacement. The median length of hospital stay was 22 days with a mortality rate of 47%.

All samples had sufficient RNA and RNA integrity numbers (RIN) were adequate. See Fleige & Pfaffl, Molecular Aspects of Medicine, 27, 126-139 (2006). The median of sequencing was 125,687,784 reads (95% Cl 122,164,763 to 135,800,242) and greater than 90% of those reads were more than thirty bases. After using FastQC, all samples had mean quality scores over 30. The reads mapped to the human genome 62-66% of the time.

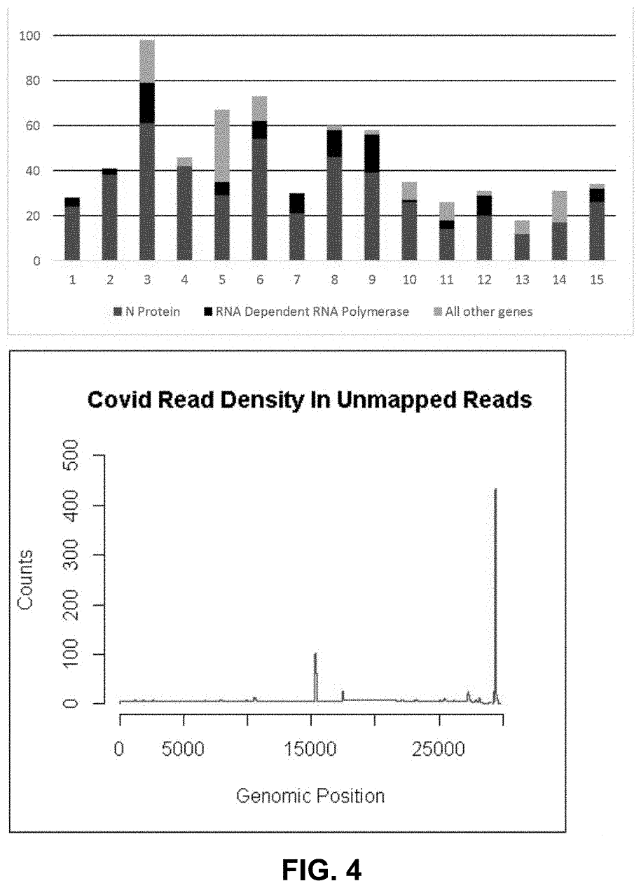

Identification of SARS-CoV-2 and other pathogens: Among the fifteen participant samples all participants had SARS-CoV-2 RNA detected. There was a total of 676 reads that align to the SARS-CoV-2 genome with each patient having between 18 and 98 reads. See FIG. 4. Most of the reads corresponded to the RNA dependent RNA polymerase and N protein genes. RNA from other pathogens including bacteria, viruses and archaea were identified in the blood of all patients. See TABLE 2. Two participants had fungal RNA identified. Despite alignment to a robust database of organisms, each participant still had hundreds of thousands of unclassified reads. TABLE 2. The taxonomy classification of “other sequences” (28384) align to elements of cellular organisms (bacterial, archaea, plant), but do not have enough specificity to identify a single species are listed in TABLE 2. The top bacterial sequences from all patients were from either Acinetobacter baumannii or Chryseobacterium gallinarum. In patients who had the most counts of C. gallinarum, A. baumannii had significantly reduced counts compared to the counts in other patients (148.1 vs. 50905.3, p<0.05). Although sequences corresponding to A. baumannii or C. gallinarum were found in all patients, none of the patients had positive blood cultures drawn around the time of these samples. No counts of bacteria, virus, archaea, (TABLE 2) or specific bacteria correlated with mortality.

Genomic differences between participants who lived and those who died. Among participants who died there were 86 genes that increased in expression and 207 that decreased in expression (top results in TABLE 3. There were 88 significant alternative splicing events occurring in 84 unique genes (Top results TABLE 3) and 2093 alternative transcription events occurring in 1769 unique genes (Top results TABLE 3). ABCA13 was the only gene that had significant expression and alternative splicing events. Twenty-seven genes had significant expression and alternative transcription start/end differences. (TABLE 3) Eighteen genes had significantly different alternative splicing and alternative transcription start/end. (TABLE 3).

The genes that were significant between groups then underwent GO term analysis to assess significant enrichment for a biological process. The top GO terms for gene expression and alternative transcription are listed in TABLE 3. There were no significant GO terms for the genes impacted by alternative splicing.

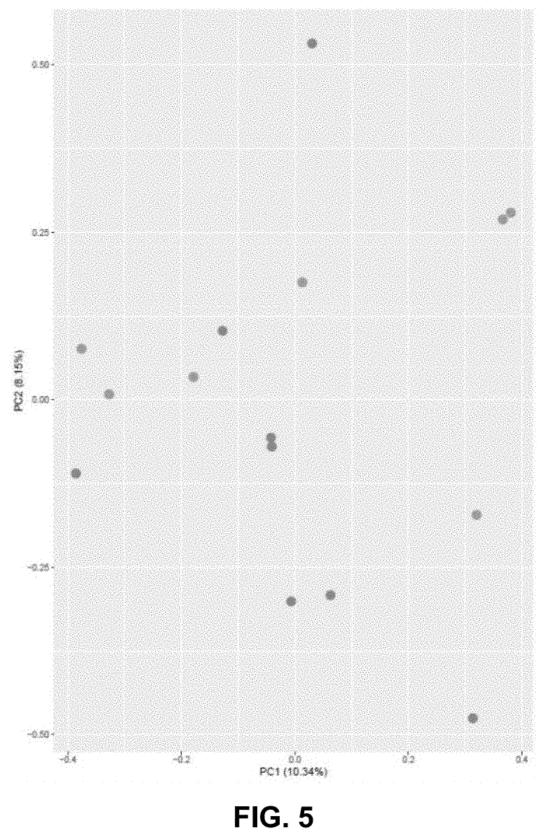

RNA entropy as a diagnostic tool. From the over 100 million RNA sequencing reads for each participant, computational analysis via Whippet assigns an entropy value for over 380,000 RNA splicing events and alternative transcription start/end events. Principal component analysis was then applied to these >380.000 entropy scores for each of the fifteen participants and the first two principal components were plotted against each other (FIG. 5). The sample points were then labeled based on their survival status. Survival status was not part of the principal component analysis itself. Participants whose PC2 value was above 0.00 had a mortality rate of 75% (6/8), up from the total group mortality of 46% (7/15) and significantly more than the 14% for those who land below that line (1/7, p=0.04).

Discussion. This project used deep RNA sequencing of whole blood from participants in the ICU with COVID-19 as a diagnostic tool. The protocol extracted RNA from the whole blood, as opposed to fractionating the whole blood specimen. Analysis of whole blood increased the breadth of RNA being sequenced, both cell associated and cell-free, and its simplicity for clinical practice. Alternatively, more complicated techniques, such as single cell sequencing may speak more to pathogenesis but adds to the complexity of the protocol and analysis. Despite its isolation from whole blood, the RNA was of high quality. A finding using RNA from whole blood from critically ill participants is that only 62-67% of the reads mapped to the human genome. This is less than the 85-97% of reads that typically map to the reference genome. See Sequencing Quality Control Consortium. Nature Biotechnology, 32, 903-914 (2014). One major drawback is the timing needed for RNA sequencing and analysis. Sequencing machines take ˜eighteen hours to generate data. The analysis can take additional time and is not yet clinically standardized. As technology advances and speed improves, this data can be increasingly accessible in the care of ICU patients.

SARS-CoV-2 RNA was identified in the unmapped reads in all patients (FIG. 1(a)). This supports that detection of SARS-CoV-2 in the serum has been associated with clinical deterioration. Chen et al., Emerging Microbes & Infections, 9, 469-473 (2020). RT-PCR identified the SARS-CoV-2 virus in the blood more often in the ICU patients than in the non-ICU patients. Fang et al., The Journal of Infection (2020). The total number of reads in the dataset did not correlate with any outcomes, including mortality, ARDS, or coagulopathy. The low number of total reads, approximately 700 from nearly two billion from all the samples, explains the lack of success from other researchers identifying the virus in the blood. In early reports. RT-PCR directed at the N protein gene identified viral RNA in the plasma in 15% of patients. Huang et al., Lancet (London, England), 395, 497-506 (2020). The data demonstrate the two most abundant genes in blood were the RNA Dependent RNA Polymerase and the N protein (FIG. 4). With this data, the inventors identified these locations (RNA dependent RNA polymerase or N protein) as potential therapeutic or diagnostic targets. See Gordon et al., A SARS-CoV-2 protein interaction map reveals targets for drug repurposing.

Other authors have called for robust testing for potential co-infections with SARS-CoV-2. Lai. Wang. & Hsueh, Journal of Microbiology, Immunology, and Infection, 53, 505-512 (2020). With deep sequencing and computational analysis, the inventors have identified the RNA from multiple bacteria, viruses, and archaea in all of the specimens, as well as fungal RNA in two participants. This suggests deep RNA sequencing with computational analysis may be a tool for the identification of co-infections. More data is required with comparison to gold standards such as blood culture and pathogen-specific PCR. RNA sequencing has the benefit of being able to identify all pathogens with known genomes, including both RNA and DNA based organisms. Unclassified reads that do not align to any known organism (TABLE 2) or the other sequences that have cellular organism elements (TABLE 2) could provide evidence of pathogens before a genome is sequenced or the pathogen is cultured.

Critically ill COVID-19 patients provide a difficult clinical dilemma as it pertains to antibiotics. In severely ill patients, clinicians are more likely to prescribe antibiotics despite there not being an identified pathogen. Feng et al., American Journal of Respiratory and Critical Care Medicine (2020). With identification of bacteria known to cause human disease from the RNA sequencing data, appropriate antibiotics could be prescribed to these patients. In this data set, the inventors show that there were significantly more counts of Acinetobacter baumannii in a portion of patients. This bacterium has been associated with COVID-19. Sharifipour et al., BMC Infectious Diseases, 20, 646 (2020). Using a precision medicine approach with these data, patients with significantly elevated levels may potentially be treated with directed antibiotics, in the absence of more time-consuming positive culture data. While there was no difference in survival in participants with versus without identified bacteria in this study, antibiotic use was not standardized or prescribed prospectively based upon the results. Analysis of the unmapped reads aligning to Acinetobacter baumannii (averaging over 50.000 among the six with increased reads) could provide insights into genes that are expressed in critical illness and provide useful diagnostic and therapeutic targets.

The immune response to SARS-CoV-2 has been the focus of much research since the pandemic started. Poland, Ovsyannikova, & Kennedy, Lancet (London, England) (2020). The successful use of corticosteroids in the critically ill with COVID-19 emphasizes the importance of the immune system in this disease. Dexamethasone in Hospitalized Patients with Covid-19—Preliminary Report. New England Journal of Medicine (2020); Prescott & Rice, JAMA, 324, 1292-1295 (2020). Because a significant proportion of COVID-19 patients do not respond to corticosteroids, there are still calls for a more precise approach. Waterer & Rello, Infectious Diseases and Therapy (2020). PD-1 expression is increased in certain cell populations in patients with COVID-19. Bellesi et al., British Journal of Haematology (2020). But the uses of immune checkpoint inhibitors in cancer patients have been associated with more severe COVID-19. Robilotti et al., Nature Medicine 26: 1218-1223 (2020). Other authors suggest that immune checkpoint inhibitors may be useful in COVID-19. Vivarelli et al., Cancers 12 (2020). The data shows that patients who died had increased expression of PD-L1 and PD-L2 (FIG. E1, CD274 and PDCD1Lg1, TABLE 3). This suggests that immune checkpoint inhibitors targeted against the PD-1 system might be considered in those patients identified to have increased expression of PD-L1 and PD-L2 because of their higher risk of death after ICU admission.

Numerous other immune targets are identified from these genomic changes. N4BP1 is induced by interferon and the interferon response has been implicated in COVID-19. Hadjadj et al., Science (New York, N.Y.), 369, 718-724 (2020); Lei et al., Nature Commun., 11, 3810 (2020). The data supports the role for interferons in COVID-19 as patients who died had 2.5-fold increase in expression of interferon 1 alpha (IFNA1). Clinical features of COVID-19 also correlate with some of the genes identified. OR6C4 is an olfactory gene which the inventors identified has exhibiting a 5 fold increased in expression in patients that died (TABLE 3). This finding suggests that loss of smell may signify milder disease among patients in the ICU. Thrombotic complications are common in COVID-19 patients (9.5%) and patients admitted to the ICU have a higher incidence of venous thromboembolism. Al-Samkari et al., Blood (2020). Patients who died have significant decrease in gene expression and multiple changes in alternative transcription end (TABLE 3) of both NRP1 and NRP2. Both these genes are associated with coagulation. See Rossignol, Gagnon, & Klagsbrun. Genomics 70: 211-222 (2000). The COVID-19 spike protein binds both these receptors. Daly et al., Science (New York, N.Y., 2020). Previous work has shown that there is increased expression in both genes in the lungs of patients with COVID-19 when compared to controls. See Ackermann et al., The New England Journal of Medicine, 383, 120-128 (2020). Here, the decrease NRP1 and NRP2 were seen in ICU patients who died compared to ICU patients who survived.

Many studies have attempted to utilize clinical data to predict mortality in COVID-19. See Tian et al., Journal of Medical Virology (2020);_Zhang et al., Journal of Thrombosis and Haemostasis (JTH), 18, 1324-1329 (2020). Some focus on cytokines. McElvaney et al., EBioMedicine 61, 103026 (2020). For simplicity all these attempt to identify a few variables to predict mortality. Here the inventors utilize over 380,000 variables with PCA to create a figure that improves mortality prediction based upon where the patient is on the graph (FIG. 5, 75% versus 14%). A limitation to this form of analysis is that the PCA cannot identify a specific gene or event most responsible for outcomes; it uses all 380.000 data points. Accurate assessment of prognosis using sequencing technology might be valuable to inform end of life care discussions in the ICU.

Despite the limitations of this single-center study with a small patient number, the inventors were still able to document that deep RNA sequencing and appropriate computational analysis yields valuable insight into the pathogenesis and host response of COVID-19 in critically ill patients. Useful drug targets were identified from SARS-CoV-2 RNA and the host response, including RNA dependent RNA polymerase, the N protein, and the PD-1 immune checkpoint pathway. The presence of pathogen RNA in the blood suggests co-infection should be reconsidered. Most importantly, PCA of the entropy of >380,000 events allowed use to group patients into those likely to die versus those likely to live, and this may be helpful in family discussions with critically ill patients. Translating these results to clinical practice can improve the diagnosis, assessment of prognosis, and therapy of COVID-19.

### Example 15

### Alternative RNA Splicing and Alternative Transcription Start/End in Acute Respiratory Distress Syndrome

Critically ill patients develop acute respiratory distress syndrome (ARDS) and despite the study of genomics of ARDS, there is little progress. The drop in the cost of sequencing has refocused genetic studies from DNA to RNA sequencing and methods to analyze this data have improved. The objective of this investigation is to utilize RNA sequencing data and analysis to identify useful gene targets in ARDS.

The human cohort generated from the GTEx consortium consisted of 25 deceased patients with ARDS identified by the presence of diffuse alveolar damage (DAD), and 74 deceased patients evaluated to not have DAD. The mouse ARDS cohort included C57BL/6 mice ages 10-12 in a model previously described and compared to controls.

Alternatively spliced RNA arises from co/post-transcriptional events facilitated by the spliceosome, introns are removed to form the mature RNA from which protein isoforms are translated. Alternatively transcribed genes are the product of changes in promoter usage, polyadenylation signals, and RNA polymerase II interactions with DNA which can lead to changes in isoform usage like alternative splicing events. These are identified from the analysis of RNA sequencing data. Significant differentially alternatively transcribed genes and alternative spliced genes were identified and where alternative transcription may have separate roles in DAD/ARDS by regulating different genes to perform distinctive functions.

In this analysis of RNA sequencing data from deceased patients with ARDS identified by the presence of DAD and a clinically relevant mouse model of ARDS, useful genes were identified. Future research is needed using on the mechanism of alternative RNA splicing and alternative transcription start/end seen in ARDS, overlapped with genes previously reported as ARDS related. Of 89 reported ARDS related genes, 38 were confirmed in at least one differential category confirming that the use of humans and mice with DAD/ARDS is appropriate and robust (p=1.25e−14). Eleven previously reported genes were present in all categories. These eleven genes were evaluated for the change in alternative splicing and alternative transcription. GO term enrichment analysis was performed on the 11 overlapping genes, revealing 20 significant biological processes including ontology related to aging, and response to abiotic/environmental stimuli. There are 1639 genes that show overlap in alternative splicing and alternative transcription that were not previously in the literature. These genes were assessed for directionality alternative splicing and alternative transcription and GO terms should provide the foundation for future work in ARDS.

Studying the underlying changes in RNA processing (alternative splicing and alternative transcription start/end) not only expands basic knowledge of pathogenicity, but also provides additional targets for therapeutics. The most enriched GO term from the alternative splicing set, carboxy-terminal domain protein kinase complex (GO: 0032806) refers to phosphorylation of the CTD of RNA polymerase II, which is vital in the regulation of transcription and RNA processing. In addition, RNA polymerase complex binding (GO: 0000993), and transport of the SLBP Independent/Dependent mature mRNA (R-HSA-159227; R-HSA-159230) are among the most enriched. This suggests alternative pre-mRNA splicing plays the dominate role in isoform usage in genes where expressions levels do not change, whereas alternative transcription may regulate isoform usage in genes that are more dynamically expressed during critical illness. Although it is possible the enrichment reflects down regulation through inhibitory genes, these data support the hypothesis that alternative splicing. Although it is possible the enrichment reflects down regulation through inhibitory genes, these data support the hypothesis that alternative splicing and alternative transcription may have separate roles in DAD/ARDS by regulating different genes to perform distinctive functions.

In this analysis of RNA sequencing data from deceased patients with ARDS identified by the presence of DAD and a clinically relevant mouse model of ARDS, useful genes were identified. Future research is needed using on the mechanism of alternative RNA splicing and alternative transcription start/end seen in ARDS.

### Example 16

### Updated Results

To translate the work described above, where SARS-CoV-2 was identified in the blood of patients using this methodology, the inventors again showed that they do this for other infections, specifically the bacterial infection Escherichia coli.

In a patient with a known Escherichia coli infection the blood of that patient was sequenced to a depth of >100 million reads. Sequencing data was aligned using STAR aligner with standard parameters to the human genome. Unmapped reads were extracted and then aligned to the Escherichia coli genome (Escherichia coli O25b:H4-ST131).

The reads aligned to Escherichia coli in a patients with an Escherichia coli infection. See TABLE 7.

TABLE 7
All E. coli reads

| Start | End | Nucleic Acid Sequence |
|---|---|---|
| 242185 | 242285 | TGACTCTTGAAATCCATAAATTCAAGCGCAGTGCCCAGCCAT |
| | | CCCGATACTGCTGCTTTCACCAAATCCTTAGTGCTTCTTTCGT |
| | | GTTTTTCTATTGTCATAATGTTATCTCTAAAAAAGAGGTAAGAT |
| | | GCGTACTACTTACTCGCCGTT |
| 242285 | 242185 | TGTTATCTCTAAAAAAGAGGTAAGATGCGTACTACTTACTCGC |
| | | CGTTATTGGTATTATTCAGAAAAAGTGAGTAAGACTTTGCAGC |
| | | AATGTTTTTGATCCTGTTCAAATAAACTAATGGCATCAGCAAC |
| | | ATGCTGGAAATCAAACGTATG |
| 1500318 | 1500404 | CGGCCTATCAACGTCGTCGTCTTCAACGTTCCTTCAGGACTC |
| | | TCAAGGAGTCAGGGAGAACTCATCTCGGGGCAAGTTTCGTG |
| | | CTTAGATGCTTTCAGCACTTATCTCTTCCGCATTTAGCTACCG |
| | | GGCAGTGCCATTGGCATGACAACC |
| 1500404 | 1500318 | AGATGCTTTCAGCACTTATCTCTTCCGCATTTAGCTACCGGG |
| | | CAGTGCCATTGGCATGACAACCCGAACACCAGTGATGCGTC |
| | | CACTCCGGTCCTCTCGTACTAGGAGCAGCCCCCCTCAGTTCT |
| | | CCAGCGCCCACGGCAGATAGGGACC 1 |
| 501520 | 1501548 | CTTGGTATTCTCTACCTGACCACCTGTGTCGGTTTGGGGTAC |
| | | GATTTGATGTTACCTGATGCTTAGAGGCTTTTCCTGGAAGCA |
| | | GGGCATTTGTCGCTTCAGCACCGTAGTGCCTCGTCATCACGC |
| | | CTCAGCCTTGATTTTCCGGATTTG |
| 1501548 | 1501520 | TCGGTTTGGGGTACGATTTGATGTTACCTGATGCTTAGAGGC |
| | | TTTTCCTGGAAGCAGGGCATTTGTCGCTTCAGCACCGTAGTG |
| | | CCTCGTCATCACGCCTCAGCCTTGATTTTCCGGATTTGCCTG |
| | | GAAAACCAGCCTACACGCTTAAAC |
| 1501563 | 1501563 | ATTTGATGTTACCTGATGCTTAGAGGCTTTTCCTGGAAGCAG |
| | | GGCATTTGTCGCTTCAGCACCGTAGTGCCTCGTCATCACGC |
| | | CTCAGCCTTGATTTTCCGGATTTGCCTGGAAAACCAGCCTAC |
| | | ACGCTTAAACAGATCGGAAGAGCGT |
| 1501563 | 1501563 | GTGCTCTTCCGATCTATTTGATGTTACCTGATGCTTAGAGGC |
| | | TTTTCCTGGAAGCAGGGCATTTGTCGCTTCAGCACCGTAGTG |
| | | CCTCGTCATCACGCCTCAGCCTTGATTTTCCGGATTTGCCTG |
| | | GAAAACCAGCCTACACGCTTAAAC |
| 1501821 | 1501900 | CACCCTGCCCCGATTAACGTTGGACAGGAACCCTTGGTCTTC |
| | | CGGCGAGCGGGCTTTTCACCCGCTTTATCGTTACTTATGTCA |
| | | GCATTCGCACTTCTGATACCTCCAGCATACCTCACAGTACAC |
| | | GTTCACAGGCTTACAGAACGCTGC |
| 1501900 | 1501821 | TGTCAGCATTCGCACTTCTGATACCTCCAGCATACCTCACAG |
| | | TACACCTTCACAGGCTTACAGAACGCTCCCCTACCCAACAAC |
| | | ACATAGTGTCGCTGCCGCAGCTTCGGTGCATGGTTTAGCCC |
| | | CGTTACATCTTCCGCGCAGGCCGAC |
| 1502166 | 1502187 | GGCGGTCTGGGTTGTTTCCCTCTTCACGACGGACGTTAGCA |
| | | CCCGCCGTGTGTCTCCCGTGATAACATTCTCCGGTATTCGCA |
| | | GTTTGCATCGGGTTGGTAAGTCGGGATGACCCCCTTGCCGA |
| | | AACAGTGCTCTACCCCCGGAGATGAA |
| 1502166 | 2227814 | GGCGGTCTGGGTTGTTTCCCTCTTCACGACGGACGTTAGCA |
| | | CCCGCCGTGTGTCTCCCGTGATAACATTCTCCGGTATTCGCA |
| | | GTTTGCATCGGGTTGGTAAGTCGGGATGACCCCCTTGCCGA |
| | | AACAGATCGGAAGAGCACACGTCTGA |
| 1502166 | 2018481 | GGCGGTCTGGGTTGTTTCCCTCTTCACGACGGACGTTAGCA |
| | | CCCGCCGTGTGTCTCCCGTGATAACATTCTCCGGTATTCGCA |
| | | GTTTGCATCGGGTTGGTAAGTCGGGATGACCCCCTTGCCGA |
| | | AACAGATCGGAAGAGCACACGTCTGA |
| 1502176 | 2317776 | GTTGTTTCCCTCTTCACGACGGACGTTAGCACCCGCCGTGT |
| | | GTCTCCCGTGATAACATTCTCCGGTATTCGCAGTTTGCATCG |
| | | GGTTGGTAAGTCGGGATGACCCCCTTGCCGAAACAGTGCTC |
| | | TACCCCCGGAGATGAATTCACGAGAT |
| 1502187 | 1502166 | CTTCACGACGGACGTTAGCACCCGCCGTGTGTCTCCCGTGA |
| | | TAACATTCTCCGGTATTCGCAGTTTGCATCGGGTTGGTAAGT |
| | | CGGGATGACCCCCTTGCCGAAACAGTGCTCTACCCCCGGAG |
| | | ATGAATTCACGAGGCGCTACCTAAAT |
| 1502487 | 1502523 | CGGGTCTATACCCTGCAACTTAACGCCCAGTTAAGACTCGGT |
| | | TTCCCTTCGGCTCCCCTATTCGGTTAACCTTGCTACAGAATAT |
| | | AAGTCGCTGACCCATTATACAAAAGGTACGCAGTCACCCCAT |
| | | TAAGAGGCTCCCACTGCTTGTAC |
| 1502523 | 1502487 | CTCGGTTTCCCTTCGGCTCCCCTATTCGGTTAACCTTGCTAC |
| | | AGAATATAAGTCGCTGACCCATTATACAAAAGGTACGCAGTC |
| | | ACCCCATTAAGAGGCTCCCACTGCTTGTACGTACACGGTTTC |
| | | AGGTTCTTTTTCACTCCCCTCGCC |
| 1502812 | 1502812 | TTTTTGTGTACGGGGCTGTCACCCTGTATCGCGCGCCTTTCC |
| | | AGACGCTTCCACTAACACACACACTGATTCAGGCTCTGGGCT |
| | | CCTCCCCGTTCGCTCGCCGCTACTGGGGGAATCTCGGTTGA |
| | | TTTCTTAGATCGGAAGAGCACACGT |
| 1502812 | 1502812 | CACGACGCTCTTCCGATCTTTTTTGTGTACGGGC5CTGTCACC |
| | | CTGTATCGCGCGCCTTTCCAGACGCTTCCACTAACACACACA |
| | | CTGATTCAGGCTCTGGGCTCCTCCCCGTTCGCTCGCCGCTA |
| | | CTGGGGGAATCTCGGTTGATTTCTT |
| 1502923 | 1502923 | GGAATCTCGGTTGATTTCTTTTCCTCGGGGTACTTAGATGTTT |
| | | CAGTTCCCCCGGTTCGCCTCATTAACCTATGGATTCAGTTAA |
| | | TGATAGTGTGTCGAAACACACTGGGTTTCCCCATTCGGAAAT |
| | | CGCCGAGATCGGAAGAGCACACG |
| 1502923 | 1502923 | ACGACGCTCTTCCGATCTGGAATCTCGGTTGATTTCTTTTCCT |
| | | CGGGGTACTTAGATGTTTCAGTTCCCCCGGTTCGCCTCATTA |
| | | ACCTATGGATTCAGTTAATGATAGTGTGTCGAAACACACTGG |
| | | GTTTCCCCATTCGGAAATCGCCG |
| 1503664 | 1503684 | CAAGGCCCGGGAACGTATTCACCGTGGCATTCTGATCCACG |
| | | ATTACTAGCGATTCCGACTTCATGGAGTCGAGTTGCAGACTC |
| | | CAATCCGGACTACGACGCACTTTATGAGGTCCGCTTGCTCTC |
| | | GCAGATCGGAAGAGCACACGTCTGA |
| 1503664 | 1503664 | CCTACACGACGCTCTTCCGATCTCAAGGCCCGGGAACGTAT |
| | | TCACCGTGGCATTCTGATCCACGATTACTAGCGATTCCGACT |
| | | TCATGGAGTCGAGTTGCAGACTCCAATCCGGACTACGACGC |
| | | ACTTTATGAGGTCCGCTTGCTCTCGC |
| 1503671 | CGGGA | GGGGAACGTATTCACCGTGGCATTCTGATCCACGATTACTAG |
| | ACG | CGATTCCGACTTCATGGAGTCGAGTTGCAGACTCCAATCCG |
| | | GACTACGACGCACTTTATGAGGTCCGCTTGCTCTCGCAGATC |
| | | GGAAGAGCGTCGTGTAGGGAAAGAG |
| 1503812 | 1503849 | CGCCATTGTAGCACGTGTGTAGCCCTGGTCGTAAGGGCCAT |
| | | GATGACTTGACGTCATCCCCACCTTCCTCCAGTTTATCACTG |
| | | GCAGTCTCCTTTGAGTTCCCGGCCGGACCGCTGGCAACAAA |
| | | GGATAAGGGTTGCGCTCGTTGCGGGA |
| 1503849 | 1503812 | CCATGATGACTTGACGTCATCCCCACCTTCCTCCAGTTTATC |
| | | ACTGGCAGTCTCCTTTGAGTTCCCGGCCGGACCGCTGGCAA |
| | | CAAAGGATAAGGGTTGCGCTCGTTGCGGGACTTAACCCAAC |
| | | ATTTCACAACACGAGCTGACGACAGC |
| 1503942 | 1503953 | CGGGTTGCGCTCGTTGCGGGACTTAACCCAACATTTCACAAC |
| | | ACGAGCTGACGACAGCCATGCAGCACCTGTCTCACGGTTCC |
| | | CGAAGGCACATTCTCATCTCTGAAAACTTCCGTGGATGTCAA |
| | | GACCAGGTAAGGTTCTTCGCGTTGC |
| 1503953 | 1503942 | GTTGCGGGACTTAACCCAACATTTCACAACACGAGCTGACGA |
| | | CAGCCATGCAGCACCTGTCTCACGGTTCCCGAAGGCACATT |
| | | CTCATCTCTGAAAACTTCCGTGGATGTCAAGACCAGGTAAGG |
| | | TTCTTCGCGTTGCATCGAATTAAAC |
| 1504092 | 1504092 | TTCGAATTAAACCACATGCTCCACCGCTTGTGCGGGCCCCC |
| | | GTCAATTCATTTGAGTTTTAACCTTGCGGCCGTACTCCCCAG |
| | | GCGGTCAACTTAATGCGTTAGCTGCGCCACTAAAAGCTCAAG |
| | | GCTTCCAACAGATCGGAAGAGCACA |
| 1504092 | 1504092 | GACGCTCTTCCGATCTTTCGAATTAAACCACATGCTCCACCG |
| | | CTTGTGCGGGCCCCCGTCAATTCATTTGAGTTTTAACCTTGC |
| | | GGCCGTACTCCCCAGGCGGTCAACTTAATGCGTTAGCTGCG |
| | | CCACTAAAAGCTCAAGGCTTCCAAC |
| 1504400 | 1504439 | CTCAAGCTTGCCAGTATCAGATGCAGTTCCCAGGTTGAGCCC |
| | | GGGGATTTCACATCTGACTTAACAAACCGCCTGCGTGCGCTT |
| | | TACGCCCAGTAATTCCGATTAACGCTTGCACCCTCCGTATTA |
| | | CCGCGGCTGCTGGCACGGAGTTAG |
| 1504439 | 1504400 | CCCGGGGATTTCACATCTGACTTAACAAACCGCCTGCGTGC |
| | | GCTTTACGCCCAGTAATTCCGATTAACGCTTGCACCCTCCGT |
| | | ATTACCGCGGCTGCTGGCACGGAGTTAGCCGGTGCTTCTTC |
| | | TGCGGGTAACGTCAATGAGCAAAGGT |
| 2018481 | 1502166 | CCTACACGACGCTCTTCCGATCTGGCGGTCTGGGTTGTTTCC |
| | | CTCTTCACGACGGACGTTAGCACCCGCCGTGTGTCTCCCGT |
| | | GATAACATTCTCCGGTATTCGCAGTTTGCATCGGGTTGGTAA |
| | | GTCGGGATGACCCCCTTGCCGAAAC |
| 2227814 | 1502166 | CCTACACGACGCTCTTCCGATCTGGCGGTCTGGGTTGTTTCC |
| | | CTCTTCACGACGGACGTTAGCACCCGCCGTGTGTCTCCCGT |
| | | GATAACATTCTCCGGTATTCGCAGTTTGCATCGGGTTGGTAA |
| | | GTCGGGATGACCCCCTTGCCGAAAC |
| 2260692 | 2260927 | GTCACGCTCAAAGACGCGGGTCATATAGCCTTTCAGCTCTTT |
| | | CGCACCCGGGCCGCTGAACTCGATGCCCAGTTGCGGCAGA |
| | | CGCCACAGCAGCGGAGCAAGATAGCAATCGACCAGGCTGAA |
| | | CTCATCGCTCAGGAAGTACGGCTTCTG |
| 2260927 | 2260692 | GTGTTCATCAGCGTGTACCAGTCTTTTTCGATGGGATGCATG |
| | | TACAGACGGCTTTCACCGCGAGCTACCGGGTAAACAGGCAT |
| | | CAGTGGCGGATGCGGGAAACGCTCATCCAGATAGTCCATAA |
| | | TGATGCGAGATTCCCACAGGGTCAGC |
| 2315078 | 2315109 | GTTGTTTGATGAGCCAACGTCGGCGCTCGATCCTGAGATGG |
| | | TGAAAGAGGTGCTGGATACGATGATTGGGCTGGCGCAGTCG |
| | | GGTATGACAATGCTGTGTGTAACACATGAGATGGGGTTTGCA |
| | | CGAACCGTCGCTGACCGGGTGATTTT |
| 2315109 | 2315078 | CCTGAGATGGTGAAAGAGGTGCTGGATACGATGATTGGGCT |
| | | GGCGCAGTCGGGTATGACAATGCTGTGTGTAACACATGAGA |
| | | TGGGGTTTGCACGAACCGTCGCTGACCGGGTGATTTTTATG |
| | | GATCGTGGGGAGATAGTGGAACAAGCT |
| 2315918 | 2316004 | CGGCCTATCAACGTCGTCGTCTTCAACGTTCCTTCAGGACTC |
| | | TCAAGGAGTCAGGGAGAACTCATCTCGGGGCAAGTTTCGTG |
| | | CTTAGATGCTTTCAGCACTTATCTCTTCCGCATTTAGCTACCG |
| | | GGCAGTGCCATTGGCATGACAACC |
| 2316004 | 2315918 | AGATGCTTTCAGCACTTATCTCTTCCGCATTTAGCTACCGGG |
| | | CAGTGCCATTGGCATGACAACCCGAACACCAGTGATGCGTC |
| | | CACTCCGGTCCTCTCGTACTAGGAGCAGCCCCCCTCAGTTC |
| | | TCCAGCGCCCACGGCAGATAGGGACC |
| 2317120 | 2317148 | CTTGGTATTCTCTACCTGACCACCTGTGTCGGTTTGGGGTAC |
| | | GATTTGATGTTACCTGATGCTTAGAGGCTTTTCCTGGAAGCA |
| | | GGGCATTTGTCGCTTCAGCACCGTAGTGCCTCGTCATCACG |
| | | CCTCAGCCTTGATTTTCCGGATTTG |
| 2317148 | 2317120 | TCGGTTTGGGGTACGATTTGATGTTACCTGATGCTTAGAGGC |
| | | TTTTCCTGGAAGCAGGGCATTTGTCGCTTCAGCACCGTAGTG |
| | | CCTCGTCATCACGCCTCAGCCTTGATTTTCCGGATTTGCCTG |
| | | GAAAACCAGCCTACACGCTTAAAC |
| 2317163 | 2317163 | ATTTGATGTTACCTGATGCTTAGAGGCTTTTCCTGGAAGCAG |
| | | GGCATTTGTCGCTTCAGCACCGTAGTGCCTCGTCATCACGC |
| | | CTCAGCCTTGATTTTCCGGATTTGCCTGGAAAACCAGCCTAC |
| | | ACGCTTAAACAGATCGGAAGAGCGT |
| 2317163 | 2317163 | GTGCTCTTCCGATCTATTTGATGTTACCTGATGCTTAGAGGC |
| | | TTTTCCTGGAAGCAGGGCATTTGTCGCTTCAGCACCGTAGTG |
| | | CCTCGTCATCACGCCTCAGCCTTGATTTTCCGGATTTGCCTG |
| | | GAAAACCAGCCTACACGCTTAAAC |
| 2317766 | 2317787 | GGCGGTCTGGGTTGTTTCCCTCTTCACGACGGACGTTAGCA |
| | | CCCGCCGTGTGTCTCCCGTGATAACATTCTCCGGTATTCGCA |
| | | GTTTGCATCGGGTTGGTAAGTCGGGATGACCCCCTTGCCGA |
| | | AACAGTGCTCTACCCCCGGAGATGAA |
| 2317776 | 1502176 | ATCTGTTGTTTCCCTCTTCACGACGGACGTTAGCACCCGCCG |
| | | TGTGTCTCCCGTGATAACATTCTCCGGTATTCGCAGTTTGCA |
| | | TCGGGTTGGTAAGTCGGGATGACCCCCTTGCCGAAACAGTG |
| | | CTCTACCCCCGGAGATGAATTCACG |
| 2317787 | 2317766 | CTTCACGACGGACGTTAGCACCCGCCGTGTGTCTCCCGTGA |
| | | TAACATTCTCCGGTATTCGCAGTTTGCATCGGGTTGGTAAGT |
| | | CGGGATGACCCCCTTGCCGAAACAGTGCTCTACCCCCGGAG |
| | | ATGAATTCACGAGGCGCTACCTAAAT |
| 2318087 | 2318123 | CGGGTCTATACCCTGCAACTTAACGCCCAGTTAAGACTCGGT |
| | | TTCCCTTCGGCTCCCCTATTCGGTTAACCTTGCTACAGAATAT |
| | | AAGTCGCTGACCCATTATACAAAAGGTACGCAGTCACCCCAT |
| | | TAAGAGGCTCCCACTGCTTGTAC |
| 2318123 | 2318087 | GTCGGTTTCCCTTCGGCTCCCCTATTCGGTTAACCTTGCTAC |
| | | AGAATATAAGTCGCTGACCCATTATACAAAAGGTACGCAGTC |
| | | ACCCCATTAAGAGGCTCCCACTGCTTGTACGTACACGGTTTC |
| | | AGGTTCTTTTTCACTCCCCTCGCC |
| 2318412 | 2318412 | TTTTTGTGTACGGGGCTGTCACCCTGTATCGCGCGCCTTTCC |
| | | AGACGCTTCCACTAACACACACACTGATTCAGGCTCTGGGCT |
| | | CCTCCCCGTTCGCTCGCCGCTACTGGGGGAATCTCGGTTGA |
| | | TTTCTTAGATCGGAAGAGCACACGT |
| 2318412 | 2318412 | CACGACGCTCTTCCGATCTTTTTTGTGTACGGGGCTGTCACC |
| | | CTGTATCGCGCGCCTTTCCAGACGCTTCCACTAACACACACA |
| | | CTGATTCAGGCTCTGGGCTCCTCCCCGTTCGCTCGCCGCTA |
| | | CTGGGGGAATCTCGGTTGATTTCTT |
| 2318523 | 2318523 | GGAATCTCGGTTGATTTCTTTTCCTCGGGGTACTTAGATGTTT |
| | | CAGTTCCCCCGGTTCGCCTCATTAACCTATGGATTCAGTTAA |
| | | TGATAGTGTGTCGAAACACACTGGGTTTCCCCATTCGGAAAT |
| | | CGCCGAGATCGGAAGAGCACACG |
| 2318523 | 2318523 | ACGACGCTCTTCCGATCTGGAATCTCGGTTGATTTCTTTTCCT |
| | | CGGGGTACTTAGATGTTTCAGTTCCCCCGGTTCGCCTCATTA |
| | | ACCTATGGATTCAGTTAATGATAGTGTGTCGAAACACACTGG |
| | | GTTTCCCCATTCGGAAATCGCCG |
| 2319355 | 2319355 | CAAGGCCCGGGAACGTATTCACCGTGGCATTCTGATCCACG |
| | | ATTACTAGCGATTCCGACTTCATGGAGTCGAGTTGCAGACTC |
| | | CAATCCGGACTACGACGCACTTTATGAGGTCCGCTTGCTCTC |
| | | GCAGATCGGAAGAGCACACGTCTGA |
| 2319355 | 2319355 | CCTACACGACGCTCTTCCGATCTCAAGGCCCGGGAACGTAT |
| | | TCACCGTGGCATTCTGATCCACGATTACTAGCGATTCCGACT |
| | | TCATGGAGTCGAGTTGCAGACTCCAATCCGGACTACGACGC |
| | | ACTTTATGAGGTCCGCTTGCTCTCGC |
| 2319362 | 1503671 | CTGGAGTTCAGACGTGTGCTCTTCCGATCTCGGGAACGTATT |
| | | CACCGTGGCATTCTGATCCACGATTACTAGCGATTCCGACTT |
| | | CATGGAGTCGAGTTGCAGACTCCAATCCGGACTACGACGCA |
| | | CTTTATGAGGTCCGCTTGCTCTCGC |
| 2319503 | 2319540 | CGCCATTGTAGCACGTGTGTAGCCCTGGTCGTAAGGGCCAT |
| | | GATGACTTGACGTCATCCCCACCTTCCTCCAGTTTATCACTG |
| | | GCAGTCTCCTTTGAGTTCCCGGCCGGACCGCTGGCAACAAA |
| | | GGATAAGGGTTGCGCTCGTTGCGGGA |
| 2319540 | 2319503 | CCATGATGACTTGACGTCATCCCCACCTTCCTCCAGTTTATC |
| | | ACTGGCAGTCTCCTTTGAGTTCCCGGCCGGACCGCTGGCAA |
| | | CAAAGGATAAGGGTTGCGCTCGTTGCGGGACTTAACCCAAC |
| | | ATTTCACAACACGAGCTGACGACAGC |
| 2319633 | 2319644 | CGGGTTGCGCTCGTTGCGGGACTTAACCCAACATTTCACAAC |
| | | ACGAGCTGACGACAGCCATGCAGCACCTGTCTCACGGTTCC |
| | | CGAAGGCACATTCTCATCTCTGAAAACTTCCGTGGATGTCAA |
| | | GACCAGGTAAGGTTCTTCGCGTTGC |
| 2319644 | 2319633 | GTTGCGGGACTTAACCCAACATTTCACAACACGAGCTGACGA |
| | | CAGCCATGCAGCACCTGTGTCACGGTTCCCGAAGGCACATT |
| | | CTCATCTCTGAAAACTTCCGTGGATGTCAAGACCAGGTAAGG |
| | | TTCTTCGCGTTGCATCGAATTAAAC |
| 2319783 | 2319783 | TTCGAATTAAACCACATGCTCCACCGCTTGTGCGGGCCCCC |
| | | GTCAATTCATTTGAGTTTTAACCTTGCGGCCGTACTCCCCAG |
| | | GCGGTCAACTTAATGCGTTAGCTGCGCCACTAAAAGCTCAAG |
| | | GCTTCCAACAGATCGGAAGAGCACA |
| 2319783 | 2319783 | GACGCTCTTCCGATCTTTCGAATTAAACCACATGCTCCACCG |
| | | CTTGTGCGGGCCCCCGTCAATTCATTTGAGTTTTAACCTTGC |
| | | GGCCGTACTCCCCAGGCGGTCAACTTAATGCGTTAGCTGCG |
| | | GCACTAAAAGCTCAAGGCTTCCAAC |
| 2320091 | 2320130 | CTCAAGCtfGCCAGTATCAGATGCAGTTCCCAGGTTGAGCCC |
| | | GGGGATTTCACATCTGACTTAACAAACCGCCTGCGTGCGCTT |
| | | TACGCCCAGTAATTCCGATTAACGCTTGCACCCTCCGTATTA |
| | | CCGCGGCTGCTGGCACGGAGTTAG |
| 2320130 | 2320091 | CCCGGGGATTTCACATCTGACTTAACAAACCGCCTGCGTGC |
| | | GCTTTACGCGCAGTAATTCCGATTAACGCTTGCACCCTCCGT |
| | | ATTACCGCGGCTGCTGGCACGGAGTTAGCCGGTGCTTCTTC |
| | | TGCGGGTAACGTCAATGAGCAAAGGT |
| 2320361 | 2320375 | GGCTTGCGCCCATTGTGCAATATTCCCCACTGCTGCCTCCC |
| | | GTAGGAGTCTGGACCGTGTCTCAGTTCCAGTGTGGCTGGTC |
| | | ATCCTCTCAGACCAGCTAGGGATCGTCGCCTAGGTGAGCCA |
| | | TTACCCCACCTACTAGCTAATCCCATC |
| 2320375 | 2320361 | GTGCAATATTCCCCACTGCTGCCTCCCGTAGGAGTCTGGAC |
| | | CGTGTCTCAGTTCCAGTGTGGCTGGTCATCCTCTCAGACCAG |
| | | CTAGGGATCGTCGCCTAGGTGAGCCATTACCCCACCTACTA |
| | | GCTAATCCCATCTGGGCACATCCGAT |
| 2345096 | 2345096 | CGGGGAGATCAGAACAGTGAAGCGCTCTTTGCGTGTCGGCA |
| | | GCGGGATCGGACCACGGACCTGCGCACCAGTGCGCTTAGA |
| | | AGTCTCGACGATTTCCGCGGTTGCTTGATCGATCAGACGATG |
| | | ATCAAACGCTTTAGATCGGAAGAGCAC |
| 2345096 | 2345096 | ACGCTCTTCCGATCTCGGGGAGATCAGAACAGTGAAGCGCT |
| | | CTTTGCGTGTCGGCAGCGGGATCGGACCACGGACCTGCGC |
| | | ACCAGTGCGCTTAGCAGTCTCGACGATTTCCGCGGTTGCTT |
| | | GATCGATCAGACGATGATCAAACGCTTT |
| 2833276 | 2833282 | CCCAGATGGGATTAGCTAGTAGGTGGGGTAACGGCTCACCT |
| | | AGGCGACGATCCCTAGCTGGTCTGAGAGGATGACCAGCCAC |
| | | ACTGGAACTGAGACACGGTCCAGACTCCTACGGGAGGCAGC |
| | | AGTGGGGAATATTGCACAATGGGCGCA |
| 2833282 | 2833276 | TGGGATTAGCTAGTAGGTGGGGTAACGGCTCACCTAGGCGA |
| | | CGATCCCTAGCTGGTCTGAGAGGATGACCAGCCACACTGGA |
| | | ACTGAGACACGGTCCAGACTCCTACGGGAGGCAGCAGTGG |
| | | GGAATATTGCACAATGGGCGCAAGCCTG |
| 2833511 | 2833550 | ACCTTTGCTCATTGACGTTACCCGCAGAAGAAGCACCGGCTA |
| | | ACTCCGTGCCAGCAGCCGCGGTAATACGGAGGGTGCAAGC |
| | | GTTAATCGGAATTACTGGGCGTAAAGCGCACGCAGGCGGTT |
| | | TGTTAAGTCAGATGTGAAATCCCCGGG |
| 2833550 | 2833511 | CTAACTCCGTGCCAGCAGCCGCGGTAATACGGAGGGTGCAA |
| | | GCGTTAATCGGAATTACTGGGCGTAAAGCGCACGCAGGCGG |
| | | TTTGTTAAGTCAGATGTGAAATCCCCGGGCTCAACCTGGGAA |
| | | CTGCATCTGATACTGGCAAGCTTGAG |
| 2833900 | 2833900 | GTTGGAAGCCTTGAGCTTTTAGTGGCGCAGCTAACGCATTAA |
| | | GTTGACCGCCTGGGGAGTACGGCCGCAAGGTTAAAACTCAA |
| | | ATGAATTGACGGGGGCCCGCACAAGCGGTGGAGCATGTGGT |
| | | TTAATTCGAAAGATCGGAAGAGCGTC |
| 2833900 | 2833900 | TGTGCTCTTCCGATCTGTTGGAAGCCTTGAGCTTTTAGTGGC |
| | | GCAGCTAACGCATTAAGTTGACCGCCTGGGGAGTACGGCCG |
| | | CAAGGTTAAAACTCAAATGAATTGACGGGGGCCCGCACAAG |
| | | CGGTGGAGCATGTGGTTTAATTCGAA |
| 2833997 | 2834009 | GTTTAATTCGATGCAACGCGAAGAACCTTACCTGGTCTTGAC |
| | | ATCCACGGAAGTTTTCAGAGATGAGAATGTGCCTTCGGGAAC |
| | | CGTGAGACAGGTGCTGCATGGCTGTCGTCAGCTCGTGTTGT |
| | | GAAATGTTGGGTTAAGTCCCGCAAC |
| 2834009 | 2833997 | GCAACGCGAAGAACCTTACCTGGTCTTGACATCCACGGAAG |
| | | TTTTCAGAGATGAGAATGTGCCTTCGGGAACCGTGAGACAG |
| | | GTGCTGCATGGCTGTCGTCAGCTCGTGTTGTGAAATGTTGG |
| | | GTTAAGTCCCGCAACGAGCGCAACCCG |
| 2834049 | 2948544 | GTTTTCAGAGATGAGAATGTGCCTTCGGGAACCGTGAGACA |
| | | GGTGCTGCATGGCTGTCGTCAGCTCGTGTTGTGAAATGTTG |
| | | GGTTAAGTCCCGCAACGAGCGCAAGATCGGAAGAGCACACG |
| | | TCTGAACTCCAGTCACAACCTACGATC |
| 2834049 | 3077599 | GTTTTCAGAGATGAGAATGTGCCTTCGGGAACCGTGAGACA |
| | | GGTGCTGCATGGCTGTCGTCAGCTCGTGTTGTGAAATGTTG |
| | | GGTTAAGTCCCGCAACGAGCGCAAGATCGGAAGAGCACACG |
| | | TCTGAACTCCAGTCACAACCTACGATC |
| 2834049 | 3117867 | GTTTTCAGAGATGAGAATGTGCCTTCGGGAACCGTGAGACA |
| | | GGTGCTGCATGGCTGTCGTCAGCTCGTGTTGTGAAATGTTG |
| | | GGTTAAGTCCCGCAACGAGCGCAAGATCGGAAGAGCACACG |
| | | TCTGAACTCCAGTCACAACCTACGATC |
| 2834101 | 2834138 | GCTGTCGTCAGCTCGTGTTGTGAAATGTTGGGTTAAGTCCCG |
| | | CAACGAGCGCAACCCTTATCCTTTGTTGCCAGCGGTCCGGC |
| | | CGGGAACTCAAAGGAGACTGCCAGTGATAAACTGGAGGAAG |
| | | GTGGGGATGACGTCAAGTCATCATGG |
| 2834138 | 2834101 | TCCCGCAACGAGCGCAACCCTTATCCTTTGTTGCCAGCGGT |
| | | CCGGCCGGGAACTCAAAGGAGACTGCCAGTGATAAACTGGA |
| | | GGAAGGTGGGGATGACGTCAAGTCATCATGGCCCTTACGAC |
| | | CAGGGCTACACACGTGCTACAATGGCG |
| 2834151 | 2948646 | GCAACCCTTATCCTTTGTTGCCAGCGGTCCGGCCGGGAACT |
| | | CAAAGGAGAGTGCCAGTGATAAACTGGAGGAAGGTGGGGAT |
| | | GACGTCAAGTCATCATGGCCCTTACGACCAGGGCTACACAC |
| | | GTGCTACAATGGCGCATACAAAGAGAT |
| 2834151 | 3077701 | GCAACCCTTATCCTTTGTTGCCAGCGGTCCGGCCGGGAACT |
| | | CAAAGGAGACTGCCAGTGATAAACTGGAGGAAGGTGGGGAT |
| | | GACGTCAAGTCATCATGGCCCTTACGACCAGGGCTACACAC |
| | | GTGCTACAATGGCGCATACAAAGAGAT |
| 2834151 | 3117969 | GCAACCCTTATCCTTTGTTGCCAGCGGTCCGGCCGGGAACT |
| | | CAAAGGAGACTGCCAGTGATAAACTGGAGGAAGGTGGGGAT |
| | | GACGTCAAGTCATCATGGCCCTTACGACCAGGGCTACACAC |
| | | GTGCTACAATGGCGCATACAAAGAGAT |
| 2834309 | 2834309 | GCGAGAGCAAGCGGACCTCATAAAGTGCGTCGTAGTCCGGA |
| | | TTGGAGTCTGCAACTCGACTCCATGAAGTCGGAATCGCTAGT |
| | | AATCGTGGATCAGAATGCCACGGTGAATACGTTCCCGGGCC |
| | | TTGAGATCGGAAGAGCGTCGTGTAGG |
| 2834309 | 2834309 | TCAGACGTGTGCTCTTCCGATCTGCGAGAGCAAGCGGACCT |
| | | CATAAAGTGCGTCGTAGTCCGGATTGGAGTCTGCAACTCGA |
| | | CTCCATGAAGTCGGAATCGCTAGTAATCGTGGATCAGAATGC |
| | | CACGGTGAATACGTTCCCGGGCCTTG |
| 2835045 | 2835045 | CGGCGATTTCCGAATGGGGAAACCCAGTGTGTTTCGACACA |
| | | CTATCATTAACTGAATCCATAGGTTAATGAGGCGAACCGGGG |
| | | GAACTGAAACATCTAAGTACCCCGAGGAAAAGAAATCAACCG |
| | | AGATTCCAGATCGGAAGAGCGTCGT |
| 2835045 | 2835045 | CGTGTGCTCTTCCGATCTCGGCGATTTCCGAATGGGGAAAC |
| | | CCAGTGTGTTTCGACACACTATCATTAACTGAATCCATAGGTT |
| | | AATGAGGCGAACCGGGGGAACTGAAACATCTAAGTACCCCG |
| | | AGGAAAAGAAATCAACCGAGATTCC |
| 2835157 | 2835157 | AAGAAATCAACCGAGATTCCCCCAGTAGCGGCGAGCGAACG |
| | | GGGAGGAGCCCAGAGCCTGAATCAGTGTGTGTGTTAGTGGA |
| | | AGCGTCTGGAAAGGCGCGCGATACAGGGTGACAGCCCCGT |
| | | ACACAAAAAAGATCGGAAGAGCGTCGTG |
| 2835157 | 2835157 | ACGTGTGCTCTTCCGATCTAAGAAATCAACCGAGATTCCCCC |
| | | AGTAGCGGCGAGCGAACGGGGAGGAGCCCAGAGCCTGAAT |
| | | CAGTGTGTGTGTTAGTGGAAGCGTCTGGAAAGGCGCGCGAT |
| | | ACAGGGTGACAGCCCCGTACACAAAAA |
| 2835427 | 2835463 | GGCGAGGGGAGTGAAAAAGAACCTGAAACCGTGTACGTACA |
| | | AGCAGTGGGAGCCTCTTAATGGGGTGACTGCGTACCTTTTGT |
| | | ATAATGGGTCAGCGACTTATATTCTGTAGCAAGGTTAACCGA |
| | | ATAGGGGAGCCGAAGGGAAACCGAG |
| 2835463 | 2835427 | GTACAAGCAGTGGGAGCCTCTTAATGGGGTGACTGCGTACC |
| | | TTTTGTATAATGGGTCAGCGACTTATATTCTGTAGCAAGGTTA |
| | | ACCGAATAGGGGAGCCGAAGGGAAACCGAGTCTTAACTGGG |
| | | CGTTAAGTTGCAGGGTATAGACCCG |
| 2835763 | 2835784 | ATTTAGGTAGCGCCTCGTGAATTCATCTCCGGGGGTAGAGC |
| | | ACTGTTTCGGCAAGGGGGTCATCCCGACTTACCAACCCGAT |
| | | GCAAACTGCGAATACCGGAGAATGTTATCACGGGAGACACA |
| | | CGGCGGGTGCTAACGTCCGTCGTGAAG |
| 2835778 | 2950271 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACAGAT |
| 2835778 | 3079417 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACAGAT |
| 2835778 | 3119594 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACAGAT |
| 2835778 | 2950271 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACCCAG |
| 2835778 | 3079417 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACCCAG |
| 2835778 | 3119594 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACCCAG |
| 2835784 | 2835763 | TTCATCTCCGGGGGTAGAGCACTGTTTCGGCAAGGGGGTCA |
| | | TCCCGACTTACCAACCCGATGCAAACTGCGAATACCGGAGA |
| | | ATGTTATCACGGGAGACACACGGCGGGTGCTAACGTCCGTC |
| | | GTGAAGAGGGAAACAACCCAGACCGCC |
| 2836402 | 2836402 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATAGATCGGAAGAGCAC |
| 2836402 | 2836402 | ACGCTCTTCCGATCTGTTTAAGCGTGTAGGCTGGTTTTCCAG |
| | | GCAAATCCGGAAAATCAAGGCTGAGGCGTGATGACGAGGCA |
| | | CTACGGTGCTGAAGCGACAAATGCCCTGCTTCCAGGAAAAG |
| | | CCTCTAAGCATCAGGTAACATCAAAT |
| 2836402 | 2836430 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATCGTACCCCAAACCGA |
| 2836430 | 2836402 | CAAATCCGGAAAATCAAGGCTGAGGCGTGATGACGAGGCAC |
| | | TACGGTGCTGAAGCGACAAATGCCCTGCTTCCAGGAAAAGC |
| | | CTCTAAGCATCAGGTAACATCAAATCGTACCCCAAACCGACA |
| | | CAGGTGGTCAGGTAGAGAATACCAAG |
| 2837546 | 2837632 | GGTCCCTATCTGCCGTGGGCGCTGGAGAACTGAGGGGGGC |
| | | TGCTCCTAGTACGAGAGGACCGGAGTGGACGCATCACTGGT |
| | | GTTCGGGTTGTCATGCCAATGGCACTGCCCGGTAGCTAAAT |
| | | GCGGAAGAGATAAGTGCTGAAAGCATCT |
| 2837632 | 2837546 | GGTTGTCATGCCAATGGCACTGCCCGGTAGCTAAATGCGGA |
| | | AGAGATAAGTGCTGAAAGCATCTAAGCACGAAACTTGCCCCG |
| | | AGATGAGTTCTCCCTGACTCCTTGAGAGTCCTGAAGGAACGT |
| | | TGAAGACGACGACGTTGATAGGCCG |
| 2947561 | 2947567 | CTCAGATTGAACGCTGGCGGCAGGCCTAACACATGCAAGTC |
| | | GAACGGTAACAGGAAGCAGCTTGCTGCTTTGCTGACGAGTG |
| | | GCGGACGGGTGAGTAATGTCTGGGAAACTGCCTGATGGAGG |
| | | GGGATAACTACTGGAAACGGTAGCTAA |
| 2947567 | 2947561 | TTGAACGCTGGCGGCAGGCCTAACACATGCAAGTCGAACGG |
| | | TAACAGGAAGCAGCTTGCTGCTTTGCTGACGAGTGGCGGAC |
| | | GGGTGAGTAATGTCTGGGAAACTGCCTGATGGAGGGGGATA |
| | | ACTACTGGAAACGGTAGCTAATACCGC |
| 2947771 | 2947777 | CCCAGATGGGATTAGCTAGTAGGTGGGGTAACGGCTCACCT |
| | | AGGCGACGATCCCTAGCTGGTCTGAGAGGATGACCAGCCAC |
| | | ACTGGAACTGAGACACGGTCCAGACTCCTACGGGAGGCAGC |
| | | AGTGGGGAATATTGCACAATGGGCGCA |
| 2947777 | 2947771 | TGGGATTAGCTAGTAGGTGGGGTAACGGCTCACCTAGGCGA |
| | | CGATCCCTAGCTGGTCTGAGAGGATGACCAGCCACACTGGA |
| | | ACTGAGACACGGTCCAGACTCCTACGGGAGGCAGCAGTGG |
| | | GGAATATTGCACAATGGGCGCAAGCCTG |
| 2948006 | 2948045 | ACCTTTGCTCATTGACGTTACCCGCAGAAGAAGCACCGGCTA |
| | | ACTCCGTGCCAGCAGCCGCGGTAATACGGAGGGTGCAAGC |
| | | GTTAATCGGAATTACTGGGCGTAAAGCGCACGCAGGCGGTT |
| | | TGTTAAGTCAGATGTGAAATCCCCGGG |
| 2948045 | 2948006 | CTAACTCCGTGCCAGCAGCCGCGGTAATACGGAGGGTGCAA |
| | | GCGTTAATCGGAATTACTGGGCGTAAAGCGCACGCAGGCGG |
| | | TTTGTTAAGTCAGATGTGAAATCCCCGGGCTCAACCTGGGAA |
| | | CTGCATCTGATACTGGCAAGCTTGAG |
| 2948395 | 2948395 | GTTGGAAGCCTTGAGCTTTTAGTGGCGCAGCTAACGCATTAA |
| | | GTTGACCGCCTGGGGAGTACGGCCGCAAGGTTAAAACTCAA |
| | | ATGAATTGACGGGGGCCCGCACAAGCGGTGGAGCATGTGGT |
| | | TTAATTCGAAAGATCGGAAGAGCGTC |
| 2948395 | 2948395 | TGTGCTCTTCCGATCTGTTGGAAGCCTTGAGCTTTTAGTGGC |
| | | GCAGCTAACGCATTAAGTTGACCGCCTGGGGAGTACGGCCG |
| | | CAAGGTTAAAACTCAAATGAATTGACGGGGGCCCGCACAAG |
| | | CGGTGGAGCATGTGGTTTAATTCGAA |
| 2948482 | 2948504 | GTTTAATTCGATGCAACGCGAAGAACCTTACCTGGTCTTGAC |
| | | ATCCACGGAAGTTTTCAGAGATGAGAATGTGCCTTCGGGAAC |
| | | CGTGAGACAGGTGCTGCATGGCTGTCGTCAGCTCGTGTTGT |
| | | GAAATGTTGGGTTAAGTCCCGCAAC |
| 2948504 | 2948492 | GCAACGCGAAGAACCTTACCTGGTCTTGAGATCCACGGAAG |
| | | TTTTCAGAGATGAGAATGTGCCTTCGGGAACCGTGAGACAG |
| | | GTGCTGCATGGCTGTCGTCAGCTCGTGTTGTGAAATGTTGG |
| | | GTTAAGTCCCGCAACGAGCGCAACCCG |
| 2948544 | 2834049 | ACACGCGTAAGAACACTCTTTCCCTACACGACGCTCTTCCGA |
| | | TCTGTTTTCAGAGATGAGAATGTGCCTTCGGGAACCGTGAGA |
| | | CAGGTGCTGCATGGCTGTCGTCAGCTCGTGTTGTGAAATGTT |
| | | GGGTTAAGTCCCGCAACGAGCGCA |
| 2948544 | 3077599 | GTTTTCAGAGATGAGAATGTGCCTTCGGGAACCGTGAGACA |
| | | GGTGCTGCATGGCTGTCGTCAGCTCGTGTTGTGAAATGTTG |
| | | GGTTAAGTCCCGCAACGAGCGCAAGATCGGAAGAGCACACG |
| | | TCTGAACTCCAGTCACAACCTACGATC |
| 2948544 | 3117867 | GTTTTCAGAGATGAGAATGTGCCTTCGGGAACCGTGAGACA |
| | | GGTGCTGCATGGCTGTCGTCAGCTCGTGTTGTGAAATGTTG |
| | | GGTTAAGTCCCGCAACGAGCGCAAGATCGGAAGAGCACACG |
| | | TCTGAACTCCAGTCACAACCTACGATC |
| 2948596 | 2948633 | GCTGTCGTCAGCTCGTGTTGTGAAATGTTGGGTTAAGTCCCG |
| | | CAACGAGCGCAACCCTTATCCTTTGTTGCCAGCGGTCCGGC |
| | | CGGGAACTCAAAGGAGACTGCCAGTGATAAACTGGAGGAAG |
| | | GTGGGGATGACGTCAAGTCATCATGG |
| 2948633 | 2948596 | TCCCGCAACGAGCGCAACCCTTATCCTTTGTTGCCAGCGGT |
| | | CCGGCCGGGAACTCAAAGGAGACTGCCAGTGATAAACTGGA |
| | | GGAAGGTGGGGATGACGTCAAGTCATCATGGCCCTTACGAC |
| | | CAGGGCTACACACGTGCTACAATGGCG |
| 2948646 | 2834151 | ATCTGCAACCCTTATCCTTTGTTGCCAGCGGTCCGGCCGGG |
| | | AACTCAAAGGAGACTGCCAGTGATAAACTGGAGGAAGGTGG |
| | | GGATGACGTCAAGTCATCATGGCCCTTACGACCAGGGCTAC |
| | | ACACGTGCTACAATGGCGCATACAAAG |
| 2948646 | 3077701 | GCAACCCTTATCCTTTGTTGCCAGCGGTCCGGCCGGGAACT |
| | | CAAAGGAGACTGCCAGTGATAAACTGGAGGAAGGTGGGGAT |
| | | GACGTCAAGTCATCATGGCCCTTACGACCAGGGCTACACAC |
| | | GTGCTACAATGGCGCATACAAAGAGAT |
| 2948646 | 3117969 | GCAACCGTTATCCTTTGTTGCCAGCGGTCCGGCCGGGAACT |
| | | CAAAGGAGACTGCCAGTGATAAACTGGAGGAAGGTGGGGAT |
| | | GACGTCAAGTCATCATGGCCCTTACGACCAGGGCTACACAC |
| | | GTGCTACAATGGCGCATACAAAGAGAT |
| 2948804 | 2948804 | GCGAGAGCAAGCGGACCTCATAAAGTGCGTCGTAGTCCGGA |
| | | TTGGAGTCTGCAACTCGACTCCATGAAGTCGGAATCGCTAGT |
| | | AATCGTGGATCAGAATGCCACGGTGAATACGTTCCCGGGCC |
| | | TTGAGATCGGAAGAGCGTCGTGTAGG |
| 2948804 | 2948804 | TCAGACGTGTGCTCTTCCGATCTGCGAGAGCAAGCGGACCT |
| | | CATAAAGTGCGTCGTAGTCCGGATTGGAGTCTGCAACTCGA |
| | | CTCCATGAAGTCGGAATCGCTAGTAATCGTGGATCAGAATGC |
| | | CACGGTGAATACGTTCCCGGGCCTTG |
| 2949540 | 2949540 | CGGCGATTTCCGAATGGGGAAACCCAGTGTGTTTCGACACA |
| | | CTATCATTAACTGAATCCATAGGTTAATGAGGCGAACCGGGG |
| | | GAACTGAAACATCTAAGTACCCCGAGGAAAAGAAATCAACCG |
| | | AGATTCCAGATCGGAAGAGCGTCGT |
| 2949540 | 2949540 | CGTGTGCTCTTCCGATCTCGGCGATTTCCGAATGGGGAAAC |
| | | CCAGTGTGTTTCGACACACTATCATTAACTGAATCCATAGGTT |
| | | AATGAGGCGAACCGGGGGAACTGAAACATCTAAGTACCCCG |
| | | AGGAAAAGAAATCAACCGAGATTCC |
| 2949652 | 2949652 | AAGAAATCAACCGAGATTCCCCCAGTAGCGGCGAGCGAACG |
| | | GGGAGGAGCCCAGAGCCTGAATCAGTGTGTGTGTTAGTGGA |
| | | AGCGTCTGGAAAGGCGCGCGATACAGGGTGACAGCCCCGT |
| | | ACACAAAAAAGATCGGAAGAGCGTCGTG |
| 2949652 | 2949652 | ACGTGTGCTCTTCCGATCTAAGAAATCAACCGAGATTCCCCC |
| | | AGTAGCGGCGAGCGAACGGGGAGGAGCCCAGAGCCTGAAT |
| | | CAGTGTGTGTGTTAGTGGAAGCGTCTGGAAAGGCGCGCGAT |
| | | ACAGGGTGACAGCCCCGTACACAAAAA |
| 2949922 | 2949958 | GGCGAGGGGAGTGAAAAAGAACCTGAAACCGTGTACGTACA |
| | | AGCAGTGGGAGCCTCTTAATGGGGTGACTGCGTACCTTTTGT |
| | | ATAATGGGTCAGCGACTTATATTCTGTAGCAAGGTTAACCGA |
| | | ATAGGGGAGCCGAAGGGAAACCGAG |
| 2949958 | 2949922 | GTACAAGCAGTGGGAGCCTCTTAATGGGGTGACTGCGTACC |
| | | TTTTGTATAATGGGTCAGCGACTTATATTCTGTAGCAAGGTTA |
| | | ACCGAATAGGGGAGCCGAAGGGAAACCGAGTCTTAACTGGG |
| | | CGTTAAGTTGCAGGGTATAGACCCG |
| 2950258 | 2950279 | ATTTAGGTAGCGCCTCGTGAATTCATCTCCGGGGGTAGAGC |
| | | ACTGTTTCGGCAAGGGGGTCATCCCGACTTACCAACCCGAT |
| | | GCAAACTGCGAATACCGGAGAATGTTATCACGGGAGACACA |
| | | CGGCGGGTGCTAACGTCCGTCGTGAAG |
| 2950271 | 2835778 | ATCTCGTGAATTCATCTCCGGGGGTAGAGCACTGTTTCGGCA |
| | | AGGGGGTCATCCCGACTTACCAACCCGATGCAAACTGCGAA |
| | | TACCGGAGAATGTTATCACGGGAGACACACGGCGGGTGCT |
| | | ACGTCCGTCGTGAAGAGGGAAACAAC |
| 2950271 | 2835778 | CTCGTGAATTCATCTCCGGGGGTAGAGCACTGTTTCGGCAA |
| | | GGGGGTCATCCCGACTTACCAACCCGATGCAAACTGCGAAT |
| | | ACCGGAGAATGTTATCACGGGAGACACACGGCGGGTGCTAA |
| | | CGTCCGTCGTGAAGAGGGAAACAACCC |
| 2950273 | 3079417 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACAGAT |
| 2950273 | 3119594 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACAGAT |
| 2950273 | 3079417 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACCCAG |
| 2950273 | 3119594 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACCCAG |
| 2950279 | 2950258 | TTCATCTCCGGGGGTAGAGCACTGTTTCGGCAAGGGGGTCA |
| | | TCCCGACTTACCAACCCGATGCAAACTGCGAATACCGGAGA |
| | | ATGTTATCACGGGAGACACACGGCGGGTGCTAACGTCCGTC |
| | | GTGAAGAGGGAAACAACCCAGACCGCC |
| 2950897 | 2950897 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATAGATCGGAAGAGCAC |
| 2950897 | 2950897 | ACGCTCTTCCGATCTGTTTAAGCGTGTAGGCTGGTTTTCCAG |
| | | GCAAATCCGGAAAATCAAGGCTGAGGCGTGATGACGAGGCA |
| | | CTACGGTGCTGAAGCGACAAATGCCCTGCTTCCAGGAAAAG |
| | | CCTCTAAGCATCAGGTAACATCAAAT |
| 2950897 | 2950925 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATCGTACCCCAAACCGA |
| 2950925 | 2950897 | CAAATCCGGAAAATCAAGGCTGAGGCGTGATGACGAGGCAC |
| | | TACGGTGCTGAAGCGACAAATGCCCTGCTTCCAGGAAAAGC |
| | | CTCTAAGCATCAGGTAACATCAAATCGTACCCCAAACCGACA |
| | | CAGGTGGTCAGGTAGAGAATACCAAG |
| 2952041 | 2952127 | GGTCCCTATCTGCCGTGGGCGCTGGAGAACTGAGGGGGGC |
| | | TGCTCCTAGTACGAGAGGACCGGAGTGGACGCATCACTGGT |
| | | GTTCGGGTTGTCATGCCAATGGCACTGCCCGGTAGCTAAAT |
| | | GCGGAAGAGATAAGTGCTGAAAGCATCT |
| 2952127 | 2952041 | GGTTGTCATGCCAATGGCACTGCCCGGTAGCTAAATGCGGA |
| | | AGAGATAAGTGCTGAAAGCATCTAAGCACGAAACTTGCCCCG |
| | | AGATGAGTTCTCCCTGACTCCTTGAGAGTCCTGAAGGAACGT |
| | | TGAAGACGACGACGTTGATAGGCCG |
| 3028158 | 3028470 | AGATTTCGTTGCTTTTTCCGTGAGGTGCTCTTTTTTCGCCGC |
| | | GAAGGTGCCGGTTGGCTGCGGCGTACATAATCTCGTTGTGC |
| | | CACTATCGTTTCGCTGTATTTATTCGTTCGTCAGCCCGCCAT |
| | | GTTACTTAAGCGGCGGGCCTTTGAC |
| 3028158 | 3028470 | AGATTTCGTTGCTTTTTCCGTGAGGTGCTCTTTTTTCGCCGC |
| | | GAAGGTGCCGGTTGGCTGCGGCGTACATAATCTCGTTGTGC |
| | | CACTATCGTTTCGCTGTATTTATTCGTTCGTCAGCCCGCCAT |
| | | GTTACTTAAGCGGCGGGCCTTTGAC |
| 3028470 | 3028158 | CGCGATGGTTGTCAGCGGCGGATCACAAAATTGCGTCAGGT |
| | | CGATGTTATCAAAACCGATTATGGAAAGGTCTTCCGGGACTT |
| | | TCAGCCCCTGGCGTTTTGCCTGAGAAAGTGCGCCGAGCGCC |
| | | ATCACATCGCTATGGCAGAAGACAGC |
| 3028470 | 3028158 | CGCGATGGTTGTCAGCGGCGGATCACAAAATTGCGTCAGGT |
| | | CGATGTTATCAAAACCGATTATGGAAAGGTCTTCCGGGACTT |
| | | TCAGCCCCTGGCGTTTTGCCTGAGAAAGTGCGCCGAGCGCC |
| | | ATCACATCGCTATGGCAGAAGACAGC |
| 3076616 | 3076622 | CTCAGATTGAACGCTGGCGGCAGGCCTAACACATGCAAGTC |
| | | GAACGGTAACAGGAAGCAGCTTGCTGCTTTGCTGACGAGTG |
| | | GCGGACGGGTGAGTAATGTCTGGGAAACTGCCTGATGGAGG |
| | | GGGATAACTACTGGAAACGGTAGCTAA |
| 3076622 | 3076616 | TTGAACGCTGGCGGCAGGCCTAACACATGCAAGTCGAACGG |
| | | TAACAGGAAGCAGCTTGCTGCTTTGCTGACGAGTGGCGGAC |
| | | GGGTGAGTAATGTCTGGGAAACTGCCTGATGGAGGGGGATA |
| | | ACTACTGGAAACGGTAGCTAATACCGC |
| 3076826 | 3076832 | CCCAGATGGGATTAGCTAGTAGGTGGGGTAACGGCTCACCT |
| | | AGGCGACGATCCCTAGCTGGTCTGAGAGGATGACCAGCCAC |
| | | ACTGGAACTGAGACACGGTCCAGACTCCTACGGGAGGCAGC |
| | | AGTGGGGAATATTGCACAATGGGCGCA |
| 3076832 | 3076826 | TGGGATTAGCTAGTAGGTGGGGTAACGGCTCACCTAGGCGA |
| | | CGATCCCTAGCTGGTCTGAGAGGATGACCAGCCACACTGGA |
| | | ACTGAGACACGGTCCAGACTCCTACGGGAGGCAGCAGTGG |
| | | GGAATATTGCACAATGGGCGCAAGCCTG |
| 3077061 | 3077100 | ACCTTTGCTCATTGACGTTACCCGCAGAAGAAGCAGCGGCTA |
| | | ACTCCGTGCCAGCAGCCGCGGTAATACGGAGGGTGCAAGC |
| | | GTTAATCGGAATTAGTGGGCGTAAAGCGCACGCAGGCGGTT |
| | | TGTTAAGTCAGATGTGAAATCCCCGGG |
| 3077100 | 3077061 | CTAACTCCGTGCCAGCAGCCGCGGTAATACGGAGGGTGCAA |
| | | GCGTTAATCGGAATTACTGGGCGTAAAGCGCACGCAGGCGG |
| | | TTTGTTAAGTCAGATGTGAAATCCCCGGGCTCAACCTGGGAA |
| | | CTGCATCTGATACTGGCAAGCTTGAG |
| 3077547 | 3077559 | GTTTAATTCGATGCAACGCGAAGAACCTTACCTGGTCTTGAC |
| | | ATCCACGGAAGTTTTCAGAGATGAGAATGTGCCTTCGGGAAC |
| | | CGTGAGACAGGTGCTGCATGGCTGTCGTCAGCTCGTGTTGT |
| | | GAAATGTTGGGTTAAGTCCCGCAAC |
| 3077559 | 3077547 | GCAACGCGAAGAACCTTACCTGGTCTTGACATCCACGGAAG |
| | | TTTTCAGAGATGAGAATGTGCCTTCGGGAACCGTGAGACAG |
| | | GTGCTGCATGGCTGTCGTCAGCTCGTGTTGTGAAATGTTGG |
| | | GTTAAGTCCCGCAACGAGCGCAACCCG |
| 3077599 | 3117867 | GTTTTCAGAGATGAGAATGTGCCTTCGGGAACCGTGAGACA |
| | | GGTGCTGCATGGCTGTCGTCAGCTCGTGTTGTGAAATGTTG |
| | | GGTTAAGTCCCGCAACGAGCGCAAGATCGGAAGAGCACACG |
| | | TCTGAACTCCAGTCACAACCTACGATC |
| 3077599 | 2948544 | ACACGCGTAAGAACACTCTTTCCCTACACGACGCTCTTCCGA |
| | | TCTGTTTTCAGAGATGAGAATGTGCCTTCGGGAACCGTGAGA |
| | | CAGGTGCTGCATGGCTGTCGTCAGCTCGTGTTGTGAAATGTT |
| | | GGGTTAAGTCCCGCAACGAGCGCA |
| 3077599 | 2834049 | ACACGCGTAAGAACACTCTTTCCCTACACGACGCTCTTCCGA |
| | | TCTGTTTTCAGAGATGAGAATGTGCCTTCGGGAACCGTGAGA |
| | | CAGGTGCTGCATGGCTGTCGTCAGCTCGTGTTGTGAAATGTT |
| | | GGGTTAAGTCCCGCAACGAGCGCA |
| 3077651 | 3077688 | GCTGTCGTCAGCTCGTGTTGTGAAATGTTGGGTTAAGTCCCG |
| | | CAACGAGCGCAACCCTTATCCTTTGTTGCCAGCGGTCCGGC |
| | | CGGGAACTCAAAGGAGACTGCCAGTGATAAACTGGAGGAAG |
| | | GTGGGGATGACGTCAAGTCATCATGG |
| 3077688 | 3077651 | TCCCGCAACGAGCGCAACCCTTATCCTTTGTTGCCAGCGGT |
| | | CCGGCCGGGAACTCAAAGGAGACTGCCAGTGATAAACTGGA |
| | | GGAAGGTGGGGATGACGTCAAGTCATCATGGCCCTTACGAC |
| | | CAGGGCTACACACGTGCTACAATGGCG |
| 3077701 | 3117969 | GCAACCCTTATCCTTTGTTGCCAGCGGTCCGGCCGGGAACT |
| | | CAAAGGAGACTGCCAGTGATAAACTGGAGGAAGGTGGGGAT |
| | | GACGTCAAGTCATCATGGCCCTTACGACCAGGGCTACACAC |
| | | GTGCTACAATGGCGCATACAAAGAGAT |
| 3077701 | 2948646 | ATCTGCAACCCTTATCCTTTGTTGCCAGCGGTCCGGCCGGG |
| | | AACTCAAAGGAGACTGCCAGTGATAAACTGGAGGAAGGTGG |
| | | GGATGACGTCAAGTCATCATGGCCCTTACGACCAGGGCTAC |
| | | ACACGTGCTACAATGGCGCATACAAAG |
| 3077701 | 2834151 | ATCTGCAACCCTTATCCTTTGTTGCCAGCGGTCCGGCCGGG |
| | | AACTCAAAGGAGACTGCCAGTGATAAACTGGAGGAAGGTGG |
| | | GGATGACGTCAAGTCATCATGGCCCTTACGACCAGGGCTAC |
| | | ACACGTGCTACAA.TGGCGCATACAAAG |
| 3077859 | 3077859 | GCGAGAGCAAGCGGACCTCATAAAGTGCGTCGTAGTCCGGA |
| | | TTGGAGTCTGCAACTCGACTCCATGAAGTCGGAATCGCTAGT |
| | | AATCGTGGATCAGAATGCCACGGTGAATACGTTCCCGGGCC |
| | | TTGAGATCGGAAGAGCGTCGTGTAGG |
| 3077859 | 3077859 | TCAGACGTGTGCTCTTCCGATCTGCGAGAGCAAGCGGACCT |
| | | CATAAAGTGCGTCGTAGTCCGGATTGGAGTCTGCAACTCGA |
| | | CTCCATGAAGTCGGAATCGCTAGTAATCGTGGATCAGAATGC |
| | | CACGGTGAATACGTTCCCGGGCCTTG |
| 3078686 | 3078686 | CGGCGATTTCCGAATGGGGAAACCCAGTGTGTTTCGACACA |
| | | CTATCATTAACTGAATCCATAGGTTAATGAGGCGAACCGGGG |
| | | GAACTGAAACATCTAAGTACCCCGAGGAAAAGAAATCAACCG |
| | | AGATTCCAGATCGGAAGAGCGTCGT |
| 3078686 | 3078686 | CGTGTGCTCTTCCGATCTCGGCGATTTCCGAATGGGGAAAC |
| | | CCAGTGTGTTTCGACACACTATCATTAACTGAATCCATAGGTT |
| | | AATGAGGCGAACCGGGGGAACTGAAACATCTAAGTACCCCG |
| | | AGGAAAAGAAATCAACCGAGATTCC |
| 3078798 | 3078798 | AAGAAATCAACCGAGATTCCCCCAGTAGCGGCGAGCGAACG |
| | | GGGAGGAGCCCAGAGCCTGAATCAGTGTGTGTGTTAGTGGA |
| | | AGCGTCTGGAAAGGCGCGCGATACAGGGTGACAGCCCCGT |
| | | ACACAAAAAAGATCGGAAGAGCGTCGTG |
| 3078798 | 3078798 | ACGTGTGCTCTTCCGATCTAAGAAATCAACCGAGATTCCCCC |
| | | AGTAGCGGCGAGCGAACGGGGAGGAGCCCAGAGCCTGAAT |
| | | CAGTGTGTGTGTTAGTGGAAGCGTCTGGAAAGGCGCGCGAT |
| | | ACAGGGTGACAGCCCCGTACACAAAAA |
| 3079068 | 3079104 | GGCGAGGGGAGTGAAAAAGAACCTGAAACCGTGTACGTACA |
| | | AGCAGTGGGAGCCTCTTAATGGGGTGACTGCGTACCTTTTGT |
| | | ATAATGGGTCAGCGACTTATATTCTGTAGCAAGGTTAACCGA |
| | | ATAGGGGAGCCGAAGGGAAACCGAG |
| 3079104 | 3079068 | GTACAAGCAGTGGGAGCCTCTTAATGGGGTGACTGCGTACC |
| | | TTTTGTATAATGGGTCAGCGACTTATATTCTGTAGCAAGGTTA |
| | | ACCGAATAGGGGAGCCGAAGGGAAACCGAGTCTTAACTGGG |
| | | CGTTAAGTTGCAGGGTATAGACCCG |
| 3079404 | 3079425 | ATTTAGGTAGCGCCTCGTGAATTCATCTCCGGGGGTAGAGC |
| | | ACTGTTTCGGCAAGGGGGTCATCCCGACTTACCAACCCGAT |
| | | GCAAACTGCGAATACCGGAGAATGTTATCACGGGAGACACA |
| | | CGGCGGGTGCTAACGTCCGTCGTGAAG |
| 3079417 | 2950273 | ATCTCGTGAATTCATCTCCGGGGGTAGAGCACTGTTTCGGCA |
| | | AGGGGGTCATCCCGACTTACCAACCCGATGCAAACTGCGAA |
| | | TACCGGAGAATGTTATCACGGGAGACACACGGCGGGTGCTA |
| | | ACGTCCGTCGTGAAGAGGGAAACAAC |
| 3079417 | 2835778 | ATCTCGTGAATTCATCTCCGGGGGTAGAGCACTGTTTGGGCA |
| | | AGGGGGTCATCCCGACTTACCAACCCGATGCAAACTGCGAA |
| | | TACCGGAGAATGTTATCACGGGAGACACACGGCGGGTGCTA |
| | | ACGTCCGTCGTGAAGAGGGAAACAAC |
| 3079417 | 2950273 | CTCGTGAATTCATCTCCGGGGGTAGAGCACTGTTTCGGCAA |
| | | GGGGGTCATCCCGACTTACCAACCCGATGCAAACTGCGAAT |
| | | ACCGGAGAATGTTATCACGGGAGACACACGGCGGGTGCTAA |
| | | CGTCCGTCGTGAAGAGGGAAACAACCC |
| 3079417 | 2835778 | CTCGTGAATTCATCTCCGGGGGTAGAGCACTGTTTCGGCAA |
| | | GGGGGTCATCCCGACTTACCAACCCGATGCAAACTGCGAAT |
| | | ACCGGAGAATGTTATCACGGGAGACACACGGCGGGTGCTAA |
| | | CGTCCGTCGTGAAGAGGGAAACAACCC |
| 3079419 | 3119594 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACAGAT |
| 3079419 | 3119594 | CGTGAATTCATCTCCGGGGGTAGAGCACTGTTTCGGCAAGG |
| | | GGGTCATCCCGACTTACCAACCCGATGCAAACTGCGAATAC |
| | | CGGAGAATGTTATCACGGGAGACACACGGCGGGTGCTAACG |
| | | TCCGTCGTGAAGAGGGAAACAACCCAG |
| 3079425 | 3079404 | TTCATCTCCGGGGGTAGAGCACTGTTTCGGCAAGGGGGTCA |
| | | TCCCGACTTACCAACCCGATGCAAACTGCGAATACCGGAGA |
| | | ATGTTATCACGGGAGACACACGGCGGGTGCTAACGTCCGTC |
| | | GTGAAGAGGGAAACAACCCAGACCGCC |
| 3080043 | 3080043 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATAGATCGGAAGAGCAC |
| 3080043 | 3080043 | ACGCTCTTCCGATCTGTTTAAGCGTGTAGGCTGGTTTTCCAG |
| | | GCAAATCCGGAAAATCAAGGCTGAGGCGTGATGACGAGGCA |
| | | CTACGGTGCTGAAGCGACAAATGCCCTGCTTCCAGGAAAAG |
| | | CCTCTAAGCATCAGGTAACATCAAAT |
| 3080043 | 3080071 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATCGTACCCCAAACCGA |
| 3080071 | 3080043 | CAAATCCGGAAAATCAAGGCTGAGGCGTGATGACGAGGCAC |
| | | TACGGTGCTGAAGCGACAAATGCCCTGCTTCCAGGAAAAGC |
| | | CTCTAAGCATCAGGTAACATCAAATCGTACCCCAAACCGACA |
| | | CAGGTGGTCAGGTAGAGAATACCAAG |
| 3081187 | 3081273 | GGTCCCTATCTGCCGTGGGCGCTGGAGAACTGAGGGGGGC |
| | | TGCTCCTAGTACGAGAGGACCGGAGTGGACGCATCACTGGT |
| | | GTTCGGGTTGTCATGCCAATGGCACTGCCCGGTAGCTAAAT |
| | | GCGGAAGAGATAAGTGCTGAAAGCATCT |
| 3081273 | 3081187 | GGTTGTCATGCCAATGGCACTGCCCGGTAGCTAAATGCGGA |
| | | AGAGATAAGTGCTGAAAGCATCTAAGCACGAAACTTGCCCCG |
| | | AGATGAGTTCTCCCTGACTCCTTGAGAGTCCTGAAGGAACGT |
| | | TGAAGACGACGACGTTGATAGGCCG |
| 3116884 | 3116890 | CTCAGATTGAACGCTGGCGGCAGGCCTAACACATGCAAGTC |
| | | GAACGGTAACAGGAAGCAGCTTGCTGCTTTGCTGACGAGTG |
| | | GCGGACGGGTGAGTAATGTCTGGGAAACTGCCTGATGGAGG |
| | | GGGATAACTACTGGAAACGGTAGCTAA |
| 3116890 | 3116884 | TTGAACGCTGGCGGCAGGCCTAACACATGCAAGTCGAACGG |
| | | TAACAGGAAGCAGCTTGCTGCTTTGCTGACGAGTGGCGGAC |
| | | GGGTGAGTAATGTCTGGGAAACTGCCTGATGGAGGGGGATA |
| | | ACTACTGGAAACGGTAGCTAATACCGC |
| 3117094 | 3117100 | CCCAGATGGGATTAGCTAGTAGGTGGGGTAACGGCTCACCT |
| | | AGGCGACGATCCCTAGCTGGTCTGAGAGGATGACCAGCCAC |
| | | ACTGGAACTGAGACACGGTCCAGACTCCTACGGGAGGCAGC |
| | | AGTGGGGAATATTGCACAATGGGCGCA |
| 3117100 | 3117094 | TGGGATTAGCTAGTAGGTGGGGTAACGGCTCACCTAGGCGA |
| | | CGATCCCTAGCTGGTCTGAGAGGATGACCAGCCACACTGGA |
| | | ACTGAGACACGGTCCAGACTCCTACGGGAGGCAGCAGTGG |
| | | GGAATATTGCACAATGGGCGCAAGCCTG |
| 3117329 | 3117368 | ACCTTTGCTCATTGACGTTACCCGCAGAAGAAGCACCGGCTA |
| | | ACTCCGTGCCAGCAGCCGCGGTAATACGGAGGGTGCAAGC |
| | | GTTAATCGGAATTACTGGGCGTAAAGCGCACGCAGGCGGTT |
| | | TGTTAAGTCAGATGTGAAATCCCCGGG |
| 3117368 | 3117329 | CTAACTCCGTGCCAGCAGCCGCGGTAATACGGAGGGTGCAA |
| | | GCGTTAATCGGAATTACTGGGCGTAAAGCGCACGCAGGCGG |
| | | TTTGTTAAGTCAGATGTGAAATCCCCGGGCTCAACCTGGGAA |
| | | CTGCATCTGATACTGGCAAGCTTGAG |
| 3117718 | 3117718 | GTTGGAAGCCTTGAGCTTTTAGTGGCGCAGCTAACGCATTAA |
| | | GTTGACCGCCTGGGGAGTACGGCCGCAAGGTTAAAACTCAA |
| | | ATGAATTGACGGGGGCCCGCACAAGCGGTGGAGCATGTGGT |
| | | TTAATTCGAAAGATCGGAAGAGCGTC |
| 3117718 | 3117718 | TGTGCTCTTCCGATCTGTTGGAAGCCTTGAGCTTTTAGTGGC |
| | | GCAGCTAACGCATTAAGTTGACCGCCTGGGGAGTACGGCCG |
| | | CAAGGTTAAAACTCAAATGAATTGACGGGGGCCCGCACAAG |
| | | CGGTGGAGCATGTGGTTTAATTCGAA |
| 3117815 | 3117827 | GTTTAATTCGATGCAACGCGAAGAACCTTACCTGGTCTTGAC |
| | | ATCCACGGAAGTTTTCAGAGATGAGAATGTGCCTTCGGGAAC |
| | | CGTGAGACAGGTGCTGCATGGCTGTCGTCAGCTCGTGTTGT |
| | | GAAATGTTGGGTTAAGTCCCGCAAC |
| 3117827 | 3117815 | GCAACGCGAAGAACCTTACCTGGTCTTGACATCCACGGAAG |
| | | TTTTCAGAGATGAGAATGTGCCTTCGGGAACCGTGAGACAG |
| | | GTGCTGCATGGCTGTCGTCAGCTCGTGTTGTGAAATGTTGG |
| | | GTTAAGTCCCGCAACGAGCGCAACCCG |
| 3117867 | 3077599 | ACACGCGTAAGAACACTCTTTCCCTAGACGACGCTCTTCCGA |
| | | TCTGTTTTCAGAGATGAGAATGTGCCTTCGGGAACCGTGAGA |
| | | CAGGTGCTGCATGGCTGTCGTCAGCTCGTGTTGTGAAATGTT |
| | | GGGTTAAGTCCCGCAACGAGCGCA |
| 3117867 | 2948544 | ACACGCGTAAGAACACTCTTTCCCTACACGACGCTCTTCCGA |
| | | TCTGTTTTCAGAGATGAGAATGTGCCTTCGGGAACCGTGAGA |
| | | CAGGTGCTGCATGGCTGTCGTCAGCTCGTGTTGTGAAATGTT |
| | | GGGTTAAGTCCCGCAACGAGCGCA |
| 3117867 | 2834049 | ACACGCGTAAGAACACTCTTTCCCTACACGACGCTCTTCCGA |
| | | TCTGTTTTCAGAGATGAGAATGTGCCTTCGGGAACCGTGAGA |
| | | CAGGTGCTGCATGGCTGTCGTCAGCTCGTGTTGTGAAATGTT |
| | | GGGTTAAGTCCCGCAACGAGCGCA |
| 3117919 | 3117956 | GCTGTCGTCAGCTCGTGTTGTGAAATGTTGGGTTAAGTCCCG |
| | | CAACGAGCGCAACCCTTATCCTTTGTTGCCAGCGGTCCGGC |
| | | CGGGAACTCAAAGGAGACTGCCAGTGATAAACTGGAGGAAG |
| | | GTGGGGATGACGTCAAGTCATCATGG |
| 3117956 | 3117919 | TCCCGCAACGAGCGCAACCCTTATCCTTTGTTGCCAGCGGT |
| | | CCGGCCGGGAACTCAAAGGAGACTGCCAGTGATAAACTGGA |
| | | GGAAGGTGGGGATGACGTCAAGTCATCATGGCCCTTACGAC |
| | | CAGGGCTACACACGTGCTACAATGGCG |
| 3117969 | 3077701 | ATCTGCAACCCTTATCCTTTGTTGCCAGCGGTCCGGCCGGG |
| | | AACTCAAAGGAGACTGCCAGTGATAAACTGGAGGAAGGTGG |
| | | GGATGACGTCAAGTCATCATGGCCCTTACGACCAGGGCTAC |
| | | ACACGTGCTACAATGGCGCATACAAAG |
| 3117969 | 2948646 | ATCTGCAACCCTTATCCTTTGTTGCCAGCGGTCCGGCCGGG |
| | | AACTCAAAGGAGACTGCCAGTGATAAACTGGAGGAAGGTGG |
| | | GGATGACGTCAAGTCATCATGGCCCTTACGACCAGGGCTAC |
| | | ACACGTGCTACAATGGCGCATACAAAG |
| 3117969 | 2834151 | ATCTGCAACCCTTATCCTTTGTTGCCAGCGGTCCGGCCGGG |
| | | AACTCAAAGGAGACTGCCAGTGATAAACTGGAGGAAGGTGG |
| | | GGATGACGTCAAGTCATCATGGCCCTTACGACCAGGGCTAC |
| | | ACACGTGCTACAATGGCGCATACAAAG |
| 3118127 | 3118127 | GCGAGAGCAAGCGGACCTCATAAAGTGCGTCGTAGTCCGGA |
| | | TTGGAGTCTGCAACTCGACTCCATGAAGTCGGAATCGCTAGT |
| | | AATCGTGGATCAGAATGCCACGGTGAATACGTTCCCGGGCC |
| | | TTGAGATCGGAAGAGCGTCGTGTAGG |
| 3118127 | 3118127 | TCAGACGTGTGCTCTTCCGATCTGCGAGAGCAAGCGGACCT |
| | | CATAAAGTGCGTCGTAGTCCGGATTGGAGTCTGCAACTCGA |
| | | CTCCATGAAGTCGGAATCGCTAGTAATCGTGGATCAGAATGC |
| | | CACGGTGAATACGTTCCCGGGCCTTG |
| 3118863 | 3118863 | CGGCGATTTCCGAATGGGGAAACCCAGTGTGTTTCGACACA |
| | | CTATCATTAACTGAATCCATAGGTTAATGAGGCGAACCGGGG |
| | | GAACTGAAACATCTAAGTACCCCGAGGAAAAGAAATCAACCG |
| | | AGATTCCAGATCGGAAGAGCGTCGT |
| 3118863 | 3118863 | CGTGTGCTCTTCCGATCTCGGCGATTTCCGAATGGGGAAAC |
| | | CCAGTGTGTTTCGACACACTATCATTAACTGAATCCATAGGTT |
| | | AATGAGGCGAACCGGGGGAACTGAAACATCTAAGTACCCCG |
| | | AGGAAAAGAAATCAACCGAGATTCC |
| 3118975 | 3118975 | AAGAAATCAACCGAGATTCCCCCAGTAGCGGCGAGCGAACG |
| | | GGGAGGAGCCCAGAGCCTGAATCAGTGTGTGTGTTAGTGGA |
| | | AGCGTCTGGAAAGGCGCGCGATACAGGGTGACAGCCCCGT |
| | | ACACAAAAAAGATCGGAAGAGCGTCGTG |
| 3118975 | 3118975 | ACGTGTGCTCTTCCGATCTAAGAAATCAACCGAGATTCCCCC |
| | | AGTAGCGGCGAGCGAACGGGGAGGAGCCCAGAGCCTGAAT |
| | | CAGTGTGTGTGTTAGTGGAAGCGTCTGGAAAGGCGCGCGAT |
| | | ACAGGGTGACAGCCCCGTACACAAAAA |
| 3119245 | 3119281 | GGCGAGGGGAGTGAAAAAGAACCTGAAACCGTGTACGTACA |
| | | AGCAGTGGGAGCCTCTTAATGGGGTGACTGCGTACCTTTTGT |
| | | ATAATGGGTCAGCGACTTATATTCTGTAGCAAGGTTAACCGA |
| | | ATAGGGGAGCCGAAGGGAAACCGAG |
| 3119281 | 3119245 | GTACAAGCAGTGGGAGCCTCTTAATGGGGTGACTGCGTACC |
| | | TTTTGTATAATGGGTCAGCGACTTATATTCTGTAGCAAGGTTA |
| | | ACCGAATAGGGGAGCCGAAGGGAAACCGAGTCTTAACTGGG |
| | | CGTTAAGTTGCAGGGTATAGACCCG |
| 3119581 | 3119602 | ATTTAGGTAGCGCCTCGTGAATTCATCTCCGGGGGTAGAGC |
| | | ACTGTTTCGGCAAGGGGGTCATCCCGACTTACCAACCCGAT |
| | | GCAAACTGCGAATACCGGAGAATGTTATCACGGGAGACACA |
| | | CGGCGGGTGCTAACGTCCGTCGTGAAG |
| 3119594 | 3079419 | ATCTCGTGAATTCATCTCCGGGGGTAGAGCACTGTTTCGGCA |
| | | AGGGGGTCATCCCGACTTACCAACCCGATGCAAACTGCGAA |
| | | TACCGGAGAATGTTATCACGGGAGACACACGGCGGGTGCTA |
| | | ACGTCCGTCGTGAAGAGGGAAACAAC |
| 3119594 | 2950273 | ATCTCGTGAATTCATCTCCGGGGGTAGAGCACTGTTTCGGCA |
| | | AGGGGGTCATCCCGACTTACCAACCCGATGCAAACTGCGAA |
| | | TACCGGAGAATGTTATCACGGGAGACACACGGCGGGTGCTA |
| | | ACGTCCGTCGTGAAGAGGGAAACAAC |
| 3119594 | 2835778 | ATCTCGTGAATTCATCTCCGGGGGTAGAGCACTGTTTCGGCA |
| | | AGGGGGTCATCCCGACTTACCAACCCGATGCAAACTGCGAA |
| | | TACCGGAGAATGTTATCACGGGAGACACACGGCGGGTGCTA |
| | | ACGTCCGTCGTGAAGAGGGAAACAAC |
| 3119594 | 3079419 | CTCGTGAATTCATCTCCGGGGGTAGAGCACTGTTTCGGCAA |
| | | GGGGGTCATCCCGACTTACCAACCCGATGCAAACTGCGAAT |
| | | ACCGGAGAATGTTATCACGGGAGACACACGGCGGGTGCTAA |
| | | CGTCCGTCGTGAAGAGGGAAACAACCC |
| 3119594 | 2950273 | CTCGTGAATTCATCTCCGGGGGTAGAGCACTGTTTCGGCAA |
| | | GGGGGTCATCCCGACTTACCAACCCGATGCAAACTGCGAAT |
| | | ACCGGAGAATGTTATCACGGGAGACACACGGCGGGTGCTAA |
| | | CGTCCGTCGTGAAGAGGGAAACAACCC |
| 3119594 | 2835778 | CTCGTGAATTCATCTCCGGGGGTAGAGCACTGTTTCGGCAA |
| | | GGGGGTCATCCCGACTTACCAACCCGATGCAAACTGCGAAT |
| | | ACCGGAGAATGTTATCACGGGAGACACACGGCGGGTGCTAA |
| | | CGTCCGTCGTGAAGAGGGAAACAACCC |
| 3119602 | 3119581 | TTCATCTCCGGGGGTAGAGCACTGTTTCGGCAAGGGGGTCA |
| | | TCCCGACTTACCAACCCGATGCAAACTGCGAATACCGGAGA |
| | | ATGTTATCACGGGAGACACACGGCGGGTGCTAACGTCCGTC |
| | | GTGAAGAGGGAAACAACCCAGACCGCC |
| 3120220 | 3120220 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATAGATCGGAAGAGCAC |
| 3120220 | 3120220 | ACGCTCTTCCGATCTGTTTAAGCGTGTAGGCTGGTTTTCCAG |
| | | GCAAATCCGGAAAATCAAGGCTGAGGCGTGATGACGAGGCA |
| | | CTACGGTGCTGAAGCGACAAATGCCCTGCTTCCAGGAAAAG |
| | | CCTCTAAGCATCAGGTAACATCAAAT |
| 3120220 | 3120248 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATCGTACCCCAAACCGA |
| 3120248 | 3120220 | CAAATCCGGAAAATCAAGGCTGAGGCGTGATGACGAGGCAC |
| | | TACGGTGCTGAAGCGACAAATGCCCTGCTTCCAGGAAAAGC |
| | | CTCTAAGCATCAGGTAACATCAAATCGTACCCCAAACCGACA |
| | | CAGGTGGTCAGGTAGAGAATACCAAG |
| 3121364 | 3121450 | GGTCCCTATCTGCCGTGGGCGCTGGAGAACTGAGGGGGGC |
| | | TGCTCCTAGTACGAGAGGACCGGAGTGGACGCATCACTGGT |
| | | GTTCGGGTTGTCATGCCAATGGCACTGCCCGGTAGCTAAAT |
| | | GCGGAAGAGATAAGTGCTGAAAGCATCT |
| 3121450 | 3121364 | GGTTGTCATGCCAATGGCACTGCCCGGTAGCTAAATGCGGA |
| | | AGAGATAAGTGCTGAAAGCATCTAAGCACGAAACTTGCCCCG |
| | | AGATGAGTTCTCCCTGACTCCTTGAGAGTCCTGAAGGAACGT |
| | | TGAAGACGACGACGTTGATAGGCCG |
| 3991338 | 3991487 | CGACGCGTAATGCGTTGGGGATTCTTGGTGGGATCCCGCGC |
| | | CGTGAATTTACTCGCGACAGCATCGAAGAGAAAGTCGCTGC |
| | | CACCACGCAGGCACAATGGCCGGTTCATGCGGTGATTACCA |
| | | ACTCCACCTATGATGGCTTGCTCTACA |
| 3991487 | 3991338 | AACACCGACTGGATCAAACAGACGCTGGATGTCCCGTCGAT |
| | | CCACTTCGATTCCGCCTGGGTGCCGTACACCCATTTTCATCC |
| | | GATCTACCAGGGGAAAAGTGGTATGAGCGGCGAGCGTGTTG |
| | | CGGGAAAAGTGATCTTCGAAACGCAA |
| 4004615 | 4004621 | CTCAGATTGAACGCTGGCGGCAGGCCTAACACATGCAAGTC |
| | | GAACGGTAACAGGAAGCAGCTTGCTGCTTTGCTGACGAGTG |
| | | GCGGACGGGTGAGTAATGTCTGGGAAACTGCCTGATGGAGG |
| | | GGGATAACTACTGGAAACGGTAGCTAA |
| 4004621 | 4004615 | TTGAACGCTGGCGGCAGGCCTAACACATGCAAGTCGAACGG |
| | | TAACAGGAAGCAGCTTGCTGCTTTGCTGACGAGTGGCGGAC |
| | | GGGTGAGTAATGTCTGGGAAACTGCCTGATGGAGGGGGATA |
| | | ACTACTGGAAACGGTAGCTAATACCGC |
| 4004825 | 4004831 | CCCAGATGGGATTAGCTAGTAGGTGGGGTAACGGCTCACCT |
| | | AGGCGACGATCCCTAGCTGGTCTGAGAGGATGACCAGCCAC |
| | | ACTGGAACTGAGACACGGTCCAGACTCCTACGGGAGGCAGC |
| | | AGTGGGGAATATTGCACAATGGGCGCA |
| 4004831 | 4004825 | TGGGATTAGCTAGTAGGTGGGGTAACGGCTCACCTAGGCGA |
| | | CGATCCCTAGCTGGTCTGAGAGGATGACCAGCCACACTGGA |
| | | ACTGAGACACGGTCCAGACTCCTACGGGAGGCAGCAGTGG |
| | | GGAATATTGCACAATGGGCGCAAGCCTG |
| 4005060 | 4005099 | ACCTTTGCTCATTGACGTTACCCGCAGAAGAAGCACCGGCTA |
| | | ACTCCGTGCCAGCAGCCGCGGTAATACGGAGGGTGCAAGC |
| | | GTTAATCGGAATTACTGGGCGTAAA.GCGCACGCAGGCGGTT |
| | | TGTTAAGTCAGATGTGAAATCCCCGGG |
| 4005099 | 4005060 | CTAACTCCGTGCCAGCAGCCGCGGTAATACGGAGGGTGCAA |
| | | GCGTTAATCGGAATTACTGGGCGTAAAGCGCACGCAGGCGG |
| | | TTTGTTAAGTCAGATGTGAAATCCCCGGGCTCAACCTGGGAA |
| | | CTGCATCTGATACTGGCAAGCTTGAG |
| 4005449 | 4005449 | GTTGGAAGCCTTGAGCTTTTAGTGGCGCAGCTAACGCATTAA |
| | | GTTGACCGCCTGGGGAGTACGGCCGCAAGGTTAAAACTCAA |
| | | ATGAATTGACGGGGGCCCGCACAAGCGGTGGAGCATGTGGT |
| | | TTAATTCGAAAGATCGGAAGAGCGTC |
| 4005449 | 4005449 | TGTGCTCTTCCGATCTGTTGGAAGCCTTGAGCTTTTAGTGGC |
| | | GCAGCTAACGCATTAAGTTGACCGCCTGGGGAGTACGGCCG |
| | | CAAGGTTAAAACTCAAATGAATTGACGGGGGCCCGCACAAG |
| | | CGGTGGAGCATGTGGTTTAATTCGAA |
| 4005598 | 4005598 | GTTfTCAGAGATGAGAATGTGCCTTCGGGAGCCGTGAGACA |
| | | GGTGCTGCATGGCTGTCGTCAGCTCGTGTTGTGAAATGTTG |
| | | GGTTAAGTCCCGGAACGAGCGGAACGCTTATCCAGATCGGA |
| | | AGAGCACACGTCTGAACTCCAGTCACA |
| 4005598 | 4005598 | GAACACTCTTTCCCTACACGACGCTCTTCCGATCTGTTTTCA |
| | | GAGATGAGAATGTGCCTTCGGGAGCCGTGAGACAGGTGCTG |
| | | CATGGCTGTCGTCAGCTCGTGTTGTGAAATGTTGGGTTAAGT |
| | | CCCGCAACGAGCGCAACCCTTATCC |
| 4005650 | 4005687 | GCTGTCGTCAGCTCGTGTTGTGAAATGTTGGGTTAAGTCCCG |
| | | CAACGAGCGCAACCCTTATCCTTTGTTGCCAGCGGTCCGGC |
| | | CGGGAACTCAAAGGAGACTGCCAGTGATAAACTGGAGGAAG |
| | | GTGGGGATGACGTCAAGTCATCATGG |
| 4005687 | 4005650 | TCCCGCAACGAGCGCAACCCTTATCCTTTGTTGCCAGCGGT |
| | | CCGGCCGGGAACTCAAAGGAGACTGCCAGTGATAAACTGGA |
| | | GGAAGGTGGGGATGACGTCAAGTCATCATGGCCCTTACGAC |
| | | CAGGGCTACACACGTGCTACAATGGCG |
| 4005858 | 4005858 | GCGAGAGCAAGCGGACCTCATAAAGTGCGTCGTAGTCCGGA |
| | | TTGGAGTCTGCAACTCGACTCCATGAAGTCGGAATCGCTAGT |
| | | AATCGTGGATCAGAATGCCACGGTGAATACGTTCCCGGGCC |
| | | TTGAGATCGGAAGAGCGTCGTGTAGG |
| 4005858 | 4005858 | TCAGACGTGTGCTCTTCCGATCTGCGAGAGCAAGCGGACCT |
| | | CATAAAGTGCGTCGTAGTCCGGATTGGAGTCTGCAACTCGA |
| | | CTCCATGAAGTCGGAATCGCTAGTAATCGTGGATCAGAATGC |
| | | CACGGTGAATACGTTCCCGGGCCTTG |
| 4006607 | 4006670 | GATGCCCTGGCAGTCAGAGGCGATGAAGGACGTGCTAATCT |
| | | GCGATAAGCGTCGGTGAGGTGATATGAACCGTTATAACCGG |
| | | CGATTTCCGAATGGGGAAACCCAGTGTGATTCGTCACACTAT |
| | | CATTAACTGAATCCATAGGTTAATGA |
| 4006670 | 4006607 | TATGAACCGTTATAACCGGCGATTTCCGAATGGGGAAACCCA |
| | | GTGTGATTCGTCACACTATCATTAACTGAATCCATAGGTTAAT |
| | | GAGGCGAACCGGGGGAACTGAAACATCTAAGTACCCCGAGG |
| | | AAAAGAAATCAACCGAGATTCCCC |
| 4006798 | 4006798 | AAGAAATCAACCGAGATTCCCCCAGTAGCGGCGAGCGAACG |
| | | GGGAGGAGCCCAGAGCCTGAATCAGTGTGTGTGTTAGTGGA |
| | | AGCGTCTGGAAAGGCGCGCGATACAGGGTGACAGCCCCGT |
| | | ACACAAAAAAGATCGGAAGAGCGTCGTG |
| 4006798 | 4006798 | ACGTGTGCTCTTCCGATCTAAGAAATCAACCGAGATTCCCCC |
| | | AGTAGCGGCGAGCGAACGGGGAGGAGCCCAGAGCCTGAAT |
| | | CAGTGTGTGTGTTAGTGGAAGCGTCTGGAAAGGCGCGCGAT |
| | | ACAGGGTGACAGCCCCGTACACAAAAA |
| 4007068 | 4007104 | GGCGAGGGGAGTGAAAAAGAACCTGAAACCGTGTACGTACA |
| | | AGCAGTGGGAGCCTCTTAATGGGGTGACTGCGTACCTTTTGT |
| | | ATAATGGGTCAGCGACTTATATTCTGTAGCAAGGTTAACCGA |
| | | ATAGGGGAGCCGAAGGGAAACCGAG |
| 4007104 | 4007068 | GTACAAGCAGTGGGAGCCTCTTAATGGGGTGACTGCGTACC |
| | | TTTTGTATAATGGGTCAGCGACTTATATTCTGTAGCAAGGTTA |
| | | ACCGAATAGGGGAGCCGAAGGGAAACCGAGTCTTAACTGGG |
| | | CGTTAAGTTGCAGGGTATAGACCCG |
| 4007404 | 4007425 | ATTTAGGTAGCGCCTCGTGAATTCATCTCCGGGGGTAGAGC |
| | | ACTGTTTCGGCAAGGGGGTCATCCCGACTTACCAACCCGAT |
| | | GCAAACTGCGAATACCGGAGAATGTTATCACGGGAGACACA |
| | | CGGCGGGTGCTAACGTCCGTCGTGAAG |
| 4007425 | 4007404 | TTCATCTCCGGGGGTAGAGCACTGTTTCGGCAAGGGGGTCA |
| | | TCCCGACTTACCAACCCGATGCAAACTGCGAATACCGGAGA |
| | | ATGTTATCACGGGAGACACACGGCGGGTGCTAACGTCCGTC |
| | | GTGAAGAGGGAAACAACCCAGACCGCC |
| 4008043 | 4008043 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATAGATCGGAAGAGCAC |
| 4008043 | 4008043 | ACGCTCTTCCGATCTGTTTAAGCGTGTAGGCTGGTTTTCCAG |
| | | GCAAATCCGGAAAATCAAGGCTGAGGCGTGATGACGAGGCA |
| | | CTACGGTGCTGAAGCGACAAATGCCCTGCTTCCAGGAAAAG |
| | | CCTCTAAGCATCAGGTAACATCAAAT |
| 4008043 | 4008071 | GTTTAAGCGTGTAGGCTGGTTTTCCAGGCAAATCCGGAAAAT |
| | | CAAGGCTGAGGCGTGATGACGAGGCACTACGGTGCTGAAGC |
| | | GACAAATGCCCTGCTTCCAGGAAAAGCCTCTAAGCATCAGGT |
| | | AACATCAAATCGTACCCCAAACCGA |
| 4008071 | 4008043 | CAAATCCGGAAAATCAAGGCTGAGGCGTGATGACGAGGCAC |
| | | TACGGTGCTGAAGCGACAAATGCCCTGCTTCCAGGAAAAGC |
| | | CTCTAAGCATCAGGTAACATCAAATCGTACCCCAAACCGACA |
| | | CAGGTGGTCAGGTAGAGAATACCAAG |
| 4009187 | 4009273 | GGTCCCTATCTGCCGTGGGCGCTGGAGAACTGAGGGGGGC |
| | | TGCTCCTAGTACGAGAGGACCGGAGTGGACGCATCACTGGT |
| | | GTTCGGGTTGTCATGCCAATGGCACTGCCCGGTAGCTAAAT |
| | | GCGGAAGAGATAAGTGCTGAAAGCATCT |
| 4009273 | 4009187 | GGTTGTCATGCCAATGGCACTGCCCGGTAGCTAAATGCGGA |
| | | AGAGATAAGTGCTGAAAGCATCTAAGCACGAAACTTGCCCCG |
| | | AGATGAGTTCTCCCTGACTCCTTGAGAGTCCTGAAGGAACGT |
| | | TGAAGACGACGACGTTGATAGGCCG |
| 4527988 | 4527990 | TTCCCTACACGACGCTCTTCCGATCTCGCCACATGATGATGG |
| | | ATTTTGGCTATCTGGAAGAAACATTCGAAGCGGGTAAACGCT |
| | | CAGCCAAAATCTCCTTTGTTATTACTGTCGTGCTTTCACTTCT |
| | | CGCAGGAGTCCTCGTATGGTAAG |
| 4527990 | 4527988 | CTCCACATGATGATGGATTTTGGCTATCTGGAAGAAACATTC |
| | | GAAGAGGGTAAACGATCCGCCAAAATCTCCTTTGTTATTACT |
| | | GTCGTGATTTCACTTCTCGCAGGAGTCATCTTATGTTAAGAG |
| | | ATCGGAAGAGCACACGTCTGAACT |

Next, target genes were identified to create custom PCR primers for identification of the pathogen. See TABLE 8.

TABLE 8
Target genes

| wcaK | Start | End | Nucleic Acid Sequence |
|---|---|---|---|
| A01587:71 | 2317120 | 2317148 | CTTGGTATTCTCTACCTGACCACCTGTGTCGGTT |
| | | | TGGGGTACGATTTGATGTTACCTGATGCTTAGAG |
| | | | GCTTTTCCTGGAAGCAGGGCATTTGTCGCTTCA |
| | | | GCACCGTAGTGCCTCGICATCACGCCTCAGCCT |
| | | | TGATTTTCCGGATTTG |
| A01587:71 | 2317148 | 2317120 | TCGGTTTGGGGTACGATTTGATGTTACCTGATGC |
| | | | TTAGAGGCTTTTCCTGGAAGCAGGGCATTTGTC |
| | | | GCTTCAGCACCGTAGTGCCTCGTCATCACGCCT |
| | | | CAGCCTTGATTTTCCGGATTTGCCTGGAAAACCA |
| | | | GCCTACACGCTTAAAC |
| A01587:71 | 2317163 | 2317163 | ATTTGATGITACCTGATGCTTAGAGGCTTTTCCT |
| | | | GGAAGCAGGGCATTTGTCGCTTCAGCACCGTAG |
| | | | TGCCTCGTCATCACGCCTCAGCCTTGATTTTCCG |
| | | | GATTTGCCTGGAAAACCAGCCTACACGCTTAAAC |
| | | | AGATCGGAAGAGCGT |
| A01587:71 | 2317163 | 2317163 | GTGCTCTTCCGATCTATTTGATGTTACCTGATGC |
| | | | TTAGAGGCTTTTCCTGGAAGCAGGGCATTTGTC |
| | | | GCTTCAGCACCGTAGTGCCTCGTCATCACGCCT |
| | | | CAGCCTTGATTTTCCGGATTTGCCTGGAAAACCA |
| | | | GCCTACACGCTTAAAC |
| wcaJ | Start | End | Nucleic Acid Sequence |
| A01587:71 | 2319355 | 2319355 | CAAGGCCCGGGAACGTATTCACCGTGGCATTCT |
| | | | GATCCACGATTACTAGCGATTCCGACTTCATGGA |
| | | | GTCGAGTTGCAGACTCCAATCCGGACTACGACG |
| | | | CACTTTATGAGGTCCGCTTGCTCTCGCAGATCG |
| | | | GAAGAGCACACGTCTGA |
| A01587:71 | 2319355 | 2319355 | CCTACACGACGCTCTTCCGATCTCAAGGCCCGG |
| | | | GAACGTATTCACCGTGGCATTCTGATCCACGATT |
| | | | ACTAGCGATTCCGACTTCATGGAGTCGAGTTGC |
| | | | AGACTCCAATCCGGACTACGACGCACTTTATGA |
| | | | GGTCCGCTTGCTCTCGC |
| A01587:71 | 2319362 | 1503671 | CTGGAGTTCAGACGTGTGCTCTTCCGATCTCGG |
| | | | GAACGTATTCACCGTGGCATTCTGATCCACGATT |
| | | | ACTAGCGATTCCGACTTCATGGAGTCGAGTTGC |
| | | | AGACTCCAATCCGGACTACGACGCACTTTATGA |
| | | | GGTCCGCTTGCTCTCGC |
| A01587:71 | 2319503 | 2319540 | CGCCATTGTAGCACGTGTGTAGCCCTGGTCGTA |
| | | | AGGGCCATGATGACTTGACGTCATCCCCACCTT |
| | | | CCTCCAGTTTATCACTGGCAGTCTCCTTTGAGTT |
| | | | CCCGGCCGGACCGCTGGCAACAAAGGATAAGG |
| | | | GTTGCGCTCGTTGCGGGA |
| A01587:71 | 2319540 | 2319503 | CCATGATGACTTGACGTCATCCCCACCTTCCTCC |
| | | | AGTTTATCACTGGCAGTCTCCTTTGAGTTCCCGG |
| | | | CCGGACCGCTGGCAACAAAGGATAAGGGTTGCG |
| | | | CTCGTTGCGGGACTTAACCCAACATTTCACAACA |
| | | | CGAGCTGACGACAGC |
| A01587:71 | 2319633 | 2319644 | CGGGTTGCGCTCGTTGCGGGACTTAACCCAACA |
| | | | TTTCACAACACGAGCTGACGACAGCCATGCAGC |
| | | | ACCTGTCTCACGGTTCCCGAAGGCACATTCTCAT |
| | | | CTCTGAAAACTTCCGTGGATGTCAAGACCAGGTA |
| | | | AGGTTCTTCGCGTTGC |
| A01587:71 | 2319644 | 2319633 | GTTGCGGGACTTAACCCAACATTTCACAACACGA |
| | | | GCTGACGACAGCCATGCAGCACCTGTCTCACGG |
| | | | TTCCCGAAGGCACATTCTCATCTCTGAAAACTTC |
| | | | CGTGGATGTCAAGACCAGGTAAGGTTCTTCGCG |
| | | | TTGCATCGAATTAAAC |
| A01587:71 | 2319783 | 2319783 | TTCGAATTAAACCACATGCTCCACCGCTTGTGCG |
| | | | GGCCCCCGTCAATTCATTTGAGTTTTAACCTTGC |
| | | | GGCCGTACTCCCCAGGCGGTCAACTTAATGCGT |
| | | | TAGCTGCGCCACTAAAAGCTCAAGGCTTCCAAC |
| | | | AGATCGGAAGAGCACA |
| A01587:71 | 2319783 | 2319783 | GACGCTCTTCCGATCTTTCGAATTAAACCACATG |
| | | | CTCCACCGCTTGTGCGGGCCCCCGTCAATTCAT |
| | | | TTGAGTTTTAACCTTGCGGCCGTACTCCCCAGG |
| | | | CGGTCAACTTAATGCGTTAGCTGCGCCACTAAAA |
| | | | GCTCAAGGCTTCCAAC |
| fdx | Star | End | Nucleic Acid Sequence |
| A01587:71 | 2833900 | 2833900 | GTTGGAAGCCTTGAGCTTTTAGTGGCGCAGCTA |
| | | | ACGCATTAAGTTGACCGCCTGGGGAGTACGGCC |
| | | | GCAAGGTTAAAACTCAAATGAATTGACGGGGGC |
| | | | CCGCACAAGCGGTGGAGCATGTGGTTTAATTCG |
| | | | AAAGATCGGAAGAGCGTC |
| A01687:71 | 2833900 | 2833900 | TGTGCTCTTCCGATCTGTTGGAAGCCTTGAGCTT |
| | | | TTAGTGGCGCAGCTAACGCATTAAGTTGACCGC |
| | | | CTGGGGAGTACGGCCGCAAGGTTAAAACTCAAA |
| | | | TGAATTGACGGGGGCCCGCACAAGCGGTGGAG |
| | | | CATGTGGTTTAATTCGAA |
| A01587:71 | 2833997 | 2834009 | GTTTAATTCGATGCAACGCGAAGAACCTTACCTG |
| | | | GTCTTGACATCCACGGAAGTTTTCAGAGATGAGA |
| | | | ATGTGCCTTCGGGAACCGTGAGACAGGTGCTGC |
| | | | ATGGCTGTCGTCAGCTCGTGTTGTGAAATGTTG |
| | | | GGTTAAGTCCCGCAAC |
| A01587:71 | 2834009 | 2833997 | GCAACGCGAAGAACCTTACCTGGTCTTGACATC |
| | | | CACGGAAGTTTTGAGAGATGAGAATGTGCCTTC |
| | | | GGGAACCGTGAGACAGGTGCTGOATGGCTGTC |
| | | | GTCAGCTCGTGTTGTGAAATGTTGGGTTAAGTCC |
| | | | CGCAACGAGCGCAACCCG |
| A01587:71 | 2834049 | 2948544 | GTTTTGAGAGATGAGAATGTGCCTTCGGGAACC |
| | | | GTGAGACAGGTGCTGGATGGCTGTCGTCAGCTC |
| | | | GTGTTGTGAAATGTTGGGTTAAGTCCCGCAACG |
| | | | AGCGCAAGATGGGAAGAGCAGACGTCTGAACTC |
| | | | CAGTCACAACGTACGATC |
| A01587:71 | 2834049 | 3077599 | GTTTTGAGAGATGAGAATGTGCCTTCGGGAACC |
| | | | GTGAGACAGGTGCTGCATGGCTGTCGTCAGCTC |
| | | | GTGTTGTGAAATGTTGGGTTAAGTCCCGCAACG |
| | | | AGCGCAAGATCGGAAGAGCACACGTCTGAACTC |
| | | | CAGTCAGAAGGTACGATC |
| A01587:71 | 2834049 | 3117867 | GTTTTCAGAGATGAGAATGTGGGTTCGGGAACC |
| | | | GTGAGACAGGTGCTGCATGGCTGTCGTCAGCTC |
| | | | GTGTTGTGAAATGTTGGGTTAAGTCCCGCAACG |
| | | | AGCGCAAGATCGGAAGAGCAGACGTCTGAACTC |
| | | | CAGTCACAACCTACGATC |
| A01587:71 | 2834101 | 2834138 | GCTGTCGTCAGCTCGTGTTGTGAAATGTTGGGTT |
| | | | AAGTCCCGCAACGAGCGCAACCCTTATCCTTTGT |
| | | | TGCCAGCGGTCCGGCCGGGAACTCAAAGGAGA |
| | | | CTGCCAGTGATAAACTGGAGGAAGGTGGGGATG |
| | | | ACGTCAAGTCATCATGG |
| A01587:71 | 2834138 | 2834101 | TCCCGCAACGAGCGCAACCCTTATCCTTTGTTGC |
| | | | CAGCGGTCCGGCCGQGAACTCAAAGGAGACTG |
| | | | CCAGTGATAAACTGGAGGAAGGTGGGGATGACG |
| | | | TCAAGTCATCATGGCCCTTACGACCAGGGCTAC |
| | | | ACACGTGCTACAATGGCG |
| A01587:71 | 2834151 | 2948646 | GCAACCCTTATCCTTTGTTGCCAGCGGTCCGGC |
| | | | CGGGAACTCAAAGGAGACTGCCAGTGATAAACT |
| | | | GGAGGAAGGTGGGGATGACGTCAAGTCATCATG |
| | | | GCCCTTACGACCAGGGCTACACACGTGCTACAA |
| | | | TGGCGCATACAAAGAGAT |
| A01587:71 | 2834151 | 3077701 | GCAACCCTTATCCTTTGTTGCCAGCGGTCCGGC |
| | | | CGGGAACTCAAAGGAGACTGCCAGTGATAAACT |
| | | | GGAGGAAGGTGGGGATGACGTCAAGTCATCATG |
| | | | GCCCTTACGACCAGGGCTACACACGTGCTACAA |
| | | | TGGCGCATACAAAGAGAT |
| A01587:71 | 2834151 | 3117969 | GCAACCCTTATCCTTTGTTGCCAGCGGTCCGGC |
| | | | CGGGAACTCAAAGGAGACTGCCAGTGATAAACT |
| | | | GGAGGAAGGTGGGGATGACGTCAAGTCATCATG |
| | | | GCCCTTACGACCAGGGCTACACACGTGCTACAA |
| | | | TGGCGCATACAAAGAGAT |
| ygaZ | Start | End | Nucleic Acid Sequence |
| A01587:71 | 2948596 | 2948633 | GCTGTCGTCAGCTCGTGTTGTGAAATGTTGGGTT |
| | | | AAGTCCCGCAACGAGCGCAACCCTTATCCTTTGT |
| | | | TGCCAGCGGTCCGGCCGGGAACTCAAAGGAGA |
| | | | CTGCCAGTGATAAACTGGAGGAAGGTGGGGATG |
| | | | ACGTCAAGTCATCATGG |
| A01587:71 | 2948633 | 2948596 | TCCCGCAACGAGCGCAACCCTTATCCTTTGTTGC |
| | | | CAGCGGTCCGGCCGGGAACTCAAAGGAGACTG |
| | | | CCAGTGATAAACTGGAGGAAGGTGGGGATGACG |
| | | | TCAAGTCATCATGGCCCTTACGACCAGGGCTAC |
| | | | ACACGTGCTACAATGGCG |
| A01587:71 | 2948646 | 2834151 | ATCTGCAACCCTTATCCTTTGTTGCCAGCGGTCC |
| | | | GGCCGGGAACTCAAAGGAGACTGCCAGTGATAA |
| | | | ACTGGAGGAAGGTGGGGATGACGTCAAGTCATC |
| | | | ATGGCCCTTACGACCAGGGCTACACACGTGCTA |
| | | | CAATGGCGCATACAAAG |
| A01587:71 | 2948646 | 3077701 | GCAACCCTTATCCTTTGTTGCCAGCGGTCCGGC |
| | | | CGGGAACTCAAAGGAGACTGCCAGTGATAAACT |
| | | | GGAGGAAGGTGGGGATGACGTCAAGTCATCATG |
| | | | GCCCTTACGACCAGGGCTACACACGTGCTACAA |
| | | | TGGCGCATACAAAGAGAT |
| A01587:71 | 2948646 | 3117969 | GCAACCCTTATCCTTTGTTGCCAGCGGTCCGGC |
| | | | CGGGAACTCAAAGGAGACTGCCAGTGATAAACT |
| | | | GGAGGAAGGTGGGGATGACGTCAAGTCATCATG |
| | | | GCCCTTACGACCAGGGCTACACACGTGCTACAA |
| | | | TGGCGCATACAAAGAGAT |
| A01587:71 | 2948804 | 2948804 | GCGAGAGCAAGCGGACCTCATAAAGTGCGTCGT |
| | | | AGTCCGGATTGGAGTCTGCAACTCGACTCCATG |
| | | | AAGTCGGAATCGCTAGTAATCGTGGATCAGAAT |
| | | | GCCACGGTGAATACGTTCCCGGGCCTTGAGATC |
| | | | GGAAGAGCGTCGTGTACG |
| A01587:71 | 2948804 | 2948804 | TCAGACGTGTGCTCTTCCGATCTGCGAGAGCAA |
| | | | GCGGACCTCATAAAGTGCGTCGTAGTCCGGATT |
| | | | GGAGTCTGCAACTCGACTCCATGAAGTCGGAAT |
| | | | CGCTAGTAATCGTGGATCAGAATGCCACGGTGA |
| | | | ATACGTTCCCGGGCCTTG |

### REFERENCES

A person of ordinary skill in the biomedical art of can use these patents, patent applications, and scientific references as guidance to predictable results when making and using the invention:

### Patent References

US20070203082A1 (Tang et al.). RNAi Agents For Anti-SARS Coronavirus Therapy. The invention provides RNA interference (RNAi) agents and delivery methods to inhibit SARS-coronavirus (SARS-CoV) activity or another virus. The invention inhibits viral production of key proteins required for replication, infection, and other functions critical to the virus lifecycle. The invention also disrupts the viral genome RNA directly. The invention provides: sequences of RNAi agent, small interfering RNA (siRNA), that can be chemically synthesized or vector expressed, in vitro transcribed and vector expressed shRNA; siRNA, miRNA and other types of siRNA molecules, having potent antiviral activity in mammalian cells and animals; agents useful for siRNA-mediated gene inhibition in mammalian cells and animal airways and lung tissues: agents useful for efficient delivery of siRNA into the airways of animal model; mechanism of action of SARS-CoV specific siRNA duplexes for inhibition of the viral infection and replication in mammals; target sequences coding for key proteins required for coronavirus replication and infection; target sequences for siRNA-mediated disruption of coronavirus viral RNA genome in coding and non-coding regions; routes and methods of delivery for nucleic acid agents and analogues for mammals; methods and reagents for RNA template-specific RNA based RT-PCR for detection of any portion of the viral RNA genome, for applications of diagnosis and prognosis; and methods for using nucleic acid agents and analogues to treat pulmonary diseases and infections.
U.S. Pat. No. 7,129,223B2 (Kung et al.). Inhibition of SARS-associated coronavirus (SCoV) infection and replication by RNA interference. The invention is based upon the inventor's use of gene therapy, such as RNA interference (RNAi) molecules to inhibit coronavirus infection and replication. The invention relates to using small interfering RNA (siRNA) or double-stranded RNA (dsRNA) as therapeutic agents to treat Severe Acute Respiratory Syndrome (SARS) in humans. The invention encompasses RNAi molecules that target different sites of the genome of the hSARS virus and inhibit infection and replication of the hSARS virus. Specifically, the invention encompasses siRNAs that target different sites of the replicase region of the hSARS virus and are useful to treat SARS in humans. The invention also encompasses small hairpin RNA (shRNA) containing plasmids under a promoter's control to treat SARS.
CA2523658A1 (Intradigm Corp.). RNAi agents for anti-SARS coronavirus therapy. This patent application is related to US20070203082A1 (Tang et al.) above.
US20060258611A1 (Kung et al.). Inhibition of SARS-associated coronavirus (SCoV) infection and replication by RNA interference. This patent application is related to U.S. Pat. No. 7,129,223B2 (Kung et al.) above.

### Non-Patent References

A comprehensive assessment of RNA-seq accuracy, reproducibility, and information content by the Sequencing Quality Control Consortium. Nature Biotechnology, 32, 903-914 (2014).
Ackermann et al., Pulmonary vascular endothelialitis, thrombosis, and angiogenesis in Covid-19. The New England Journal of Medicine, 383, 120-128 (2020).
Al-Samkari et al., COVID and coagulation: Bleeding and thrombotic manifestations of SARS-CoV2 infection. Blood (2020).
Andrews, A quality control tool for high throughput sequence data. FastQC. In: Editor\hat{ }Book, A quality control tool for high throughput sequence data. FastQC (2014).
Ashburner et al., Gene ontology: tool for the unification of biology. The Gene Ontology Consortium. Nature Genetics, 25, 25-29 (2000).
Ayala et al., Shock-induced neutrophil mediated priming for acute lung injury in mice: divergent effects of TLR-4 and TLR-4/FasL deficiency. The American Journal of Pathology, 161, 2283-2294 (2002).
Beigel et al., Remdesivir for the treatment of Covid-19—Preliminary Report. New England Journal of Medicine (2020).
Bellesi et al., Increased CD95 (Fas) and PD-1 expression in peripheral blood T lymphocytes in COVID-19 patients. British Journal of Haematology (2020).
Benz et al., Circulating microRNAs as biomarkers for sepsis. Int. J. Mol. Sci., 17(1) (Jan. 9, 2016).
Biron et al., Biomarkers for Sepsis, What Is and What Might Be? Biomarker Insights, 10(Suppl 4), 7-17 (Sep. 15, 2015).
Bloos & Reinhart, Rapid diagnosis of sepsis. Virulence, 5(1), 154-60 (Jan. 1, 2014).
Boratyn et al., Magic-BLAST, an accurate RNA-seq aligner for long and short reads. BMC Bioinformatics, 20, 405 (2019).
Bouadma et al., Immune Alterations in a Patient with SARS-CoV-2-Related Acute Respiratory Distress Syndrome. Journal of Clinical Immunology, 1-11 (2020).
Carithers et al., A novel approach to high-quality postmortem tissue procurement: The GTEx Project. Biopreservation and Biobanking, 13(5), 311-9 (2015).
Cass & Xiao X, mountainClimber identifies alternative transcription start and polyadenylation sites in RNA-Seq. Cell Systems. 9(4), 23, 393-400.e6 (October 2019).
Chang et al., High-throughput binding analysis determines the binding specificity of ASF/SF2 on alternatively spliced human pre-mRNAs. Combinatorial Chemistry & High Throughput Screening, 13(3), 242-52 (2010).
Charles et al., The components of the immune system. Immunobiol. Immune Syst. Health Dis. 5th Ed. (2001).
Chen et al., Detectable 2019-nCoV viral RNA in blood is a strong indicator for the further clinical severity. Emerging Microbes & Infections 9: 469-473 (2020).
Chikani et al., Racial/ethnic disparities in rates of traumatic injury in Arizona, 2011-2012. Public Health Reports, 131(5), 704-10 (2016).
Clinical and virologic characteristics of the first 12 patients with coronavirus disease 2019 (COVID-19) in the United States. Nature Medicine (2020)
Daly et al., Neuropilin-1 is a host factor for SARS-CoV-2 infection. Science (New York, N.Y., USA) (2020).
Dexamethasone in hospitalized patients with Covid-19—Preliminary Report. New England Journal of Medicine (2020).
Dobin et al., STAR: Ultrafast universal RNA-seq aligner. Bioinformatics (Oxford, England), 29(1), 15-21 (January 2013).
Dolin et al., A novel combination of biomarkers to herald the onset of sepsis prior to the manifestation of symptoms. Shock, 49(4), 364-70 (April 2018).
Dong, Du, & Gardner, An interactive web-based dashboard to track COVID-19 in real time. The Lancet, Infectious Diseases (2020).
Duggal et al., Innate and adaptive immune dysregulation in critically ill ICU patients. Sci. Rep., 8(1), 1-11 (Jul. 5, 2018).
Elias & Dias, Microenvironment changes (in pH) affect VEGF alternative splicing. Cancer Microenvironment, 1(1),131-9 (2008).
Fang et al., Comparisons of viral shedding time of SARS-CoV-2 of different samples in ICU and non-ICU patients. The Journal of Infection (2020).
Feng et al., COVID-19 with different severity: A multi-center study of clinical features. American Journal of Respiratory and Critical Care Medicine (2020).
Ferguson et al., The Berlin definition of ARDS: An expanded rationale, justification, and supplementary material. Intensive Care Medicine, 38, 1573-1582 (2012).
Fink & Warren, Strategies to improve drug development for sepsis. Nature Reviews Drug Discovery, 13(10), 741-58 (2014).
Fleige & Pfaffl, RNA integrity and the effect on the real-time qRT-PCR performance. Molecular Aspects of Medicine, 27: 126-139 (2006).
Fredericks et al., Alternative RNA splicing and alternative transcription start/end in acute respiratory distress syndrome. Intensive Care Medicine (2020).
Fredericks et al., RNA-binding proteins: Splicing factors and disease. Biomolecules, 5(2), 893-909 (2015).
Gordon et al., A SARS-CoV-2 protein interaction map reveals targets for drug repurposing. Nature (Apr. 30, 2020).
Gultyaev et al., P element temperature-specific transposition, a model for possible regulation of mobile elements activity by pre-mRNA secondary structure. TSitologiia i Genetika, 48(6), 40-4 (2014).
Hadjadj et al., Impaired type I interferon activity and inflammatory responses in severe COVID-19 patients. Science (New York, N.Y.), 369, 718-724 (2020).
Hardwick et al., Getting the entire message: Progress in isoform sequencing. Frontiers in Genetics, 10, 709 (2019).
Huang et al., Clinical features of patients infected with 2019 novel coronavirus in Wuhan, China. Lancet (London, England) 395: 497-606, (2020).
Iacobellis et al., Perforated appendicitis, assessment with multidetector computed tomography. Seminars in Ultrasound, CT, and MR, 37(1), 31-6 (2016).
Kasim et al., Shutdown of achaete-scute homolog-1 expression by heterogeneous nuclear ribonucleoprotein (hnRNP)-A2/B1 in hypoxia. The Journal of Biological Chemistry, 289(39), 26973-88 (2014).
Kujawski et al., Clinical and virologic characteristics of the first 12 patients with coronavirus disease 2019 (COVID-19) in the United States. Nature Medicine (2020).
Kukurba & Montgomery, RNA sequencing and analysis. Cold Spring Harb. Protoc., 2015(11), 951-69 (Apr. 13, 2015).
Lai, Wang, & Hsueh, Co-infections among patients with COVID-19: The need for combination therapy with non-anti-SARS-CoV-2 agents? Journal of Microbiology, Immunology, and Infection=Wei mian yu gan ran za zhi, 53, 505-512 (2020).
Lee et al., The importance of standardization on analyzing circulating RNA. Mol. Diagn. Ther., 21(3), 259-68 (June 2017).
Lei et al., Activation and evasion of type I interferon responses by SARS-CoV-2. Nature Commun., 11, 3810 (2020).
Lemieux et al., A function for the hnRNP A1/A2 proteins in transcription elongation. PloS One, 10(5), e0126654 (2015).
Lomas-Neira et al., Blockade of endothelial growth factor, angiopoietin-2, reduces indices of ARDS and mortality in mice resulting from the dual-insults of hemorrhagic shock and sepsis. Shock, 45(2), 157-65 (2016).
Mahen et al., mRNA secondary structures fold sequentially but exchange rapidly in vivo. PLoS Biology, 8(2), e1000307 (2010).
Mangul et al., ROP: Dumpster diving in RNA-sequencing to find the source of 1 trillion reads across diverse adult human tissues. Genome Biol., 19 (Feb. 15, 2018).
McElvaney et al., A linear prognostic score based on the ratio of interleukin-6 to interleukin-10 predicts outcomes in COVID-19. EBioMedicine, 61, 103026 (2020).
Mi, Muruganujan, Casagrande, & Thomas, Large-scale gene function analysis with the PANTHER classification system. Nature Protocols, 8, 1551-1566 (2013).
Monaghan et al. Mechanisms of indirect acute lung injury: A novel role for the coinhibitory receptor, programmed death-1. Annals of Surgery, 255(1), 158-64 (2012).
Monaghan et al., Changes in the process of alternative RNA splicing results in soluble B and T lymphocyte attenuator with biological and clinical implications in critical illness. Mol Med., 24(1), 32 (Jun. 18, 2018).
Monaghan et al., Novel anti-inflammatory mechanism in critically ill: Soluble programmed cell death receptor-1 (sPD-1). Journal of the American College of Surgeons, 213(3), S54-S5 (2011).
Monaghan et al., Programmed death 1 expression as a marker for immune and physiological dysfunction in the critically ill surgical patient. Shock, 38(2), 117-22 (2012).
Monaghan et al., Soluble programmed cell death receptor-1 (sPD-1), a potential biomarker with anti-inflammatory properties in human and experimental acute respiratory distress syndrome (ARDS). J. Transl. Med., 14(1), 312 (2016).
Pan et al., Deep surveying of alternative splicing complexity in the human transcriptome by high-throughput sequencing. Nature Genetics, 40(12), 1413-5 (2008).
Poland, Ovsyannikova, & Kennedy, SARS-CoV-2 immunity: Review and applications to phase 3 vaccine candidates. Lancet (London, England) (2020).
Prescott & Rice. Corticosteroids in COVID-19 ARDS: Evidence and hope during the pandemic. JAMA, 324, 1292-1295 (2020).
Prösch et al., A novel link between stress and human cytomegalovirus (HCMV) infection: Sympathetic hyperactivity stimulates HCMV activation. Virology, 272(2), 357-65 (Jul. 5, 2000).
R Core Team. R: A language and environment for statistical computing. (R Foundation for Statistical Computing, 2017).
Rhee et al. Increasing trauma deaths in the United States. Annals of Surgery 260, 13 (2014)
Rhodes et al., The surviving sepsis campaign bundles and outcome, results from the International Multicentre Prevalence Study on Sepsis (the IMPreSS study). Intensive Care Medicine, 41(9), 1620-8 (2015).
Ritchie et al., Entropy measures quantify global splicing disorders in cancer. PLoS Computational Biology, 4(3), e1000011 (2008).
Robilotti et al., Determinants of COVID-19 disease severity in patients with cancer. Nature Medicine, 26, 1218-1223 (2020).
Rossignol. Gagnon, & Klagsbrun, Genomic organization of human neuropilin-1 and neuropilin-2 genes: identification and distribution of splice variants and soluble isoforms. Genomics, 70, 211-222 (2000).
SEQC Consortium, A comprehensive assessment of RNA-seq accuracy, reproducibility, and information content by the Sequencing Quality Control Consortium. Nature Biotechnology, 32: 903-914 (2014).
Sethuraman, Jeremiah, & Ryo. Interpreting diagnostic tests for SARS-CoV-2. JAMA (2020).
Sharifipour et al., Evaluation of bacterial co-infections of the respiratory tract in COVID-19 patients admitted to ICU. BMC Infectious Diseases, 20: 646. (2020).
Singer et al. (2016) The third international consensus definitions for sepsis and septic shock (Sepsis-3). JAMA, 315: 801-810.
Sterne-Weiler et al., Efficient and accurate quantitative profiling of alternative splicing patterns of any complexity on a laptop. Molecular Cell, 72(1), 187-200.e6 (2018).
Swanson et al., What human sperm RNA-Seq tells us about the microbiome. Journal of Assisted Reproduction and Genetics (Feb. 4, 2020). This study was designed to assess the capacity of human sperm RNA-seq data to gauge the diversity of the associated microbiome within the ejaculate. Semen samples were collected, and semen parameters evaluated at time of collection. Sperm RNA was isolated and subjected to RNA-seq. Microbial composition was determined by aligning sequencing reads not mapped to the human genome to the NCBI RefSeq bacterial, viral, and archaeal genomes following RNA-Seq. Analysis of microbial assignments utilized phyloseq and vegan. Microbial composition within each sample was characterized as a function of microbial associated RNAs. Bacteria known to be associated with the male reproductive tract were present at similar levels in all samples representing eleven genera from four phyla with one exception, an outlier. Shannon diversity index (p<0.001) and beta diversity (unweighted UniFrac distances, p=9.99e−4; beta dispersion, p=0.006) indicated the outlier was significantly different from all other samples. The outlier sample exhibited a dramatic increase in Streptococcus. Multiple testing indicated two operational taxonomic units, S. agalactiae and S. dysgalactiae (p=0.009), were present. These results provide a first look at the microbiome as a component of human sperm RNA sequencing that has sufficient sensitivity to identify contamination or potential pathogenic bacterial colonization at least among the known contributors.
Tacke et al., Levels of circulating miR-133a are elevated in sepsis and predict mortality in critically ill patients. Crit. Care Med., 42(5):1096-104 (May 2014).
The Gene Ontology Resource: 20 years and still GOing strong. Nucleic Acids Research, 47, D330-d338 (2019).
The RECOVERY Collaborative Group, Dexamethasone in hospitalized patients with Covid-19—Preliminary report. New England Journal of Medicine (2020).
Tian et al., Predictors of mortality in hospitalized COVID-19 patients, A systematic review and meta-analysis. Journal of Medical Virology (2020).
Tompkins, Genomics of injury: The Glue Grant experience. The Journal of Trauma and Acute Care Surgery, 78(4), 671-86 (2015).
Traylen et al., Virus reactivation: A panoramic view in human infections. Future Virol., 6(4), 451-63 (April 2011).
Vijayan et al., Procalcitonin: A promising diagnostic marker for sepsis and antibiotic therapy. J. Intensive Care, Aug. 3, 2017.
Vivarelli et al. Cancer management during COVID-19 pandemic: Is immune checkpoint inhibitors-based immunotherapy harmful or beneficial? Cancers 12 (2020).
Walton et al., Reactivation of Multiple Viruses in Patients with Sepsis. PLoS One, 9(6), e98819 (Jun. 11, 2014).
Wang et al., MicroRNA as biomarkers and diagnostics. J. Cell Physiol., 231(1), 25-30 (2016).
Waterer & Rello, Steroids and COVID-19: We need a precision approach, not one size fits all. Infectious Diseases and Therapy (2020).
Wickham, ggplot2: Elegant graphics for data analysis. (Springer New York, 2009).
Wood, Lu, & Langmead, Improved metagenomic analysis with Kraken 2. Genome Biology 20: 257 (2019).
Wu et al., Profiling circulating microRNA expression in experimental sepsis using cecal ligation and puncture. PLoS One, 8(10) (2013).
Xiao et al. A genomic storm in critically injured humans. J Exp Med 208, 2581-2590 (2011).
Zander & Farver, Pulmonary pathology e-book: A volume in foundations in diagnostic pathology series. (Elsevier Health Sciences, 2016).
Zhang et al., D-dimer levels on admission to predict in-hospital mortality in patients with Covid-19. Journal of Thrombosis and Haemostasis (JTH), 18, 1324-1329 (2020).

### LIST OF EMBODIMENTS

Specific compositions and methods of RNA sequencing to diagnose sepsis have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as those skilled in the art will recognize. When the specification or claims recite method steps or functions in order, alternative embodiments may perform the tasks in a different order or substantially concurrently. The inventive subject matter is not to be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by one of ordinary skill in the art to which this invention belongs. This invention is not limited to the particular methodology, protocols, reagents, and the like described in this specification and, as such, can vary in practice. The terminology used in this specification is not intended to limit the scope of the invention, which is defined solely by the claims.

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

The terms comprise and comprising should be interpreted as referring to elements, components, or steps in a non-exclusive manner, indicating that the referenced elements, components, or steps may be present, used, or combined with other elements, components, or steps. The singular terms a, an, and the include plural referents unless context indicates otherwise. Similarly, the word or should cover and unless the context indicates otherwise. The abbreviation e.g., is used to indicate a non-limiting example and is synonymous with the term for example.

When a range of values is provided, each intervening value, to the tenth of the unit of the lower limit, unless the context dictates otherwise, between the upper and lower limit of that range and any other stated or intervening value in that range of values.

Some embodiments of the technology described can be defined according to the following numbered paragraphs:

1. A method of using unmapped bacterial RNA reads to identify bacteria causing sepsis.

2. A method of using unmapped viral reads to identify sepsis or viral reactivation.

3. A method of using unmapped B/T V(D)J to identify sepsis.

4. A method of using a Principal Component Analysis of RNA splicing entropy to identify sepsis.

5. A method of using RNA lariats to identify sepsis.

6. A method of using a Principal Component Analysis of gene expression, alternative RNA splicing, or alternative transcription start and end to identify sepsis.

1. A method of treatment of sepsis, comprising the steps of:
(a) identifying the sepsis patient to be treated using a diagnostic step, the diagnostic step comprising:
(1) obtaining a body sample from the patient;
(2) assaying the sample for bacterial RNA reads that identify bacteria causing sepsis; and
(3) identifying the bacteria causing sepsis by aligning the RNA reads to a genome of interest; and
(b) treating the identified sepsis patient with a treatment for sepsis.

2. A method of treatment of sepsis, comprising the steps of:
(a) identifying the sepsis patient to be treated using a diagnostic step, the diagnostic step comprising:
(1) obtaining a body sample from the patient;
(2) assaying the sample for viral RNA reads that identify bacteria causing sepsis; and
(3) identifying the bacteria causing sepsis by aligning the RNA reads to a genome of interest; and
(b) treating the identified sepsis patient with a treatment for sepsis.

3. A method of treatment of sepsis, comprising the steps of:
(a) identifying the sepsis patient to be treated using a diagnostic step, the diagnostic step comprising:
(1) obtaining a body sample from the patient;
(2) assaying the sample for RNA reads that identify bacteria causing sepsis using a Principal Component Analysis of RNA splicing entropy to identify sepsis; and
(3) identifying the bacteria causing sepsis by aligning the RNA reads to a genome of interest; and
(b) treating the identified sepsis patient with a treatment for sepsis.

4. A method of treatment of COVID-19 infection, comprising the steps of:
(a) identifying the COVID-19 patient to be treated using a diagnostic step, the diagnostic step comprising:
(1) obtaining a body sample from the patient;
(2) assaying the sample for RNA reads that identify the patient as having a COVID-19 infection; and
(3) identifying the patient as having a COVID-19 infection by aligning the RNA reads to a genome of interest; and
(b) treating the identified COVID-19 patient with a treatment for COVID-19 infection.

5. A method of treatment of acute respiratory distress syndrome (ARDS), comprising the steps of:
(a) identifying the ARDS patient to be treated using a diagnostic step, the diagnostic step comprising:
(1) obtaining a body sample from the patient;
(2) assaying the sample for bacterial RNA reads that identify the patient as having ARDS; and
(3) identifying the patient as having ARDS by aligning the RNA reads to a genome of interest; and
(b) treating the identified ARDS patient with a treatment for ARDS.
